(19) United States
(12) Patent Application Publication
(10) Pub. No.: US 2023/0192795 A1
(43) Pub. Date: Jun. 22, 2023

(54) IMMUNOCONJUGATES

(71) Applicant: Hoffmann-La Roche Inc., Little Falls, NJ (US)

(72) Inventors: Laura Codarri Deak, Au (CH); Anne Freimoser-Grundschober, Zurich (CH); Christian Klein, Bonstetten (CH); Laura Lauener, Zurich (CH); Ekkehard Moessner, Kreuzlingen (CH); Cindy Schulenburg, Zurich (CH); Pablo Umaña, Wollerau, Schwyz (CH); Eleni Maria Varypataki, Zurich (CH)

(73) Assignee: Hoffmann-La Roche Inc., Little Falls, NJ (US)

(21) Appl. No.: 17/996,338

(22) PCT Filed: Apr. 13, 2021
(86) PCT No.: PCT/EP2021/059473

§ 371 Date: Oct. 14, 2022

(30) Foreign Application Priority Data
Apr. 15, 2020 (EP) 20169510.3

(51) Int. Cl. C07K 14/54; C07K 16/28; A61P 35/00

(52) U.S. Cl. CPC C07K 14/5418; A61P 35/00; C07K 16/2818; A61K 2039/505

(57) ABSTRACT

The present invention generally relates to mutant interleukin-7 polypeptides, immunoconjugates, particularly immunoconjugates comprising a mutant interleukin-7 polypeptide and an antibody that binds to PD-1. In addition, the invention relates to polynucleotide molecules encoding the mutant interleukin-7 polypeptides or the immunoconjugates, and vectors and host cells comprising such polynucleotide molecules. The invention further relates to methods for producing the mutant interleukin-7 polypeptides, immunoconjugates, pharmaceutical compositions comprising the same, and uses thereof.

### FIELD OF THE INVENTION

The present invention generally relates to mutant interleukin-7 polypetides, immunoconjugates, particularly immunoconjugates comprising a mutant interleukin-7 polypeptide and an antibody that binds to PD-1. In addition, the invention relates to polynucleotide molecules encoding the mutant interleukin-7 polypeptide or immunoconjugates, and vectors and host cells comprising such polynucleotide molecules. The invention further relates to methods for producing the mutant interleukin-7 polypeptide or immunoconjugates, pharmaceutical compositions comprising the same, and uses thereof.

### BACKGROUND

Interleukin-7 (IL-7) is a cytokine mainly secreted by stromal cells in lymphoid tissues. It is involved in the maturation of lymphocytes, e.g. by stimulating the differentiation of multipotent hematopoetic stem cells to lymphoblasts. IL-7 is essential for T-cell development and survival, as well as for mature T-cell homeostasis. A lack of IL-7 causes immature immune cell arrest (Lin J. et al. (2017), Anticancer Res. 37(3):963-967).

IL-7 binds to the IL-7 receptor, which is composed of the IL-7R alpha chain (IL-7Rα, CD127) as well as the common gamma chain (γc, CD132, IL-2Rγ), that is mutual to the interleukines IL-2, IL-4, IL-7, IL-9, IL-15 and IL-21 (Rochman Y. et al., (2009) Nat Rev Immunol. 9:480-490). Whereas γc is expressed by most haematopoietic cells, IL-7Rα is almost exclusively expressed by cells of the lymphoid lineage (Mazzucchelli R. and Durum S.K. (2007) Nat Rev Immunol. 7(2): 144-54). IL-7Rα is found on the surface of T cells across their differentiation from naive to effector while its expression is reduced on terminally differentiated T cells and is virtually absent from the surface of regulatory T cells. IL-7Rα mRNA and protein expression levels are negatively regulated by IL-2, therefore IL-7Rα is downregulated in recently activated T cells expressing the IL-2Rα (CD25) (Xue H.H, et al. 2002, PNAS. 99(21): 13759-64), this mechanism ensures the IL-2 mediated rapid clonal expansion of recently primed T cells while IL-7 role is to equally maintain all T cell clones. IL-7Rα has also been recently described on a newly characterized precursor population of CD8 T cells, TCF-1+ PD-1+ stem-like CD8 T cells, which is found in the tumor of cancer patients responding to PD-1 blockade (Hudson et al., 2019, Immunity 51, 1043-1058; Im et al., PNAS, vol. 117, no. 8, 4292-4299; Siddiqui et al., 2019, Immunity 50, 195-211; Held et al., Sci. , Transl. Med. 11; eaay6863 (2019); Vodnala and Restifo, Nature, Vol 576, 19/26 Dec. 2019). Although, until today, there are no scientific descriptions of the effect of IL-7 on the stem like CD8 T cells, IL-7 could be used to expand this population of tumor reactive T cells in order to increase the number of patients responding to check point inhibitors.

IL-7, IL-7Rα and γc form a ternary complex, which signals over the JAK/STAT (Janus kinase (JAK)-signal transducer and activator of transcription (STAT)) pathway as well as the PI3K/Akt (Phosphatidylinositol 3-kinase (PI3K), serine/threonine protein kinase, protein kinase B (AKT)) signaling cascade, leading to the development and homeostasis of B- and T-cells (Niu N. and Qin X. (2013) Cell Mol Immunol. 10(3):187-189, Jacobs et al., (2010), J Immunol. 184(7): 3461-3469).

IL-7 is a 25 kDa 4-helix bundle, monomeric protein. The helix length varies from 13 to 22 amino acids, which is similar to the helix length of other common gamma chain (γc, CD132, IL-2Rγ) binding interleukines. However, IL-7 shows a unique turn motif in the A helix, which was shown to stabilize the IL-7/IL-7Rα interaction (McElroy, C.A. et al., (2009) Structure 17: 54-65). Whereas the A helix interacts with both receptor chains IL-7Rα and γc, the C helix interacts predominantly with IL-7Rα and the D helix with the γc chain (sequence and structural alignments based on PDB:3DI2 and PDB:2ERJ).

Programmed cell death protein 1 (PD-1 or CD279) is an inhibitory member of the CD28 family of receptors, that also includes CD28, CTLA-4, ICOS and BTLA. PD-1 is a cell surface receptor and is expressed on activated B cells, T cells, and myeloid cells (Okazaki et al (2002) Curr. Opin. Immunol. 14: 391779-82; Bennett et al. (2003) J Immunol 170:711-8). The structure of PD-1 is a monomeric type 1 transmembrane protein, consisting of one immunoglobulin variable-like extracellular domain and a cytoplasmic domain containing an immunoreceptor tyrosine-based inhibitory motif (ITIM) and an immunoreceptor tyrosine-based switch motif (ITSM). Two ligands for PD-1 have been identified, PD-L1 and PD-L2, that have been shown to downregulate T cell activation upon binding to PD-1 (Freeman et al (2000) J Exp Med 192: 1027-34; Latchman et al (2001) Nat Immunol 2:261-8; Carter etal (2002) Eur J Immunol 32:634-43). Both PD-L1 and PD-L2 are B7 homologs that bind to PD-1, but do not bind to other CD28 family members. One ligand for PD-1, PD-L1 is abundant in a variety of human cancers (Dong et al (2002) Nat. Med 8:787-9). The interaction between PD-1 and PD-L1 results in a decrease in tumor infiltrating lymphocytes, a decrease in T-cell receptor mediated proliferation allowing immune evasion by the cancerous cells (Dong et al. (2003) J. MoI. Med. 81:281-7; Blank et al. (2005) Cancer Immunol. Immunother. 54:307-314; Konishi et al. (2004) Clin. Cancer Res. 10:5094-100). Immune suppression can be reversed by inhibiting the local interaction of PD-1 with PD-L1, and the effect is additive when the interaction of PD-1 with PD-L2 is blocked as well (Iwai et al. (2002) Proc. Nat 7. Acad. ScL USA 99: 12293-7; Brown et al. (2003) J. Immunol. 170:1257-66).

Antibodies that bind to PD-1 are described e.g. in WO 2017/055443 A1.

### SUMMARY OF THE INVENTION

The present invention provides a novel approach of targeting a mutant form of IL-7 with advantageous properties for immunotherapy directly to immune effector cells, such as cytotoxic T lymphocytes, rather than tumor cells, through conjugation of the mutant IL-7 polypeptide to an antibody that binds to PD-1. This results in cis-delivery of the IL-7 mutant to PD-1 expressing immune subsets, especially tumor reactive T cells e.g. CD8+ PD1+ TCF+ T cell subsets and their progeny.

The IL-7 mutants used in the present invention have been designed to overcome the problems associated with cytokine immunotherapy, in particular toxicity caused by the induction of VLS, tumor tolerance caused by the induction of AICD, and immunosuppression caused by activation of T_{reg} cells. In addition to circumventing escape of tumors from tumor-targeting as mentioned above, targeting of the IL-7 mutant to immune effector cells may further increase the preferential activation of tumor specific CTLs over immunosuppressive T_{reg} cells due to lower PD-1 and IL-7Rα expressing levels on Tregs than CTLs. By using an antibody that binds to PD-1, the suppression of T-cell activity induced by the interaction of PD-1 with its ligand PD-L1 may additionally be reversed, thus further enhancing the immune response.

In a general aspect, the invention provides a mutant interleukin-7 (IL-7) polypeptide comprising at least one amino acid substitution in a position selected from the group of E13, V15, V18, D21, Q22, D25, T72, L77, K81, E84, G85, I88, Q136, K139, N143 and M147 of human IL-7 according to SEQ ID NO:52; i.e. the numbering is relative to the human IL-7 sequence SEQ ID NO:52. In some embodiments of the invention, the mutant interleukin-7 polypeptide comprises at least one amino acid selected from the group of E13A, E13K, V15A, V15K, V18A, V18K, D21A, D21K, Q22A, Q22K, D25A, D25K, T72A, D74K, L77A, L77K, K81A, K81E, E84A, G85K, G85E, I88K, Q136A, Q136K, K139A, K139E, N143K and M147A. In some embodiments of the invention the mutant interleukin-7 polypetide comprises at least one amino acid substitution selected from the group of V15A, V15K, V18A, V18K, L77A, L77K, K81E, G85K, G85E, I88K and N143K. In some embodimenets of the invention the mutant interleukin-7 polypeptide comprises an amino acid sequence selected from the group of SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 135 and SEQ ID NO: 136.

In some embodiments of the invention the mutant interleukin-7 polypetide comprises an amino acid sequence selected from the group of SEQ ID NO: 55, SEQ IN NO: 56, SEQ ID NO: 57, SEQ ID NO: 58, SEQ ID NO: 67, SEQ ID NO: 68, SEQ ID NO: 70, SEQ ID NO: 72, SEQ ID NO: 73, SEQ ID NO: 74, SEQ ID NO: 79, SEQ ID NO: 135 and SEQ ID NO: 136.

In another aspect, the invention provides a mutant interleukin-7 polypeptide comprising an amino acid substitution, which eliminates the N-Glycosylation site of IL-7 at a position selected from the group of position 72, 93 and 118. The substitution may be selected from the group of T72A, T93A and S118A. In another aspect, the invention provide a mutant interleukin-7 polypeptide comprising the amino acid subsitutions T72A, T93A and S118A. In some embodiments of the invention the mutant interleukin 7 polypeptide comprises an amino acid sequence selected from the group of SEQ ID NO: 81, SEQ ID NO: 82, SEQ ID NO: 83 and SEQ ID NO: 84.

In another aspect, the invention provides for a mutant interleukin-7 polypeptide comprising at least the amino acid substitutions K81E and G85K or K81E and G85E. In some embodimenets of the invention, the mutant interleukin-7 polypeptide comprises an amino acid sequence of SEQ ID NO: 135 or SEQ ID NO: 136.

In a further aspect, the invention provides for a mutant interleukin-7 polypeptide as disclosed herein, wherein said mutant IL-7 polypeptide is linked to a non-IL-7 moiety. The mutant interleukin-7 polypeptide may be linked to a first and a second non-IL-7 moiety. The mutant IL-7 polypeptide may share a carboxy-terminal peptide bond with said first non-IL-7 moiety and an aminoterminal peptide bond with said second non-IL-7 moiety. The non-IL-7 moiety may be an antigen binding moiety or an immune effector cell binding moiety, preferably a PD-1 binding moiety.

In another aspect, the invention provides an immunoconjugate comprising (i) a mutant IL-7 polypeptide as disclosed herein and (ii) an antibody that binds to PD-1. In some embodiments of the immunoconjugate according to the invention, the antibody comprises (a) a heavy chain variable region (VH) comprising a HVR-H1 comprising the amino acid sequence of SEQ ID NO:1, a HVR-H2 comprising the amino acid sequence of SEQ ID NO:2, a HVR-H3 comprising the amino acid sequence of SEQ ID NO:3, and a FR-H3 comprising the amino acid sequence of SEQ ID NO:7 at positions 71-73 according to Kabat numbering, and (b) a light chain variable region (VL) comprising a HVR-L1 comprising the amino acid sequence of SEQ ID NO:4, a HVR-L2 comprising the amino acid sequence of SEQ ID NO:5, and a HVR-L3 comprising the amino acid sequence of SEQ ID NO:6.

In some embodiments of the immunoconjugate according to the invention, the antibody comprises (a) a heavy chain variable region (VH) comprising a HVR-H1 comprising the amino acid sequence of SEQ ID NO:8, a HVR-H2 comprising the amino acid sequence of SEQ ID NO:9, and a HVR-H3 comprising the amino acid sequence of SEQ ID NO: 10, and (b) a light chain variable region (VL) comprising a HVR-L1 comprising the amino acid sequence of SEQ ID NO: 11, a HVR-L2 comprising the amino acid sequence of SEQ ID NO: 12, and a HVR-L3 comprising the amino acid sequence of SEQ ID NO: 13.

In some embodiments of the immunoconjugate according to the invention, the antibody comprises (a) a heavy chain variable region (VH) comprising an amino acid sequence that is at least about 95%, 96%, 97%, 98%, 99% or 100% identical to the amino acid sequence of SEQ ID NO: 14, and (b) a light chain variable region (VL) comprising an amino acid sequence that is at least about 95%, 96%, 97%, 98%, 99% or 100% identical to an amino acid sequence selected from the group consisting of SEQ ID NO: 15, SEQ ID NO: 16, SEQ ID NO: 17, and SEQ ID NO:18.

In preferred embodiments of the immunoconjugate of the invention, the mutant IL-7 polypeptide comprises a sequence selected from the group of SEQ ID NO: 55, SEQ IN NO: 56, SEQ ID NO: 57, SEQ ID NO: 58, SEQ ID NO: 67, SEQ ID NO: 68, SEQ ID NO: 70, SEQ ID NO: 72, SEQ ID NO: 73, SEQ ID NO: 74, SEQ ID NO: 79, SEQ ID NO: 135 and SEQ ID NO: 136.

In some embodiments, the immunoconjugate comprises not more than one mutant IL-7 polypeptide. In some embodiments, the antibody comprises an Fc domain composed of a first and a second subunit. In some such embodiments, the Fc domain is an IgG class, particularly an IgG1 subclass, Fc domain, and/or the Fc domain is a human Fc domain. In some embodiments, the antibody is an IgG class, particularly an IgG1 subclass immunoglobulin.

In some embodiments wherein the immunoconjugate comprises an Fc domain, the Fc domain comprises a modification promoting the association of the first and the second subunit of the Fc domain.

In some embodiments, in the CH3 domain of the first subunit of the Fc domain an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable. In some embodiments, in the first subunit of the Fc domain the threonine residue at position 366 is replaced with a tryptophan residue (T366W), and in the second subunit of the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numberings according to Kabat EU index). In some such embodiments, in the first subunit of the Fc domain additionally the serine residue at position 354 is replaced with a cysteine residue (S354C) or the glutamic acid residue at position 356 is replaced with a cysteine residue (E356C), and in the second subunit of the Fc domain additionally the tyrosine residue at position 349 is replaced by a cysteine residue (Y349C) (numberings according to Kabat EU index). In some embodiments, the mutant IL-7 polypeptide is fused at its amino-terminal amino acid to the carboxy-terminal amino acid of one of the subunits of the Fc domain, particularly the first subunit of the Fc domain, optionally through a linker peptide. In some such embodiments, the linker peptide has the amino acid sequence of SEQ ID NO:21

In some embodiments wherein the immunoconjugate comprises an Fc domain, the Fc domain comprises one or more amino acid substitution that reduces binding to an Fc receptor, particularly an Fcy receptor, and/or effector function, particularly antibody-dependent cell-mediated cytotoxicity (ADCC). In some such embodiments, said one or more amino acid substitution is at one or more position selected from the group of L234, L235, and P329 (Kabat EU index numbering). In some embodiments, each subunit of the Fc domain comprises the amino acid substitutions L234A, L235A and P329G (Kabat EU index numbering).

In some embodiments, the immunoconjugate according to the invention comprises a polypeptide comprising an amino acid sequence that is at least about 80%, 85%, 90%, 95%, 96%, 97%, 98%, 99% or 100% identical to the sequence of SEQ ID NO: 85, a polypeptide comprising an amino acid sequence that is at least about 80%, 85%, 90%, 95%, 96%, 97%, 98%, 99% or 100% identical to the sequence of SEQ ID NO: 86, and a polypeptide comprising an amino acid sequence that is at least about 80%, 85%, 90%, 95%, 96%, 97%, 98%, 99% or 100% identical to a sequence selected from the group of SEQ ID NO: 90, SEQ ID NO: 91, SEQ ID NO: 92, SEQ ID NO: 93, SEQ ID NO: 102, SEQ ID NO: 103, SEQ ID NO: 105, SEQ ID NO: 107, SEQ ID NO: 108, SEQ ID NO: 109, SEQ ID NO: 114, SEQ ID NO137 and SEQ ID NO: 138. In some embodiments, the immunoconjugate essentially consists of a mutant IL-7 polypeptide and an IgG1 immunoglobulin molecule, joined by a linker sequence.

Also provided is a method of producing a mutant IL-7 polypeptide or an immunoconjugate comprising a mutant IL-7 polypeptide and an antibody that binds to PD-1, comprising (a) culturing the host of the invention under conditions suitable for the expression of the mutant IL-7 polypetide or the immunoconjugate, and optionally (b) recovering the mutant IL-7 polypetide or the immunoconjugate. Also provided by the invention is a mutant IL-7 polypetide or an immunoconjugate comprising a mutant IL-7 polypeptide and an antibody that binds to PD-1, produced by said method.

The invention further provides a pharmaceutical composition comprising the mutant IL-7 polypetide of the inivention or the immunoconjugate of the invention and a pharmaceutically acceptable carrier, and methods of using a mutant IL-7 polypeptide or an immunoconjugate of the invention.

In particular, the invention encompasses a mutant IL-7 polypeptide according to the invention or an immunoconjugate according to the invention fur use as a medicmanet, and for use in the treatment of a disease. In a particular embodiment, said disease is cancer.

Also encompasses by the invention is the used of a mutant IL-7 polypeptide according to the invention or an immunoconjugate according to the invention in the manufacture of a medicament for the treatment of a disease. In a particular embodiment, said disease is cancer.

Further provided is a method of treating a disease in an individual comprising administering to said individual a therapeutically effective amount of a composition comprising the mutant IL-7 polypeptide according to the invention or the immunoconjugate according to the invention in a pharmaceutically acceptable form. In a particular embodiment, said disease is cancer.

Also provided is a method of stimulating the immune system of an individual comprising administering to said individual an effective amount of a composition comprising the mutant IL-7 polypetide according to the invention or the immunoconjugate according to the invention in a pharmaceutically acceptable form.

### BRIEF DESCRIPTION OF THE DRAWINGS

FIG. 1A: Schematic representation of an IgG-IL-7 immunoconjugate format, comprising two Fab domains (variable domain, constant domain), a heterodimeric Fc domain and a mutant IL-7 polypeptide fused to a C-terminus of the Fc domain.

FIG. 1B: Schematic representation of another IgG-IL-7 immunoconjugate format, comprising two Fab domains (variable domain, constant domain), a homoodimeric Fc domain and two mutant IL-7 polypeptides fused to the C-termini of the Fc domain.

FIG. 1C: Schematic representation of another IgG-IL-7 immunoconjugate format, comprising one Fab domain (variable domain, constant domain), a heterodimeric Fc domain and one mutant IL-7 polypeptide fused to a C-terminus of the Fc domain.

FIG. 1D: Schematic representation of another IgG-IL-7 immunoconjugate format, comprising two Fab domain (variable domain, constant domain), a heterodimeric Fc domain and one mutant IL-7 polypeptide fused to an N-terminus of one of the Fab domains.

FIG. 1E: Schematic representation of another IgG-IL-7 immunoconjugate format, comprising two Fab domain (variable domain, constant domain), a homodimeric Fc domain and two mutant IL-7 polypeptide fused to the N-termini of the Fab domains.

FIGS. 2A-H: IL-7R signaling by STAT5-phosphrylation upon treatment of PD-1+ CD4 Tcells with increasing doses of PD1-IL7 variants. The IL-7 moiety of the PD1-IL7 variants contain a mutation to reduce the affinity to the IL7Rα. STAT5-P is depicted as normalized STAT5-P, where 100% is equal to the frequency of STAT5-P+ cells upon treatment with 66 nM of PD1-IL7 wt.

FIG. 2A shows normalized STAT-5 phosphorylation for variants 1-4.

FIG. 2B shows normalized STAT-5 phosphorylation for variants 5-8.

FIG. 2C shows normalized STAT-5 phosphorylation for variants 9-12.

FIG. 2D shows normalized STAT-5 phosphorylation for variants 13-16.

FIG. 2E shows normalized STAT-5 phosphorylation for variants 17-20.

FIG. 2F shows normalized STAT-5 phosphorylation for variants 21-24.

FIG. 2G shows normalized STAT-5 phosphorylation for variants 25-28.

FIG. 2H shows normalized STAT-5 phosphorylation for variants 29-32.

FIGS. 3A-C: Assessment of cis delivery of mutant IL-7 polypeptides to PD-1+ CD4 Tcells to the IL-7Rα/IL-2Rγ upon PD-1 anchoring by PD1-IL7v.

FIG. 3A shows IL-7R signaling (STAT5-P) depicted as frequency of STAT-5P in human activated PD1+ CD4 T cells upon stimulation with 0.66 nM of PD1-IL7 mutants and PD1-IL7wt. Each symbol represents a separate donor, horizontal lines indicate medians with N=4.

FIG. 3B shows normalized STAT5-P in PD1 pre-blocked activated CD4 Tcells, showing the PD1 independent delivery of IL7v. Normalized STAT5-P of 100% is defined as the frequency of STAT5-P+ in the CTV labelled PD1+ Tcells, which were co-cultured with the PD1 pre-blocked CFSE labelled CD4 Tcells.

FIG. 3C shows the correlation of the normalized STAT5-P on the PD1 blocked cells (x-axis) vs the frequency of STAT5+ on PD1+ Tcells (y-axis). Each dot represents the mean ± SEM of one PD1-IL7v, where the mutants of interest are depicted in black and labelled.

FIGS. 4A-F: Potency assessment by STAT5-phosphorylation with respect to cis delivery of IL-7v to the IL-7Rα/IL-2Rγ of PD-1+ CD4 T cells upon PD-1 anchoring by PD1-IL7v.

FIG. 4A shows IL-7R signaling (STAT5-P) of PD1-IL7-VAR3 and PD1-IL7-VAR4 depicted as frequency of STAT5-P in co-cultured human PD1+ non-blocked (solid line) and PD-1 pre-blocked (dotted line) CD4 T cells upon 12 min exposure to PD1-IL7 mutants. Mean ± SEM of 4 donors.

FIG. 4B shows IL-7R signaling (STAT5-P) of PD1-IL7-VAR6 and PD1-IL7-VAR16 depicted as frequency of STAT5-P in co-cultured human PD1+ non-blocked (solid line) and PD-1 pre-blocked (dotted line) CD4 T cells upon 12 min exposure to PD1-IL7 mutants. Mean ± SEM of 4 donors.

FIG. 4C shows IL-7R signaling (STAT5-P) of PD1-IL7-VAR18 and PD1-IL7-VAR20 depicted as frequency of STAT5-P in co-cultured human PD1+ non-blocked (solid line) and PD-1 pre-blocked (dotted line) CD4 T cells upon 12 min exposure to PD1-IL7 mutants. Mean ± SEM of 4 donors.

FIG. 4D shows IL-7R signaling (STAT5-P) of PD1-IL7-VAR21 and PD1-IL7-VAR27 depicted as frequency of STAT5-P in co-cultured human PD1+ non-blocked (solid line) and PD-1 pre-blocked (dotted line) CD4 T cells upon 12 min exposure to PD1-IL7 variants. Mean ± SEM of 4 donors.

FIG. 4E shows IL-7R signaling (STAT5-P) depicted as frequency of STAT-5 in human activated PD1+ CD4 Tcells upon stimulation with 0.66 nM of PD1-IL7 mutants and PD1-IL7wt.

FIG. 4F shows normalized STAT5-P in PD1+ pre-blocked activated CD4 Tcells, showing the impact of PD1 independent delivery of IL7v. Normalized STAT5-P of 100% is defined as the frequency of STAT5-P+ in the PD1+ Tcells, which were co-cultured with the PD1 pre-blocked activated CD4 Tcells. For FIGS. 4E and 4F each symbol represents a separate donor, horizontal lines indicate medians with N=8.

FIGS. 5A-F: IL-7R signaling (STAT5-P) in PD1-blocked and PD-1 expressing CD4+ Tcells cultured separately.

FIG. 5A: IL-7R signaling (STAT5-P) of PD1-IL7-VAR3 and PD1-IL7-VAR4 depicted as frequency of STAT5-P in human PD1+ (solid line) and PD-1 pre-blocked (dotted line) CD4 T cells upon 12 min exposure to PD1-IL7 mutants. Mean ± SEM of 4 donors.

FIG. 5B: IL-7R signaling (STAT5-P) of PD1-IL7-VAR5 and PD1-IL7-VAR6 depicted as frequency of STAT5-P in human PD1+ (solid line) and PD-1 pre-blocked (dotted line) CD4 T cells upon 12 min exposure to PD1-IL7 mutants. Mean ± SEM of 4 donors.

FIG. 5C: IL-7R signaling (STAT5-P) of PD1-II,7-VAR15 and PD1-IL7-VAR16 depicted as frequency of STAT5-P in human PD1+ (solid line) and PD-1 pre-blocked (dotted line) CD4 T cells upon 12 min exposure to PD1-IL7 mutants. Mean ± SEM of 4 donors.

FIG. 5D: IL-7R signaling (STAT5-P) of PD1-II,7-VAR18 and PD1-IL7-VAR20 depicted as frequency of STAT5-P in human PD1+ (solid line) and PD-1 pre-blocked (dotted line) CD4 T cells upon 12 min exposure to PD1-IL7 mutants. Mean ± SEM of 4 donors.

FIG. 5E: IL-7R signaling (STAT5-P) of PD1-II,7-VAR21 and PD1-IL7-VAR22 depicted as frequency of STAT5-P in human PD1+ (solid line) and PD-1 pre-blocked (dotted line) CD4 T cells upon 12 min exposure to PD1-IL7 mutants. Mean ± SEM of 4 donors.

FIG. 5F: IL-7R signaling (STAT5-P) of PD1-IL7-VAR27 depicted as frequency of STAT5-P in human PD1+ (solid line) and PD-1 pre-blocked (dotted line) CD4 T cells upon 12 min exposure to PD1-IL7 mutants. Mean ± SEM of 4 donors.

FIG. 6 shows rescue of Tconv effector function from Treg suppression upon PDl-IL7v treatment. Percentage of suppression by Tregs of granzyme B secreted by Tconv after 5 days of coculture in presence of PD1-IL7 single mutants. Each symbol represents a separate donor, horizontal lines indicate medians with N=5. P was calculated using one-way ANOVA (*p<0.05, **p<0.01, ***p<0.001, ****p<0.0001).

FIG. 7 shows IL-7R signaling (STAT5-P) upon treatment with increasing doses of reference PD1-IL7 mutants on activated PD-1+ and PD-1- CD4 T cells. IL-7R signaling (STAT5-P) in co-cultured PD-1- (pre-treated with anti-PD-1) and PD-1+ CD4 T cells upon treatment with reference PD1-IL7 mutants. IL-7R signaling (STAT5-P) depicted as frequency of STAT5-P in co-cultured PD-1+ (solid line) and PD-1 (pre-treated with anti-PD-1) (dotted line) CD4 T cells 12 min upon exposure. Mean ± SEM of 6 donors.

FIG. 8A shows IL-7R signaling (STAT5-P) upon treatment with increasing doses of PD1-IL7 single and double mutants on activated PD-1+ and PD-1- CD4 T cells. IL-7R signaling (STAT5-P) in co-cultured PD-1- (pre-treated with anti-PD-1) and PD-1+ CD4 T cells upon treatment with PD1-IL7 mutants (VAR18, VAR21). IL-7R signaling (STAT5-P) depicted as frequency of STAT5-P in co-cultured PD-1+ (solid line) and PD-1 (pre-treated with anti-PD-1) (dotted line) CD4 T cells 12 min upon exposure. Mean ± SEM of 4 donors.

FIG. 8B shows IL-7R signaling (STAT5-P) upon treatment with increasing doses of PD1-IL7 single and double mutants on activated PD-1+ and PD-1- CD4 T cells. IL-7R signaling (STAT5-P) in co-cultured PD-1- (pre-treated with anti-PD-1) and PD-1+ CD4 T cells upon treatment with PD1-IL7 mutants (Reference molecule 2, VAR18/20, VAR18/21). IL-7R signaling (STAT5-P) depicted as frequency of STAT5-P in co-cultured PD-1+ (solid line) and PD-1 (pre-treated with anti-PD-1) (dotted line) CD4 T cells 12 min upon exposure. Mean ± SEM of 4 donors.

FIG. 9A shows IL-7R signaling (STAT5-P) on activated PD-1+ versus freshly isolated IL-7Ra+ CD4 T cells upon treatment with increasing doses of PD1-IL7 mutants (VAR18, VAR21). IL-7R signaling (STAT5-P) in co-cultured activated PD1+ IL-7Ralow and freshly isolated PD1low IL-7Rahigh CD4 T cells upon exposure to PD1-IL7 mutants. IL-7R signaling (STAT5-P) depicted as frequency of STAT5-P in activated PD1+ T cells (solid line) and freshly isolated IL-7Ra+ T cells (dotted line) 12 min upon exposure. Mean ± SEM of 3 donors.

FIG. 9B shows IL-7R signaling (STAT5-P) on activated PD-1+ versus freshly isolated IL-7Ra+ CD4 T cells upon treatment with increasing doses of PD1-IL7 mutants (Reference molecule 2, VAR18/20, VAR18/21). IL-7R signaling (STAT5-P) in co-cultured activated PD1+ IL-7Ralow and freshly isolated PD1low IL-7Rahigh CD4 T cells upon exposure to PD1-IL7 mutants. IL-7R signaling (STAT5-P) depicted as frequency of STAT5-P in activated PD1+ T cells (solid line) and freshly isolated IL-7Ra+ T cells (dotted line) 12 min upon exposure. Mean ± SEM of 3 donors.

FIGS. 10A-F: PD1-IL7 single and double mutants functional activity on cytotoxic effector functions and proliferation of allo-specific PD-1+ CD4 T cells. Cytotoxic effector function as granzyme B (GrzB) secretion and proliferation of CD4 T cells towards B cell lymphoblastoid cell line (ARH-77) after 5 days in presence of PD1-IL7 single and double mutants. FIGS. 10A-C show fold change of GrzB+ CTV- CD4 T cell frequency normalized to untreated (FIG. 10A: PD1-IL7wt, PD1-IL7VAR18, PD1-IL7VAR21, PD1, PD1 + FAP-IL7wt, PD1 + FAP-IL7VAR18, PD1+FAP-IL7VAR21, FAP-IL7wt, FAP-IL7VAR18, FAP-IL7VAR21; FIG. 10B: PD1-IL7wt, PD1-IL7VAR18/20, PD1-IL7VAR18/21, PD1, PD1 + FAP-IL7wt, PD1 + FAP-IL7VAR18/20, PD1 + FAP-IL7VAR18/21, FAP-IL7wt, FAP-IL7VAR18/20, FAP-IL7VAR18/21; FIG. 10C: PD1-IL7wt, Rference molecule 2, PD1, PD1 + FAP-IL7wt, PD1 + FAP-IL7 SS2(1×), PD1 + FAP-IL7 SS2(2×), FAP-IL7wt, FAP-IL7 SS2(1×)). FIG. 10D-4 show proliferation measured by extracting the MFI of CTV normalized to untreated (FIG. 10D: PD1-IL7wt, PD1-IL7VAR18, PD1-IL7VAR21, PD1, PD1 + FAP-IL7wt, PD1 + FAP-IL7VAR18, PD1+FAP-IL7VAR21, FAP-IL7wt, FAP-IL7VAR18, FAP-IL7VAR21; FIG. 10E: PD1-IL7wt, PD1-IL7VAR18/20, PD1-IL7VAR18/21, PD1, PD1 + FAP-IL7wt, PD1 + FAP-IL7VAR18/20, PD1 + FAP-IL7VAR18/21, FAP-IL7wt, FAP-IL7VAR18/20, FAP-IL7VAR18/21; FIG. 10F: PD1-IL7wt, Rference molecule 2, PD1, PD1 + FAP-IL7wt, PD1 + FAP-IL7 SS2(1×), PD1 + FAP-IL7 SS2(2×), FAP-IL7wt, FAP-IL7 SS2(1×)). Mean ± SEM of 9 donors.

FIG. 11: Targeting of stem-like T cells, Tregs and naive T cells by PD-1 based versus untargeted IL-7 mutants and IL7wt. Binding of PD1-IL7 mutants and wildtype versus untargeted IL-7 mutants and wildtype to stem-like T cells, Tregs and naive T cells at unsaturating concentrations. Binding of unsaturating concentrations of PD-1 targeted versus FAP-targeted VAR18, VAR21 and wild-type to healthy donor PBMCs for 30 min at 37° C.

FIG. 12: Cross-reactivity of PD1-IL7 single, double mutants and wt to mouse IL-7Ra and IL-2Rg of human PD-1 transgenic mice. IL-7R signaling (STAT5-P) in activated huPD1+ CD4 T cell from spleen of huPD1-transgenic mice upon treatment with PD1-IL7 single and double mutants. IL-7R signaling (STAT5-P) depicted as MFI of STAT5-P normalized to PD1-IL7wt 30 min upon exposure. Mean ± SEM of 2 mice.

FIGS. 13A-B: IL-7R signaling (STAT5-P) on activated PD-1+ and PD-1- CD4 T cells upon treatment with increasing doses of IL-7 VAR18 (K81E), VAR21 and wild type fused to C- and N-Terminus of the PD-1 blocking antibody. IL-7R signaling (STAT5-P) in co-cultured PD1-(pre-treated with anti-PD-1) and PD1+ CD4 T cells upon stimulation with PD1-IL7 having the IL-7 fused to the N- or C-Terminus of the antibody. IL-7R signaling (STAT5-P) depicted as frequency of STAT5-P in co-cultured PD1+ (solid line) and PD-1- (pre-treated with anti-PD-1) (dotted line) CD4 T cells 12 min upon exposure (FIG. 13A: PD1-IL7wt, PD1-IL7VAR18, IL7wt G4SG5 PD1, IL7VAR18 G4SG5 PD1, PD1 + PD1-IL7wt, PD1 + PD1-IL7VAR18, PD1 + IL7wt G4SG5 PD1, PD1 + IL7VAR18 G4SG5 PD1; FIG. 13B: PD1-IL7wt, PD1-IL7VAR21, IL7wt G4SG5 PD1, IL7VAR21 G4SG5 PD1, PD1 + PD1-IL7wt, PD1 + PD1-IL7VAR21, PD1 + IL7wt G4SG5 PD1, PD1 + IL7VAR21 G4SG5 PD1). Mean ± SEM of 3 donors.

FIG. 14: IL-7R signaling (STAT5-P) in co-cultured PD1 pre-blocked and PD1+ CD4+ Tcells upon treatment with PD1-IL7v reference molecules. IL-7R signaling (STAT5-P) depicted as frequency of STAT5-P in co-cultured PD1+ (solid line) and PD-1 pre-blocked (dotted line) CD4 T cells 12 min upon exposure. Mean ± SEM of 6 donors.

FIGS. 15A-C presents the results of an efficacy experiment with PD1-IL7v variant 18 (FIG. 15A), PD1-IL7v variant 21 (FIG. 15B) and PD-IL7wt (FIG. 15C) as single agents. The Panc02-Fluc pancreatic carcinoma cell line was injected subcutaneously in Black 6-huPD1 transgenics mice to study tumor growth inhibition (TGI) in a subcutaneous model. Tumor size was measured using a caliper. Therapy started when tumors reached 150 mm³. The amount of antibodies injected per mouse was 1 mg/kg for muPD1-IL7v variant 18 and variant 21 and PD1-IL7wt qw. The treatment lasted 2 weeks. The PD1-IL7v variants 21 and 18 mediated significant superior efficacy in terms of tumor growth inhibition compared to vehicle group. The PD1-IL7wt molecule was not well tolerated and the mice need to be sacrificed after the second administration, thus TGI could not be calculated.

### DETAILED DESCRIPTION OF THE INVENTION

### Definitions

Terms are used herein as generally used in the art, unless otherwise defined in the following.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g. reduced binding to IL-7Rα and/or IL-2Rγ. Amino acid sequence deletions and insertions include amino- and/or carboxy-terminal deletions and insertions of amino acids. An example of a terminal deletion is the deletion of the residue in position 1 of full-length human IL-7. Preferred amino acid mutations are amino acid substitutions. For the purpose of altering e.g. the binding characteristics of an IL-7 polypeptide, non-conservative amino acid substitutions, i.e. replacing one amino acid with another amino acid having different structural and/or chemical properties, are particularly preferred. Preferred amino acid substitions include replacing a hydrophobic by a hydrophilic amino acid. Amino acid substitutions include replacement by non-naturally occurring amino acids or by naturally occurring amino acid derivatives of the twenty standard amino acids (e.g. 4-hydroxyproline, 3-methylhistidine, ornithine, homoserine, 5-hydroxylysine). Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful.

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., an antigen binding moiety and an antigen, or a receptor and its ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well established methods known in the art, including those described herein. A particular method for measuring affinity is Surface Plasmon Resonance (SPR).

IL-7 binds to the IL-7 receptor, which is composed of the IL-7R alpha chain (also refered to as IL-7Ralpha, IL-7Rα, IL7Rα, IL-7a, IL7Ra or CD127 herein) as well as the common gamma chain (also refered to as γc, CD 132, IL-2Rgamma, IL-2Rg, IL2Rg, IL-2Rγ or IL2Rγ herein), that is mutual to the interleukines IL-2, IL-4, IL-7, IL-9, IL-15 and IL-21 (Rochman Y. et al., (2009) Nat Rev Immunol. 9:480-490).

The affinity of the mutant or wild-type IL-7 polypeptide for the IL-7 receptor can be determined in accordance with the method set forth in the WO 2012/107417 by surface plasmon resonance (SPR), using standard instrumentation such as a BIAcore instrument (GE Healthcare) and receptor subunits such as may be obtained by recombinant expression (see e.g. Shanafelt et al., Nature Biotechnol 18, 1197-1202 (2000)). Alternatively, binding affinity of IL-7 mutants for the IL-7 receptor may be evaluated using cell lines known to express one or the other such form of the receptor. Specific illustrative and exemplary embodiments for measuring binding affinity are described hereinafter.

The term “interleukin-7” or “IL-7” as used herein, refers to any native IL7 from any vertebrate source, including mammals such as primates (e.g. humans) and rodents (e.g., mice and rats), unless otherwise indicated. The term encompasses unprocessed IL-7 as well as any form of IL-7 that results from processing in the cell. The term also encompasses naturally occurring variants of IL-7, e.g. splice variants or allelic variants. The amino acid sequence of an exemplary human IL-7 is shown in SEQ ID NO: 52.

The term “IL-7 mutant” or “mutant IL-7 polypeptide” as used herein is intended to encompass any mutant forms of various forms of the IL-7 molecule including full-length IL-7, truncated forms of IL-7 and forms where IL-7 is linked to another molecule such as by fusion or chemical conjugation. “Full-length” when used in reference to IL-7 is intended to mean the mature, natural length IL-7 molecule. For example, full-length human IL-7 refers to a molecule that has a polpypetide sequence according to SEQ ID NO: 52. The various forms of IL-7 mutants are characterized in having a at least one amino acid mutation affecting the interaction of IL-7 with IL7Ralpha and/or IL2Rgamma. This mutation may involve substitution, deletion, truncation or modification of the wild-type amino acid residue normally located at that position. Mutants obtained by amino acid substitution are preferred. Unless otherwise indicated, an IL-7 mutant may be referred to herein as a mutant IL-7 peptide sequence, a mutant IL-7 polypeptide, a mutant IL-7 protein, a mutant IL-7 analog or a IL-7 variant.

Designation of various forms of IL-7 is herein made with respect to the sequence shown in SEQ ID NO: 52. Various designations may be used herein to indicate the same mutation. For example a mutation from Valine at position 15 to Alanine can be indicated as 15A, A15, A₁₅, V15A, or Val15Ala.

By a “human IL-7 molecule” as used herein is meant an IL-7 molecule comprising an amino acid sequence that is at least about 90%, at least about 91%, at least about 92%, at least about 93%, at least about 94%, at least about 95% or at least about 96% identical to the human IL-7 sequence of SEQ ID NO:52. Particularly, the sequence identity is at least about 95%, more particularly at least about 96%. In particular embodiments, the human IL-7 molecule is a full-length IL-7 molecule.

As used herein, a “wild-type” form of IL-7 is a form of IL-7 that is otherwise the same as the mutant IL-7 polypeptide except that the wild-type form has a wild-type amino acid at each amino acid position of the mutant IL-7 polypeptide. For example, if the IL-7 mutant is the full-length IL-7 (i.e. IL-7 not fused or conjugated to any other molecule), the wild-type form of this mutant is full-length native IL-7. If the IL-7 mutant is a fusion between IL-7 and another polypeptide encoded downstream of IL-7 (e.g. an antibody chain) the wild-type form of this IL-7 mutant is IL-7 with a wild-type amino acid sequence, fused to the same downstream polypeptide. Furthermore, if the IL-7 mutant is a truncated form of IL-7 (the mutated or modified sequence within the non-truncated portion of IL-7) then the wild-type form of this IL-7 mutant is a similarly truncated IL-7 that has a wild-type sequence. For the purpose of comparing IL-7 receptor binding affinity, IL-7 receptor binding or biological activity of various forms of IL-7 mutants to the corresponding wild-type form of IL-7, the term wild-type encompasses forms of IL-7 comprising one or more amino acid mutation that does not affect IL-7 receptor binding compared to the naturally occurring, native IL-7. In certain embodiments according to the invention the wild-type IL-7 polypeptide to which the mutant IL-7 polypeptide is compared comprises the amino acid sequence of SEQ ID NO: 52.

By “regulatory T cell” or “T_{reg} cell” is meant a specialized type of CD4⁺ T cell that can suppress the responses of other T cells, called peripheral tolerance. T_{reg} cells are characterized by elevated expression of the α-subunit of the IL-2 receptor (CD25), low or absent IL-7Rα (CD127) and the transcription factor forkhead box P3 (FOXP3) (Sakaguchi, Annu Rev Immunol 22, 531-62 (2004)) and play a critical role in the induction and maintenance of peripheral self-tolerance to antigens, including those expressed by tumors. As used herein, the term “effector cells” refers to a population of lymphocytes which survival and/or homeostasis are affected by IL-7. Effector cells include memory CD4+ and CD8+ cells and recently primed T cells including tumor reactive stem-like T cells.

As used herein, the term “PD1”, “human PD1”, “PD-1” or “human PD-1” (also known as Programmed cell death protein 1, or Programmed Death 1) refers to the human protein PD1 (SEQ ID NO: 27, protein without signal sequence) / (SEQ ID NO: 28, protein with signal sequence). See also UniProt entry no. Q15116 (version 156). As used herein, an antibody “binding to PD-1”, “specifically binding to PD-1”, “that binds to PD-1” or “anti-PD-1 antibody” refers to an antibody that is capable of binding PD-1, especially a PD-1 polypeptide expressed on a cell surface, with sufficient affinity such that the antibody is useful as a diagnostic and/or therapeutic agent in targeting PD-1. In one embodiment, the extent of binding of an anti-PD-1 antibody to an unrelated, non-PD-1 protein is less than about 10% of the binding of the antibody to PD-1 as measured, e.g., by radioimmunoassay (RIA) or flow cytometry (FACS) or by a Surface Plasmon Resonance assay using a biosensor system such as a Biacore® system. In certain embodiments, an antibody that binds to PD-1 has a KD value of the binding affinity for binding to human PD-1 of ≤ 1 µM, ≤ 100 nM, ≤ 10 nM, ≤ 1 nM, ≤ 0.1 nM, ≤ 0.01 nM, or ≤ 0.001 nM (e.g. 10⁻⁸ M or less, e.g. from 10⁻⁸ M to 10⁻¹³ M, e.g., from 10⁻⁹ M to 10⁻¹³ M). In one embodiment, the KD value of the binding affinity is determined in a Surface Plasmon Resonance assay using the Extracellular domain (ECD) of human PD-1 (PD-1-ECD, see SEQ ID NO: 43) as antigen.

By “specific binding” is meant that the binding is selective for the antigen and can be discriminated from unwanted or non-specific interactions. The ability of an antibody to bind to a specific antigen (e.g. PD-1) can be measured either through an enzyme-linked immunosorbent assay (ELISA) or other techniques familiar to one of skill in the art, e.g. surface plasmon resonance (SPR) technique (analyzed e.g. on a BIAcore instrument) (Liljeblad et al., Glyco J 17, 323-329 (2000)), and traditional binding assays (Heeley, Endocr Res 28, 217-229 (2002)). In one embodiment, the extent of binding of an antibody to an unrelated protein is less than about 10% of the binding of the antibody to the antigen as measured, e.g., by SPR. The antibody comprised in the immunoconjugate described herein specifically binds to PD-1.

As used herein, term “polypeptide” refers to a molecule composed of monomers (amino acids) linearly linked by amide bonds (also known as peptide bonds). The term “polypeptide” refers to any chain of two or more amino acids, and does not refer to a specific length of the product. Thus, peptides, dipeptides, tripeptides, oligopeptides, “protein”, “amino acid chain”, or any other term used to refer to a chain of two or more amino acids, are included within the definition of “polypeptide”, and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

By an “isolated” polypeptide or a variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for the purpose of the invention, as are native or recombinant polypeptides which have been separated, fractionated, or partially or substantially purified by any suitable technique.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. For purposes herein, however, % amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227- 258; and Pearson et. al. (1997) Genomics 46:24-36, and is publicly available from http://fasta.bioch.virginia.edu/fasta_www2/fasta_down.shtml. Alternatively, a public server accessible at http://fasta.bioch.virginia.edu/fasta_www2/index.cgi can be used to compare the sequences, using the ggsearch (global protein:protein) program and default options (BLOSUM50; open: -10; ext: -2; Ktup = 2) to ensure a global, rather than local, alignment is performed. Percent amino acid identity is given in the output alignment header.

The term “polynucleotide” refers to an isolated nucleic acid molecule or construct, e.g. messenger RNA (mRNA), virally-derived RNA, or plasmid DNA (pDNA). A polynucleotide may comprise a conventional phosphodiester bond or a non-conventional bond (e.g. an amide bond, such as found in peptide nucleic acids (PNA). The term “nucleic acid molecule” refers to any one or more nucleic acid segments, e.g. DNA or RNA fragments, present in a polynucleotide.

By “isolated” nucleic acid molecule or polynucleotide is intended a nucleic acid molecule, DNA or RNA, which has been removed from its native environment. For example, a recombinant polynucleotide encoding a polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present invention, as well as positive and negative strand forms, and double-stranded forms. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

“Isolated polynucleotide (or nucleic acid) encoding [e.g. an immunoconjugate of the invention]” refers to one or more polynucleotide molecules encoding antibody heavy and light chains and/or IL-7 polypeptides (or fragments thereof), including such polynucleotide molecule(s) in a single vector or separate vectors, and such nucleic acid molecule(s) present at one or more locations in a host cell.

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette comprises polynucleotide sequences that encode immunoconjugates of the invention or fragments thereof.

The term “vector” or “expression vector” refers to a DNA molecule that is used to introduce and direct the expression of a specific gene to which it is operably associated in a cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the cell, the ribonucleic acid molecule or protein that is encoded by the gene is produced by the cellular transcription and/or translation machinery. In one embodiment, the expression vector of the invention comprises an expression cassette that comprises polynucleotide sequences that encode immunoconjugates of the invention or fragments thereof.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the immunoconjugates of the present invention. Host cells include cultured cells, e.g. mammalian cultured cells, such as HEK cells, CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

The term “antibody” herein is used in the broadest sense and encompasses various antibody structures, including but not limited to monoclonal antibodies, polyclonal antibodies, multispecific antibodies (e.g. bispecific antibodies), and antibody fragments so long as they exhibit the desired antigen binding activity.

The term “monoclonal antibody” as used herein refers to an antibody obtained from a population of substantially homogeneous antibodies, i.e. the individual antibodies comprised in the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

An “isolated” antibody is one which has been separated from a component of its natural environment, i.e. that is not in its natural milieu. No particular level of purification is required. For example, an isolated antibody can be removed from its native or natural environment. Recombinantly produced antibodies expressed in host cells are considered isolated for the purpose of the invention, as are native or recombinant antibodies which have been separated, fractionated, or partially or substantially purified by any suitable technique. As such, the immunoconjugates of the present invention are isolated. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For review of methods for assessment of antibody purity, see, e.g., Flatman et al., J. Chromatogr. B 848:79-87 (2007).

The terms “full-length antibody,” “intact antibody,” and “whole antibody” are used herein interchangeably to refer to an antibody having a structure substantially similar to a native antibody structure.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g. scFv), and single-domain antibodies. For a review of certain antibody fragments, see Holliger and Hudson, Nature Biotechnology 23:1126-1136 (2005). For a review of scFv fragments, see e.g. Plückthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable domain (VH), also called a variable heavy domain or a heavy chain variable region, followed by three constant domains (CH1, CH2, and CH3), also called a heavy chain constant region. Similarly, from N- to C-terminus, each light chain has a variable domain (VL), also called a variable light domain or a light chain variable region, followed by a constant light (CL) domain, also called a light chain constant region. The heavy chain of an immunoglobulin may be assigned to one of five types, called α (IgA), δ (IgD), ε (IgE), γ (IgG), or µ (IgM), some of which may be further divided into subtypes, e.g. _{γ1} (IgG₁), _{γ2} (IgG₂), _{γ3} (IgG₃), _{γ4} (IgG₄), _{α1} (IgA₁) and _{α2} (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The term “antigen binding domain” refers to the part of an antibody that comprises the area which specifically binds to and is complementary to part or all of an antigen. An antigen binding domain may be provided by, for example, one or more antibody variable domains (also called antibody variable regions). Particularly, an antigen binding domain comprises an antibody light chain variable domain (VL) and an antibody heavy chain variable domain (VH).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6^{th} ed., W.H. Freeman and Co., page 91 (2007). A single VH or VL domain may be sufficient to confer antigen-binding specificity. As used herein in connection with variable region sequences, “Kabat numbering” refers to the numbering system set forth by Kabat et al., Sequences of Proteins of Immunological Interest, 5th Ed. Public Health Service, National Institutes of Health, Bethesda, MD (1991).

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, MD (1991), referred to as “numbering according to Kabat” or “Kabat numbering” herein. Specifically the Kabat numbering system (see pages 647-660 of Kabat, et al., Sequences of Proteins of Immunological Interest, 5th ed., Public Health Service, National Institutes of Health, Bethesda, MD (1991)) is used for the light chain constant domain CL of kappa and lambda isotype and the Kabat EU index numbering system (see pages 661-723) is used for the heavy chain constant domains (CH1, Hinge, CH2 and CH3), which is herein further clarified by referring to “numbering according to Kabat EU index” in this case.

The term “hypervariable region” or “HVR”, as used herein, refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs; three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following order in VH (or VL): FR1-H1(L1)-FR2-H2(L2)-FR3-H3(L3)-FR4.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. Such variable domains are referred to herein as “humanized variable region”. A humanized antibody optionally may comprise at least a portion of an antibody constant region derived from a human antibody. In some embodiments, some FR residues in a humanized antibody are substituted with corresponding residues from a non-human antibody (e.g., the antibody from which the HVR residues are derived), e.g., to restore or improve antibody specificity or affinity. A “humanized form” of an antibody, e.g. of a non-human antibody, refers to an antibody that has undergone humanization. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. In certain embodiments, a human antibody is derived from a non-human transgenic mammal, for example a mouse, a rat, or a rabbit. In certain embodiments, a human antibody is derived from a hybridoma cell line. Antibodies or antibody fragments isolated from human antibody libraries are also considered human antibodies or human antibody fragments herein.

The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgAi, and IgA₂. The heavy chain constant domains that correspond to the different classes of immunoglobulins are called α, δ, ε, γ, and µ, respectively.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain (also referred to herein as a “cleaved variant heavy chain”). This may be the case where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (K447), of the Fc region may or may not be present. Amino acid sequences of heavy chains including Fc domains (or a subunit of an Fc domain as defined herein) are denoted herein without C-terminal glycine-lysine dipeptide if not indicated otherwise. In one embodiment of the invention, a heavy chain including a subunit of an Fc domain as specified herein, comprised in an immunoconjugate according to the invention, comprises an additional C-terminal glycine-lysine dipeptide (G446 and K447, numbering according to EU index of Kabat). In one embodiment of the invention, a heavy chain including a subunit of an Fc domain as specified herein, comprised in an immunoconjuate according to the invention, comprises an additional C-terminal glycine residue (G446, numbering according to EU index of Kabat). Compositions of the invention, such as the pharmaceutical compositions described herein, comprise a population of immunoconjugates of the invention. The population of immunoconjugates may comprise molecules having a full-length heavy chain and molecules having a cleaved variant heavy chain. The population of immunoconjugates may consist of a mixture of molecules having a full-length heavy chain and molecules having a cleaved variant heavy chain, wherein at least 50%, at least 60%, at least 70%, at least 80% or at least 90% of the immunoconjugates have a cleaved variant heavy chain. In one embodiment of the invention, a composition comprising a population of immunoconjugates of the invention comprises an immunoconjugate comprising a heavy chain including a subunit of an Fc domain as specified herein with an additional C-terminal glycine-lysine dipeptide (G446 and K447, numbering according to EU index of Kabat). In one embodiment of the invention, a composition comprising a population of immunoconjugates of the invention comprises an immunoconjugate comprising a heavy chain including a subunit of an Fc domain as specified herein with an additional C-terminal glycine residue (G446, numbering according to EU index of Kabat). In one embodiment of the invention, such a composition comprises a population of immunoconjugates comprised of molecules comprising a heavy chain including a subunit of an Fc domain as specified herein; molecules comprising a heavy chain including a subunit of a Fc domain as specified herein with an additional C-terminal glycine residue (G446, numbering according to EU index of Kabat); and molecules comprising a heavy chain including a subunit of an Fc domain as specified herein with an additional C-terminal glycine-lysine dipeptide (G446 and K447, numbering according to EU index of Kabat). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991 (see also above). A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which might be non-identical in the sense that further components fused to each of the subunits (e.g. antigen binding moieties) are not the same. In some embodiments the modification promoting association comprises an amino acid mutation in the Fc domain, specifically an amino acid substitution. In a particular embodiment, the modification promoting association comprises a separate amino acid mutation, specifically an amino acid substitution, in each of the two subunits of the Fc domain.

The term “effector functions” when used in reference to antibodies refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

Antibody-dependent cell-mediated cytotoxicity (ADCC) is an immune mechanism leading to the lysis of antibody-coated target cells by immune effector cells. The target cells are cells to which antibodies or derivatives thereof comprising an Fc region specifically bind, generally via the protein part that is N-terminal to the Fc region. As used herein, the term “reduced ADCC” is defined as either a reduction in the number of target cells that are lysed in a given time, at a given concentration of antibody in the medium surrounding the target cells, by the mechanism of ADCC defined above, and/or an increase in the concentration of antibody in the medium surrounding the target cells, required to achieve the lysis of a given number of target cells in a given time, by the mechanism of ADCC. The reduction in ADCC is relative to the ADCC mediated by the same antibody produced by the same type of host cells, using the same standard production, purification, formulation and storage methods (which are known to those skilled in the art), but that has not been engineered. For example the reduction in ADCC mediated by an antibody comprising in its Fc domain an amino acid substitution that reduces ADCC, is relative to the ADCC mediated by the same antibody without this amino acid substitution in the Fc domain. Suitable assays to measure ADCC are well known in the art (see e.g. PCT Publication No. WO 2006/082515 or PCT Publication No. WO 2012/130831).

An “activating Fc receptor” is an Fc receptor that following engagement by an Fc domain of an antibody elicits signaling events that stimulate the receptor-bearing cell to perform effector functions. Human activating Fc receptors include FcyRIIIa (CD16a), FcyRI (CD64), FcyRIIa (CD32), and FcαRI (CD89).

As used herein, the terms “engineer, engineered, engineering”, are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches.

“Reduced binding”, for example reduced binding to an Fc receptor or CD25, refers to a decrease in affinity for the respective interaction, as measured for example by SPR. For clarity, the term includes also reduction of the affinity to zero (or below the detection limit of the analytic method), i.e. complete abolishment of the interaction. Conversely, “increased binding” refers to an increase in binding affinity for the respective interaction.

As used herein, the term “immunoconjugate” refers to a polypeptide molecule that includes at least one IL-7 molecule and at least one antibody. The IL-7 molecule can be joined to the antibody by a variety of interactions and in a variety of configurations as described herein. In particular embodiments, the IL-7 molecule is fused to the antibody via a peptide linker. Particular immunoconjugates according to the invention essentially consist of one IL-7 molecule and an antibody joined by one or more linker sequences.

By “fused” is meant that the components (e.g. an antibody and an IL-7 molecule) are linked by peptide bonds, either directly or via one or more peptide linkers.

As used herein, the terms “first” and “second” with respect to Fc domain subunits etc., are used for convenience of distinguishing when there is more than one of each type of moiety. Use of these terms is not intended to confer a specific order or orientation of the immunoconjugate unless explicitly so stated.

An “effective amount” of an agent refers to the amount that is necessary to result in a physiological change in the cell or tissue to which it is administered.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” refers to a preparation which is in such form as to permit the biological activity of an active ingredient contained therein to be effective, and which contains no additional components which are unacceptably toxic to a subject to which the composition would be administered.

A “pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, immunoconjugates of the invention are used to delay development of a disease or to slow the progression of a disease.

### DETAILED DESCRIPTION OF THE EMBODIMENTS

### Mutant IL-7 Polypeptide

The IL-7 variants according to the present inverntion have advantageous properties for immunotherapy.

The mutant interleukin-7 (IL-7) polypeptide according to the invention comprises at least one amino acid mutation that reduces affinity of the mutant IL-7 polypeptide to the α-subunit of the IL-7 receptor and/or the IL-2Ry subunit.

Mutants of human IL-7 (hIL-7) with decreased affinity to IL-7Rα and/or IL-2Rγ may for example be generated by amino acid substitution at amino acid position 13, 15, 18, 21, 22, 25, 72, , 77, 81, 84, 85, 88 , 136, 139, 143 or 147 or combinations thereof (numbering relative to the human IL-7 sequence SEQ ID NO: 52). Exemplary amino acid substitutions include E13A, E13K, V15A, V15K, V18A, V18K, D21A, D21K, Q22A, Q22K, D25A, D25K, T72A, L77A, L77K, K81A, K81E, E84A, G85K, G85E, I88K, Q136A, Q136K, K139A, K139E, N143K and M147A.

The mutant interleukin-7 (IL-7) polypeptide according to the invention may comprise at least one amino acid mutation that improves the homonogeneity of the polypetide, preferably in one of the amino acid positions 74, 93 and 118 or combinations thereof. Exemplary amino acid substitutions include D74A, D74K, T93A and S118A.

In some embodimenets of the invention the mutant interleukin-7 polypeptide comprises an amino acid sequence of SEQ ID NO: 53. In some embodimenets of the invention the mutant interleukin-7 polypeptide comprises an amino acid sequence of SEQ ID NO: 54. In some embodimenets of the invention the mutant interleukin-7 polypeptide comprises an amino acid sequence of SEQ ID NO: 55. In some embodimenets of the invention the mutant interleukin-7 polypeptide comprises an amino acid sequence of SEQ ID NO: 56. In some embodimenets of the invention the mutant interleukin-7 polypeptide comprises an amino acid sequence of SEQ ID NO: 57. In some embodimenets of the invention the mutant interleukin-7 polypeptide comprises an amino acid sequence of SEQ ID NO: 58. In some embodimenets of the invention the mutant interleukin-7 polypeptide comprises an amino acid sequence of SEQ ID NO: 59. In some embodimenets of the invention the mutant interleukin-7 polypeptide comprises an amino acid sequence of SEQ ID NO: 60. In some embodimenets of the invention the mutant interleukin-7 polypeptide comprises an amino acid sequence of SEQ ID NO: 61. In some embodimenets of the invention the mutant interleukin-7 polypeptide comprises an amino acid sequence of SEQ ID NO: 62. In some embodimenets of the invention the mutant interleukin-7 polypeptide comprises an amino acid sequence of SEQ ID NO: 63. In some embodimenets of the invention the mutant interleukin-7 polypeptide comprises an amino acid sequence of SEQ ID NO: 64. In some embodimenets of the invention the mutant interleukin-7 polypeptide comprises an amino acid sequence of SEQ ID NO: 65. In some embodimenets of the invention the mutant interleukin-7 polypeptide comprises an amino acid sequence of SEQ ID NO: 66. In some embodimenets of the invention the mutant interleukin-7 polypeptide comprises an amino acid sequence of SEQ ID NO: 67. In some embodimenets of the invention the mutant interleukin-7 polypeptide comprises an amino acid sequence of SEQ ID NO: 68. In some embodimenets of the invention the mutant interleukin-7 polypeptide comprises an amino acid sequence of SEQ ID NO: 69. In some embodimenets of the invention the mutant interleukin-7 polypeptide comprises an amino acid sequence of SEQ ID NO: 70. In some embodimenets of the invention the mutant interleukin-7 polypeptide comprises an amino acid sequence of SEQ ID NO: 71. In some embodimenets of the invention the mutant interleukin-7 polypeptide comprises an amino acid sequence of SEQ ID NO: 72. In some embodimenets of the invention the mutant interleukin-7 polypeptide comprises an amino acid sequence of SEQ ID NO: 73. In some embodimenets of the invention the mutant interleukin-7 polypeptide comprises an amino acid sequence of SEQ ID NO: 74. In some embodimenets of the invention the mutant interleukin-7 polypeptide comprises an amino acid sequence of SEQ ID NO: 75. In some embodimenets of the invention the mutant interleukin-7 polypeptide comprises an amino acid sequence of SEQ ID NO: 76. In some embodimenets of the invention the mutant interleukin-7 polypeptide comprises an amino acid sequence of SEQ ID NO: 77. In some embodimenets of the invention the mutant interleukin-7 polypeptide comprises an amino acid sequence of SEQ ID NO: 78. In some embodimenets of the invention the mutant interleukin-7 polypeptide comprises an amino acid sequence of SEQ ID NO: 79. In some embodimenets of the invention the mutant interleukin-7 polypeptide comprises an amino acid sequence of SEQ ID NO: 80. In some embodimenets of the invention the mutant interleukin-7 polypeptide comprises an amino acid sequence of SEQ ID NO: 81. In some embodimenets of the invention the mutant interleukin-7 polypeptide comprises an amino acid sequence of SEQ ID NO: 135. In some embodimenets of the invention the mutant interleukin-7 polypeptide comprises an amino acid sequence of SEQ ID NO: 136.

Particular IL-7 mutants of the invention comprise an amino acid mutation selected from the group of V15A, V15K, V18A, V18K, L77A, L77K, K81E, G85K, G85E, I88K and N143K of human IL-7 according to SEQ ID NO: 52. A particular IL-7 mutant of the invention comprises the amino acid sequence of SEQ ID NO: 55. A particular IL-7 mutant of the invention comprises the amino acid sequence of SEQ IN NO: 56. A particular IL-7 mutant of the invention comprises the amino acid sequence of SEQ ID NO: 57. A particular IL-7 mutant of the invention comprises the amino acid sequence of SEQ ID NO: 58. A particular IL-7 mutant of the invention comprises the amino acid sequence of SEQ ID NO: 67. A particular IL-7 mutant of the invention comprises the amino acid sequence of SEQ ID NO: 68. A particular IL-7 mutant of the invention comprises the amino acid sequence of SEQ ID NO: 70. A particular IL-7 mutant of the invention comprises the amino acid sequence of SEQ ID NO: 72. A particular IL-7 mutant of the invention comprises the amino acid sequence of SEQ ID NO: 73. A particular IL-7 mutant of the invention comprises the amino acid sequence of SEQ ID NO: 74. A particular IL-7 mutant of the invention comprises the amino acid sequence of SEQ ID NO: 79. These mutants exhibit substantially reduced affinity to the interleukin 7 receptor compared to a wild-type form of the IL-7 mutant.

Particular IL-7 mutants of the invention comprise at least two amino acid substitutions, wherein the two amino acid substitutions are K81E and G85K or G85E of human IL-7 according to SEQ ID NO: 52. A particular IL-7 mutant of the invention comprises the amino acid sequence of SEQ ID NO: 135. A particular IL-7 mutant of the invention comprises the amino acid sequence of SEQ IN NO: 136.

Other characteristics of IL-7 mutants as disclosed herein include reduced affinity to IL-7Rα to allow PD-1 mediated delivery of IL-7 in cis (on the same cell) on PD-1 expressing CD4 T cells, compared to wild-type IL-7 which is mainly delivered in trans (on cell in close proximity) when in a PD1-IL-7 immunoconjugate.

In certain embodiments said amino acid mutation reduces the affinity of the mutant IL-7 polypeptide to the IL-R_{α} and/or the IL-2Rγ by at least 5-fold, specifically at least 10-fold, more specifically at least 25-fold .

Reduction of the affinity of IL-7 for the IL-7Rα and/or the IL-2Rγ in combination with elimination of the N-glycosylation of IL-7 results in an IL-7 protein with improved properties. For example, elimination of the N-glycosylation site results in a more homogenous product when the mutant IL-7 polypeptide is expressed in mammalian cells such as CHO or HEK cells.

Thus, in certain embodiments the mutant IL-7 polypeptide comprises an additional amino acid mutation which eliminates the N-glycosylation site of IL-7 at a position corresponding to residue 72, 93 or 118 of human IL-7. In one embodiment said additional amino acid mutation which eliminates the N-glycosylation site of IL-7 at a position corresponding to residue 72, 93 or 118 of human IL-7 is an amino acid substitution. In a specific embodiment, said additional amino acid mutation is the amino acid substitution T72A. In another specific embodiment, said additional amino acid mutation is the amino acid substitution T93A. In another specific embodiment, said additional amino acid mutation is the amino acid substitution S118A. In another specific embodiment, the mutant IL-7 polypeptide comprises the amino acid substitutions T72A, T93A and S118A. In certain embodiments the mutant IL-7 polypeptide is essentially a full-length IL-7 molecule. In certain embodiments the mutant IL-7 polypeptide is a human IL-7 molecule. In one embodiment the mutant IL-7 polypeptide comprises the sequence of SEQ ID NO: 52 with at least one amino acid mutation that reduces affinity of the mutant IL-7 polypeptide to IL-7Rα or IL-2Rγ compared to an IL-7 polypeptide comprising SEQ ID NO: 52 without said mutation. In one embodiment the mutant IL-7 polypeptide comprises the sequence of SEQ ID NO: 52 with at least one amino acid mutation that reduces affinity of the mutant IL-7 polypeptide to IL-7Rα and IL-2Rγ compared to an IL-7 polypeptide comprising SEQ ID NO: 52 without said mutation. In one embodiment the mutant IL-7 polypeptide comprises the sequence of SEQ ID NO: 52 with at least one amino acid mutation that reduces affinity of the mutant IL-7 polypeptide to IL-7Rα and/or IL-2Rγ compared to an IL-7 polypeptide comprising SEQ ID NO: 52 without said mutation.

In a specific embodiment, the mutant IL-7 polypeptide can still elicit one or more of the cellular responses selected from the group consisting of: proliferation in T lymphocyte cells, effector functions in an primed T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity.

In one embodiment, the mutant IL-7 polypeptide comprises no more than 12, no more than 11, no more than 10, no more than 9, no more than 8, no more than 7, no more than 6, or no more than 5 amino acid mutations as compared to the corresponding wild-type IL-2 sequence, e.g. the human IL-7 sequence of SEQ ID NO: 52. In a particular embodiment, the mutant IL-7 polypeptide comprises no more than 5 amino acid mutations as compared to the corresponding wild-type IL-7 sequence, e.g. the human IL-7 sequence of SEQ ID NO: 52.

### Immunoconjugates

Immunoconjugates as described herein comprise an IL-molecule and an antibody. Such immunoconjugates significantly increase the efficacy of IL-7 therapy by directly targeting IL-7 e.g. into a tumor microenvironment. According to the invention, an antibody comprised in the immunoconjugate can be a whole antibody or immunoglobulin, or a portion or variant thereof that has a biological function such as antigen specific binding affinity.

The general benefits of immunoconjugate therapy are readily apparent. For example, an antibody comprised in an immunoconjugate recognizes a tumor-specific epitope and results in targeting of the immunoconjugate molecule to the tumor site. Therefore, high concentrations of IL-7 can be delivered into the tumor microenvironment, thereby resulting in activation and proliferation of a variety of immune effector cells mentioned herein using a much lower dose of the immunoconjugate than would be required for unconjugated IL-7. However, this characteristic of IL-7 immunoconjugates may again aggravate potential side effects of the IL-7 molecule: Because of the significantly longer circulating half-life of IL-7 immunoconjugate in the bloodstream relative to unconjugated IL-7, the probability for IL-7 or other portions of the fusion protein molecule to activate components generally present in the vasculature is increased. The same concern applies to other fusion proteins that contain IL-7 fused to another moiety such as Fc or albumin, resulting in an extended half-life of IL-7 in the circulation. Therefore immunoconjugates comprising a mutant IL-7 polypeptide as described herein with reduced toxicity compared to wild-type forms of IL-7, is particularly advantageous.

As described hereinabove, targeting IL-7 directly to immune effector cells rather than tumor cells may be advantageous for IL-7 immunotherapy.

Accordingly, the invention provides a mutant IL-7 polypeptide as described hereinbefore, and an antibody that binds to PD-1. In one embodiment the mutant IL-7 polypeptide and the antibody form a fusion protein, i.e. the mutant IL-7 polypeptide shares a peptide bond with the antibody. In some embodiments, the antibody comprises an Fc domain composed of a first and a second subunit. In a specific embodiment the mutant IL-7 polypeptide is fused at its amino-terminal amino acid to the carboxy-terminal amino acid of one of the subunits of the Fc domain, optionally through a linker peptide. In some embodiments, the antibody is a full-length antibody. In some embodiments, the antibody is an immunoglobulin molecule, particularly an IgG class immunoglobulin molecule, more particularly an IgG₁ subclass immunoglobulin molecule. In one such embodiment, the mutant IL-7 polypeptide shares an amino-terminal peptide bond with one of the immunoglobulin heavy chains. In certain embodiments the antibody is an antibody fragment. In some embodiments the antibody is a Fab molecule or a scFv molecule. In one embodiment the antibody is a Fab molecule. In another embodiment the antibody is a scFv molecule. The immunoconjugate may also comprise more than one antibody. Where more than one antibody is comprised in the immunoconjugate, e.g. a first and a second antibody, each antibody can be independently selected from various forms of antibodies and antibody fragments. For example, the first antibody can be a Fab molecule and the second antibody can be a scFv molecule. In a specific embodiment each of said first and said second antibodies is a scFv molecule or each of said first and said second antibodies is a Fab molecule. In a particular embodiment each of said first and said second antibodies is a Fab molecule. In one embodiment each of said first and said second antibodies binds to PD-1.

### Immunoconjugate Formats

Exemplary immunoconjugate formats are described in PCT Publication No. WO 2011/020783, which is incorporated herein by reference in its entirety. These immunoconjugates comprise at least two antibodies. Thus, in one embodiment, the immunoconjugate according to the present invention comprises a mutant IL-7 polypeptide as described herein, and at least a first and a second antibody. In a particular embodiment, said first and second antibody are independently selected from the group consisting of an Fv molecule, particularly a scFv molecule, and a Fab molecule. In a specific embodiment, said mutant IL-7 polypeptide shares an amino- or carboxy-terminal peptide bond with said first antibody and said second antibody shares an amino- or carboxy-terminal peptide bond with either i) the mutant IL-7 polypeptide or ii) the first antibody. In a particular embodiment, the immunoconjugate consists essentially of a mutant IL-7 polypeptide and first and second antibodies, particularly Fab molecules, joined by one or more linker sequences. Such formats have the advantage that they bind with high affinity to the target antigen (PD-1), but provide only monomeric binding to the IL-7 receptor, thus avoiding targeting the immunoconjugate to IL-7 receptor bearing immune cells at other locations than the target site. In a particular embodiment, a mutant IL-7 polypeptide shares a carboxy-terminal peptide bond with a first antibody, particularly a first Fab molecule, and further shares an amino-terminal peptide bond with a second antibody, particularly a second Fab molecule. In another embodiment, a first antibody, particularly a first Fab molecule, shares a carboxy-terminal peptide bond with a mutant IL-7 polypeptide, and further shares an amino-terminal peptide bond with a second antibody, particularly a second Fab molecule. In another embodiment, a first antibody, particularly a first Fab molecule, shares an amino-terminal peptide bond with a first mutant IL-7 polypeptide, and further shares a carboxy-terminal peptide with a second antibody, particularly a second Fab molecule. In a particular embodiment, a mutant IL-7 polypeptide shares a carboxy-terminal peptide bond with a first heavy chain variable region and further shares an amino-terminal peptide bond with a second heavy chain variable region. In another embodiment a mutant IL-7 polypeptide shares a carboxy-terminal peptide bond with a first light chain variable region and further shares an amino-terminal peptide bond with a second light chain variable region. In another embodiment, a first heavy or light chain variable region is joined by a carboxy-terminal peptide bond to a mutant IL-7 polypeptide and is further joined by an amino-terminal peptide bond to a second heavy or light chain variable region. In another embodiment, a first heavy or light chain variable region is joined by an amino-terminal peptide bond to a mutant IL-7 polypeptide and is further joined by a carboxy-terminal peptide bond to a second heavy or light chain variable region. In one embodiment, a mutant IL-7 polypeptide shares a carboxy-terminal peptide bond with a first Fab heavy or light chain and further shares an amino-terminal peptide bond with a second Fab heavy or light chain. In another embodiment, a first Fab heavy or light chain shares a carboxy-terminal peptide bond with a mutant IL-7 polypeptide and further shares an amino-terminal peptide bond with a second Fab heavy or light chain. In other embodiments, a first Fab heavy or light chain shares an amino-terminal peptide bond with a mutant IL-7 polypeptide and further shares a carboxy-terminal peptide bond with a second Fab heavy or light chain. In one embodiment, the immunoconjugate comprises a mutant IL-7 polypeptide sharing an amino-terminal peptide bond with one or more scFv molecules and further sharing a carboxy-terminal peptide bond with one or more scFv molecules.

Particularly suitable formats for the immunoconjugates according to the present invention, however comprise an immunoglobulin molecule as antibody. Such immunoconjugate formats are described in WO 2012/146628, which is incorporated herein by reference in its entirety.

Accordingly, in particular embodiments, the immunoconjugate comprises a mutant IL-7 polypeptide as described herein and an immunoglobulin molecule that binds to PD-1, particularly an IgG molecule, more particularly an IgG₁ molecule. In one embodiment the immunoconjugate comprises not more than one mutant IL-7 polypeptide. In one embodiment the immunoglobulin molecule is human. In one embodiment, the immunoglobulin molecule comprises a human constant region, e.g. a human CH1, CH2, CH3 and/or CL domain. In one embodiment, the immunoglobulin comprises a human Fc domain, particularly a human IgG₁ Fc domain. In one embodiment the mutant IL-7 polypeptide shares an amino- or carboxy-terminal peptide bond with the immunoglobulin molecule. In one embodiment, the immunoconjugate essentially consists of a mutant IL-7 polypeptide and an immunoglobulin molecule, particularly an IgG molecule, more particularly an IgG₁ molecule, joined by one or more linker sequences. In a specific embodiment the mutant IL-7 polypeptide is fused at its amino-terminal amino acid to the carboxy-terminal amino acid of one of the immunoglobulin heavy chains, optionally through a linker peptide.

The mutant IL-7 polypeptide may be fused to the antibody directly or through a linker peptide, comprising one or more amino acids, typically about 2-20 amino acids. Linker peptides are known in the art and are described herein. Suitable, non-immunogenic linker peptides include, for example, (G₄S)ₙ, (SG₄)ₙ, (G₄S)ₙ or G₄(SG₄)ₙ linker peptides. “n” is generally an integer from 1 to 10, typically from 2 to 4. In one embodiment the linker peptide has a length of at least 5 amino acids, in one embodiment a length of 5 to 100, in a further embodiment of 10 to 50 amino acids. In a particular embodiment, the linker peptide has a length of 15 amino acids. In one embodiment the linker peptide is (GxS)ₙ or (GxS)ₙGₘ with G=glycine, S=serine, and (x=3, n= 3, 4, 5 or 6, and m=0, 1, 2 or 3) or (x=4, n=2, 3, 4 or 5 and m= 0, 1, 2 or 3), in one embodiment x=4 and n=2 or 3, in a further embodiment x=4 and n=3. In a particular embodiment the linker peptide is (G₄S)₃ (SEQ ID NO: 21). In one embodiment, the linker peptide has (or consists of) the amino acid sequence of SEQ ID NO: 21.

In a particular embodiment, the immunoconjugate comprises a mutant IL-7 molecule and an immunoglobulin molecule, particularly an IgG₁ subclass immunoglobulin molecule, that binds to PD-1, wherein the mutant IL-7 molecule is fused at its amino-terminal amino acid to the carboxy-terminal amino acid of one of the immunoglobulin heavy chains through the linker peptide of SEQ ID NO: 21.

In a particular embodiment, the immunoconjugate comprises a mutant IL-7 molecule and an antibody that binds to PD-1, wherein the antibody comprises an Fc domain, particularly a human IgG₁ Fc domain, composed of a first and a second subunit, and the mutant IL-7 molecule is fused at its amino-terminal amino acid to the carboxy-terminal amino acid of one of the subunits of the Fc domain through the linker peptide of SEQ ID NO: 21.

### PD-1 Antibodies

The antibody comprised in the immunoconjugate of the invention binds to PD-1, particularly human PD-1, and is able to direct the mutant IL-7 polypeptide to a target site where PD-1 is expressed, particularly to a T cell that expresses PD-1, for example associated with a tumor.

Suitable PD-1 antibodies that may be used in the immunoconjugate of the invention are described in WO 2017/055443 A1, which is incorporated herein by reference in its entirety.

The immunoconjugate of the invention may comprise two or more antibodies, which may bind to the same or to different antigens. In particular embodiments, however, each of these antibodies binds to PD-1. In one embodiment, the antibody comprised in the immunoconjugate of the invention is monospecific. In a particular embodiment, the immunoconjugate comprises a single, monospecific antibody, particularly a monospecific immunoglobulin molecule.

The antibody can be any type of antibody or fragment thereof that retains specific binding to PD-1, particularly human PD-1. Antibody fragments include, but are not limited to, Fv molecules, scFv molecule, Fab molecule, and F(ab′)₂ molecules. In particular embodiments, however, the antibody is a full-length antibody. In some embodiments, the antibody comprises an Fc domain, composed of a first and a second subunit. In some embodiments, the antibody is an immunoglobulin, particularly an IgG class, more particularly an IgG₁ subclass immunoglobulin.

In some embodiments, the antibody is a monoclonal antibody.

In some embodiments, the antibody comprises a HVR-H1 comprising the amino acid sequence of SEQ ID NO:1, a HVR-H2 comprising the amino acid sequence of SEQ ID NO:2, a HVR-H3 comprising the amino acid sequence of SEQ ID NO:3, a FR-H3 comprising the amino acid sequence of SEQ ID NO:7 at positions 71-73 according to Kabat numbering, a HVR-L1 comprising the amino acid sequence of SEQ ID NO:4, a HVR-L2 comprising the amino acid sequence of SEQ ID NO:5, and a HVR-L3 comprising the amino acid sequence of SEQ ID NO:6.

In some embodiments, the antibody comprises (a) a heavy chain variable region (VH) comprising a HVR-H1 comprising the amino acid sequence of SEQ ID NO:1, a HVR-H2 comprising the amino acid sequence of SEQ ID NO:2, a HVR-H3 comprising the amino acid sequence of SEQ ID NO:3, and a FR-H3 comprising the amino acid sequence of SEQ ID NO:7 at positions 71-73 according to Kabat numbering, and (b) a light chain variable region (VL) comprising a HVR-L1 comprising the amino acid sequence of SEQ ID NO:4, a HVR-L2 comprising the amino acid sequence of SEQ ID NO:5, and a HVR-L3 comprising the amino acid sequence of SEQ ID NO:6. In some embodiments, the heavy and/or light chain variable region is a humanized variable region. In some embodiments, the heavy and/or light chain variable region comprises human framework regions (FR).

In some embodiments, the antibody comprises a HVR-H1 comprising the amino acid sequence of SEQ ID NO:8, a HVR-H2 comprising the amino acid sequence of SEQ ID NO:9, a HVR-H3 comprising the amino acid sequence of SEQ ID NO: 10, a HVR-L1 comprising the amino acid sequence of SEQ ID NO: 11, a HVR-L2 comprising the amino acid sequence of SEQ ID NO: 12, and a HVR-L3 comprising the amino acid sequence of SEQ ID NO: 13.

In some embodiments, the antibody comprises (a) a heavy chain variable region (VH) comprising a HVR-H1 comprising the amino acid sequence of SEQ ID NO:8, a HVR-H2 comprising the amino acid sequence of SEQ ID NO:9, and a HVR-H3 comprising the amino acid sequence of SEQ ID NO: 10, and (b) a light chain variable region (VL) comprising a HVR-L1 comprising the amino acid sequence of SEQ ID NO: 11, a HVR-L2 comprising the amino acid sequence of SEQ ID NO: 12, and a HVR-L3 comprising the amino acid sequence of SEQ ID NO: 13. In some embodiments, the heavy and/or light chain variable region is a humanized variable region. In some embodiments, the heavy and/or light chain variable region comprises human framework regions (FR).

In some embodiments, the antibody comprises a heavy chain variable region (VH) comprising an amino acid sequence that is at least about 95%, 96%, 97%, 98%, 99% or 100% identical to the amino acid sequence of SEQ ID NO:14. In some embodiments, the antibody comprises a light chain variable region (VL) comprising an amino acid sequence that is at least about 95%, 96%, 97%, 98%, 99% or 100% identical to an amino acid sequence selected from the group consisting of SEQ ID NO:15, SEQ ID NO:16, SEQ ID NO: 17, and SEQ ID NO:18. In some embodiments, the antibody comprises (a) a heavy chain variable region (VH) comprising an amino acid sequence that is at least about 95%, 96%, 97%, 98%, 99% or 100% identical to the amino acid sequence of SEQ ID NO:14, and (b) a light chain variable region (VL) comprising an amino acid sequence that is at least about 95%, 96%, 97%, 98%, 99% or 100% identical to an amino acid sequence selected from the group consisting of SEQ ID NO:15, SEQ ID NO:16, SEQ ID NO: 17, and SEQ ID NO:18.

In a particular embodiment, the antibody comprises (a) a heavy chain variable region (VH) comprising the amino acid sequence of SEQ ID NO: 14, and (b) a light chain variable region (VL) comprising the amino acid sequence of SEQ ID NO: 15.

In some embodiments, the antibody is a humanized antibody. In one embodiment, the antibody is an immunoglobulin molecule comprising a human constant region, particularly an IgG class immunoglobulin molecule comprising a human CH1, CH2, CH3 and/or CL domain. Exemplary sequences of human constant domains are given in SEQ ID NOs 31 and 32 (human kappa and lambda CL domains, respectively) and SEQ ID NO: 33 (human IgG1 heavy chain constant domains CH1-CH2-CH3). In some embodiments, the antibody comprises a light chain constant region comprising the amino acid sequence of SEQ ID NO: 31 or SEQ ID NO: 32, particularly the amino acid sequence of SEQ ID NO: 31. In some embodiments, the antibody comprises a heavy chain constant region comprising an amino acid sequence that is at least about 95%, 96%, 97%, 98%, 99% or 100% identical to the amino acid sequence of SEQ ID NO: 33. Particularly, the heavy chain constant region may comprise amino acid mutations in the Fc domain as described herein.

### Fc Domain

In particular embodiments, the antibody comprised in the immunconjugates according to the invention comprises an Fc domain, composed of a first and a second subunit. The Fc domain of an antibody consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other. In one embodiment the immunoconjugate of the invention comprises not more than one Fc domain. In one embodiment the Fc domain of the antibody comprised in the immunoconjugate is an IgG Fc domain. In a particular embodiment the Fc domain is an IgG₁ Fc domain. In another embodiment the Fc domain is an IgG₄ Fc domain. In a more specific embodiment, the Fc domain is an IgG₄ Fc domain comprising an amino acid substitution at position S228 (Kabat EU index numbering), particularly the amino acid substitution S228P. This amino acid substitution reduces in vivo Fab arm exchange of IgG₄ antibodies (see Stubenrauch et al., Drug Metabolism and Disposition 38, 84-91 (2010)). In a further particular embodiment the Fc domain is a human Fc domain. In an even more particular embodiment, the Fc domain is a human IgG₁ Fc domain. An exemplary sequence of a human IgG₁ Fc region is given in SEQ ID NO: 30.

### Fc Domain Modifications Promoting Heterodimerization

Immunoconjugates according to the invention comprise a mutant IL-7 polypeptide, particularly a single (not more than one) mutant IL-7 polypeptide, fused to one or the other of the two subunits of the Fc domain, thus the two subunits of the Fc domain are typically comprised in two non-identical polypeptide chains. Recombinant co-expression of these polypeptides and subsequent dimerization leads to several possible combinations of the two polypeptides. To improve the yield and purity of the immunoconjugate in recombinant production, it will thus be advantageous to introduce in the Fc domain of the antibody a modification promoting the association of the desired polypeptides.

Accordingly, in particular embodiments, the Fc domain of the antibody comprised in the immunoconjugate according to the invention comprises a modification promoting the association of the first and the second subunit of the Fc domain. The site of most extensive protein-protein interaction between the two subunits of a human IgG Fc domain is in the CH3 domain of the Fc domain. Thus, in one embodiment said modification is in the CH3 domain of the Fc domain.

There exist several approaches for modifications in the CH3 domain of the Fc domain in order to enforce heterodimerization, which are well described e.g. in WO 96/27011, WO 98/050431, EP 1870459, WO 2007/110205, WO 2007/147901, WO 2009/089004, WO 2010/129304, WO 2011/90754, WO 2011/143545, WO 2012058768, WO 2013157954, WO 2013096291. Typically, in all such approaches the CH3 domain of the first subunit of the Fc domain and the CH3 domain of the second subunit of the Fc domain are both engineered in a complementary manner so that each CH3 domain (or the heavy chain comprising it) can no longer homodimerize with itself but is forced to heterodimerize with the complementarily engineered other CH3 domain (so that the first and second CH3 domain heterodimerize and no homodimers between the two first or the two second CH3 domains are formed).

In a specific embodiment said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain.

The knob-into-hole technology is described e.g. in US 5,731,168; US 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in a particular embodiment, in the CH3 domain of the first subunit of the Fc domain of the antibody comprised in the immunoconjugate an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

Preferably said amino acid residue having a larger side chain volume is selected from the group consisting of arginine (R), phenylalanine (F), tyrosine (Y), and tryptophan (W).

Preferably said amino acid residue having a smaller side chain volume is selected from the group consisting of alanine (A), serine (S), threonine (T), and valine (V).

The protuberance and cavity can be made by altering the nucleic acid encoding the polypeptides, e.g. by site-specific mutagenesis, or by peptide synthesis.

In a specific embodiment, in the CH3 domain of the first subunit of the Fc domain (the “knobs” subunit) the threonine residue at position 366 is replaced with a tryptophan residue (T366W), and in the CH3 domain of the second subunit of the Fc domain (the “hole” subunit) the tyrosine residue at position 407 is replaced with a valine residue (Y407V). In one embodiment, in the second subunit of the Fc domain additionally the threonine residue at position 366 is replaced with a serine residue (T366S) and the leucine residue at position 368 is replaced with an alanine residue (L368A) (numberings according to Kabat EU index).

In yet a further embodiment, in the first subunit of the Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second subunit of the Fc domain additionally the tyrosine residue at position 349 is replaced by a cysteine residue (Y349C) (numberings according to Kabat EU index). Introduction of these two cysteine residues results in formation of a disulfide bridge between the two subunits of the Fc domain, further stabilizing the dimer (Carter, J Immunol Methods 248, 7-15 (2001)).

In a particular embodiment, the first subunit of the Fc domain comprises the amino acid substitutions S354C and T366W, and the second subunit of the Fc domain comprises the amino acid substitutions Y349C, T366S, L368A and Y407V (numbering according to Kabat EU index). In some embodiments, the second subunit of the Fc domain additionally comprises the amino acid substitutions H435R and Y436F (numbering according to Kabat EU index).

In a particular embodiment the mutant IL-7 polypeptide is fused (optionally through a linker peptide) to the first subunit of the Fc domain (comprising the “knob” modification). Without wishing to be bound by theory, fusion of the mutant IL-7 polypeptide to the knob-containing subunit of the Fc domain will (further) minimize the generation of immunoconjugates comprising two mutant IL-7 polypeptides (steric clash of two knob-containing polypeptides). Other techniques of CH3-modification for enforcing the heterodimerization are contemplated as alternatives according to the invention and are described e.g. in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, WO 2013/096291.

In one embodiment the heterodimerization approach described in EP 1870459, is used alternatively. This approach is based on the introduction of charged amino acids with opposite charges at specific amino acid positions in the CH3/CH3 domain interface between the two subunits of the Fc domain. One preferred embodiment for the antibody comprised in the immunoconjugate of the invention are amino acid mutations R409D; K370E in one of the two CH3 domains (of the Fc domain) and amino acid mutations D399K; E357K in the other one of the CH3 domains of the Fc domain (numbering according to Kabat EU index).

In another embodiment, the antibody comprised in the immunoconjugate of the invention comprises amino acid mutation T366W in the CH3 domain of the first subunit of the Fc domain and amino acid mutations T366S, L368A, Y407V in the CH3 domain of the second subunit of the Fc domain, and additionally amino acid mutations R409D; K370E in the CH3 domain of the first subunit of the Fc domain and amino acid mutations D399K; E357K in the CH3 domain of the second subunit of the Fc domain (numberings according to Kabat EU index).

In another embodiment, the antibody comprised in the immunoconjugate of the invention comprises amino acid mutations S354C, T366W in the CH3 domain of the first subunit of the Fc domain and amino acid mutations Y349C, T366S, L368A, Y407V in the CH3 domain of the second subunit of the Fc domain, or said antibody comprises amino acid mutations Y349C, T366W in the CH3 domain of the first subunit of the Fc domain and amino acid mutations S354C, T366S, L368A, Y407V in the CH3 domains of the second subunit of the Fc domain and additionally amino acid mutations R409D; K370E in the CH3 domain of the first subunit of the Fc domain and amino acid mutations D399K; E357K in the CH3 domain of the second subunit of the Fc domain (all numberings according to Kabat EU index).

In one embodiment, the heterodimerization approach described in WO 2013/157953 is used alternatively. In one embodiment, a first CH3 domain comprises amino acid mutation T366K and a second CH3 domain comprises amino acid mutation L351D (numberings according to Kabat EU index). In a further embodiment, the first CH3 domain comprises further amino acid mutation L351K. In a further embodiment, the second CH3 domain comprises further an amino acid mutation selected from Y349E, Y349D and L368E (preferably L368E) (numberings according to Kabat EU index).

In one embodiment, the heterodimerization approach described in WO 2012/058768 is used alternatively. In one embodiment, a first CH3 domain comprises amino acid mutations L351Y, Y407A and a second CH3 domain comprises amino acid mutations T366A, K409F. In a further embodiment, the second CH3 domain comprises a further amino acid mutation at position T411, D399, S400, F405, N390, or K392, e.g. selected from a) T411N, T411R, T411Q, T411K, T411D, T411E or T411W, b) D399R, D399W, D399Y or D399K, c) S400E, S400D, S400R, or S400K, d) F405I, F405M, F405T, F405S, F405V or F405W, e) N390R, N390K or N390D, f) K392V, K392M, K392R, K392L, K392F or K392E (numberings according to Kabat EU index). In a further embodiment, a first CH3 domain comprises amino acid mutations L351Y, Y407A and a second CH3 domain comprises amino acid mutations T366V, K409F. In a further embodiment a first CH3 domain comprises amino acid mutation Y407A and a second CH3 domain comprises amino acid mutations T366A, K409F. In a further embodiment, the second CH3 domain further comprises amino acid mutations K392E, T411E, D399R and S400R (numberings according to Kabat EU index).

In one embodiment the heterodimerization approach described in WO 2011/143545 is used alternatively, e.g. with the amino acid modification at a position selected from the group consisting of 368 and 409 (numbering according to Kabat EU index).

In one embodiment, the heterodimerization approach described in WO 2011/090762, which also uses the knobs-into-holes technology described above, is used alternatively. In one embodiment, a first CH3 domain comprises amino acid mutation T366W and a second CH3 domain comprises amino acid mutation Y407A. In one embodiment, a first CH3 domain comprises amino acid mutation T366Y and a second CH3 domain comprises amino acid mutation Y407T (numberings according to Kabat EU index).

In one embodiment, the antibody comprised in the immunoconjugate or its Fc domain is of IgG₂ subclass and the heterodimerization approach described in WO 2010/129304 is used alternatively.

In an alternative embodiment, a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable. In one such embodiment, a first CH3 domain comprises amino acid substitution of K392 or N392 with a negatively charged amino acid (e.g. glutamic acid (E), or aspartic acid (D), preferably K392D or N392D) and a second CH3 domain comprises amino acid substitution of D399, E356, D356, or E357 with a positively charged amino acid (e.g. lysine (K) or arginine (R), preferably D399K, E356K, D356K, or E357K, and more preferably D399K and E356K). In a further embodiment, the first CH3 domain further comprises amino acid substitution of K409 or R409 with a negatively charged amino acid (e.g. glutamic acid (E), or aspartic acid (D), preferably K409D or R409D). In a further embodiment, the first CH3 domain further or alternatively comprises amino acid substitution of K439 and/or K370 with a negatively charged amino acid (e.g. glutamic acid (E), or aspartic acid (D)) (all numberings according to Kabat EU index).

In yet a further embodiment, the heterodimerization approach described inWO 2007/147901 is used alternatively. In one embodiment, a first CH3 domain comprises amino acid mutations K253E, D282K, and K322D and a second CH3 domain comprises amino acid mutations D239K, E240K, and K292D (numberings according to Kabat EU index).

In still another embodiment, the heterodimerization approach described in WO 2007/110205 can be used alternatively.

In one embodiment, the first subunit of the Fc domain comprises amino acid substitutions K392D and K409D, and the second subunit of the Fc domain comprises amino acid substitutions D356K and D399K (numbering according to Kabat EU index).

### Fc Domain Modifications Reducing Fc Receptor Binding and/or Effector Function

The Fc domain confers to the immunoconjugate favorable pharmacokinetic properties, including a long serum half-life which contributes to good accumulation in the target tissue and a favorable tissue-blood distribution ratio. At the same time it may, however, lead to undesirable targeting of the immunoconjugate to cells expressing Fc receptors rather than to the preferred antigen-bearing cells. Moreover, the co-activation of Fc receptor signaling pathways may lead to cytokine release which, in combination with the IL-7 polypeptide and the long half-life of the immunoconjugate, results in excessive activation of cytokine receptors and severe side effects upon systemic administration. Accordingly, in particular embodiments, the Fc domain of the antibody comprised in the immunoconjugate according to the invention exhibits reduced binding affinity to an Fc receptor and/or reduced effector function, as compared to a native IgG₁ Fc domain. In one such embodiment the Fc domain (or the antibody comprising said Fc domain) exhibits less than 50%, preferably less than 20%, more preferably less than 10% and most preferably less than 5% of the binding affinity to an Fc receptor, as compared to a native IgG₁ Fc domain (or an antibody comprising a native IgG₁ Fc domain), and/or less than 50%, preferably less than 20%, more preferably less than 10% and most preferably less than 5% of the effector function, as compared to a native IgG₁ Fc domain domain (or an antibody comprising a native IgG₁ Fc domain). In one embodiment, the Fc domain domain (or an antibody comprising said Fc domain) does not substantially bind to an Fc receptor and/or induce effector function. In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of CDC, ADCC, ADCP, and cytokine secretion. In a particular embodiment the effector function is ADCC. In one embodiment the Fc domain domain exhibits substantially similar binding affinity to neonatal Fc receptor (FcRn), as compared to a native IgG₁ Fc domain domain. Substantially similar binding to FcRn is achieved when the Fc domain (or an antibody comprising said Fc domain) exhibits greater than about 70%, particularly greater than about 80%, more particularly greater than about 90% of the binding affinity of a native IgG₁ Fc domain (or an antibody comprising a native IgG₁ Fc domain) to FcRn. In certain embodiments the Fc domain is engineered to have reduced binding affinity to an Fc receptor and/or reduced effector function, as compared to a non-engineered Fc domain. In particular embodiments, the Fc domain of the antibody comprised in the immunoconjugate comprises one or more amino acid mutation that reduces the binding affinity of the Fc domain to an Fc receptor and/or effector function. Typically, the same one or more amino acid mutation is present in each of the two subunits of the Fc domain. In one embodiment the amino acid mutation reduces the binding affinity of the Fc domain to an Fc receptor. In one embodiment the amino acid mutation reduces the binding affinity of the Fc domain to an Fc receptor by at least 2-fold, at least 5-fold, or at least 10-fold. In embodiments where there is more than one amino acid mutation that reduces the binding affinity of the Fc domain to the Fc receptor, the combination of these amino acid mutations may reduce the binding affinity of the Fc domain to an Fc receptor by at least 10-fold, at least 20-fold, or even at least 50-fold. In one embodiment the antibody comprising an engineered Fc domain exhibits less than 20%, particularly less than 10%, more particularly less than 5% of the binding affinity to an Fc receptor as compared to an antibody comprising a non-engineered Fc domain. In a particular embodiment the Fc receptor is an Fcγ receptor. In some embodiments the Fc receptor is a human Fc receptor. In some embodiments the Fc receptor is an activating Fc receptor. In a specific embodiment the Fc receptor is an activating human Fcγ receptor, more specifically human FcγRIIIa, FcγRI or FcγRIIa, most specifically human FcγRIIIa. Preferably, binding to each of these receptors is reduced. In some embodiments binding affinity to a complement component, specifically binding affinity to C1q, is also reduced. In one embodiment binding affinity to neonatal Fc receptor (FcRn) is not reduced. Substantially similar binding to FcRn, i.e. preservation of the binding affinity of the Fc domain to said receptor, is achieved when the Fc domain (or an antibody comprising said Fc domain) exhibits greater than about 70% of the binding affinity of a non-engineered form of the Fc domain (or an antibody comprising said non-engineered form of the Fc domain) to FcRn. The Fc domain, or antibody comprised in the immunoconjugate of the invention comprising said Fc domain, may exhibit greater than about 80% and even greater than about 90% of such affinity. In certain embodiments the Fc domain of the antibody comprised in the immunoconjugate is engineered to have reduced effector function, as compared to a non-engineered Fc domain. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced crosslinking of target-bound antibodies, reduced dendritic cell maturation, or reduced T cell priming. In one embodiment the reduced effector function is one or more selected from the group of reduced CDC, reduced ADCC, reduced ADCP, and reduced cytokine secretion. In a particular embodiment the reduced effector function is reduced ADCC. In one embodiment the reduced ADCC is less than 20% of the ADCC induced by a non-engineered Fc domain (or an antibody comprising a non-engineered Fc domain).

In one embodiment the amino acid mutation that reduces the binding affinity of the Fc domain to an Fc receptor and/or effector function is an amino acid substitution. In one embodiment the Fc domain comprises an amino acid substitution at a position selected from the group of E233, L234, L235, N297, P331 and P329 (numberings according to Kabat EU index). In a more specific embodiment the Fc domain comprises an amino acid substitution at a position selected from the group of L234, L235 and P329 (numberings according to Kabat EU index). In some embodiments the Fc domain comprises the amino acid substitutions L234A and L235A (numberings according to Kabat EU index). In one such embodiment, the Fc domain is an IgG₁ Fc domain, particularly a human IgG₁ Fc domain. In one embodiment the Fc domain comprises an amino acid substitution at position P329. In a more specific embodiment the amino acid substitution is P329A or P329G, particularly P329G (numberings according to Kabat EU index). In one embodiment the Fc domain comprises an amino acid substitution at position P329 and a further amino acid substitution at a position selected from E233, L234, L235, N297 and P331 (numberings according to Kabat EU index). In a more specific embodiment the further amino acid substitution is E233P, L234A, L235A, L235E, N297A, N297D or P331S. In particular embodiments the Fc domain comprises amino acid substitutions at positions P329, L234 and L235 (numberings according to Kabat EU index). In more particular embodiments the Fc domain comprises the amino acid mutations L234A, L235A and P329G (“P329G LALA”, “PGLALA” or “LALAPG”). Specifically, in particular embodiments, each subunit of the Fc domain comprises the amino acid substitutions L234A, L235A and P329G (Kabat EU index numbering), i.e. in each of the first and the second subunit of the Fc domain the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index). In one such embodiment, the Fc domain is an IgG₁ Fc domain, particularly a human IgG₁ Fc domain. The “P329G LALA” combination of amino acid substitutions almost completely abolishes Fcγ receptor (as well as complement) binding of a human IgG₁ Fc domain, as described in PCT Publication No. WO 2012/130831, which is incorporated herein by reference in its entirety. WO 2012/130831 also describes methods of preparing such mutant Fc domains and methods for determining its properties such as Fc receptor binding or effector functions.

IgG₄ antibodies exhibit reduced binding affinity to Fc receptors and reduced effector functions as compared to IgG₁ antibodies. Hence, in some embodiments the Fc domain of the antibody comprised in the immunoconjugate of the invention is an IgG₄ Fc domain, particularly a human IgG₄ Fc domain. In one embodiment the IgG₄ Fc domain comprises amino acid substitutions at position S228, specifically the amino acid substitution S228P (numberings according to Kabat EU index). To further reduce its binding affinity to an Fc receptor and/or its effector function, in one embodiment the IgG₄ Fc domain comprises an amino acid substitution at position L235, specifically the amino acid substitution L235E (numberings according to Kabat EU index). In another embodiment, the IgG₄ Fc domain comprises an amino acid substitution at position P329, specifically the amino acid substitution P329G (numberings according to Kabat EU index). In a particular embodiment, the IgG₄ Fc domain comprises amino acid substitutions at positions S228, L235 and P329, specifically amino acid substitutions S228P, L235E and P329G (numberings according to Kabat EU index). Such IgG₄ Fc domain mutants and their Fcγ receptor binding properties are described in PCT Publication No. WO 2012/130831, incorporated herein by reference in its entirety.

In a particular embodiment, the Fc domain exhibiting reduced binding affinity to an Fc receptor and/or reduced effector function, as compared to a native IgG₁ Fc domain, is a human IgG₁ Fc domain comprising the amino acid substitutions L234A, L235A and optionally P329G, or a human IgG₄ Fc domain comprising the amino acid substitutions S228P, L235E and optionally P329G (numberings according to Kabat EU index).

In certain embodiments N-glycosylation of the Fc domain has been eliminated. In one such embodiment, the Fc domain comprises an amino acid mutation at position N297, particularly an amino acid substitution replacing asparagine by alanine (N297A) or aspartic acid (N297D) (numberings according to Kabat EU index).

In addition to the Fc domains described hereinabove and in PCT Publication No. WO 2012/130831, Fc domains with reduced Fc receptor binding and/or effector function also include those with substitution of one or more of Fc domain residues 238, 265, 269, 270, 297, 327 and 329 (U.S. Pat. No. 6,737,056) (numberings according to Kabat EU index). Such Fc mutants include Fc mutants with substitutions at two or more of amino acid positions 265, 269, 270, 297 and 327, including the so-called “DANA” Fc mutant with substitution of residues 265 and 297 to alanine (US Pat. No. 7,332,581).

Mutant Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. Alternatively, binding affinity of Fc domains or antibodies comprising an Fc domain for Fc receptors may be evaluated using cell lines known to express particular Fc receptors, such as human NK cells expressing FcγIIIa receptor.

Effector function of an Fc domain, or an antibody comprising an Fc domain, can be measured by methods known in the art. Examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S.

Patent No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, CA); and CytoTox 96® non-radioactive cytotoxicity assay (Promega, Madison, WI)). Useful effector cells for such assays include peripheral blood mononuclear cells (PBMC) and Natural Killer (NK) cells. Alternatively, or additionally, ADCC activity of the molecule of interest may be assessed in vivo, e.g. in a animal model such as that disclosed in Clynes et al., Proc Natl Acad Sci USA 95, 652-656 (1998).

In some embodiments, binding of the Fc domain to a complement component, specifically to C1q, is reduced. Accordingly, in some embodiments wherein the Fc domain is engineered to have reduced effector function, said reduced effector function includes reduced CDC. C1q binding assays may be carried out to determine whether the Fc domain, or antibody comprising the Fc domain, is able to bind C1q and hence has CDC activity. See e.g., C1q and C3c binding ELISA in WO 2006/029879 and WO 2005/100402. To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

FcRn binding and in vivo clearance/half life determinations can also be performed using methods known in the art (see, e.g., Petkova, S.B. et al., Int′l. Immunol. 18(12): 1759-1769 (2006); WO 2013/120929).

In one aspect, the invention provides an immunoconjugate comprising a mutant IL-7 polypeptide and an antibody that binds to PD-1, wherein the mutant IL-7 polypeptide is a human IL-7 molecule comprising the amino acid substitutions V15A (numbering relative to the human IL-7 sequence SEQ ID NO: 52); and wherein the antibody comprises (a) a heavy chain variable region (VH) comprising the amino acid sequence of SEQ ID NO:14, and (b) a light chain variable region (VL) comprising the amino acid sequence of SEQ ID NO: 15.

In one aspect, the invention provides an immunoconjugate comprising a mutant IL-7 polypeptide and an antibody that binds to PD-1, wherein the mutant IL-7 polypeptide is a human IL-7 molecule comprising the amino acid substitutions V15K (numbering relative to the human IL-7 sequence SEQ ID NO: 52); and wherein the antibody comprises (a) a heavy chain variable region (VH) comprising the amino acid sequence of SEQ ID NO:14, and (b) a light chain variable region (VL) comprising the amino acid sequence of SEQ ID NO: 15.

In one aspect, the invention provides an immunoconjugate comprising a mutant IL-7 polypeptide and an antibody that binds to PD-1, wherein the mutant IL-7 polypeptide is a human IL-7 molecule comprising the amino acid substitutions V18A (numbering relative to the human IL-7 sequence SEQ ID NO: 52); and wherein the antibody comprises (a) a heavy chain variable region (VH) comprising the amino acid sequence of SEQ ID NO:14, and (b) a light chain variable region (VL) comprising the amino acid sequence of SEQ ID NO:15.

In one aspect, the invention provides an immunoconjugate comprising a mutant IL-7 polypeptide and an antibody that binds to PD-1, wherein the mutant IL-7 polypeptide is a human IL-7 molecule comprising the amino acid substitutions V18K (numbering relative to the human IL-7 sequence SEQ ID NO: 52); and wherein the antibody comprises (a) a heavy chain variable region (VH) comprising the amino acid sequence of SEQ ID NO:14, and (b) a light chain variable region (VL) comprising the amino acid sequence of SEQ ID NO:15.

In one aspect, the invention provides an immunoconjugate comprising a mutant IL-7 polypeptide and an antibody that binds to PD-1, wherein the mutant IL-7 polypeptide is a human IL-7 molecule comprising the amino acid substitutions L77A (numbering relative to the human IL-7 sequence SEQ ID NO: 52); and wherein the antibody comprises (a) a heavy chain variable region (VH) comprising the amino acid sequence of SEQ ID NO:14, and (b) a light chain variable region (VL) comprising the amino acid sequence of SEQ ID NO:15.

In one aspect, the invention provides an immunoconjugate comprising a mutant IL-7 polypeptide and an antibody that binds to PD-1, wherein the mutant IL-7 polypeptide is a human IL-7 molecule comprising the amino acid substitutions L77K (numbering relative to the human IL-7 sequence SEQ ID NO: 52); and wherein the antibody comprises (a) a heavy chain variable region (VH) comprising the amino acid sequence of SEQ ID NO:14, and (b) a light chain variable region (VL) comprising the amino acid sequence of SEQ ID NO:15.

In one aspect, the invention provides an immunoconjugate comprising a mutant IL-7 polypeptide and an antibody that binds to PD-1, wherein the mutant IL-7 polypeptide is a human IL-7 molecule comprising the amino acid substitutions K81E (numbering relative to the human IL-7 sequence SEQ ID NO: 52); and wherein the antibody comprises (a) a heavy chain variable region (VH) comprising the amino acid sequence of SEQ ID NO:14, and (b) a light chain variable region (VL) comprising the amino acid sequence of SEQ ID NO:15.

In one aspect, the invention provides an immunoconjugate comprising a mutant IL-7 polypeptide and an antibody that binds to PD-1, wherein the mutant IL-7 polypeptide is a human IL-7 molecule comprising the amino acid substitutions G85K (numbering relative to the human IL-7 sequence SEQ ID NO: 52); and wherein the antibody comprises (a) a heavy chain variable region (VH) comprising the amino acid sequence of SEQ ID NO:14, and (b) a light chain variable region (VL) comprising the amino acid sequence of SEQ ID NO:15.

In one aspect, the invention provides an immunoconjugate comprising a mutant IL-7 polypeptide and an antibody that binds to PD-1, wherein the mutant IL-7 polypeptide is a human IL-7 molecule comprising the amino acid substitutions G85E (numbering relative to the human IL-7 sequence SEQ ID NO: 52); and wherein the antibody comprises (a) a heavy chain variable region (VH) comprising the amino acid sequence of SEQ ID NO:14, and (b) a light chain variable region (VL) comprising the amino acid sequence of SEQ ID NO:15.

In one aspect, the invention provides an immunoconjugate comprising a mutant IL-7 polypeptide and an antibody that binds to PD-1, wherein the mutant IL-7 polypeptide is a human IL-7 molecule comprising the amino acid substitutions I88K (numbering relative to the human IL-7 sequence SEQ ID NO: 52); and wherein the antibody comprises (a) a heavy chain variable region (VH) comprising the amino acid sequence of SEQ ID NO:14, and (b) a light chain variable region (VL) comprising the amino acid sequence of SEQ ID NO:15.

In one aspect, the invention provides an immunoconjugate comprising a mutant IL-7 polypeptide and an antibody that binds to PD-1, wherein the mutant IL-7 polypeptide is a human IL-7 molecule comprising the amino acid substitutions N143K (numbering relative to the human IL-7 sequence SEQ ID NO: 52); and wherein the antibody comprises (a) a heavy chain variable region (VH) comprising the amino acid sequence of SEQ ID NO:14, and (b) a light chain variable region (VL) comprising the amino acid sequence of SEQ ID NO:15.

In one aspect, the invention provides an immunoconjugate comprising a mutant IL-7 polypeptide and an antibody that binds to PD-1, wherein the mutant IL-7 polypeptide is a human IL-7 molecule comprising the amino acid substitutions K81E and G85K (numbering relative to the human IL-7 sequence SEQ ID NO: 52); and wherein the antibody comprises (a) a heavy chain variable region (VH) comprising the amino acid sequence of SEQ ID NO:14, and (b) a light chain variable region (VL) comprising the amino acid sequence of SEQ ID NO:15.

In one aspect, the invention provides an immunoconjugate comprising a mutant IL-7 polypeptide and an antibody that binds to PD-1, wherein the mutant IL-7 polypeptide is a human IL-7 molecule comprising the amino acid substitutions K81E and G85E (numbering relative to the human IL-7 sequence SEQ ID NO: 52); and wherein the antibody comprises (a) a heavy chain variable region (VH) comprising the amino acid sequence of SEQ ID NO:14, and (b) a light chain variable region (VL) comprising the amino acid sequence of SEQ ID NO:15

In one aspect, the invention provides an immunoconjugate comprising a mutant IL-7 polypeptide and an antibody that binds to PD-1, wherein the mutant IL-7 polypeptide comprises the amino acid sequence of SEQ ID NO: 55, and wherein the antibody comprises (a) a heavy chain variable region (VH) comprising the amino acid sequence of SEQ ID NO:14, and (b) a light chain variable region (VL) comprising the amino acid sequence of SEQ ID NO:15.

In one aspect, the invention provides an immunoconjugate comprising a mutant IL-7 polypeptide and an antibody that binds to PD-1, wherein the mutant IL-7 polypeptide comprises the amino acid sequence of SEQ IN NO: 56, and wherein the antibody comprises (a) a heavy chain variable region (VH) comprising the amino acid sequence of SEQ ID NO:14, and (b) a light chain variable region (VL) comprising the amino acid sequence of SEQ ID NO:15.

In one aspect, the invention provides an immunoconjugate comprising a mutant IL-7 polypeptide and an antibody that binds to PD-1, wherein the mutant IL-7 polypeptide comprises the amino acid sequence of SEQ ID NO: 57, and wherein the antibody comprises (a) a heavy chain variable region (VH) comprising the amino acid sequence of SEQ ID NO:14, and (b) a light chain variable region (VL) comprising the amino acid sequence of SEQ ID NO:15.

In one aspect, the invention provides an immunoconjugate comprising a mutant IL-7 polypeptide and an antibody that binds to PD-1, wherein the mutant IL-7 polypeptide comprises the amino acid sequence of SEQ ID NO: 58, and wherein the antibody comprises (a) a heavy chain variable region (VH) comprising the amino acid sequence of SEQ ID NO:14, and (b) a light chain variable region (VL) comprising the amino acid sequence of SEQ ID NO:15.

In one aspect, the invention provides an immunoconjugate comprising a mutant IL-7 polypeptide and an antibody that binds to PD-1, wherein the mutant IL-7 polypeptide comprises the amino acid sequence of SEQ ID NO: 67, and SEQ ID NO: 79; and wherein the antibody comprises (a) a heavy chain variable region (VH) comprising the amino acid sequence of SEQ ID NO:14, and (b) a light chain variable region (VL) comprising the amino acid sequence of SEQ ID NO:15.

In one aspect, the invention provides an immunoconjugate comprising a mutant IL-7 polypeptide and an antibody that binds to PD-1, wherein the mutant IL-7 polypeptide comprises the amino acid sequence of SEQ ID NO: 68, and wherein the antibody comprises (a) a heavy chain variable region (VH) comprising the amino acid sequence of SEQ ID NO:14, and (b) a light chain variable region (VL) comprising the amino acid sequence of SEQ ID NO:15.

In one aspect, the invention provides an immunoconjugate comprising a mutant IL-7 polypeptide and an antibody that binds to PD-1, wherein the mutant IL-7 polypeptide comprises the amino acid sequence of SEQ ID NO: 70, and wherein the antibody comprises (a) a heavy chain variable region (VH) comprising the amino acid sequence of SEQ ID NO:14, and (b) a light chain variable region (VL) comprising the amino acid sequence of SEQ ID NO:15.

In one aspect, the invention provides an immunoconjugate comprising a mutant IL-7 polypeptide and an antibody that binds to PD-1, wherein the mutant IL-7 polypeptide comprises the amino acid sequence of SEQ ID NO: 72, and wherein the antibody comprises (a) a heavy chain variable region (VH) comprising the amino acid sequence of SEQ ID NO:14, and (b) a light chain variable region (VL) comprising the amino acid sequence of SEQ ID NO:15.

In one aspect, the invention provides an immunoconjugate comprising a mutant IL-7 polypeptide and an antibody that binds to PD-1, wherein the mutant IL-7 polypeptide comprises the amino acid sequence of SEQ ID NO: 73, and wherein the antibody comprises (a) a heavy chain variable region (VH) comprising the amino acid sequence of SEQ ID NO:14, and (b) a light chain variable region (VL) comprising the amino acid sequence of SEQ ID NO:15.

In one aspect, the invention provides an immunoconjugate comprising a mutant IL-7 polypeptide and an antibody that binds to PD-1, wherein the mutant IL-7 polypeptide comprises the amino acid sequence of SEQ ID NO: 74, and wherein the antibody comprises (a) a heavy chain variable region (VH) comprising the amino acid sequence of SEQ ID NO:14, and (b) a light chain variable region (VL) comprising the amino acid sequence of SEQ ID NO:15.

In one aspect, the invention provides an immunoconjugate comprising a mutant IL-7 polypeptide and an antibody that binds to PD-1, wherein the mutant IL-7 polypeptide comprises the amino acid sequence of SEQ ID NO: 79, and wherein the antibody comprises (a) a heavy chain variable region (VH) comprising the amino acid sequence of SEQ ID NO:14, and (b) a light chain variable region (VL) comprising the amino acid sequence of SEQ ID NO:15.

In one aspect, the invention provides an immunoconjugate comprising a mutant IL-7 polypeptide and an antibody that binds to PD-1, wherein the mutant IL-7 polypeptide comprises the amino acid sequence of SEQ ID NO: 135, and wherein the antibody comprises (a) a heavy chain variable region (VH) comprising the amino acid sequence of SEQ ID NO:14, and (b) a light chain variable region (VL) comprising the amino acid sequence of SEQ ID NO:15.

In one aspect, the invention provides an immunoconjugate comprising a mutant IL-7 polypeptide and an antibody that binds to PD-1, wherein the mutant IL-7 polypeptide comprises the amino acid sequence of SEQ ID NO: 136, and wherein the antibody comprises (a) a heavy chain variable region (VH) comprising the amino acid sequence of SEQ ID NO:14, and (b) a light chain variable region (VL) comprising the amino acid sequence of SEQ ID NO: 15.

In one embodiment according to any of the above aspects of the invention, the antibody is an IgG class immunoglobulin, comprising a human IgG₁ Fc domain composed of a first and a second subunit, wherein in the first subunit of the Fc domain the threonine residue at position 366 is replaced with a tryptophan residue (T366W), and in the second subunit of the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numberings according to Kabat EU index), and wherein further each subunit of the Fc domain comprises the amino acid substitutions L234A, L235A and P329G (Kabat EU index numbering). In this embodiment, the mutant IL-7 polypeptide may be fused at its amino-terminal amino acid to the carboxy-terminal amino acid of the first subunit of the Fc domain, through a linker peptide of SEQ ID NO: 21.

In one aspect, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO:85, a polypeptide comprising an amino acid sequence that is at least about 80%, 85%, 90%, 95%, 96%, 97%, 98%, 99% or 100% identical to the sequence of SEQ ID NO:86, and a polypeptide comprising an amino acid sequence that is at least about 80%, 85%, 90%, 95%, 96%, 97%, 98%, 99% or 100% identical to the sequence of SEQ ID NO:90.

In one aspect, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO:85, a polypeptide comprising an amino acid sequence that is at least about 80%, 85%, 90%, 95%, 96%, 97%, 98%, 99% or 100% identical to the sequence of SEQ ID NO:86, and a polypeptide comprising an amino acid sequence that is at least about 80%, 85%, 90%, 95%, 96%, 97%, 98%, 99% or 100% identical to the sequence of SEQ ID NO:91.

In one aspect, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO:85, a polypeptide comprising an amino acid sequence that is at least about 80%, 85%, 90%, 95%, 96%, 97%, 98%, 99% or 100% identical to the sequence of SEQ ID NO:86, and a polypeptide comprising an amino acid sequence that is at least about 80%, 85%, 90%, 95%, 96%, 97%, 98%, 99% or 100% identical to the sequence of SEQ ID NO:92.

In one aspect, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO:85, a polypeptide comprising an amino acid sequence that is at least about 80%, 85%, 90%, 95%, 96%, 97%, 98%, 99% or 100% identical to the sequence of SEQ ID NO:86, and a polypeptide comprising an amino acid sequence that is at least about 80%, 85%, 90%, 95%, 96%, 97%, 98%, 99% or 100% identical to the sequence of SEQ ID NO:93.

In one aspect, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO:85, a polypeptide comprising an amino acid sequence that is at least about 80%, 85%, 90%, 95%, 96%, 97%, 98%, 99% or 100% identical to the sequence of SEQ ID NO:86, and a polypeptide comprising an amino acid sequence that is at least about 80%, 85%, 90%, 95%, 96%, 97%, 98%, 99% or 100% identical to the sequence of SEQ ID NO:102.

In one aspect, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO:85, a polypeptide comprising an amino acid sequence that is at least about 80%, 85%, 90%, 95%, 96%, 97%, 98%, 99% or 100% identical to the sequence of SEQ ID NO:86, and a polypeptide comprising an amino acid sequence that is at least about 80%, 85%, 90%, 95%, 96%, 97%, 98%, 99% or 100% identical to the sequence of SEQ ID NO:103.

In one aspect, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO:85, a polypeptide comprising an amino acid sequence that is at least about 80%, 85%, 90%, 95%, 96%, 97%, 98%, 99% or 100% identical to the sequence of SEQ ID NO:86, and a polypeptide comprising an amino acid sequence that is at least about 80%, 85%, 90%, 95%, 96%, 97%, 98%, 99% or 100% identical to the sequence of SEQ ID NO:105.

In one aspect, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO:85, a polypeptide comprising an amino acid sequence that is at least about 80%, 85%, 90%, 95%, 96%, 97%, 98%, 99% or 100% identical to the sequence of SEQ ID NO:86, and a polypeptide comprising an amino acid sequence that is at least about 80%, 85%, 90%, 95%, 96%, 97%, 98%, 99% or 100% identical to the sequence of SEQ ID NO:107.

In one aspect, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO:85, a polypeptide comprising an amino acid sequence that is at least about 80%, 85%, 90%, 95%, 96%, 97%, 98%, 99% or 100% identical to the sequence of SEQ ID NO:86, and a polypeptide comprising an amino acid sequence that is at least about 80%, 85%, 90%, 95%, 96%, 97%, 98%, 99% or 100% identical to the sequence of SEQ ID NO:108.

In one aspect, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO:85, a polypeptide comprising an amino acid sequence that is at least about 80%, 85%, 90%, 95%, 96%, 97%, 98%, 99% or 100% identical to the sequence of SEQ ID NO:86, and a polypeptide comprising an amino acid sequence that is at least about 80%, 85%, 90%, 95%, 96%, 97%, 98%, 99% or 100% identical to the sequence of SEQ ID NO:109.

In one aspect, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO:85, a polypeptide comprising an amino acid sequence that is at least about 80%, 85%, 90%, 95%, 96%, 97%, 98%, 99% or 100% identical to the sequence of SEQ ID NO:86, and a polypeptide comprising an amino acid sequence that is at least about 80%, 85%, 90%, 95%, 96%, 97%, 98%, 99% or 100% identical to the sequence of SEQ ID NO:114.

In one aspect, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO:85, a polypeptide comprising an amino acid sequence that is at least about 80%, 85%, 90%, 95%, 96%, 97%, 98%, 99% or 100% identical to the sequence of SEQ ID NO:86, and a polypeptide comprising an amino acid sequence that is at least about 80%, 85%, 90%, 95%, 96%, 97%, 98%, 99% or 100% identical to the sequence of SEQ ID NO: 137.

In one aspect, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO:85, a polypeptide comprising an amino acid sequence that is at least about 80%, 85%, 90%, 95%, 96%, 97%, 98%, 99% or 100% identical to the sequence of SEQ ID NO:86, and a polypeptide comprising an amino acid sequence that is at least about 80%, 85%, 90%, 95%, 96%, 97%, 98%, 99% or 100% identical to the sequence of SEQ ID NO:138.

### POLYNUCLEOTIDES

The invention further provides isolated polynucleotides encoding an immunoconjugate as described herein or a fragment thereof. In some embodiments, said fragment is an antigen binding fragment.

The polynucleotides encoding immunoconjugates of the invention may be expressed as a single polynucleotide that encodes the entire immunoconjugate or as multiple (e.g., two or more) polynucleotides that are co-expressed. Polypeptides encoded by polynucleotides that are co-expressed may associate through, e.g., disulfide bonds or other means to form a functional immunoconjugate. For example, the light chain portion of an antibody may be encoded by a separate polynucleotide from the portion of the immunoconjugate comprising the heavy chain portion of the antibody and the mutant IL-7 polypeptide. When co-expressed, the heavy chain polypeptides will associate with the light chain polypeptides to form the immunoconjugate. In another example, the portion of the immunoconjugate comprising one of the two Fc domain subunits and the mutant IL-7 polypeptide could be encoded by a separate polynucleotide from the portion of the immunoconjugate comprising the the other of the two Fc domain subunits. When co-expressed, the Fc domain subunits will associate to form the Fc domain.

In some embodiments, the isolated polynucleotide encodes the entire immunoconjugate according to the invention as described herein. In other embodiments, the isolated polynucleotide encodes a polypeptide comprised in the immunoconjugate according to the invention as described herein.

In one embodiment, an isolated polynucleotide of the invention encodes the heavy chain of the antibody comprised in the immunoconjugate (e.g. an immunoglobulin heavy chain), and the mutant IL-7 polypeptide. In another embodiment, an isolated polynucleotide of the invention encodes the light chain of the antibody comprised in the immunoconjugate.

In certain embodiments the polynucleotide or nucleic acid is DNA. In other embodiments, a polynucleotide of the present invention is RNA, for example, in the form of messenger RNA (mRNA). RNA of the present invention may be single stranded or double stranded.

### RECOMBINANT METHODS

Mutant IL-7 polypeptides useful in the invention can be prepared by deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.. The sequence of native human IL-7 is shown in SEQ ID NO: 52. Substitution or insertion may involve natural as well as non-natural amino acid residues. Amino acid modification includes well known methods of chemical modification such as the addition of glycosylation sites or carbohydrate attachments, and the like.

Immunoconjugates of the invention may be obtained, for example, by solid-state peptide synthesis (e.g. Merrifield solid phase synthesis) or recombinant production. For recombinant production one or more polynucleotide encoding the immunoconjugate (fragment), e.g., as described above, is isolated and inserted into one or more vectors for further cloning and/or expression in a host cell. Such polynucleotide may be readily isolated and sequenced using conventional procedures. In one embodiment a vector, preferably an expression vector, comprising one or more of the polynucleotides of the invention is provided. Methods which are well known to those skilled in the art can be used to construct expression vectors containing the coding sequence of an immunoconjugate (fragment) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y (1989). The expression vector can be part of a plasmid, virus, or may be a nucleic acid fragment. The expression vector includes an expression cassette into which the polynucleotide encoding the immunoconjugate (fragment) (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g. on a single vector, or in separate polynucleotide constructs, e.g. on separate (different) vectors. Furthermore, any vector may contain a single coding region, or may comprise two or more coding regions, e.g. a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the immunoconjugate of the invention, or variant or derivative thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as inducible promoters (e.g. promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present invention may be associated with additional coding regions which encode secretory or signal peptides, which direct the secretion of a polypeptide encoded by a polynucleotide of the present invention. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

DNA encoding a short protein sequence that could be used to facilitate later purification (e.g. a histidine tag) or assist in labeling the immunoconjugate may be included within or at the ends of the immunoconjugate (fragment) encoding polynucleotide.

In a further embodiment, a host cell comprising one or more polynucleotides of the invention is provided. In certain embodiments a host cell comprising one or more vectors of the invention is provided. The polynucleotides and vectors may incorporate any of the features, singly or in combination, described herein in relation to polynucleotides and vectors, respectively. In one such embodiment a host cell comprises (e.g. has been transformed or transfected with) one or more vector comprising one or more polynucleotide that encodes the immunoconjugate of the invention. As used herein, the term “host cell” refers to any kind of cellular system which can be engineered to generate the immunoconjugates of the invention or fragments thereof. Host cells suitable for replicating and for supporting expression of immunoconjugates are well known in the art. Such cells may be transfected or transduced as appropriate with the particular expression vector and large quantities of vector containing cells can be grown for seeding large scale fermenters to obtain sufficient quantities of the immunoconjugate for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006). Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. US Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr⁻ CHO cells (Urlaub et al., Proc Natl Acad Sci USA 77, 4216 (1980)); and myeloma cell lines such as YO, NS0, P3X63 and Sp2/0. For a review of certain mammalian host cell lines suitable for protein production, see, e.g., Yazaki and Wu, Methods in Molecular Biology, Vol. 248 (B.K.C. Lo, ed., Humana Press, Totowa, NJ), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or a lymphoid cell (e.g., Y0, NS0, Sp20 cell).

Standard technologies are known in the art to express foreign genes in these systems. Cells expressing a mutant-IL-7 polypeptide fused to either the heavy or the light chain of an antibody may be engineered so as to also express the other of the antibody chains such that the expressed mutant IL-7 fusion product is an antibody that has both a heavy and a light chain.

In one embodiment, a method of producing an immunoconjugate according to the invention is provided, wherein the method comprises culturing a host cell comprising one or more polynucleotide encoding the immunoconjugate, as provided herein, under conditions suitable for expression of the immunoconjugate, and optionally recovering the immunoconjugate from the host cell (or host cell culture medium).

In the immunoconjugate of the invention, the mutant IL-7 polypeptide may be genetically fused to the antibody, or may be chemically conjugated to the antibody. Genetic fusion of the IL-7 polypeptide to the antibody can be designed such that the IL-7 sequence is fused directly to the polypeptide or indirectly through a linker sequence. The composition and length of the linker may be determined in accordance with methods well known in the art and may be tested for efficacy. Particular linker peptides are described herein. Additional sequences may also be included to incorporate a cleavage site to separate the individual components of the fusion if desired, for example an endopeptidase recognition sequence. In addition, an IL-7 fusion protein may also be synthesized chemically using methods of polypeptide synthesis as is well known in the art (e.g. Merrifield solid phase synthesis). Mutant IL-7 polypeptides may be chemically conjugated to other molecules, e.g. antibodies, using well known chemical conjugation methods. Bi-functional cross-linking reagents such as homofunctional and heterofunctional cross-linking reagents well known in the art can be used for this purpose. The type of cross-linking reagent to use depends on the nature of the molecule to be coupled to IL-7 and can readily be identified by those skilled in the art. Alternatively, or in addition, mutant IL-7 and/or the molecule to which it is intended to be conjugated may be chemically derivatized such that the two can be conjugated in a separate reaction as is also well known in the art.

The immunoconjugates of the invention comprise an antibody. Methods to produce antibodies are well known in the art (see e.g. Harlow and Lane, “Antibodies, a laboratory manual”, Cold Spring Harbor Laboratory, 1988). Non-naturally occurring antibodies can be constructed using solid phase-peptide synthesis, can be produced recombinantly (e.g. as described in U.S. Pat. No. 4,186,567) or can be obtained, for example, by screening combinatorial libraries comprising variable heavy chains and variable light chains (see e.g. U.S. Pat.. No. 5,969,108 to McCafferty). Immunoconjugates, antibodies, and methods for producing the same are also described in detail e.g. in PCT Publication Nos. WO 2011/020783, WO 2012/107417, and WO 2012/146628, each of which are incorporated herein by reference in their entirety.

Any animal species of antibody may be used in the immunoconjugates of the invention. Non-limiting antibodies useful in the present invention can be of murine, primate, or human origin. If the immunoconjugate is intended for human use, a chimeric form of antibody may be used wherein the constant regions of the antibody are from a human. A humanized or fully human form of the antibody can also be prepared in accordance with methods well known in the art (see e. g. U.S. Pat. No. 5,565,332 to Winter). Humanization may be achieved by various methods including, but not limited to (a) grafting the non-human (e.g., donor antibody) CDRs onto human (e.g. recipient antibody) framework and constant regions with or without retention of critical framework residues (e.g. those that are important for retaining good antigen binding affinity or antibody functions), (b) grafting only the non-human specificity-determining regions (SDRs or a-CDRs; the residues critical for the antibody-antigen interaction) onto human framework and constant regions, or (c) transplanting the entire non-human variable domains, but “cloaking” them with a human-like section by replacement of surface residues. Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat′l Acad. Sci. USA 86:10029-10033 (1989); US Pat. Nos. 5, 821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall’ Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling). Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

Human antibodies can be produced using various techniques known in the art. Human antibodies are described generally in van Dijk and van de Winkel, Curr Opin Pharmacol 5, 368-74 (2001) and Lonberg, Curr Opin Immunol 20, 450-459 (2008). Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal’s chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HuMAB@ technology; U.S. Pat. No. 7,041,870 describing K-M MOUSE® technology, and U.S. Pat. Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolation from human antibody libraries, as described herein.

Antibodies useful in the invention may be isolated by screening combinatorial libraries for antibodies with the desired activity or activities. Methods for screening combinatorial libraries are reviewed, e.g., in Lerner et al. in Nature Reviews 16:498-508 (2016). For example, a variety of methods are known in the art for generating phage display libraries and screening such libraries for antibodies possessing the desired binding characteristics. Such methods are reviewed, e.g., in Frenzel et al. in mAbs 8:1177-1194 (2016); Bazan et al. in Human Vaccines and Immunotherapeutics 8:1817-1828 (2012) and Zhao et al. in Critical Reviews in Biotechnology 36:276-289 (2016) as well as in Hoogenboom et al. in Methods in Molecular Biology 178:1-37 (O′Brien et al., ed., Human Press, Totowa, NJ, 2001) and in Marks and Bradbury in Methods in Molecular Biology 248:161-175 (Lo, ed., Human Press, Totowa, NJ, 2003).

In certain phage display methods, repertoires of VH and VL genes are separately cloned by polymerase chain reaction (PCR) and recombined randomly in phage libraries, which can then be screened for antigen-binding phage as described in Winter et al. in Annual Review of Immunology 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al. in EMBO Journal 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter in Journal of Molecular Biology 227: 381-388 (1992). Patent publications describing human antibody phage libraries include, for example: U.S. Pat. Nos. 5,750,373; 7,985,840; 7,785,903 and 8,679,490 as well as U.S. Pat. Publication Nos. 2005/0079574, 2007/0117126, 2007/0237764 and 2007/0292936. Further examples of methods known in the art for screening combinatorial libraries for antibodies with a desired activity or activities include ribosome and mRNA display, as well as methods for antibody display and selection on bacteria, mammalian cells, insect cells or yeast cells. Methods for yeast surface display are reviewed, e.g., in Scholler et al. in Methods in Molecular Biology 503:135-56 (2012) and in Cherf et al. in Methods in Molecular biology 1319:155-175 (2015) as well as in the Zhao et al. in Methods in Molecular Biology 889:73-84 (2012). Methods for ribosome display are described, e.g., in He et al. in Nucleic Acids Research 25:5132-5134 (1997) and in Hanes et al. in PNAS 94:4937-4942 (1997).

Further chemical modification of the immunoconjugate of the invention may be desirable. For example, problems of immunogenicity and short half-life may be improved by conjugation to substantially straight chain polymers such as polyethylene glycol (PEG) or polypropylene glycol (PPG) (see e.g. WO 87/00056).

Immunoconjugates prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the immunoconjugate binds. For example, an antibody which specifically binds the mutant IL-7 polypeptide may be used. For affinity chromatography purification of immunoconjugates of the invention, a matrix with protein A or protein G may be used. For example, sequential Protein A or G affinity chromatography and size exclusion chromatography can be used to isolate an immunoconjugate essentially as described in the Examples. The purity of the immunoconjugate can be determined by any of a variety of well known analytical methods including gel electrophoresis, high pressure liquid chromatography, and the like.

### COMPOSITIONS, FORMULATIONS, AND ROUTES OF ADMINISTRATION

In a further aspect, the invention provides pharmaceutical compositions comprising an immunoconjugate as described herein, e.g., for use in any of the below therapeutic methods. In one embodiment, a pharmaceutical composition comprises any of the immunoconjugates provided herein and a pharmaceutically acceptable carrier. In another embodiment, a pharmaceutical composition comprises any of the immunoconjugates provided herein and at least one additional therapeutic agent, e.g., as described below.

Further provided is a method of producing an immunoconjugate of the invention in a form suitable for administration in vivo, the method comprising (a) obtaining an immunoconjugate according to the invention, and (b) formulating the immunoconjugate with at least one pharmaceutically acceptable carrier, whereby a preparation of immunoconjugate is formulated for administration in vivo.

Pharmaceutical compositions of the present invention comprise a therapeutically effective amount of immunoconjugate dissolved or dispersed in a pharmaceutically acceptable carrier. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains immunoconjugate and optionally an additional active ingredient will be known to those of skill in the art in light of the present disclosure, as exemplified by Remington’s Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards or corresponding authorities in other countries. Preferred compositions are lyophilized formulations or aqueous solutions. As used herein, “pharmaceutically acceptable carrier” includes any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington’s Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

An immunoconjugate of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic.

Parenteral compositions include those designed for administration by injection, e.g. subcutaneous, intradermal, intralesional, intravenous, intraarterial intramuscular, intrathecal or intraperitoneal injection. For injection, the immunoconjugates of the invention may be formulated in aqueous solutions, preferably in physiologically compatible buffers such as Hanks’ solution, Ringer’s solution, or physiological saline buffer. The solution may contain formulatory agents such as suspending, stabilizing and/or dispersing agents. Alternatively, the immunoconjugates may be in powder form for constitution with a suitable vehicle, e.g., sterile pyrogen-free water, before use. Sterile injectable solutions are prepared by incorporating the immunoconjugates of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. Suitable pharmaceutically acceptable carriers include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions. Such techniques are disclosed in Remington’s Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g. films, or microcapsules. In particular embodiments, prolonged absorption of an injectable composition can be brought about by the use in the compositions of agents delaying absorption, such as, for example, aluminum monostearate, gelatin or combinations thereof.

In addition to the compositions described previously, the immunoconjugates may also be formulated as a depot preparation. Such long acting formulations may be administered by implantation (for example subcutaneously or intramuscularly) or by intramuscular injection. Thus, for example, the immunoconjugates may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising the immunoconjugates of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The immunoconjugates may be formulated into a composition in a free acid or base, neutral or salt form. Pharmaceutically acceptable salts are salts that substantially retain the biological activity of the free acid or base. These include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

### THERAPEUTIC METHODS AND COMPOSITIONS

Any of the mutant IL-7 polypeptides and immunoconjugates provided herein may be used in therapeutic methods. Mutant IL-7 polypeptides and immunoconjugates of the invention may be used as immunotherapeutic agents, for example in the treatment of cancers.

For use in therapeutic methods, mutant IL-7 polypeptides and immunoconjugates of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

Mutant IL-7 polypeptides and immunoconjugates of the invention may be particularly useful in treating disease states where stimulation of the immune system of the host is beneficial, in particular conditions where an enhanced cellular immune response is desirable. These may include disease states where the host immune response is insufficient or deficient. Disease states for which the mutant IL-7 polypeptides and the immunoconjugates of the invention may be administered comprise, for example, a tumor or infection where a cellular immune response would be a critical mechanism for specific immunity. The mutant IL-7 polypeptides and the immunoconjugates of the invention may be administered per se or in any suitable pharmaceutical composition.

In one aspect, mutant IL-7 polypeptides and immunoconjugates of the invention for use as a medicament are provided. In further aspects, mutant IL-7 polypeptides and immunoconjugates of the invention for use in treating a disease are provided. In certain embodiments, mutant IL-7 polypeptides and immunoconjugates of the invention for use in a method of treatment are provided. In one embodiment, the invention provides an immunoconjugate as described herein for use in the treatment of a disease in an individual in need thereof. In one embodiment, the invention provides a mutant IL-7 poypeptide as described herein for use in the treatment of a disease in an individual in need thereof. In certain embodiments, the invention provides a mutant IL-7 and an immunoconjugate for use in a method of treating an individual having a disease comprising administering to the individual a therapeutically effective amount of the immunoconjugate. In certain embodiments the disease to be treated is a proliferative disorder. In a particular embodiment the disease is cancer. In certain embodiments the method further comprises administering to the individual a therapeutically effective amount of at least one additional therapeutic agent, e.g., an anti-cancer agent if the disease to be treated is cancer. In further embodiments, the invention provides an immunoconjugate for use in stimulating the immune system. In certain embodiments, the invention provides a mutant IL-7 and/or an immunoconjugate for use in a method of stimulating the immune system in an individual comprising administering to the individual an effective amount of the immunoconjugate to stimulate the immune system. An “individual” according to any of the above embodiments is a mammal, preferably a human. “Stimulation of the immune system” according to any of the above embodiments may include any one or more of a general increase in immune function, an increase in T cell function, an increase in B cell function, a restoration of lymphocyte function, an increase in the expression of IL-2 receptors, an increase in T cell responsiveness, an increase in natural killer cell activity or lymphokine-activated killer (LAK) cell activity, and the like.

In a further aspect, the invention provides for the use of a mutant IL-7 and/or an immunconjugate of the invention in the manufacture or preparation of a medicament. In one embodiment, the medicament is for the treatment of a disease in an individual in need thereof. In one embodiment, the medicament is for use in a method of treating a disease comprising administering to an individual having the disease a therapeutically effective amount of the medicament. In certain embodiments the disease to be treated is a proliferative disorder. In a particular embodiment the disease is cancer. In one embodiment, the method further comprises administering to the individual a therapeutically effective amount of at least one additional therapeutic agent, e.g., an anti-cancer agent if the disease to be treated is cancer. In a further embodiment, the medicament is for stimulating the immune system. In a further embodiment, the medicament is for use in a method of stimulating the immune system in an individual comprising administering to the individual an effective amount of the medicament to stimulate the immune system. An “individual” according to any of the above embodiments may be a mammal, preferably a human. “Stimulation of the immune system” according to any of the above embodiments may include any one or more of a general increase in immune function, an increase in T cell function, an increase in B cell function, a restoration of lymphocyte function, an increase in the expression of IL-2 receptors, an increase in T cell responsiveness, an increase in natural killer cell activity or lymphokine-activated killer (LAK) cell activity, and the like.

In a further aspect, the invention provides a method for treating a disease in an individual. In one embodiment, the method comprises administering to an individual having such disease a therapeutically effective amount of a mutant IL-7 and/or an immunoconjugate of the invention. In one embodiment a composition is administered to said invididual, comprising the mutant IL-7 and/or the immunoconjugate of the invention in a pharmaceutically acceptable form. In certain embodiments the disease to be treated is a proliferative disorder. In a particular embodiment the disease is cancer. In certain embodiments the method further comprises administering to the individual a therapeutically effective amount of at least one additional therapeutic agent, e.g., an anti-cancer agent if the disease to be treated is cancer. In a further aspect, the invention provides a method for stimulating the immune system in an individual, comprising administering to the individual an effective amount of a mutant IL-7 and/or an immunoconjugate to stimulate the immune system. An “individual” according to any of the above embodiments may be a mammal, preferably a human. “Stimulation of the immune system” according to any of the above embodiments may include any one or more of a general increase in immune function, an increase in T cell function, an increase in B cell function, a restoration of lymphocyte function, an increase in the expression of IL-2 receptors, an increase in T cell responsiveness, an increase in natural killer cell activity or lymphokine-activated killer (LAK) cell activity, and the like.

In certain embodiments the disease to be treated is a proliferative disorder, particularly cancer. Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other cell proliferation disorders that may be treated using an immunoconjugate of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments the cancer is chosen from the group consisting of kidney cancer, skin cancer, lung cancer, colorectal cancer, breast cancer, brain cancer, head and neck cancer, prostate cancer and bladder cancer. A skilled artisan readily recognizes that in many cases the immunoconjugates may not provide a cure but may only provide partial benefit. In some embodiments, a physiological change having some benefit is also considered therapeutically beneficial. Thus, in some embodiments, an amount of immunoconjugate that provides a physiological change is considered an “effective amount” or a “therapeutically effective amount”. The subject, patient, or individual in need of treatment is typically a mammal, more specifically a human.

In some embodiments, an effective amount of an immunoconjugate of the invention is administered to a cell. In other embodiments, a therapeutically effective amount of an immunoconjugates of the invention is administered to an individual for the treatment of disease.

For the prevention or treatment of disease, the appropriate dosage of an immunoconjugate of the invention (when used alone or in combination with one or more other additional therapeutic agents) will depend on the type of disease to be treated, the route of administration, the body weight of the patient, the type of molecule (e.g. comprising an Fc domain or not), the severity and course of the disease, whether the immunoconjugate is administered for preventive or therapeutic purposes, previous or concurrent therapeutic interventions, the patient’s clinical history and response to the immunoconjugate, and the discretion of the attending physician.. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The immunoconjugate is suitably administered to the patient at one time or over a series of treatments. Depending on the type and severity of the disease, about 1 µg/kg to 15 mg/kg (e.g. 0.1 mg/kg - 10 mg/kg) of immunoconjugate can be an initial candidate dosage for administration to the patient, whether, for example, by one or more separate administrations, or by continuous infusion. One typical daily dosage might range from about 1 µg/kg to 100 mg/kg or more, depending on the factors mentioned above. For repeated administrations over several days or longer, depending on the condition, the treatment would generally be sustained until a desired suppression of disease symptoms occurs. One exemplary dosage of the immunoconjugate would be in the range from about 0.005 mg/kg to about 10 mg/kg. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above. Thus, one or more doses of about 0.5 mg/kg, 2.0 mg/kg, 5.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the immunoconjugate). An initial higher loading dose, followed by one or more lower doses may be administered. However, other dosage regimens may be useful. The progress of this therapy is easily monitored by conventional techniques and assays.

The immunoconjugates of the invention will generally be used in an amount effective to achieve the intended purpose. For use to treat or prevent a disease condition, the immunoconjugates of the invention, or pharmaceutical compositions thereof, are administered or applied in a therapeutically effective amount. Determination of a therapeutically effective amount is well within the capabilities of those skilled in the art, especially in light of the detailed disclosure provided herein.

For systemic administration, a therapeutically effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the immunoconjugates which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by HPLC.

In cases of local administration or selective uptake, the effective local concentration of the immunoconjugates may not be related to plasma concentration. One having skill in the art will be able to optimize therapeutically effective local dosages without undue experimentation.

A therapeutically effective dose of the immunoconjugates described herein will generally provide therapeutic benefit without causing substantial toxicity. Toxicity and therapeutic efficacy of an immunoconjugate can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. Immunoconjugates that exhibit large therapeutic indices are preferred. In one embodiment, the immunoconjugate according to the present invention exhibits a high therapeutic index. The data obtained from cell culture assays and animal studies can be used in formulating a range of dosages suitable for use in humans. The dosage lies preferably within a range of circulating concentrations that include the ED₅₀ with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient’s condition. (See, e.g., Fingl et al., 1975, In: The Pharmacological Basis of Therapeutics, Ch. 1, p. 1, incorporated herein by reference in its entirety).

The attending physician for patients treated with immunoconjugates of the invention would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

The maximum therapeutic dose of an immunoconjugate comprising a mutant IL-7 polypeptide as described herein may be increased from those used for an immunoconjugate comprising wild-type IL-7.

### OTHER AGENTS AND TREATMENTS

The immunoconjugates according to the invention may be administered in combination with one or more other agents in therapy. For instance, an immunoconjugate of the invention may be co-administered with at least one additional therapeutic agent. The term “therapeutic agent” encompasses any agent administered to treat a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers. In a particular embodiment, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent.

Such other agents are suitably present in combination in amounts that are effective for the purpose intended. The effective amount of such other agents depends on the amount of immunoconjugate used, the type of disorder or treatment, and other factors discussed above. The immunoconjugates are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the immunoconjugate of the invention can occur prior to, simultaneously, and/or following, administration of the additional therapeutic agent and/or adjuvant. Immunoconjugates of the invention may also be used in combination with radiation therapy.

### ARTICLES OF MANUFACTURE

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an immunoconjugate of the invention. The label or package insert indicates that the composition is used for treating the condition of choice. Moreover, the article of manufacture may comprise (a) a first container with a composition contained therein, wherein the composition comprises an immunoconjugate of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer’s solution and dextrose solution. It may further include other materials desirable from a commercial and user standpoint, including other buffers, diluents, filters, needles, and syringes.

Amino Acid Sequences

| | Amino Acid Sequence | SEQ ID NO |
|---|---|---|
| PD-1 minimal HVR-H1 | SSYT | 1 |
| PD-1 minimal HVR-H2 | SGGGRDIY | 2 |
| PD-1 minimal HVR-H3 | GRVYF | 3 |
| PD-1 minimal HVR-L1 | TSDNSF | 4 |
| PD-1 minimal HVR-L2 | RSSTLES | 5 |
| PD-1 minimal HVR-L3 | NYDVPW | 6 |
| fragment of FR-H3 (RDN at Kabat pos. 71-73) | RDN | 7 |
| PD-1 HVR-H1 | GFSFSSY | 8 |
| PD-1 HVR-H2 | GGR | 9 |
| PD-1 HVR-H3 | TGRVYFALD | 10 |
| PD-1 HVR-L1 | SESVDTSDNSF | 11 |
| PD-1 HVR-L2 | RSS | 12 |
| PD-1 HVR-L3 | NYDVPW | 13 |
| PD-1 VH (1, 2, 3, 4) | EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSS | 14 |
| PD-1 VL (1) | DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIK | 15 |
| PD-1 VL (2) | DVVMTQSPLSLPVTLGQPASISCRASESVDTSDNSFIHWYQQRPGQSPRLLIYRSSTLESGVPDRFSGSGSGTDFTLKISRVEAEDVGVYYCQQNYDVPWTFGQGTKVEIK | 16 |
| PD-1 VL (3) | EIVLTQSPATLSLSPGERATLSCRASESVDTSDNSFIHWYQQKPGQSPRLLIYRSSTLESGIPARFSGSGSGTDFTLTISSLEPEDFAVYYCQQNYDVPWTFGQGTKVEIK | 17 |
| PD- 1 VL (4) | EIVLTQSPATLSLSPGERATLSCRASESVDTSDNSFIHWYQQKPGQSPRLLIYRSSTLESGIPARFSGSGSGTDFTLTISSLEPEDFAVYYCQQNYDVPWTFGQGTKVEIK | 18 |
| Human IL-2 | APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT | 19 |
| Human IL-2 (T3A, F42A, Y45Y, L72G, C125A) | APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT | 20 |
| linker | GGGGSGGGGSGGGGS | 21 |
| PD-1 IL2v -HC with IL2v (Fc knob, LALAPG) | evqllesggglvqpggslrlscaasgfsfssytmswvrqapgkglewvatisgggrdiyypdsvkgrftisrdnskntlylqmnslraedtavyycvlltgrvyfaldswgqgtlvtvssastkgpsvfplapsskstsggtaalgclvkdyfpepvtvswnsgaltsgvhtfpavlqssglyslssvvtvpssslgtqtyicnvnhkpsntkvdkkvepkscdkthtcppcpapeaaggpsvflfppkpkdtlmisrtpevtcvvvdvshedpevkfnwyvdgvevhnaktkpreeqynstyrvvsvltvlhqdwlngkeykckvsnkalgapiektiskakgqprepqvytlppcrdeltknqvslwclvkgfypsdiavewesngqpennykttppvldsdgsfflyskltvdksrwqqgnvfscsvmhealhnhytqkslslspgggggsggggsggggsapassstkktqlqlehllldlqmilnginnyknpkltrmltakfampkkatelkhlqcleeelkpleevlngaqsknfhlrprdlisninvivlelkgsettfmceyadetativeflnrwitfaqsiistlt | 22 |
| PD-1 IL2v -HC without IL2v (Fc hole, LALAPG) | evqllesggglvqpggslrlscaasgfsfssytmswvrqapgkglewvatisgggrdiyypdsvkgrftisrdnskntlylqmnslraedtavyycvlltgrvyfaldswgqgtlvtvssastkgpsvfplapsskstsggtaalgclvkdyfpepvtvswnsgaltsgvhtfpavlqssglyslssvvtvpssslgtqtyicnvnhkpsntkvdkkvepkscdkthtcppcpapeaaggpsvflfppkpkdtlmisrtpevtcvvvdvshedpevkfnwyvdgvevhnaktkpreeqynstyrvvsvltvlhqdwlngkeykckvsnkalgapiektiskakgqprepqvctlppsrdeltknqvslscavkgfypsdiavewesngqpennykttppvldsdgsfflvskltvdks rwqqgnvfscsvmhealhnhytqkslslsp | 23 |
| PD-1 IL2v -HC without IL2v (Fc hole, LALAPG, HYRF) | evqllesggglvqpggslrlscaasgfsfssytmswvrqapgkglewvatisgggrdiyypdsvkgrftisrdnskntlylqmnslraedtavyycvlltgrvyfaldswgqgtlvtvssastkgpsvfplapsskstsggtaalgclvkdyfpepvtvswnsgaltsgvhtfpavlqssglyslssvvtvpssslgtqtyicnvnhkpsntkvdkkvepkscdkthtcppcpapeaaggpsvflfppkpkdtlmisrtpevtcvvvdvshedpevkfnwyvdgvevhnaktkpreeqynstyrvvsvltvlhqdwlngkeykckvsnkalgapiektiskakgqprepqvctlppsrdeltknqvslscavkgfypsdiavewesngqpennykttppvldsdgsfflvskltvdks rwqqgnvfscsvmhealhnrftqkslslsp | 24 |
| PD-1 IL2v -LC | divmtqspdslavslgeratinckasesvdtsdnsfihwyqqkpgqspklliyrsstlesgvpdrfsgsgsgtdfdtisslqaedvavyycqqnydvpwtfgqgtkveikrtvaapsvfifppsdeqlksgtasvvcllnnfypreakvqwkvdnalqsgnsqesvteqdskdstyslsstltlskadyekhkvyacevthqglsspvtksffirgec | 25 |
| hIL-2 signal peptide | MYRMQLLSCIALSLALVTNS | 26 |
| hPD-1 (without signal sequence) | PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGT SSPARRGSADGPRSAQPLRPEDGHCSWPL | 27 |
| hPD-1 (with signal sequence) | MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEP PVPCVPEQTEYATIVFPSGMGTSSPARRGSADGPRSAQPLRPEDGHCSWPL | 28 |
| Human IL-2 (C125A) | APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT | 29 |
| Human IgG1 Fc domain | DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP | 30 |
| Human kappa CL domain | RTVAAPSVFIFPPSDEQLKSGTASVVCLLNNFYPREAKVQWKVDNALQSGNSQESVTEQDSKDSTYSLSSTLTLSKADYEKHKVYACEVTHQGLS SPVTKSFNRGEC | 31 |
| Human lambda CL domain | QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS | 32 |
| Human IgG1 heavy chain constant region (CH1-CH2-CH3) | ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP | 33 |
| Murine surrogate PD-1 IL2v - HC with IL2v | evqlqesgpglvkpsqslsltcsvtgysitssyrwnwirkfpgnrlewmgyinsagisnynpslkrrisitrdtsknqfflqvnsvttedaatyycarsdnmgttpftywgqgtlvtvssakttppsvyplapgsaaqtnsmvtlgclvkgyfpepvtvtwnsgslssgvhtfpavlqsdlytlsssvtvpsstwpsqtvtcnvahpasstkvdkkivprdcgckpcictvpevssvfifppkpkdvltitltpkvtcvvvaiskddpevqfswfvddvevhtaqtkpreeqinstfrsvselpimhqdwlngkefkcrvnsaafgapiektisktkgrpkapqvytipppkeqmakdkvsltcmitnffpeditvewqwngqpaenydntqpimdtdgsyfvysdlnvqksnweagntftcsvlheglhnhhtekslshspgggggsggggsggggsapassstssstaeaqqqqqqqqqqqqhleqllmdlqellsrmenyrnlklprmltakfalpkqatelkdlqcledelgplrhvldgtqsksfqledaenfisnirvtvvklkgsdntfecqfddesatvvdflrrwiafaqsiistspq | 34 |
| Murine surrogate PD-1 IL2v - HC without IL2v | evqlqesgpglvkpsqslsltcsvtgysitssyrwnwirkfpgnrlewmgyinsagisnynpslkrrisitrdtsknqfflqvnsvttedaatyycarsdnmgttpftywgqgtlvtvssakttppsvyplapgsaaqtnsmvtlgclvkgyfpepvtvtwnsgslssgvhtfpavlqsdlytlsssvtvpsstwpsqtvtcnvahpasstkvdkkivprdcgckpcictvpevssvfifppkpkdvltitltpkvtcvvvaiskddpevqfswfvddvevhtaqtkpreeqinstfrsvselpimhqdwlngkefkcrvnsaafgapiektisktkgrpkapqvytipppkkqmakdkvsltcmitnffpeditvewqwngqpaenykntqpimktdgsyfvysklnvqksnweag ntftcsvlheglhnhhtekslshsp | 35 |
| Murine surrogate PD-1 IL2v - LC | divmtqgtlpnpvpsgesvsitcrssksllysdgktylnwylqrpgqspqlliywmstrasgvsdrfsgsgsgtdftlkisgveaedvgiyycqqglefptfgggtklelkrtdaaptvsifppsseqltsggasvvcflnnfypkdinvkwkidgserqngvlnswtdqdskdstysmsstltltkdeyerhnsytceathktstspivksffimec | 36 |
| PD-L1 IL2v -HC with IL2v (Fc knob, LALAPG) | EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEBLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT | 37 |
| PD-L1 IL2v -HC without IL2v (Fc hole, LALAPG) | EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHE ALHNHYTQKSLSLSP | 38 |
| PD-L1 IL2v -LC | DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC | 39 |
| Murine surrogate PD-L1 IL2v - HC with IL2v | EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFGAPIEKTISKTKGRPKAPQVYTIPPPKEQMAKDKVSLTCMITDFFPEDITVEWQWNGQPAENYDNTQPIMDTDGSYFVYSDLNVQKSNWEAGNTFTCSVLHEGLHNHHTEKSLSHSPGGGGGSGGGGSGGGGSAPASSSTSSSTAEAQQQQQQQQQQQQHLEQLLMDLQELLSRMENYRNLKLPRMLTAKFALPKQATELKDLQCLEDELGPLRHVLDGTQSKSFQLEDAENFISNIRVTVVKLKGSDNTFECQFDDESATVVDFLRRWIAFAQSIISTSPQ | 40 |
| Murine surrogate PD-L1 IL2v - HC without IL2v | EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFGAPIEKTISKTKGRPKAPQVYTIPPPKKQMAKDKVSLTCMITDFFPEDITVEWQWNGQPAENYKNTQPIMKTDGSYFVYSKLNVQKSNWEAGNTFTCSVLHEGLHNHHTEKSLSHSP | 41 |
| Murine surrogate PD-L1 IL2v - LC | DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC | 42 |
| hPD-1 Extracellular Domain (ECD) | PGWFLDSPDRPWNPPTFSPALLVVTEGDNATFTCSFSNTS ESFVLNWYRMSPSNQTDKLAAFPEDRSQPGQDCRFRVTQ LPNGRDFHMSVVRARRNDSGTYLCGAISLAPKAQIKESLR AELRVTERRAEVPTAHPSPSPRPAGQFQTLV | 43 |
| muCEA HC-Fc (DD)-muIL2v | QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGGGSGGGGSGGGGSAPASSSTSSSTAEAQQQQQQQQQQQQHLEQLLMDLQELLSRMENYRNLKLPRMLTAKFALPKQATELKDLQCLEDELGPLRHVLDGTQSKSFQLEDAENFISNIRVTVVKLKGSDNTFECQFDDESATVVDFLRRWIAFAQSIISTSPQ | 44 |
| muCEA HC-Fc (KK)-muIL2v | QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGK | 45 |
| muCEA LC | DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC | 46 |
| muFAP HC-Fc (DD)-muIL2v | EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGGGSGGGGSGGGGSAPASSSTSSSTAEAQQQQQQQQQQQQHLEQLLMDLQELLSRMENYRNLKLPRMLTAKFALPKQATELKDLQCLEDELGPLRHVLDGTQSKSFQLEDAENFISNIRVTVVKLKGSDNTFECQFDDESATVVDFLRRWIAFAQSIISTSPQ | 47 |
| muFAP HC-Fc (KK) | EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGK | 48 |
| muFAP LC | EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC | 49 |
| anti-muPD1-HC_mIgG2a-LALAPG | EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSASTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK | 50 |
| anti muPD1-LC | DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC | 51 |

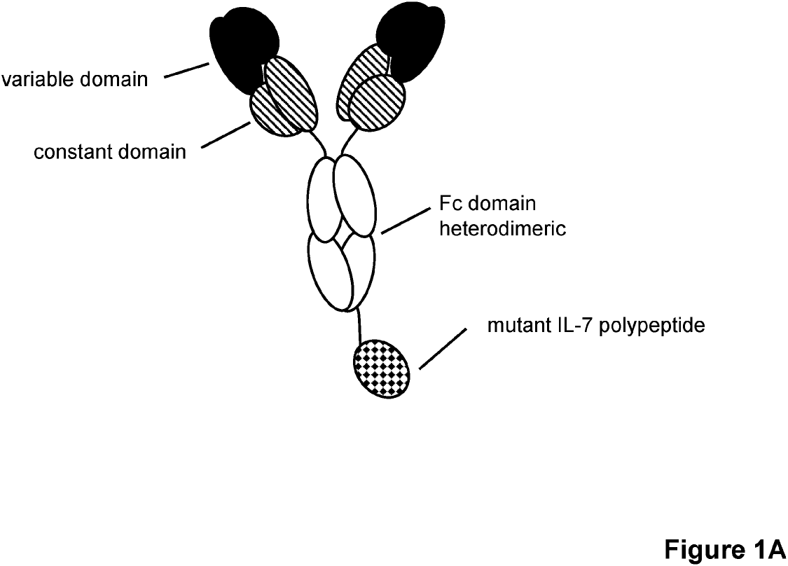

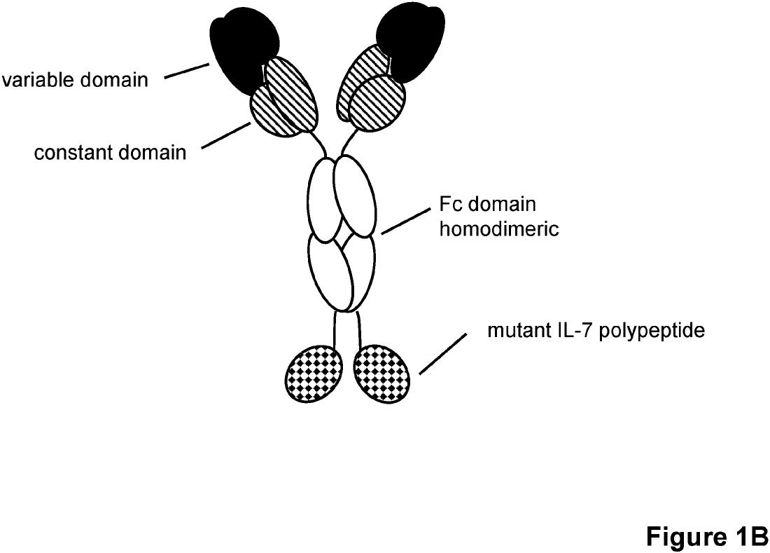

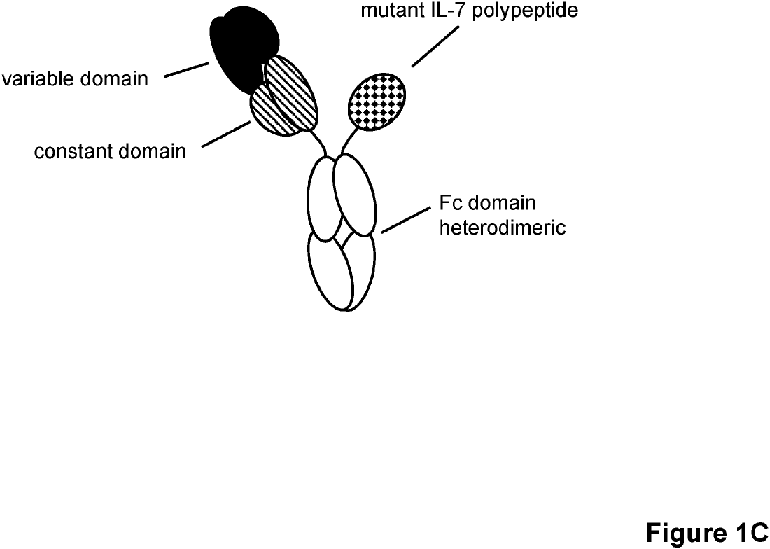

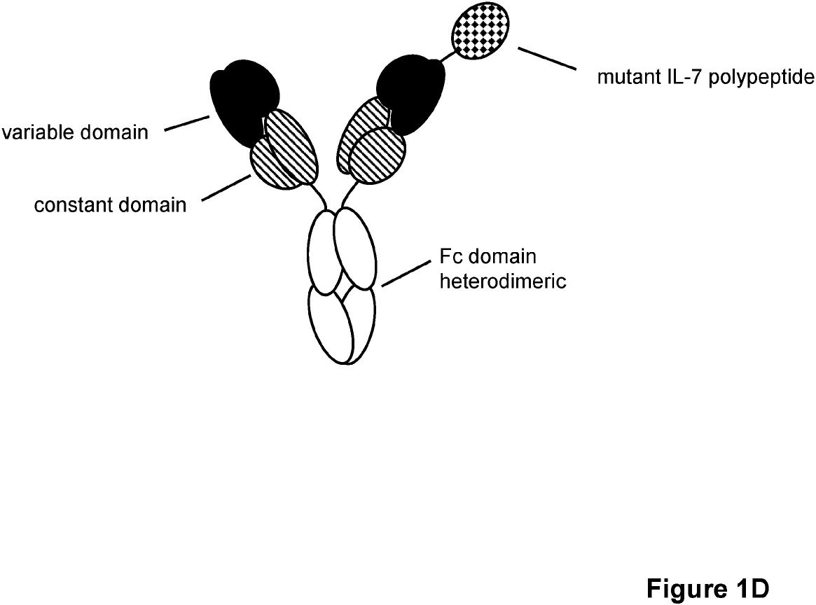

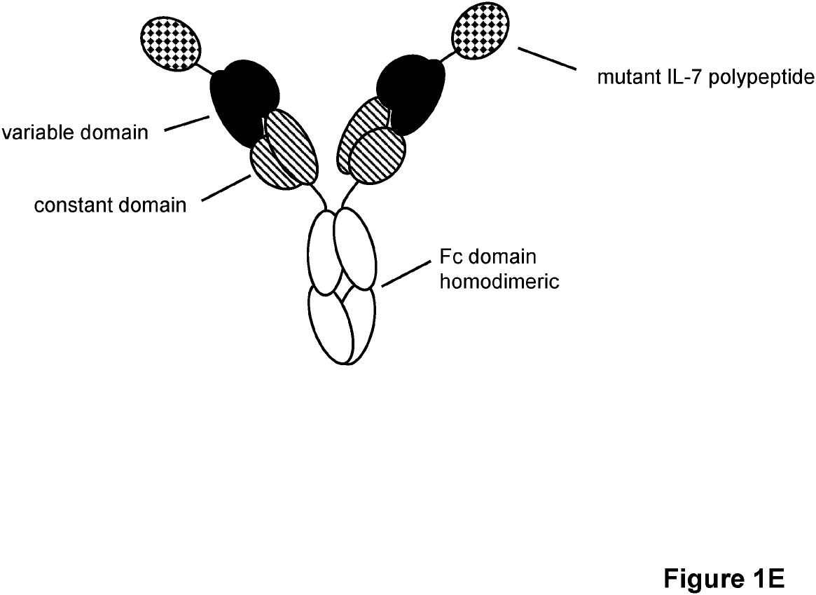

Amino acid sequences re IL-7

| | Modification | Amino acid sequece | SEQ ID NO |
|---|---|---|---|
| Human IL7 wild type | - | DCDIEG KDGKQYESVL MVSIDQLLDS MKEIGSNCLN NEFNFFKRHI CDANKEGMFL FRAARKLRQF LKMNSTGDFD LHLLKVSEGT TILLNCTGQV KGRKPAALGE AQPTKSLEEN KSLKEQKKLN DLCFLKRLLQ EIKTCWNKIL MGTKEH | 52 |
| IL7-VAR1 | E13A | DCDIEG KDGKQYASVL MVSIDQLLDS MKEIGSNCLN NEFNFFKRHI CDANKEGMFL FRAARKLRQF LKMNSTGDFD LHLLKVSEGT TILLNCTGQV KGRKPAALGE AQPTKSLEEN KSLKEQKKLN DLCFLKRLLQ EIKTCWNKIL MGTKEH | 53 |
| IL7-VAR2 | E13K | DCDIEG KDGKQYKSVL MVSIDQLLDS MKEIGSNCLN NEFNFFKRHI CDANKEGMFL FRAARKLRQF LKMNSTGDFD LHLLKVSEGT TILLNCTGQV KGRKPAALGE AQPTKSLEEN KSLKEQKKLN DLCFLKRLLQ EIKTCWNKIL MGTKEH | 54 |
| IL7-VAR3 | V15A | DCDIEG KDGKQYESAL MVSIDQLLDS MKEIGSNCLN NEFNFFKRHI CDANKEGMFL FRAARKLRQF LKMNSTGDFD LHLLKVSEGT TILLNCTGQV KGRKPAALGE AQPTKSLEEN KSLKEQKKLN DLCFLKRLLQ EIKTCWNKIL MGTKEH | 55 |
| IL7-VAR4 | V15K | DCDIEG KDGKQYESKL MVSIDQLLDS MKEIGSNCLN NEFNFFKRHI CDANKEGMFL FRAARKLRQF LKMNSTGDFD LHLLKVSEGT TILLNCTGQV KGRKPAALGE AQPTKSLEEN KSLKEQKKLN DLCFLKRLLQ EIKTCWNKIL MGTKEH | 56 |
| IL7-VAR5 | V18A | DCDIEG KDGKQYESVL MASIDQLLDS MKEIGSNCLN NEFNFFKRHI CDANKEGMFL FRAARKLRQF LKMNSTGDFD LHLLKVSEGT TILLNCTGQV KGRKPAALGE AQPTKSLEEN KSLKEQKKLN DLCFLKRLLQ EIKTCWNKIL MGTKEH | 57 |
| IL7-VAR6 | V18K | DCDIEG KDGKQYESVL MKSIDQLLDS MKEIGSNCLN NEFNFFKRHI CDANKEGMFL FRAARKLRQF LKMNSTGDFD LHLLKVSEGT TILLNCTGQV KGRKPAALGE AQPTKSLEEN KSLKEQKKLN DLCFLKRLLQ EIKTCWNKIL MGTKEH | 58 |
| IL7-VAR7 | D21A | DCDIEG KDGKQYESVL MVSIAQLLDS MKEIGSNCLN NEFNFFKRHI CDANKEGMFL FRAARKLRQF LKMNSTGDFD LHLLKVSEGT TILLNCTGQV KGRKPAALGE AQPTKSLEEN KSLKEQKKLN DLCFLKRLLQ EIKTCWNKIL MGTKEH | 59 |
| IL7-VAR8 | D21K | DCDIEG KDGKQYESVL MVSIKQLLDS MKEIGSNCLN NEFNFFKRHI CDANKEGMFL FRAARKLRQF LKMNSTGDFD LHLLKVSEGT TILLNCTGQV KGRKPAALGE AQPTKSLEEN KSLKEQKKLN DLCFLKRLLQ EIKTCWNKIL MGTKEH | 60 |
| IL7-VAR9 | Q22A | DCDIEG KDGKQYESVL MVSIDALLDS MKEIGSNCLN NEFNFFKRHI CDANKEGMFL FRAARKLRQF LKMNSTGDFD LHLLKVSEGT TILLNCTGQV KGRKPAALGE AQPTKSLEEN KSLKEQKKLN DLCFLKRLLQ EIKTCWNKIL MGTKEH | 61 |
| IL7-VAR10 | Q22K | DCDIEG KDGKQYESVL MVSIDKLLDS MKEIGSNCLN NEFNFFKRHI CDANKEGMFL FRAARKLRQF LKMNSTGDFD LHLLKVSEGT TILLNCTGQV KGRKPAALGE AQPTKSLEEN KSLKEQKKLN DLCFLKRLLQ EIKTCWNKIL MGTKEH | 62 |
| IL7-VAR11 | D25A | DCDIEG KDGKQYESVL MVSIDQLLAS MKEIGSNCLN NEFNFFKRHI CDANKEGMFL FRAARKLRQF LKMNSTGDFD LHLLKVSEGT TILLNCTGQV KGRKPAALGE AQPTKSLEEN KSLKEQKKLN DLCFLKRLLQ EIKTCWNKIL MGTKEH | 63 |
| IL7-VAR12 | D25K | DCDIEG KDGKQYESVL MVSIDQLLKS MKEIGSNCLN NEFNFFKRHI CDANKEGMFL FRAARKLRQF LKMNSTGDFD LHLLKVSEGT TILLNCTGQV KGRKPAALGE AQPTKSLEEN KSLKEQKKLN DLCFLKRLLQ EIKTCWNKIL MGTKEH | 64 |
| IL7-VAR13 | D74A | DCDIEG KDGKQYESVL MVSIDQLLDS MKEIGSNCLN NEFNFFKRHI CDANKEGMFL FRAARKLRQF LKMNSTGAFD LHLLKVSEGT TILLNCTGQV KGRKPAALGE AQPTKSLEEN KSLKEQKKLN DLCFLKRLLQ EIKTCWNKIL MGTKEH | 65 |
| IL7-VAR14 | D74K | DCDIEG KDGKQYESVL MVSIDQLLDS MKEIGSNCLN NEFNFFKRHI CDANKEGMFL FRAARKLRQF LKMNSTGKFD LHLLKVSEGT TILLNCTGQV KGRKPAALGE AQPTKSLEEN KSLKEQKKLN DLCFLKRLLQ EIKTCWNKIL MGTKEH | 66 |
| IL7-VAR15 | L77A | DCDIEG KDGKQYESVL MVSIDQLLDS MKEIGSNCLN NEFNFFKRHI CDANKEGMFL FRAARKLRQF LKMNSTGDFD AHLLKVSEGT TILLNCTGQV KGRKPAALGE AQPTKSLEEN KSLKEQKKLN DLCFLKRLLQ EIKTCWNKIL MGTKEH | 67 |
| IL7-VAR16 | L77K | DCDIEG KDGKQYESVL MVSIDQLLDS MKEIGSNCLN NEFNFFKRHI CDANKEGMFL FRAARKLRQF LKMNSTGDFD KHLLKVSEGT TILLNCTGQV KGRKPAALGE AQPTKSLEEN KSLKEQKKLN DLCFLKRLLQ EIKTCWNKIL MGTKEH | 68 |
| IL7-VAR17 | K81A | DCDIEG KDGKQYESVL MVSIDQLLDS MKEIGSNCLN NEFNFFKRHI CDANKEGMFL FRAARKLRQF LKMNSTGDFD LHLLAVSEGT TILLNCTGQV KGRKPAALGE AQPTKSLEEN KSLKEQKKLN DLCFLKRLLQ EIKTCWNKIL MGTKEH | 69 |
| IL7-VAR18 | K81E | DCDIEG KDGKQYESVL MVSIDQLLDS MKEIGSNCLN NEFNFFKRHI CDANKEGMFL FRAARKLRQF LKMNSTGDFD LHLLFVSEGT TILLNCTGQV KGRKPAALGE AQPTKSLEEN KSLKEQKKLN DLCFLKRLLQ EIKTCWNKIL MGTKEH | 70 |
| IL7-VAR19 | E84A | DCDIEG KDGKQYESVL MVSIDQLLDS MKEIGSNCLN NEFNFFKRHI CDANKEGMFL FRAARKLRQF LKMNSTGDFD LHLLKVSAGT TILLNCTGQV KGRKPAALGE AQPTKSLEEN KSLKEQKKLN DLCFLKRLLQ EIKTCWNKIL MGTKEH | 71 |
| IL7-VAR20 | G85K | DCDIEG KDGKQYESVL MVSIDQLLDS MKEIGSNCLN NEFNFFKRHI CDANKEGMFL FRAARKLRQF LKMNSTGDFD LHLLKVSEKT TILLNCTGQV KGRKPAALGE AQPTKSLEEN KSLKEQKKLN DLCFLKRLLQ EIKTCWNKIL MGTKEH | 72 |
| IL7-VAR21 | G85E | DCDIEG KDGKQYESVL MVSIDQLLDS MKEIGSNCLN NEFNFFKRHI CDANKEGMFL FRAARKLRQF LKMNSTGDFD LHLLKVSEET TILLNCTGQV KGRKPAALGE AQPTKSLEEN KSLKEQKKLN DLCFLKRLLQ EIKTCWNKIL MGTKEH | 73 |
| IL7-VAR22 | I88K | DCDIEG KDGKQYESVL MVSIDQLLDS MKEIGSNCLN NEFNFFKRHI CDANKEGMFL FRAARKLRQF LKMNSTGDFD LHLLKVSEGT TKLLNCTGQV KGRKPAALGE AQPTKSLEEN KSLKEQKKLN DLCFLKRLLQ EIKTCWNKIL MGTKEH | 74 |
| IL7-VAR23 | Q136A | DCDIEG KDGKQYESVL MVSIDQLLDS MKEIGSNCLN NEFNFFKRHI CDANKEGMFL FRAARKLRQF LKMNSTGDFD LHLLKVSEGT TILLNCTGQV KGRKPAALGE AQPTKSLEEN KSLKEQKKLN DLCFLKRLLA EIKTCWNKIL MGTKEH | 75 |
| IL7-VAR24 | Q136K | DCDIEG KDGKQYESVL MVSIDQLLDS MKEIGSNCLN NEFNFFKRHI CDANKEGMFL FRAARKLRQF LKMNSTGDFD LHLLKVSEGT TILLNCTGQV KGRKPAALGE AQPTKSLEEN KSLKEQKKLN DLCFLKRLLK EIKTCWNKIL MGTKEH | 76 |
| IL7-VAR25 | K139A | DCDIEG KDGKQYESVL MVSIDQLLDS MKEIGSNCLN NEFNFFKRHI CDANKEGMFL FRAARKLRQF LKMNSTGDFD LHLLKVSEGT TILLNCTGQV KGRKPAALGE AQPTKSLEEN KSLKEQKKLN DLCFLKRLLQ EIATCWNKIL MGTKEH | 77 |
| IL7-VAR26 | K139E | DCDIEG KDGKQYESVL MVSIDQLLDS MKEIGSNCLN NEFNFFKRHI CDANKEGMFL FRAARKLRQF LKMNSTGDFD LHLLKVSEGT TILLNCTGQV KGRKPAALGE AQPTKSLEEN KSLKEQKKLN DLCFLKRLLQ EIETCWNKIL MGTKEH | 78 |
| IL7-VAR27 | N143K | DCDIEG KDGKQYESVL MVSIDQLLDS MKEIGSNCLN NEFNFFKRHI CDANKEGMFL FRAARKLRQF LKMNSTGDFD LHLLKVSEGT TILLNCTGQV KGRKPAALGE AQPTKSLEEN KSLKEQKKLN DLCFLKRLLQ EIKTCWKKIL MGTKEH | 79 |
| IL7-VAR28 | M147A | DCDIEG KDGKQYESVL MVSIDQLLDS MKEIGSNCLN NEFNFFKRHI CDANKEGMFL FRAARKLRQF LKMNSTGDFD LHLLKVSEGT TILLNCTGQV KGRKPAALGE AQPTKSLEEN KSLKEQKKLN DLCFLKRLLQ EIKTCWNKIL AGTKEH | 80 |
| IL7-VAR29 | N-Glc KO, T72A | DCDIEG KDGKQYESVL MVSIDQLLDS MKEIGSNCLN NEFNFFKRHI CDANKEGMFL FRAARKLRQF LKMNSAGDFD LHLLKVSEGT TILLNCTGQV KGRKPAALGE AQPTKSLEEN KSLKEQKKLN DLCFLKRLLQ EIKTCWNKIL MGTKEH | 81 |
| IL7-VAR30 | N-Glc KO, T93A | DCDIEG KDGKQYESVL MVSIDQLLDS MKEIGSNCLN NEFNFFKRHI CDANKEGMFL FRAARKLRQF LKMNSTGDFD LHLLKVSEGT TILLNCAGQV KGRKPAALGE AQPTKSLEEN KSLKEQKKLN DLCFLKRLLQ EIKTCWNKIL MGTKEH | 82 |
| IL7-VAR31 | N-Glc KO, S118A | DCDIEG KDGKQYESVL MVSIDQLLDS MKEIGSNCLN NEFNFFKRHI CDANKEGMFL FRAARKLRQF LKMNSTGDFD LHLLKVSEGT TILLNCTGQV KGRKPAALGE AQPTKSLEEN KALKEQKKLN DLCFLKRLLQ EIKTCWNKIL MGTKEH | 83 |
| IL7-VAR32 | N-Glc KO, T72A, T93A, S118A | DCDIEG KDGKQYESVL MVSIDQLLDS MKEIGSNCLN NEFNFFKRHI CDANKEGMFL FRAARKLRQF LKMNSAGDFD LHLLKVSEGT TILLNCAGQV KGRKPAALGE AQPTKSLEEN KALKEQKKLN DLCFLKRLLQ EIKTCWNKIL MGTKEH | 84 |
| Chain A | - | DIVMTQSPDSLAVSLGERATINCKASESVDTSDNSFIHWYQ QKPGQSPKLLIYRSSTLESGVPDRFSGSGSGTDFTLTISSLQA EDVAVYYCQQNYDVPWTFGQGTKVEIKRTVAAPSVFIFPP SDEQLKSGTASWCLLNNFYPREAKVQWKVDNALQSGNS QESVTEQDSKDSTYSLSSTLTLSKADYEKHKVYACEVTHQ GLSSPVTKSFNRGEC | 85 |
| Chain H | - | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVCTLPPSRDELTK NQVSLSCAVKGFYPSDIAVEWESNGQPENNYKTTPPVLDS DGSFFLVSKLTVDKSRWQQGNVFSCSVMHEALHNHYTQK SLSLSPGK | 86 |
| Chain K of PD1-IL7-wt | - | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYESVLMV SIDQLLDSMKEIGSNCLNNEFNFFKRHICDANKEGMFLFRA ARKLRQFLKMNSTGDFDLHLLKVSEGTTILLNCTGQVKGR KPAALGEAQPTKSLEENKSLKEQKKLNDLCFLKRLLQEIKT CWNKILMGTKEH | 87 |
| Chain K of PD1-IL7-VAR1 | E13A | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYASVLM VSIDQLLD SMKEIGSNCLNNEFNFFKRHICDANKEGMFLFR AARKLRQFLKMNSTGDFDLHLLKVSEGTTILLNCTGQVKG RKPAALGEAQPTKSLEENKSLKEQKKLNDLCFLKRLLQEIK TCWNKILMGTKEH | 88 |
| Chain K of PD1-IL7-VAR2 | E13K | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYKSVLM VSIDQLLD SMKEIGSNCLNNEFNFFKRHICDANKEGMFLFR AARKLRQFLKMNSTGDFDLHLLKVSEGTTILLNCTGQVKG RKPAALGEAQPTKSLEENKSLKEQKKLNDLCFLKRLLQEIK TCWNKILMGTKEH | 89 |
| Chain K of PD1-IL7-VAR3 | V15A | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYESALMV SIDQLLDSMKEIGSNCLNNEFNFFKRHICDANKEGMFLFRA ARKLRQFLKMNSTGDFDLHLLKVSEGTTILLNCTGQVKGR KPAALGEAQPTKSLEENKSLKEQKKLNDLCFLKRLLQEIKT CWNKILMGTKEH | 90 |
| Chain K of PD1-IL7-VAR4 | V15K | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYESKLMV SIDQLLDSMKEIGSNCLNNEFNFFKRHICDANKEGMFLFRA ARKLRQFLKMNSTGDFDLHLLKVSEGTTILLNCTGQVKGR KPAALGEAQPTKSLEENKSLKEQKKLNDLCFLKRLLQEIKT CWNKILMGTKEH | 91 |
| Chain K of PD1-IL7-VAR5 | V18A | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYESVLMA SIDQLLDSMKEIGSNCLNNEFNFFKRHICDANKEGMFLFRA ARKLRQFLKMNSTGDFDLHLLKVSEGTTILLNCTGQVKGR KPAALGEAQPTKSLEENKSLKEQKKLNDLCFLKRLLQEIKT CWNKILMGTKEH | 92 |
| Chain K of PD1-IL7-VAR6 | V18K | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYESVLMK SIDQLLDSMKEIGSNCLNNEFNFFKRHICDANKEGMFLFRA ARKLRQFLKMNSTGDFDLHLLKVSEGTTILLNCTGQVKGR KPAALGEAQPTKSLEENKSLKEQKKLNDLCFLKRLLQEIKT CWNKILMGTKEH | 93 |
| Chain K of PD1-IL7-VAR7 | D21A | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYESVLMV SIAQLLDSMKEIGSNCLNNEFNFFKRHICDANKEGMFLFRA ARKLRQFLKMNSTGDFDLHLLKVSEGTTILLNCTGQVKGR KPAALGEAQPTKSLEENKSLKEQKKLNDLCFLKRLLQEIKT CWNKILMGTKEH | 94 |
| Chain K of PD1-IL7-VAR8 | D21K | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYESVLMV SIKQLLDSMKEIGSNCLNNEFNFFKRHICDANKEGMFLFRA ARKLRQFLKMNSTGDFDLHLLKVSEGTTILLNCTGQVKGR KPAALGEAQPTKSLEENKSLKEQKKLNDLCFLKRLLQEIKT CWNKILMGTKEH | 95 |
| Chain K of PD1-IL7-VAR9 | Q22A | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYESVLMV SIDALLDSMKEIGSNCLNNEFNFFKRHICDANKEGMFLFRA ARKLRQFLKMNSTGDFDLHLLKVSEGTTILLNCTGQVKGR KPAALGEAQPTKSLEENKSLKEQKKLNDLCFLKRLLQEIKT CWNKILMGTKEH | 96 |
| Chain K of PD1-IL7-VAR10 | Q22K | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYESVLMV SIDKLLDSMKEIGSNCLNNEFNFFKRHICDANKEGMFLFRA ARKLRQFLKMNSTGDFDLHLLKVSEGTTILLNCTGQVKGR KPAALGEAQPTKSLEENKSLKEQKKLNDLCFLKRLLQEIKT CWNKILMGTKEH | 97 |
| Chain K of PD1-IL7-VAR11 | D25A | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYESVLMV SIDQLLASMKEIGSNCLNNEFNFFKRHICDANKEGMFLFRA ARKLRQFLKMNSTGDFDLHLLKVSEGTTILLNCTGQVKGR KPAALGEAQPTKSLEENKSLKEQKKLNDLCFLKRLLQEIKT CWNKILMGTKEH | 98 |
| Chain K of PD1-IL7-VAR12 | D25K | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYESVLMV SIDQLLKSMKEIGSNCLNNEFNFFKRHICDANKEGMFLFRA ARKLRQFLKMNSTGDFDLHLLKVSEGTTILLNCTGQVKGR KPAALGEAQPTKSLEENKSLKEQKKLNDLCFLKRLLQEIKT CWNKILMGTKEH | 99 |
| Chain K of PD1-IL7-VAR13 | D74A | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYESVLMV SIDQLLDSMKEIGSNCLNNEFNFFKRHICDANKEGMFLFRA ARKLRQFLKMNSTGAFDLHLLKVSEGTTILLNCTGQVKGR KPAALGEAQPTKSLEENKSLKEQKKLNDLCFLKRLLQEIKT CWNKILMGTKEH | 100 |
| Chain K of PD1-IL7-VAR14 | D74K | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYESVLMV SIDQLLDSMKEIGSNCLNNEFNFFKRHICDANKEGMFLFRA ARKLRQFLKMNSTGKFDLHLLKVSEGTTILLNCTGQVKGR KPAALGEAQPTKSLEENKSLKEQKKLNDLCFLKRLLQEIKT CWNKILMGTKEH | 101 |
| Chain K of PD1-IL7-VAR15 | L77A | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYESVLMV SIDQLLDSMKEIGSNCLNNEFNFFKRHICDANKEGMFLFRA ARKLRQFLKMNSTGDFDAHLLKVSEGTTILLNCTGQVKGR KPAALGEAQPTKSLEENKSLKEQKKLNDLCFLKRLLQEIKT CWNKILMGTKEH | 102 |
| Chain K of PD1-IL7-VAR16 | L77K | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYESVLMV SIDQLLDSMKEIGSNCLNNEFNFFKRHICDANKEGMFLFRA ARKLRQFLKMNSTGDFDKHLLKVSEGTTILLNCTGQVKGR KPAALGEAQPTKSLEENKSLKEQKKLNDLCFLKRLLQEIKT CWNKILMGTKEH | 103 |
| Chain K of PD1-IL7-VAR17 | K81A | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYESVLMV SIDQLLDSMKEIGSNCLNNEFNFFKRHICDANKEGMFLFRA ARKLRQFLKMNSTGDFDLHLLAVSEGTTILLNCTGQVKGR KPAALGEAQPTKSLEENKSLKEQKKLNDLCFLKRLLQEIKT CWNKILMGTKEH | 104 |
| Chain K of PD1-IL7-VAR18 | K81E | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYESVLMV SIDQLLDSMKEIGSNCLNNEFNFFKRHICDANKEGMFLFRA ARKLRQFLKMNSTGDFDLHLLEVSEGTTILLNCTGQVKGR KPAALGEAQPTKSLEENKSLKEQKKLNDLCFLKRLLQEIKT CWNKILMGTKEH | 105 |
| Chain K of PD1-IL7-VAR19 | E84A | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYESVLMV SIDQLLDSMKEIGSNCLNNEFNFFKRHICDANKEGMFLFRA ARKLRQFLKMNSTGDFDLHLLKVSAGTTILLNCTGQVKGR KPAALGEAQPTKSLEENKSLKEQKKLNDLCFLKRLLQEIKT CWNKILMGTKEH | 106 |
| Chain K of PD1-IL7-VAR20 | G85K | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYESVLMV SIDQLLDSMKEIGSNCLNNEFNFFKRHICDANKEGMFLFRA ARKLRQFLKMNSTGDFDLHLLKVSEKTTILLNCTGQVKGR KPAALGEAQPTKSLEENKSLKEQKKLNDLCFLKRLLQEIKT CWNKILMGTKEH | 107 |
| Chain K of PD1-IL7-VAR21 | G85E | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYESVLMV SIDQLLDSMKEIGSNCLNNEFNFFKRHICDANKEGMFLFRA ARKLRQFLKMNSTGDFDLHLLKVSEETTILLNCTGQVKGR KPAALGEAQPTKSLEENKSLKEQKKLNDLCFLKRLLQEIKT CWNKILMGTKEH | 108 |
| Chain K of PD1-IL7-VAR22 | 188K | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYESVLMV SIDQLLDSMKEIGSNCLNNEFNFFKRHICDANKEGMFLFRA ARKLRQFLKMNSTGDFDLHLLKVSEGTTKLLNCTGQVKG RKPAALGEAQPTKSLEENKSLKEQKKLNDLCFLKRLLQEIK TCWNKILMGTKEH | 109 |
| Chain K of PD1-IL7-VAR23 | Q136A | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYESVLMV SIDQLLDSMKEIGSNCLNNEFNFFKRHICDANKEGMFLFRA ARKLRQFLKMNSTGDFDLHLLKVSEGTTILLNCTGQVKGR KPAALGEAQPTKSLEENKSLKEQKKLNDLCFLKRLLAEIKT CWNKILMGTKEH | 110 |
| Chain K of PD1-IL7-VAR24 | Q136K | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYESVLMV SIDQLLDSMKEIGSNCLNNEFNFFKRHICDANKEGMFLFRA ARKLRQFLKMNSTGDFDLHLLKVSEGTTILLNCTGQVKGR KPAALGEAQPTKSLEENKSLKEQKKLNDLCFLKRLLKEIKT CWNKILMGTKEH | 111 |
| Chain K of PD1-IL7-VAR25 | K139A | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYESVLMV SIDQLLDSMKEIGSNCLNNEFNFFKRHICDANKEGMFLFRA ARKLRQFLKMNSTGDFDLHLLKVSEGTTILLNCTGQVKGR KPAALGEAQPTKSLEENKSLKEQKKLNDLCFLKRLLQEIAT CWNKILMGTKEH | 112 |
| Chain K of PD1-IL7-VAR26 | K139E | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYESVLMV SIDQLLDSMKEIGSNCLNNEFNFFKRHICDANKEGMFLFRA ARKLRQFLKMNSTGDFDLHLLKVSEGTTILLNCTGQVKGR KPAALGEAQPTKSLEENKSLKEQKKLNDLCFLKRLLQEIET CWNKILMGTKEH | 113 |
| Chain K of PD1-IL7-VAR27 | N143K | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYESVLMV SIDQLLDSMKEIGSNCLNNEFNFFKRHICDANKEGMFLFRA ARKLRQFLKMNSTGDFDLHLLKVSEGTTILLNCTGQVKGR KPAALGEAQPTKSLEENKSLKEQKKLNDLCFLKRLLQEIKT CWKKILMGTKEH | 114 |
| Chain K of PD1-IL7-VAR28 | M147A | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYESVLMV SIDQLLDSMKEIGSNCLNNEFNFFKRHICDANKEGMFLFRA ARKLRQFLKMNSTGDFDLHLLKVSEGTTILLNCTGQVKGR KPAALGEAQPTKSLEENKSLKEQKKLNDLCFLKRLLQEIKT CWNKILAGTKEH | 115 |
| Chain K of PD1-IL7-VAR29 | N-Glc KO, T72A | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYESVLMV SIDQLLDSMKEIGSNCLNNEFNFFKRHICDANKEGMFLFRA ARKLRQFLKMNSAGDFDLHLLKVSEGTTILLNCTGQVKGR KPAALGEAQPTKSLEENKSLKEQKKLNDLCFLKRLLQEIKT CWNKILMGTKEH | 116 |
| Chain K of PD1-IL7-VAR30 | N-Glc KO, T93A | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFLYSKLTVDKSRWQQGNVFSCSVMHEALHNHYTQK SLSLSPGGGGGSGGGGSGGGGSDCDIEGKDGKQYESVLMV SIDQLLDSMKEIGSNCLNNEFNFFKRHICDANKEGMFLFRA ARKLRQFLKMNSTGDFDLHLLKVSEGTTILLNCAGQVKGR KPAALGEAQPTKSLEENKSLKEQKKLNDLCFLKRLLQEIKT CWNKILMGTKEH | 117 |
| Chain K of PD1-IL7-VAR31 | N-Glc KO, S118A | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRWSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFL YSKL TVDKSRWQQGNVFSCSVMHEALHNHYTQK SLSLSPGGGGGSGGGGSGGGGSDCDIEGKDGKQYESVLMV SIDQLLDSMKEIGSNCLNNEFNFFKRHICDANKEGMFLFRA ARKLRQFLKMNSTGDFDLHLLKVSEGTTILLNCTGQVKGR KPAALGEAQPTKSLEENKALKEQKKLNDLCFLKRLLQEIKT CWNKILMGTKEH | 118 |
| Chain K of PD1-IL7-VAR32 | N-Glc KO, T72A, T93A, S118A | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRWSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFL YSKL TVDKSRWQQGNVFSCSVMHEALHNHYTQK SLSLSPGGGGGSGGGGSGGGGSDCDIEGKDGKQYESVLMV SIDQLLDSMKEIGSNCLNNEFNFFKRHICDANKEGMFLFRA ARKLRQFLKMNSAGDFDLHLLKVSEGTTILLNCAGQVKGR KPAALGEAQPTKSLEENKALKEQKKLNDLCFLKRLLQEIKT CWNKILMGTKEH | 119 |
| IL7Ralpha-Fc knob-avi | - | ESGYAQNGDLEDAELDDYSFSCYSQLEVNGSQHSLTCAFE DPDVNITNLEFEICGALVEVKCLNFRKLQEIYF1E1KKFLLIG KSNICVKVGEKSLTCKKIDLTTIVKPEAPFDLSVVYREGAN DFVVTFNTSHLQKKYVKVLMHDVAYRQEKDENKWTHVN LSSTKLTLLQRKLQPAAMYEIKVRSIPDHYFKGFWSEWSPS YYFRTPEINNSSGEMDASGSDKTHTCPPCPAPELLGGPSVFL FPPKPKDTLMISRTPEVTCVVVDVSHEDPEVKFNWYVDGV EVHNAKTKPREEQYNSTYRVVSVLTVLHQDWLNGKEYKC KVSNKALPAPIEKTISKAKGQPREPQVYTLPPCRDELTKNQ VSLWCLVKGFYPSDIAVEWESNGQPENNYKTTPPVLDSDG SFFLYSKLTVDKSRWQQGNVFSCSVMHEALHNHYTQKSLS LSPGKSGGLNDIFEAQKIEWHE | 120 |
| IL2Rgamma-Fc hole | | LNTTILTPNGNEDTTADFFLTTMPTDSLSVSTLPLPEVQCFV FNVEYMNCTWNSSSEPQPTNLTLHYWYKNSDNDKVQKCS HYLFSEEITSGCQLQKKEIHLYQTFWQLQDPREPRRQATQ MLKLQNLVIPWAPENLTLHKLSESQLELNWNNRFLNHCLE HLVQYRTDWDHSWTEQSVDYRHKFSLPSVDGQKRYTFRV GSDKTHTCPPCPAPELLGGPSVFLFPPKPKDTLMISRTPEVT CVVVDVSHEDPEVKFNWYVDGVEVHNAKTKPREEQYNST YRWS VLTVLHQD WLNGKEYKCKVSNKALPAPIEKTISKA KGQPREPQVCTLPPSRDELTKNQVSLSCAVKGFYPSDIAVE WESNGQPENNYKTTPPVLDSDGSFFLVSKLTVDKSRWQQG NVFSCSVM:HEALHNHYTQKSLSLSPGK | 121 |
| Chain A -FIG. 1A | | DIVMTQSPDSLAVSLGERATINCKASESVDTSDNSFIHWYQ QKPGQSPKLLIYRSSTLESGVPDRFSGSGSGTDFTLTISSLQA EDVAVYYCQQNYDVPWTFGQGTKVEIKRTVAAPSVFIFPP SDEQLKSGTASWCLLNNFYPREAKVQWKVDNALQSGNS QESVTEQDSKDSTYSLSSTLTLSKADYEKHKVYACEVTHQ GLSSPVTKSFNRGEC | 122 |
| Chain H -FIG. 1A | | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVCTLPPSRDELTK NQVSLSCAVKGFYPSDIAVEWESNGQPENNYKTTPPVLDS DGSFFLVSKLTVDKSRWQQGNVFSCSVMHEALHNHYTQK SLSLSPG | 123 |
| Chain K of PD 1-IL7-_{W}t -FIG. 1A | | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFL YSKL TVDKSRWQQGNVFSCSVMHEALHNHYTQK SLSLSPGGGGGSGGGGSGGGGSDCDIEGKDGKQYESVLMV SIDQLLDSMKEIGSNCLNNEFNFFKRHICDANKEGMFLFRA ARKLRQFLKMNSTGDFDLHLLKVSEGTTILLNCTGQVKGR KPAALGEAQPTKSLEENKSLKEQKKLNDLCFLKRLLQEIKT CWNKILMGTKEH | 124 |
| Chain A -FIG. 1B | | DIVMTQSPDSLAVSLGERATINCKASESVDTSDNSFIHWYQ QKPGQSPKLLIYRSSTLESGVPDRFSGSGSGTDFTLTISSLQA EDVAVYYCQQNYDVPWTFGQGTKVEIKRTVAAPSVFIFPP SDEQLKSGTASWCLLNNFYPREAKVQWKVDNALQSGNS QESVTEQDSKDSTYSLSSTLTLSKADYEKHKVYACEVTHQ GLSSPVTKSFNRGEC | 125 |
| Chain B -FIG. 1B | | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPSRDELTK NQVSLTCLVKGFYPSDIAVEWESNGQPENNYKTTPPVLDS DGSFFL YSKL TVDKSRWQQGNVFSCSVMHEALHNHYTQK SLSLSPGGGGGSGGGGSGGGGSDCDIEGKDGKQYESVLMV SIDQLLDSMKEIGSNCLNNEFNFFKRHICDANKEGMFLFRA ARKLRQFLKMNSTGDFDLHLLKVSEGTTILLNCTGQVKGR KPAALGEAQPTKSLEENKSLKEQKKLNDLCFLKRLLQEIKT CWNKILMGTKEH | 126 |
| Chain A -FIG. 1C | | DIVMTQSPDSLAVSLGERATINCKASESVDTSDNSFIHWYQ QKPGQSPKLLIYRSSTLESGVPDRFSGSGSGTDFTLTISSLQA EDVAVYYCQQNYDVPWTFGQGTKVEIKRTVAAPSVFIFPP SDEQLKSGTASWCLLNNFYPREAKVQWKVDNALQSGNS QESVTEQDSKDSTYSLSSTLTLSKADYEKHKVYACEVTHQ GLSSPVTKSFNRGEC | 127 |
| Chain H -FIG. 1C | | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVCTLPPSRDELTK NQVSLSCAVKGFYPSDIAVEWESNGQPENNYKTTPPVLDS DGSFFLVSKLTVDKSRWQQGNVFSCSVMHEALHNHYTQK SLSLSPG | 128 |
| Chain K of IL7wt-Fc -FIG. 1C | | DCDIEGKDGKQYESVLMVSIDQLLDSMKEIGSNCLNNEFNF FKRHICDANKEGMFLFRAARKLRQFLKMNSTGDFDLHLLK VSEGTTILLNCTGQVKGRKPAALGEAQPTKSLEENKSLKEQ KKLNDLCFLKRLLQEIKTCWNKILMGTKEHGEPKSCDKTH TCPPCP APEAAGGPSVFLFPPKPKDTLMISR TPEVTCVVVD VSHEDPEVKFNWYVDGVEVHNAKTKPREEQYNSTYRWS VLTVLHQDWLNGKEYKCKVSNKALGAPIEKTISKAKGQPR EPQVYTLPPCRDELTKNQVSLWCLVKGFYPSDIAVEWESN GQPENNYKTTPPVLDSDGSFFLYSKLTVDKSRWQQGNVFS CSVM:HEALHNHYTQKSLSLSPG | 129 |
| Chain A -FIG. 1D | | DIVMTQSPDSLAVSLGERATINCKASESVDTSDNSFIHWYQ QKPGQSPKLLIYRSSTLESGVPDRFSGSGSGTDFTLTISSLQA EDVAVYYCQQNYDVPWTFGQGTKVEIKRTVAAPSVFIFPP SDEQLKSGTASWCLLNNFYPREAKVQWKVDNALQSGNS QESVTEQDSKDSTYSLSSTLTLSKADYEKHKVYACEVTHQ GLSSPVTKSFNRGEC | 130 |
| Chain H -FIG. 1D | | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVCTLPPSRDELTK NQVSLSCAVKGFYPSDIAVEWESNGQPENNYKTTPPVLDS DGSFFLVSKLTVDKSRWQQGNVFSCSVMHEALHNHYTQK SLSLSPG | 131 |
| Chain K of IL7wt-PD1 -FIG. 1D | | DCDIEGKDGKQYESVLMVSIDQLLDSMKEIGSNCLNNEFNF FKRHICDANKEGMFLFRAARKLRQFLKMNSTGDFDLHLLK VSEGTTILLNCTGQVKGRKPAALGEAQPTKSLEENKSLKEQ KKLNDLCFLKRLLQEIKTCWNKILMGTKEHGGGGGSGGG GSGGGGSEVQLLESGGGLVQPGGSLRLSCAASGFSFSSYTM SWVRQAPGKGLEW VATISGGGRDIYYPD S VKGRFTISRDN SKNTLYLQMNSLRAEDTAVYYCVLLTGRVYFALDSWGQG TLVTVSSASTKGPSVFPLAPSSKSTSGGTAALGCLVKDYFP EPVTVSWNSGAL TSGVHTFP A VLQSSGL YSLSSVVTVPSSS LGTQTYICNVNHKPSNTKVDKKVEPKSCDKTHTCPPCPAPE AAGGPSVFLFPPKPKDTLMISRTPEVTCVWDVSHEDPEVK FNWYVDGVEVHNAKTKPREEQYNSTYRWSVLTVLHQD WLNGKEYKCKVSNKALGAPIEKTISKAKGQPREPQVYTLP PCRDELTKNQVSLWCLVKGFYPSDIAVEWESNGQPENNYK TTPPVLD SD GSFFL YSKL TVDKSR WQQGNVFSCSVMHEAL HNHYTQKSLSLSPG | 132 |
| Chain A -FIG. 1E | | DIVMTQSPDSLAVSLGERATINCKASESVDTSDNSFIHWYQ QKPGQSPKLLIYRSSTLESGVPDRFSGSGSGTDFTLTISSLQA EDVAVYYCQQNYDVPWTFGQGTKVEIKRTVAAPSVFIFPP SDEQLKSGTASWCLLNNFYPREAKVQWKVDNALQSGNS QESVTEQDSKDSTYSLSSTLTLSKADYEKHKVYACEVTHQ GLSSPVTKSFNRGEC | 133 |
| Chain B -FIG. 1E | | DCDIEGKDGKQYESVLMVSIDQLLDSMKEIGSNCLNNEFNF FKRHICDANKEGMFLFRAARKLRQFLKMNSTGDFDLHLLK VSEGTTILLNCTGQVKGRKPAALGEAQPTKSLEENKSLKEQ KKLNDLCFLKRLLQEIKTCWNKILMGTKEHGGGGGSGGG GSGGGGSEVQLLESGGGLVQPGGSLRLSCAASGFSFSSYTM SWVRQAPGKGLEWVATISGGGRDIYYPDSVKGRFTISRDN SKNTLYLQMNSLRAEDTAVYYCVLLTGRVYFALDSWGQG TLVTVSSASTKGPSVFPLAPSSKSTSGGTAALGCLVKDYFP EPVTVSWNSGALTSGVHTFPAVLQSSGLYSLSSVVTVPSSS LGTQTYICNVNHKPSNTKVDKKVEPKSCDKTHTCPPCPAPE AAGGPSVFLFPPKPKDTLMISRTPEVTCVWDVSHEDPEVK FNWYVDGVEVHNAKTKPREEQYNSTYRWSVLTVLHQD WLNGKEYKCKVSNKALGAPIEKTISKAKGQPREPQVYTLP PSRDELTKNQVSLTCLVKGFYPSDIAVEWESNGQPENNYK TTPPVLDSDGSFFLYSKLTVDKSRWQQGNVFSCSVMHEAL HNHYTQKSLSLSPG | 134 |
| IL7-VAR18/VAR 20 | K81E, G85K | DCDIEG KDGKQYESVL MVSIDQLLDS MKEIGSNCLN NEFNFFKRHI CDANKEGMFL FRAARKLRQF LKMNSTGDFD L HL L ERVSEKT TILLNCTGQV KGRKPAALGE AQPTKSLEEN KSLKEQKKLN DLCFLKRLLQ EIKTCWNKIL, MGTKEH | 135 |
| IL7-VAR18/21 | K81E, G85E | DCDIEG KDGKQYESVL MVSIDQLLDS MKEIGSNCLN NEFNFFKRHI CDANKEGMFL FRAARKLRQF LKMNSTGDFD LHI LFVSEET TILLNCTGQV KGRKPAALGE AQPTKSLEEN KSLKEQKKLN DLCFLKRLLQ EIKTCWNKIL, MGTKEH | 136 |
| Chain K of PD1-IL7-VAR18/VAR 20 | | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFL YSKL TVDKSRWQQGNVFSCSVMHEALHNHYTQK SLSLSPGGGGGSGGGGSGGGGSDCDIEGKDGKQYESVLMV SIDQLLDSMKEIGSNCLNNEFNFFKRHICDANKEGMFLFRA ARKLRQFLKMNSTGDFDLHLLEVSEKTTILLNCTGQVKGR KPAALGEAQPTKSLEENKSLKEQKKLNDLCFLKRLLQEIKT CWNKILMGTKEH | 137 |
| Chain K of PD1-IL7-VAR18/VAR 21 | | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFL YSKL TVDKSRWQQGNVFSCSVMHEALHNHYTQK SLSLSPGGGGGSGGGGSGGGGSDCDIEGKDGKQYESVLMV SIDQLLDSMKEIGSNCLNNEFNFFKRHICDANKEGMFLFRA ARKLRQFLKMNSTGDFDLHLLEVSEETTILLNCTGQVKGR KPAALGEAQPTKSLEENKSLKEQKKLNDLCFLKRLLQEIKT CWNKILMGTKEH | 138 |
| PD1-IL7 (Ref Mol 1-4) Chain A | | DVVMTQSPLSLPVTLGQPASISCRSSQSLVHANTNTYLEWY QQRPGQSPRLLIYKVSNRFSGVPDRFSGSGSGTDFTLKISRV EAEDVGVYYCFQGTHVPNTFGQGTKLEIKRTVAAPSVFIFP PSDEQLKSGTASWCLLNNFYPREAKVQWKVDNALQSGN SQESVTEQDSKDSTYSLSSTLTLSKADYEKHKVYACEVTH QGLSSPVTKSFNRGEC | 139 |
| PD1-IL7 (Ref Mol 1) Chain S | D74E | QIQLVQSGSELKKPGASVKVSCKASGYTFTHYAMNWVRQ APGQGLEWMGWINTNTGEPTYAQGFTGRFVFSLDTSVSTA YLQISSLKAEDTAVYYCAREREPGMDNWGQGTLVTVSSAS TKGPSVFPLAPSSKSTSGGTAALGCLVKDYFPEPVTVSWNS GALTSGVHTFPAVLQSSGLYSLSSWTVPSSSLGTQTYICNV NHKPSNTKVDKKVEPKSCDKTHTCPPCPAPELLGGPSVFLF PPKPKDTLMISRTPEVTCVWDVSHEDPEVKFNWYVDGVE VHNAKTKPREEQYASTYRVVSVLTVLHQDWLNGKEYKCK VSNKALPAPIEKTISKAKGQPREPQVYTLPPSRDELTKNQVS LTCLVKGFYPSDIAVEWESNGQPENNYKTTPPVLDSDGSFF LYSKLTVDKSRWQQGNVFSCSVMHEALHNHYTQKSLSLSP GKGGGGSGGGGSGGGGSDCDIEGKDGKQYESVLMVSIDQ LLD SMKEIGSNCLNNEFNFFKRHICDANKEGMFLFRAARK LRQFLKMNSTGEFDLHLLKVSEGTTILLNCTGQVKGRKPA ALGEAQPTKSLEENKSLKEQKKLNDLCFLKRLLQEIKTCW NKILMGTKEH | 140 |
| PD1-IL7 (Ref Mol 2) Chain S | SS2 (C2S/C141S, C47S/C92S) | QIQLVQSGSELKKPGASVKVSCKASGYTFTHYAMNWVRQ APGQGLEWMGWINTNTGEPTYAQGFTGRFVFSLDTSVSTA YLQISSLKAEDTAVYYCAREREPGMDNWGQGTLVTVSSAS TKGPSVFPLAPSSKSTSGGTAALGCLVKDYFPEPVTVSWNS GALTSGVHTFPAVLQSSGLYSLSSWTVPSSSLGTQTYICNV NHKPSNTKVDKKVEPKSCDKTHTCPPCPAPELLGGPSVFLF PPKPKDTLMISRTPEVTCVWDVSHEDPEVKFNWYVDGVE VHNAKTKPREEQYASTYRVVSVLTVLHQDWLNGKEYKCK VSNKALPAPIEKTISKAKGQPREPQVYTLPPSRDELTKNQVS LTCLVKGFYPSDIAVEWESNGQPENNYKTTPPVLDSDGSFF LYSKLTVDKSRWQQGNVFSCSVMHEALHNHYTQKSLSLSP GKGGGGSGGGGSGGGGSDSDIEGKDGKQYESVLMVSIDQ LLD SMKEIGSNCLNNEFNFFKRHISDANKEGMFLFRAARKL RQFLKMNSTGDFDLHLLKVSEGTTILLNSTGQVKGRKPAA LGEAQPTKSLEENKSLKEQKKLNDLCFLKRLLQEIKTSWN KILMGTKEH | 141 |
| PD1-IL7 (Ref Mol 3) Chain S | SS3 (C47S/C92S, C34S/C129S) | QIQLVQSGSELKKPGASVKVSCKASGYTFTHYAMNWVRQ APGQGLEWMGWINTNTGEPTYAQGFTGRFVFSLDTSVSTA YLQISSLKAEDTAVYYCAREREPGMDNWGQGTLVTVSSAS TKGPSVFPLAPSSKSTSGGTAALGCLVKDYFPEPVTVSWNS GALTSGVHTFPAVLQSSGLYSLSSWTVPSSSLGTQTYICNV NHKPSNTKVDKKVEPKSCDKTHTCPPCPAPELLGGPSVFLF PPKPKDTLMISRTPEVTCVWDVSHEDPEVKFNWYVDGVE VHNAKTKPREEQYASTYRVVSVLTVLHQDWLNGKEYKCK VSNKALPAPIEKTISKAKGQPREPQVYTLPPSRDELTKNQVS LTCLVKGFYPSDIAVEWESNGQPENNYKTTPPVLDSDGSFF LYSKLTVDKSRWQQGNVFSCSVMHEALHNHYTQKSLSLSP GKGGGGSGGGGSGGGGSDCDIEGKDGKQYESVLMVSIDQ LLDSMKEIGSNSLNNEFNFFKRHISDANKEGMFLFRAARKL RQFLKMNSTGDFDLHLLKVSEGTTILLNSTGQVKGRKPAA LGEAQPTKSLEENKSLKEQKKLNDLSFLKRLLQEIKTCWN KILMGTKEH | 142 |
| PD1-IL7 (Ref Mol 4) Chain S | W142H | QIQLVQSGSELKKPGASVKVSCKASGYTFTHYAMNWVRQ APGQGLEWMGWINTNTGEPTYAQGFTGRFVFSLDTSVSTA YLQISSLKAEDTAVYYCAREREPGMDNWGQGTLVTVSSAS TKGPSVFPLAPSSKSTSGGTAALGCLVKDYFPEPVTVSWNS GALTSGVHTFPAVLQSSGLYSLSSWTVPSSSLGTQTYICNV NHKPSNTKVDKKVEPKSCDKTHTCPPCPAPELLGGPSVFLF PPKPKDTLMISRTPEVTCVWDVSHEDPEVKFNWYVDGVE VHNAKTKPREEQYASTYRVVSVLTVLHQDWLNGKEYKCK VSNKALPAPIEKTISKAKGQPREPQVYTLPPSRDELTKNQVS LTCLVKGFYPSDIAVEWESNGQPENNYKTTPPVLDSDGSFF LYSKLTVDKSRWQQGNVFSCSVMHEALHNHYTQKSLSLSP GKGGGGSGGGGSGGGGSDCDIEGKDGKQYESVLMVSIDQ LLD SMKEIGSNCLNNEFNFFKRHICDANKEGMFLFRAARK LRQFLKMNSTGDFDLHLLKVSEGTTILLNCTGQVKGRKPA ALGEAQPTKSLEENKSLKEQKKLNDLCFLKRLLQEIKTCHN KILMGTKEH | 143 |
| Chain K of Ref Mol 5 | D74E | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFL YSKL TVDKSRWQQGNVFSCSVMHEALHNHYTQK SLSLSPGGGGGSGGGGSGGGGSDCDIEGKDGKQYESVLMV SIDQLLDSMKEIGSNCLNNEFNFFKRHICDANKEGMFLFRA ARKLRQFLKMNSTGEFDLHLLKVSEGTTILLNCTGQVKGR KPAALGEAQPTKSLEENKSLKEQKKLNDLCFLKRLLQEIKT CWNKILMGTKEH | 144 |
| Chain K of Ref Mol 6 | SS2 (C2S/C141S, C47S/C92S) | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFL YSKL TVDKSRWQQGNVFSCSVMHEALHNHYTQK SLSLSPGGGGGSGGGGSGGGGSDSDIEGKDGKQYESVLMV SIDQLLDSMKEIGSNCLNNEFNFFKRHISDANKEGMFLFRA ARKLRQFLKMNSTGDFDLHLLKVSEGTTILLNSTGQVKGR KPAALGEAQPTKSLEENKSLKEQKKLNDLCFLKRLLQEIKT SWNKILMGTKEH | 145 |
| Chain K of Ref Mol 7 | SS3 (C47S/C92S, C34S/C129S) | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFL YSKL TVDKSRWQQGNVFSCSVMHEALHNHYTQK SLSLSPGGGGGSGGGGSGGGGSDCDIEGKDGKQYESVLMV SIDQLLD SMKEIGSNSLNNEFNFFKRHISDANKEGMFLFRA ARKLRQFLKMNSTGDFDLHLLKVSEGTTILLNSTGQVKGR KPAALGEAQPTKSLEENKSLKEQKKLNDLSFLKRLLQEIKT CWNKILMGTKEH | 146 |
| Chain K of Ref Mol 8 | W142H | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPCRDELTK NQVSLWCLVKGFYPSDIAVEWESNGQPENNYKTTPPVLDS DGSFFL YSKL TVDKSRWQQGNVFSCSVMHEALHNHYTQK SLSLSPGGGGGSGGGGSGGGGSDCDIEGKDGKQYESVLMV SIDQLLDSMKEIGSNCLNNEFNFFKRHICDANKEGMFLFRA ARKLRQFLKMNSTGDFDLHLLKVSEGTTILLNCTGQVKGR KPAALGEAQPTKSLEENKSLKEQKKLNDLCFLKRLLQEIKT CHNKILMGTKEH | 147 |
| FAP (4B9) IgG kh PGLALA, Chain A | | EIVLTQSPGTLSLSPGERATLSCRASQSVTSSYLAWYQQKP GQAPRLLINVGSRRATGIPDRFSGSGSGTDFTLTISRLEPEDF AVYYCQQGIMLPPTFGQGTKVEIKRTVAAPSVFIFPPSDEQL KSGTASVVCLLNNFYPREAKVQWKVDNALQSGNSQESVT EQDSKDSTYSLSSTLTLSKADYEKHKVYACEVTHQGLSSP VTKSFNRGEC | 148 |
| FAP (4B9) IgG kh PGLALA, Chain H | | EVQLLESGGGLVQPGGSLRLSCAASGFTFSSYAMSWVRQA PGKGLEWVSAIIGSGASTYYADSVKGRFTISRDNSKNTLYL QMNSLRAEDTAVYYCAKGWFGGFNYWGQGTLVTVSSAS TKGPSVFPLAPSSKSTSGGTAALGCLVKDYFPEPVTVSWNS GALTSGVHTFPAVLQSSGLYSLSSWTVPSSSLGTQTYICNV NHKPSNTKVDKKVEPKSCDKTHTCPPCPAPEAAGGPSVFLF PPKPKDTLMISRTPEVTCVWDVSHEDPEVKFNWYVDGVE VHNAKTKPREEQYNSTYRWSVLTVLHQDWLNGKEYKCK VSNKALGAPIEKTISKAKGQPREPQVCTLPPSRDELTKNQV SLSCAVKGFYPSDIAVEWESNGQPENNYKTTPPVLDSDGSF FLVSKLTVDKSRWQQGNVFSCSVMHEALHNHYTQKSLSLS PGK | 149 |
| FAP (4B9) IgG kh PGLALA huIL7wt, Chain K | | EVQLLESGGGLVQPGGSLRLSCAASGFTFSSYAMSWVRQA PGKGLEWVSAIIGSGASTYYADSVKGRFTISRDNSKNTLYL QMNSLRAEDTAVYYCAKGWFGGFNYWGQGTLVTVSSAS TKGPSVFPLAPSSKSTSGGTAALGCLVKDYFPEPVTVSWNS GALTSGVHTFPAVLQSSGLYSLSSWTVPSSSLGTQTYICNV NHKPSNTKVDKKVEPKSCDKTHTCPPCPAPEAAGGPSVFLF PPKPKDTLMISRTPEVTCVWDVSHEDPEVKFNWYVDGVE VHNAKTKPREEQYNSTYRWSVLTVLHQDWLNGKEYKCK VSNKALGAPIEKTISKAKGQPREPQVYTLPPCRDELTKNQV SLWCLVKGFYPSDIAVEWESNGQPENNYKTTPPVLDSDGS FFLYSKLTVDKSRWQQGNVFSCSVMHEALHNHYTQKSLSL SPGGGGGSGGGGSGGGGSDCDIEGKDGKQYESVLMVSIDQ LLD SMKEIGSNCLNNEFNFFKRHICDANKEGMFLFRAARK LRQFLKMNSTGDFDLHLLKVSEGTTILLNCTGQVKGRKPA ALGEAQPTKSLEENKSLKEQKKLNDLCFLKRLLQEIKTCW NKILMGTKEH | 150 |
| FAP (4B9) IgG kh PGLALA huIL7-VAR3, Chain K | V15A | EVQLLESGGGLVQPGGSLRLSCAASGFTFSSYAMSWVRQA PGKGLEWVSAIIGSGASTYYADSVKGRFTISRDNSKNTLYL QMNSLRAEDTAVYYCAKGWFGGFNYWGQGTLVTVSSAS TKGPSVFPLAPSSKSTSGGTAALGCLVKDYFPEPVTVSWNS GALTSGVHTFPAVLQSSGLYSLSSWTVPSSSLGTQTYICNV NHKPSNTKVDKKVEPKSCDKTHTCPPCPAPEAAGGPSVFLF PPKPKDTLMISRTPEVTCVWDVSHEDPEVKFNWYVDGVE VHNAKTKPREEQYNSTYRWSVLTVLHQDWLNGKEYKCK VSNKALGAPIEKTISKAKGQPREPQVYTLPPCRDELTKNQV SLWCLVKGFYPSDIAVEWESNGQPENNYKTTPPVLDSDGS FFLYSKLTVDKSRWQQGNVFSCSVMHEALHNHYTQKSLSL SPGGGGGSGGGGSGGGGSDCDIEGKDGKQYESALMVSIDQ LLD SMKEIGSNCLNNEFNFFKRHICDANKEGMFLFRAARK LRQFLKMNSTGDFDLHLLKVSEGTTILLNCTGQVKGRKPA ALGEAQPTKSLEENKSLKEQKKLNDLCFLKRLLQEIKTCW NKILMGTKEH | 151 |
| FAP (4B9) IgG kh PGLALA huIL7-VAR4, Chain K | V15K | EVQLLESGGGLVQPGGSLRLSCAASGFTFSSYAMSWVRQA PGKGLEWVSAIIGSGASTYYADSVKGRFTISRDNSKNTLYL QMNSLRAEDTAVYYCAKGWFGGFNYWGQGTLVTVSSAS TKGPSVFPLAPSSKSTSGGTAALGCLVKDYFPEPVTVSWNS GALTSGVHTFPAVLQSSGLYSLSSWTVPSSSLGTQTYICNV NHKPSNTKVDKKVEPKSCDKTHTCPPCPAPEAAGGPSVFLF PPKPKDTLMISRTPEVTCVWDVSHEDPEVKFNWYVDGVE VHNAKTKPREEQYNSTYRWSVLTVLHQDWLNGKEYKCK VSNKALGAPIEKTISKAKGQPREPQVYTLPPCRDELTKNQV SLWCLVKGFYPSDIAVEWESNGQPENNYKTTPPVLDSDGS FFLYSKLTVDKSRWQQGNVFSCSVMHEALHNHYTQKSLSL SPGGGGGSGGGGSGGGGSDCDIEGKDGKQYESKLMVSIDQ LLD SMKEIGSNCLNNEFNFFKRHICDANKEGMFLFRAARK LRQFLKMNSTGDFDLHLLKVSEGTTILLNCTGQVKGRKPA ALGEAQPTKSLEENKSLKEQKKLNDLCFLKRLLQEIKTCW NKILMGTKEH | 152 |
| FAP (4B9) IgG kh PGLALA huIL7 -VAR18, Chain K | K81E | EVQLLESGGGLVQPGGSLRLSCAASGFTFSSYAMSWVRQA PGKGLEWVSAIIGSGASTYYADSVKGRFTISRDNSKNTLYL QMNSLRAEDTAVYYCAKGWFGGFNYWGQGTLVTVSSAS TKGPSVFPLAPSSKSTSGGTAALGCLVKDYFPEPVTVSWNS GALTSGVHTFPAVLQSSGLYSLSSWTVPSSSLGTQTYICNV NHKPSNTKVDKKVEPKSCDKTHTCPPCPAPEAAGGPSVFLF PPKPKDTLMISRTPEVTCVWDVSHEDPEVKFNWYVDGVE VHNAKTKPREEQYNSTYRWSVLTVLHQDWLNGKEYKCK VSNKALGAPIEKTISKAKGQPREPQVYTLPPCRDELTKNQV SLWCLVKGFYPSDIAVEWESNGQPENNYKTTPPVLDSDGS FFLYSKLTVDKSRWQQGNVFSCSVMHEALHNHYTQKSLSL SPGGGGGSGGGGSGGGGSDCDIEGKDGKQYESVLMVSIDQ LLD SMKEIGSNCLNNEFNFFKRHICDANKEGMFLFRAARK LRQFLKMNSTGDFDLHLLEVSEGTTILLNCTGQVKGRKPA ALGEAQPTKSLEENKSLKEQKKLNDLCFLKRLLQEIKTCW NKILMGTKEH | 153 |
| FAP (4B9) IgG kh PGLALA huIL7 -VAR20, Chain K | G85K | EVQLLESGGGLVQPGGSLRLSCAASGFTFSSYAMSWVRQA PGKGLEWVSAIIGSGASTYYADSVKGRFTISRDNSKNTLYL QMNSLRAEDTAVYYCAKGWFGGFNYWGQGTLVTVSSAS TKGPSVFPLAPSSKSTSGGTAALGCLVKDYFPEPVTVSWNS GALTSGVHTFPAVLQSSGLYSLSSWTVPSSSLGTQTYICNV NHKPSNTKVDKKVEPKSCDKTHTCPPCPAPEAAGGPSVFLF PPKPKDTLMISRTPEVTCVWDVSHEDPEVKFNWYVDGVE VHNAKTKPREEQYNSTYRWSVLTVLHQDWLNGKEYKCK VSNKALGAPIEKTISKAKGQPREPQVYTLPPCRDELTKNQV SLWCLVKGFYPSDIAVEWESNGQPENNYKTTPPVLDSDGS FFLYSKLTVDKSRWQQGNVFSCSVMHEALHNHYTQKSLSL SPGGGGGSGGGGSGGGGSDCDIEGKDGKQYESVLMVSIDQ LLD SMKEIGSNCLNNEFNFFKRHICDANKEGMFLFRAARK LRQFLKMNSTGDFDLHLLKVSEKTTILLNCTGQVKGRKPA ALGEAQPTKSLEENKSLKEQKKLNDLCFLKRLLQEIKTCW NKILMGTKEH | 154 |
| FAP (4B9) IgG kh PGLALA huIL7 -VAR21, Chain K | G85E | EVQLLESGGGLVQPGGSLRLSCAASGFTFSSYAMSWVRQA PGKGLEWVSAIIGSGASTYYADSVKGRFTISRDNSKNTLYL QMNSLRAEDTAVYYCAKGWFGGFNYWGQGTLVTVSSAS TKGPSVFPLAPSSKSTSGGTAALGCLVKDYFPEPVTVSWNS GALTSGVHTFPAVLQSSGLYSLSSWTVPSSSLGTQTYICNV NHKPSNTKVDKKVEPKSCDKTHTCPPCPAPEAAGGPSVFLF PPKPKDTLMISRTPEVTCVWDVSHEDPEVKFNWYVDGVE VHNAKTKPREEQYNSTYRWSVLTVLHQDWLNGKEYKCK VSNKALGAPIEKTISKAKGQPREPQVYTLPPCRDELTKNQV SLWCLVKGFYPSDIAVEWESNGQPENNYKTTPPVLDSDGS FFLYSKLTVDKSRWQQGNVFSCSVMHEALHNHYTQKSLSL SPGGGGGSGGGGSGGGGSDCDIEGKDGKQYESVLMVSIDQ LLD SMKEIGSNCLNNEFNFFKRHICDANKEGMFLFRAARK LRQFLKMNSTGDFDLHLLKVSEETTILLNCTGQVKGRKPA ALGEAQPTKSLEENKSLKEQKKLNDLCFLKRLLQEIKTCW NKILMGTKEH | 155 |
| FAP-IL7 SS2, Chain K | SS2 (C2S/C141S, C47S/C92S) | EVQLLESGGGLVQPGGSLRLSCAASGFTFSSYAMSWVRQA PGKGLEWVSAIIGSGASTYYADSVKGRFTISRDNSKNTLYL QMNSLRAEDTAVYYCAKGWFGGFNYWGQGTLVTVSSAS TKGPSVFPLAPSSKSTSGGTAALGCLVKDYFPEPVTVSWNS GALTSGVHTFPAVLQSSGLYSLSSWTVPSSSLGTQTYICNV NHKPSNTKVDKKVEPKSCDKTHTCPPCPAPEAAGGPSVFLF PPKPKDTLMISRTPEVTCVWDVSHEDPEVKFNWYVDGVE VHNAKTKPREEQYNSTYRWSVLTVLHQDWLNGKEYKCK VSNKALGAPIEKTISKAKGQPREPQVYTLPPCRDELTKNQV SLWCLVKGFYPSDIAVEWESNGQPENNYKTTPPVLDSDGS FFLYSKLTVDKSRWQQGNVFSCSVMHEALHNHYTQKSLSL SPGGGGGSGGGGSGGGGSDSDIEGKDGKQYESVLMVSIDQ LLD SMKEIGSNCLNNEFNFFKRHISDANKEGMFLFRAARKL RQFLKMNSTGDFDLHLLKVSEGTTILLNSTGQVKGRKPAA LGEAQPTKSLEENKSLKEQKKLNDLCFLKRLLQEIKTSWN KILMGTKEH | 156 |
| FAP-targeted IL-2 (Knob/hole), Chain K | | EVQLLESGGGLVQPGGSLRLSCAASGFTFSSYAMSWVRQA PGKGLEWVSAIIGSGASTYYADSVKGRFTISRDNSKNTLYL QMNSLRAEDTAVYYCAKGWFGGFNYWGQGTLVTVSSAS TKGPSVFPLAPSSKSTSGGTAALGCLVKDYFPEPVTVSWNS GALTSGVHTFPAVLQSSGLYSLSSWTVPSSSLGTQTYICNV NHKPSNTKVDKKVEPKSCDKTHTCPPCPAPEAAGGPSVFLF PPKPKDTLMISRTPEVTCVWDVSHEDPEVKFNWYVDGVE VHNAKTKPREEQYNSTYRWSVLTVLHQDWLNGKEYKCK VSNKALGAPIEKTISKAKGQPREPQVYTLPPCRDELTKNQV SLWCLVKGFYPSDIAVEWESNGQPENNYKTTPPVLDSDGS FFLYSKLTVDKSRWQQGNVFSCSVMHEALHNHYTQKSLSL SPGGGGGSGGGGSGGGGSAPASSSTKKTQLQLEHLLLDLQ MILNGINNYKNPKLTRMLTAKFAMPKKATELKHLQCLEEE LKPLEEVLNGAQSKNFHLRPRDLISNINVIVLELKGSETTFM CEYADETATIVEFLNRWITFAQSIISTLT | 157 |
| Pembrolizum ab fusions Chain A | | DIVLTQSPASLAVSLGQRAAISCRASKGVSTSGYSYLHWYQ QKPGQSPKLLIYLASYLESGVPARFSGSGSGTDFTLNIHPVE EEDAATYYCQHSRDLPLTFGTGTKLELKRTVAAPSVFIFPPS DEQLKSGTASWCLLNNFYPREAKVQWKVDNALQSGNSQ ESVTEQDSKDSTYSLSSTLTLSKADYEKHKVYACEVTHQG LSSPVTKSFNRGEC | 158 |
| Pembrolizum ab fusions Chain H | | QVQLQQPGAELVKPGTSVKLSCKASGYTFTNYYMYWVKQ RPGQGLEWIGGINPSNGGTNFNEKFKNKATLTVDSSSSTTY MQLSSLTSEDSAVYYCTRRDYRFDMGFDYWGQGTTLTVS SASTKGPSVFPLAPSSKSTSGGTAALGCLVKDYFPEPVTVS WNSGALTSGVHTFPAVLQSSGLYSLSSVVTVPSSSLGTQTY ICNVNHKPSNTKVDKKVEPKSCDKTHTCPPCPAPEAAGGPS VFLFPPKPKDTLMISRTPEVTCVWDVSHEDPEVKFNWYV DGVEVHNAKTKPREEQYNSTYRWSVLTVLHQDWLNGKE YKCKVSNKALGAPIEKTISKAKGQPREPQVCTLPPSRDELT KNQVSLSCAVKGFYPSDIAVEWESNGQPENNYKTTPPVLD SDGSFFLVSKLTVDKSRWQQGNVFSCSVMHEALHNRFTQK SLSLSPG | 159 |
| Pembrolizum ab₋huIL7 wt Chain K | | QVQLQQPGAELVKPGTSVKLSCKASGYTFTNYYMYWVKQ RPGQGLEWIGGINPSNGGTNFNEKFKNKATLTVDSSSSTTY MQLSSLTSEDSAVYYCTRRDYRFDMGFDYWGQGTTLTVS SASTKGPSVFPLAPSSKSTSGGTAALGCLVKDYFPEPVTVS WNSGALTSGVHTFPAVLQSSGLYSLSSVVTVPSSSLGTQTY ICNVNHKPSNTKVDKKVEPKSCDKTHTCPPCPAPEAAGGPS VFLFPPKPKDTLMISRTPEVTCVWDVSHEDPEVKFNWYV DGVEVHNAKTKPREEQYNSTYRWSVLTVLHQDWLNGKE YKCKVSNKALGAPIEKTISKAKGQPREPQVYTLPPCRDELT KNQVSLWCLVKGFYPSDIAVEWESNGQPENNYKTTPPVLD SDGSFFLYSKLTVDKSRWQQGNVFSCSVMHEALHNHYTQ KSLSLSPGGGGGSGGGGSGGGGDCDIEGKDGKQYESVLM VSIDQLLD SMKEIGSNCLNNEFNFFKRHICDANKEGMFLFR AARKLRQFLKMNSTGDFDLHLLKVSEGTTILLNCTGQVKG RKPAALGEAQPTKSLEENKSLKEQKKLNDLCFLKRLLQEIK TCWNKILMGTKEH | 160 |
| Pembrolizum ab_huIL7-VAR18, Chain K | K81E | QVQLQQPGAELVKPGTSVKLSCKASGYTFTNYYMYWVKQ RPGQGLEWIGGINPSNGGTNFNEKFKNKATLTVDSSSSTTY MQLSSLTSEDSAVYYCTRRDYRFDMGFDYWGQGTTLTVS SASTKGPSVFPLAPSSKSTSGGTAALGCLVKDYFPEPVTVS WNSGALTSGVHTFPAVLQSSGLYSLSSVVTVPSSSLGTQTY ICNVNHKPSNTKVDKKVEPKSCDKTHTCPPCPAPEAAGGPS VFLFPPKPKDTLMISRTPEVTCVWDVSHEDPEVKFNWYV DGVEVHNAKTKPREEQYNSTYRWSVLTVLHQDWLNGKE YKCKVSNKALGAPIEKTISKAKGQPREPQVYTLPPCRDELT KNQVSLWCLVKGFYPSDIAVEWESNGQPENNYKTTPPVLD SDGSFFLYSKLTVDKSRWQQGNVFSCSVMHEALHNHYTQ KSLSLSPGGGGGSGGGGSGGGGDCDIEGKDGKQYESVLM VSIDQLLD SMKEIGSNCLNNEFNFFKRHICDANKEGMFLFR AARKLRQFLKMNSTGDFDLHLLEVSEGTTILLNCTGQVKG RKPAALGEAQPTKSLEENKSLKEQKKLNDLCFLKRLLQEIK TCWNKILMGTKEH | 161 |
| Pembrolizum ab_huIL7-VAR21, Chain K | G85E | QVQLQQPGAELVKPGTSVKLSCKASGYTFTNYYMYWVKQ RPGQGLEWIGGINPSNGGTNFNEKFKNKATLTVDSSSSTTY MQLSSLTSEDSAVYYCTRRDYRFDMGFDYWGQGTTLTVS SASTKGPSVFPLAPSSKSTSGGTAALGCLVKDYFPEPVTVS WNSGALTSGVHTFPAVLQSSGLYSLSSVVTVPSSSLGTQTY ICNVNHKPSNTKVDKKVEPKSCDKTHTCPPCPAPEAAGGPS VFLFPPKPKDTLMISRTPEVTCVWDVSHEDPEVKFNWYV DGVEVHNAKTKPREEQYNSTYRWSVLTVLHQDWLNGKE YKCKVSNKALGAPIEKTISKAKGQPREPQVYTLPPCRDELT KNQVSLWCLVKGFYPSDIAVEWESNGQPENNYKTTPPVLD SDGSFFLYSKLTVDKSRWQQGNVFSCSVMHEALHNHYTQ KSLSLSPGGGGGSGGGGSGGGGDCDIEGKDGKQYESVLM VSIDQLLD SMKEIGSNCLNNEFNFFKRHICDANKEGMFLFR AARKLRQFLKMNSTGDFDLHLLKVSEETTILLNCTGQVKG RKPAALGEAQPTKSLEENKSLKEQKKLNDLCFLKRLLQEIK TCWNKILMGTKEH | 162 |
| Pembrolizum ab₋huIL7- VAR18/VAR 20, Chain K | K81E, G85K | QVQLQQPGAELVKPGTSVKLSCKASGYTFTNYYMYWVKQ RPGQGLEWIGGINPSNGGTNFNEKFKNKATLTVDSSSSTTY MQLSSLTSEDSAVYYCTRRDYRFDMGFDYWGQGTTLTVS SASTKGPSVFPLAPSSKSTSGGTAALGCLVKDYFPEPVTVS WNSGALTSGVHTFPAVLQSSGLYSLSSVVTVPSSSLGTQTY ICNVNHKPSNTKVDKKVEPKSCDKTHTCPPCPAPEAAGGPS VFLFPPKPKDTLMISRTPEVTCVWDVSHEDPEVKFNWYV DGVEVHNAKTKPREEQYNSTYRWSVLTVLHQDWLNGKE YKCKVSNKALGAPIEKTISKAKGQPREPQVYTLPPCRDELT KNQVSLWCLVKGFYPSDIAVEWESNGQPENNYKTTPPVLD SDGSFFLYSKLTVDKSRWQQGNVFSCSVMHEALHNHYTQ KSLSLSPGGGGGSGGGGSGGGGDCDIEGKDGKQYESVLM VSIDQLLD SMKEIGSNCLNNEFNFFKRHICDANKEGMFLFR AARKLRQFLKMNSTGDFDLHLLEVSEKTTILLNCTGQVKG RKPAALGEAQPTKSLEENKSLKEQKKLNDLCFLKRLLQEIK TCWNKILMGTKEH | 163 |
| Pembrolizum ab_huIL7-VAR18/ VAR21, Chain K | K81E, G85E | QVQLQQPGAELVKPGTSVKLSCKASGYTFTNYYMYWVKQ RPGQGLEWIGGINPSNGGTNFNEKFKNKATLTVDSSSSTTY MQLSSLTSEDSAVYYCTRRDYRFDMGFDYWGQGTTLTVS SASTKGPSVFPLAPSSKSTSGGTAALGCLVKDYFPEPVTVS WNSGALTSGVHTFPAVLQSSGLYSLSSVVTVPSSSLGTQTY ICNVNHKPSNTKVDKKVEPKSCDKTHTCPPCPAPEAAGGPS VFLFPPKPKDTLMISRTPEVTCVWDVSHEDPEVKFNWYV DGVEVHNAKTKPREEQYNSTYRWSVLTVLHQDWLNGKE YKCKVSNKALGAPIEKTISKAKGQPREPQVYTLPPCRDELT KNQVSLWCLVKGFYPSDIAVEWESNGQPENNYKTTPPVLD SDGSFFLYSKLTVDKSRWQQGNVFSCSVMHEALHNHYTQ KSLSLSPGGGGGSGGGGSGGGGDCDIEGKDGKQYESVLM VSIDQLLD SMKEIGSNCLNNEFNFFKRHICDANKEGMFLFR AARKLRQFLKMNSTGDFDLHLLEVSEETTILLNCTGQVKG RKPAALGEAQPTKSLEENKSLKEQKKLNDLCFLKRLLQEIK TCWNKILMGTKEH | 164 |
| huPD 1 ab, chain A | | DIVMTQSPDSLAVSLGERATINCKASESVDTSDNSFIHWYQ QKPGQSPKLLIYRSSTLESGVPDRFSGSGSGTDFTLTISSLQA EDVAVYYCQQNYDVPWTFGQGTKVEIKRTVAAPSVFIFPP SDEQLKSGTASWCLLNNFYPREAKVQWKVDNALQSGNS QESVTEQDSKDSTYSLSSTLTLSKADYEKHKVYACEVTHQ GLSSPVTKSFNRGEC | 165 |
| huPD 1 ab, chain S | | EVQLLESGGGLVQPGGSLRLSCAASGFSFSSYTMSWVRQA PGKGLEWVATISGGGRDIYYPDSVKGRFTISRDNSKNTLYL QMNSLRAEDTAVYYCVLLTGRVYFALDSWGQGTLVTVSS ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPEAAGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALGAPIEKTISKAKGQPREPQVYTLPPSRDELTK NQVSLTCLVKGFYPSDIAVEWESNGQPENNYKTTPPVLDS DGSFFL YSKL TVDKSRWQQGNVFSCSVMHEALHNHYTQK SLSLSPGK | 166 |

### EXAMPLES

The following are examples of methods and compositions of the invention. It is understood that various other embodiments may be practiced, given the general description provided above.

Exemplary formats are shown as schematic representations in FIGS. 1A, 1B, 1C, 1D and 1E. In FIG. 1A, the IgG-IL,7 immunoconjugate comprises two Fab domains (variable domain, constant domain), a heterodimeric Fc domain and a mutant IL-7 polypeptide fused to a C-terminus of the Fc domain. The IgG-IL,7 immunoconjugate is composed of polypeptides of amino acid sequences according to SEQ ID NO: 122, SEQ ID NO: 123 and SEQ ID NO: 124.

In FIG. 1B, the IgG-IL,7 immunoconjugate comprises two Fab domains (variable domain, constant domain), a homodimeric Fc domain and two mutant IL-7 polypeptides fused to the C-termini of the Fc domain. The IgG-IL,7 immunoconjugate is composed of polypeptides of amino acid sequences according to SEQ ID NO: 125 and SEQ ID NO: 126. In FIG. 1C, the IgG-IL7 immunoconjugate comprises one Fab domain (variable domain, constant domain), a heterodimeric Fc domain and one mutant IL-7 polypeptide fused to a C-terminus of the Fc domain. The IgG-IL7 immunoconjugate is composed of polypeptides of amino acid sequences according to SEQ ID NO: 127, SEQ ID NO: 128 and SEQ ID NO: 129. In FIG. 1D, the IgG-IL7 immunoconjugate format, comprising two Fab domain (variable domain, constant domain), a heterodimeric Fc domain and one mutant IL-7 polypeptide fused to an N-terminus of one of the Fab domains. The IgG-IL,7 immunoconjugate is composed of polypeptides of amino acid sequences according to SEQ ID NO: 130, SEQ ID NO: 131 and SEQ ID NO: 132. In FIG. 1E, the IgG-IL-7 immunoconjugate comprises two Fab domain (variable domain, constant domain), a homodimeric Fc domain and two mutant IL-7 polypeptide fused to the N-termini of the Fab domains. The IgG-IL7 immunoconjugate is composed of polypeptides of amino acid sequences according to SEQ ID NO: 133 and SEQ ID NO: 134. The sequences provided for the exemplary formats relate to immunoconjugates with an IL-7 wild-type sequences. However, any mutant IL-7 polpypetide as disclosed herein may be incorporated in said formats instead of a wild-type IL-7.

### Example 1

### Example 1.1 Production and Analytics of PD1-IL7v Fusion Proteins

The antibody IL7 fusion constructs, such as the PD1-IL7 variants (PD1-IL7v), as in table 1 were produced in CHO cells. After harvest, the titer of PD1-II,7 constructs present in the supernatants was determined by ProteinA-HPLC. The supernatants were directly used in the assays (cell assays and surface plasmon resonance) without prior purification. A micro-purification (one-step ProteinA purification) was performed and the eluate subjected to analytics (analytical size exclusion chromatography and capillary SDS electrophoresis: CE-SDS) to assess the quality of the molecules present in the supernatants.

TABLE 1

Polypeptide amino acid sequences of tested PD1-IL7 fusion proteins

| | Interaction with receptor chain | SEQ ID NOs |
|---|---|---|
| PD 1-IL7wt | - | 85, 86, 87 |
| PD1-IL7-VAR1 (E13A) | IL-7Ra and IL-2Rg | 85, 86, 88 |
| PD1-IL7-VAR2 (E13K) | IL-7Ra and IL-2Rg | 85, 86, 89 |
| PD1-IL7-VAR3 (V15A) | IL-7Ra | 85, 86, 90 |
| PD1-IL7-VAR4 (V15K) | IL-7Ra | 85, 86, 91 |
| PD1-IL7-VAR5 (V18A) | IL-7Ra | 85, 86, 92 |
| PD1-IL7-VAR6 (V18K) | IL-7Ra | 85, 86, 93 |
| PD1-IL7-VAR7 (D21A) | IL-2Rg | 85, 86, 94 |
| PD1-IL7-VAR8 (D21K) | IL-2Rg | 85, 86, 95 |
| PD1-IL7-VAR9 (Q22A) | IL-7Ra and IL-2Rg | 85, 86, 96 |
| PD1-IL7-VAR10 (Q22K) | IL-7Ra and IL-2Rg | 85, 86, 97 |
| PD1-IL7-VAR11 (D25A) | IL-2Rg | 85, 86, 98 |
| PD1-IL7-VAR12 (D25K) | IL-2Rg | 85, 86, 99 |
| PD1-IL7-VAR13 (D74A) | IL-7Ra | 85, 86, 100 |
| PD1-IL7-VAR14 (D74K) | IL-7Ra | 85, 86, 101 |
| PD1-IL7-VAR15 (L77A) | IL-7Ra | 85, 86, 102 |
| PD1-IL7-VAR16 (L77K) | IL-7Ra | 85, 86, 103 |
| PD1-IL7-VAR17 (K81A) | IL-7Ra | 85, 86, 104 |
| PD1-IL7-VAR18 (K81E) | IL-7Ra | 85, 86, 105 |
| PD1-IL7-VAR19 (E84A) | IL-7Ra | 85, 86, 106 |
| PD1-IL7-VAR20 (G85K) | IL-7Ra | 85, 86, 107 |
| PD1-IL7-VAR21 (G85E) | IL-7Ra | 85, 86, 108 |
| PD1-IL7-VAR22 (I88K) | IL-7Ra | 85, 86, 109 |
| PD1-IL7-VAR23 (Q136A) | IL-2Rg | 85, 86, 110 |
| PD1-IL7-VAR24 (Q136K) | IL-2Rg | 85, 86, 111 |
| PD1-IL7-VAR25 (K139A) | IL-2Rg | 85, 86, 112 |
| PD1-IL7-VAR26 (K139E) | IL-2Rg | 85, 86, 113 |
| PD1-IL7-VAR27 (N143K) | IL-2Rg | 85, 86, 114 |
| PD1-IL7-VAR28 (M147A) | IL-2Rg | 85, 86, 115 |
| PD1-IL7-VAR29 (N-Glc KO, T72A) | - | 85, 86, 116 |
| PD1-IL7-VAR30 (N-Glc KO, T93A) | - | 85,86, 117 |
| PD1-IL7-VAR31 (N-Glc KO, S118A) | - | 85, 86, 118 |
| PD1-IL7-VAR32 (N-Glc KO, T72A, T93A, S118A) | - | 85, 86, 119 |

### Example 1.1. Production of IgG-Like Proteins in CHO Kl Cells

The PD-IL7v constructs were prepared by Evitria using their proprietary vector system with conventional (non-PCR based) cloning techniques and using suspension-adapted CHO K1 cells (originally received from ATCC and adapted to serum-free growth in suspension culture at Evitria). For the production, Evitria used its proprietary, animal-component free and serum-free media (eviGrow and eviMake2) and its proprietary transfection reagent (eviFect). The supernatant was harvested by centrifugation and subsequent filtration (0.2 µm filter).

### Example 1.2. Titer Determination by ProteinA-HPLC

Quantification of Fc containing constructs present in supernatants was performed by Protein A -HPLC on an Agilent HPLC System with UV detector. The supernatants were injected on a POROS 20 A column (Applied Biosystems), washed with 10 mM Tris, 50 mM Glycine, 100 mM NaCl, pH 8.0 and eluted in the same buffer at pH 2.0. The eluted peak area at 280 nm was integrated and converted to concentration by use of a calibration curve generated with standards analyzed in the same run (see Table 2).

TABLE 2

Titer determination of harvested CHO supernatants determined by ProteinA-HPLC

| | Concentration (µg/µ1) |
|---|---|
| PD1-IL7-VAR1 (E13A) | 0.19 |
| PD1-IL7-VAR2 (E13K) | 0.22 |
| PD1-IL7-VAR3 (V15A) | 0.24 |
| PD1-IL7-VAR4 (V15K) | 0.25 |
| PD1-IL7-VAR5 (V18A) | 0.25 |
| PD1-IL7-VAR6 (V18K) | 0.25 |
| PD1-IL7-VAR7 (D21A) | 0.24 |
| PD1-IL7-VAR8 (D21K) | 0.27 |
| PD1-IL7-VAR9 (Q22A) | 0.24 |
| PD1-IL7-VAR10 (Q22K) | 0.23 |
| PD1-IL7-VAR11 (D25A) | 0.25 |
| PD1-IL7-VAR12 (D25K) | 0.23 |
| PD1-IL7-VAR13 (D74A) | 0.25 |
| PD1-IL7-VAR14 (D74K) | 0.23 |
| PD1-IL7-VAR15 (L77A) | 0.23 |
| PD1-IL7-VAR16 (L77K) | 0.26 |
| PD1-IL7-VAR17 (K81A) | 0.24 |
| PD1-IL7-VAR18 (K81E) | 0.25 |
| PD1-IL7-VAR19 (E84A) | 0.23 |
| PD1-IL7-VAR20 (G85K) | 0.22 |
| PD1-IL7-VAR21 (G85E) | 0.24 |
| PD1-IL7-VAR22 (I88K) | 0.25 |
| PD1-IL7-VAR23 (Q136A) | 0.26 |
| PD1-IL7-VAR24 (Q136K) | 0.25 |
| PD1-IL7-VAR25 (K139A) | 0.25 |
| PD1-IL7-VAR26 (K139E) | 0.22 |
| PD1-IL7-VAR27 (N143K) | 0.22 |
| PD1-IL7-VAR28 (M147A) | 0.22 |
| PD1-IL7-VAR29 (N-Glc KO, T72A) | 0.23 |
| PD1-IL7-VAR30 (N-Glc KO, T93A) | 0.22 |
| PD1-IL7-VAR31 (N-Glc KO, S118A) | 0.20 |
| PD1-IL7-VAR32 (N-Glc KO, T72A, T93A, S118A) | 0.23 |
| PD1-IL7wt | 0.24 |

### Example 1.3. Purification of IgG-Like Proteins

Proteins were purified from filtered cell culture supernatants on a liquid handling platform in 96 well format using a one-step Protein A affinity chromatography. In brief, the supernatants were loaded on ProPlus PhyTip Columns (MabSelect SuRe™, Phynexus) and washed with 20 mM sodium phosphate, 20 mM sodium citrate, pH 7.5. Target proteins are eluted in 20 mM sodium citrate, 100 mM sodium chloride, 100 mM glycine, pH 3.0 and neutralized with 0.5 M sodium phosphate, pH 8.0.

### Example 1.4. Analytics of IgG-Like Proteins

Determination of the monomer product peak versus high molecular weight and low molecular weight side product content was performed by HPLC chromatography at 25° C. using analytical size-exclusion column (TSKgel G3000 SW XL or UP-SW3000) equilibrated in running buffer (200 mM KH₂PO₄, 250 mM KC1 pH 6.2, 0.02% NaN_{d}). Purity and molecular weight of the proteins were analyzed by CE-SDS in the presence and absence of a reducing agent using a LabChipGXII or LabChip GX Touch (Perkin Elmer).

TABLE 3

Monomer product peak, high molecular weight (HMW) and low molecular weight (LMW) side products after ProteinA micro-purification determined by analytical size exclusion chromatography

| IL7 variant | Monomer peak (%) | HMW peak (%) | LMW peak (%) |
|---|---|---|---|
| PD1-IL7-VAR1 (E13A) | 81 | 5 | 14 |
| PD1-IL7-VAR2 (E13K) | 67 | 7 | 24 |
| PD1-IL7-VAR3 (V15A) | 77 | 6 | 17 |
| PD1-IL7-VAR4 (V15K) | 79 | 7 | 14 |
| PD1-IL7-VAR5 (V18A) | 79 | 5 | 16 |
| PD1-IL7-VAR6 (V18K) | 80 | 5 | 15 |
| PD1-IL7-VAR7 (D21A) | 78 | 8 | 14 |
| PD1-IL7-VAR8 (D21K) | 77 | 13 | 10 |
| PD1-IL7-VAR9 (Q22A) | 77 | 6 | 17 |
| PD1-IL7-VAR10 (Q22K) | 82 | 5 | 13 |
| PD1-IL7-VAR11 (D25A) | 77 | 8 | 15 |
| PD1-IL7-VAR12 (D25K) | 79 | 7 | 14 |
| PD1-IL7-VAR13 (D74A) | 74 | 10 | 16 |
| PD1-IL7-VAR14 (D74K) | 76 | 11 | 13 |
| PD1-IL7-VAR15 (L77A) | 78 | 6 | 16 |
| PD1-IL7-VAR16 (L77K) | 79 | 6 | 15 |
| PD1-IL7-VAR17 (K81A) | 72 | 7 | 21 |
| PD1-IL7-VAR18 (K81E) | 75 | 12 | 13 |
| PD1-IL7-VAR19 (E84A) | 78 | 10 | 12 |
| PD1-IL7-VAR20 (G85K) | 73 | 10 | 17 |
| PD1-IL7-VAR21 (G85E) | 77 | 8 | 15 |
| PD1-IL7-VAR22 (I88K) | 81 | 5 | 14 |
| PD1-IL7-VAR23 (Q136A) | 80 | 7 | 13 |
| PD1-IL7-VAR24 (Q136K) | 84 | 5 | 11 |
| PD1-IL7-VAR25 (K139A) | 63 | 20 | 17 |
| PD1-IL7-VAR26 (K139E) | 61 | 25 | 14 |
| PD1-IL7-VAR27 (N143K) | 76 | 6 | 18 |
| PD1-IL7-VAR28 (M147A) | 70 | 8 | 22 |
| PD1-IL7-VAR29 (N-Glc KO, T72A) | 66 | 16 | 18 |
| PD1-IL7-VAR30 (N-Glc KO, T93A) | 76 | 7 | 17 |
| PD1-IL7-VAR31 (N-Glc KO, S118A) | 73 | 8 | 19 |
| PD1-IL7-VAR32 (N-Glc KO, T72A, T93A, S118A) | 66 | 19 | 15 |
| PD1-IL7wt | 66 | 4 | 30 |

TABLE 4

Main product peak and size after ProteinA micro-purification determined by non-reduced CE-SDS

| IL7 variant | Main peak (%) | Size (kDa) |
|---|---|---|
| PD1-IL7-VAR1 (E13A) | 90 | 176 |
| PD1-IL7-VAR2 (E13K) | 83 | 177 |
| PD1-IL7-VAR3 (V15A) | 90 | 176 |
| PD1-IL7-VAR4 (V15K) | 92 | 176 |
| PD1-IL7-VAR5 (V18A) | 89 | 176 |
| PD1-IL7-VAR6 (V18K) | 90 | 174 |
| PD1-IL7-VAR7 (D21A) | 90 | 174 |
| PD1-IL7-VAR8 (D21K) | 89 | 176 |
| PD1-IL7-VAR9 (Q22A) | 88 | 177 |
| PD1-IL7-VAR10 (Q22K) | 92 | 175 |
| PD1-IL7-VAR11 (D25A) | 90 | 176 |
| PD1-IL7-VAR12 (D25K) | 90 | 177 |
| PD1-IL7-VAR13 (D74A) | 85 | 175 |
| PD1-IL7-VAR14 (D74K) | 59 | 175 |
| PD1-IL7-VAR15 (L77A) | 90 | 174 |
| PD1-IL7-VAR16 (L77K) | 88 | 175 |
| PD1-IL7-VAR17 (K81A) | 82 | 176 |
| PD1-IL7-VAR18 (K81E) | 83 | 178 |
| PD1-IL7-VAR19 (E84A) | 86 | 177 |
| PD1-IL7-VAR20 (G85K) | 83 | 176 |
| PD1-IL7-VAR21 (G85E) | 83 | 177 |
| PD1-IL7-VAR22 (I88K) | 91 | 176 |
| PD1-IL7-VAR23 (Q136A) | 87 | 175 |
| PD1-IL7-VAR24 (Q136K) | 92 | 177 |
| PD1-IL7-VAR25 (K139A) | 74 | 177 |
| PD1-IL7-VAR26 (K139E) | 64 | 179 |
| PD1-IL7-VAR27 (N143K) | 90 | 179 |
| PD1-IL7-VAR28 (M147A) | 89 | 177 |
| PD1-IL7-VAR29 (N-Glc KO, T72A) | 86 | 170 |
| PD1-IL7-VAR30 (N-Glc KO, T93A) | 85 | 173 |
| PD1-IL7-VAR31 (N-Glc KO, S118A) | 88 | 178 |
| PD1-IL7-VAR32 (N-Glc KO, T72A, T93A, S118A) | 81 | 165 |
| huIL7wt | 77 | 177 |

### Results

The IgG-IL,7 constructs produced in CHO cells were tested in cell assays and surface plasmon resonance without prior purification, but after quantification by ProteinA titer determination (Table 2). The quality was determined after small scale one-step ProteinA purification and revealed that the product peak was between 61 and 84% by analytical size exclusion chromatography analysis (Table 3) and between 59 and 92% by non-reduced capillary electrophoresis (Table 4).

### Conclusion

The PD1-IL7 variants and PD1-IL7wt were produced with similar titers and with good quality profiles and therefore could be compared in assays without prior purification. The N-glycosylation site knock-out variants showed a reduced size per CE-SDS due to the removal of the carbohydrates, except for variant 31 (S118A). This N-glycosylation (N116) site may not be occupied.

### Example 1.5 Production an Analytics of Further PD1-IL7v Fusion Proteins

The antibody IL7 variants fusion constructs, such as the PDI-IL7 variants (PDI-IL7v), as in table 5 were produced in CHO cells. The proteins were purified by ProteinA affinity chromatography and size exclusion chromatography. The end-product analytics consists of monomer content determination by analytical size exclusion chromatography and percentage of main peak determined by non-reduced capillary SDS electrophoresis: CE-SDS.

TABLE 5

Polypeptide amino acid sequences of tested PD1-IL7 fusion proteins

| | Interaction with receptor chain | SEQ ID NOs |
|---|---|---|
| PD 1-IL7wt | - | 85, 86, 87 |
| PD1-IL7-VAR4 (V15K) | IL-7Ra | 85, 86, 91 |
| PD1-IL7-VAR18 (K81E) | IL-7Ra | 85, 86, 105 |
| PD1-IL7-VAR20 (G85K) | IL-7Ra | 85, 86, 107 |
| PD1-IL7-VAR21 (G85E) | IL-7Ra | 85, 86, 108 |
| PD1-IL7-VAR18 (K81E) VAR20 (G85K) | IL-7Ra | 85, 86, 137 |
| PD1-IL7-VAR18 (K81E) VAR21 (G85E) | IL-7Ra | 85, 86, 138 |

Production of IgG-like proteins in CHO cells. The antibody IL7 fusion constructs described herein were prepared either by Wuxi Biologics with expression in their proprietary CHO expression system and purification by proteinA affinity and size exclusion chromatography or were produced by Evitria using their proprietary vector system with conventional (non-PCR based) cloning techniques and using suspension-adapted CHO K1 cells (originally received from ATCC and adapted to serum-free growth in suspension culture at Evitria). For the production, Evitria used its proprietary, animal-component free and serum-free media (eviGrow and eviMake2) and its proprietary transfection reagent (eviFect). The supernatants were harvested by centrifugation and subsequent filtration (0.2 µm filter).

Purification of IgG-like proteins. Proteins were purified from filtered cell culture supernatants referring to standard protocols. In brief, Fc containing proteins were purified from cell culture supernatants by Protein A-affinity chromatography (equilibration buffer: 20 mM sodium citrate, 20 mM sodium phosphate, pH 7.5; elution buffer: 20 mM sodium citrate, pH 3.0). Elution was achieved at pH 3.0 followed by immediate pH neutralization of the sample. The protein was concentrated by centrifugation ( Millipore Amicon® ULTRA-15 (Art.Nr.: UFC903096), and aggregated protein was separated from monomeric protein by size exclusion chromatography in 20 mM histidine, 140 mM sodium chloride, pH 6.0.

Analytics of IgG-like proteins. The concentrations of purified proteins were determined by measuring the absorption at 280 nm using the mass extinction coefficient calculated on the basis of the amino acid sequence according to Pace, et al., Protein Science, 1995, 4, 2411-1423. Purity and molecular weight of the proteins were analyzed by CE-SDS in the presence and absence of a reducing agent using a LabChipGXII or LabChip GX Touch (Perkin Elmer) (Perkin Elmer). Determination of the aggregate content was performed by HPLC chromatography at 25° C. using analytical size-exclusion column (TSKgel G3000 SW XL or UP-SW3000) equilibrated in running buffer (200 mM KH2P04, 250 mM KC1 pH 6.2, 0.02% NaN3).

TABLE 6

Monomer product peak, high molecular weight (HMW) and low molecular weight (LMW) side products determined by analytical size exclusion chromatography

| PD1-IL7 variants | TAPIR ID | Monomer peak (%) | HMW peak (%) | LMW peak (%) |
|---|---|---|---|---|
| PD1-IL7-VAR4 (V15K) | PIAF5541-006 | 99.7 | 0.3 | 0 |
| PD1-IL,7-VAR18 (K81E) | P1AF5555-006 | 99.6 | 0.4 | 0 |
| PD1-IL7-VAR20 (G85K) | P1AF5557-006 | 100 | 0 | 0 |
| PD1-IL7-VAR21 (G85E) | P1AF5558-006 | 99.8 | 0 | 0.2 |
| PD1-IL,7-VAR18 (K81E) VAR20 (G85K) | P1AF9696-035 | 99.7 | 0.3 | 0 |
| PD1-IL,7-VAR18 (K81E) VAR21 (G85E) | P1AG0950-001 | 99 | 1 | 0 |
| PDI-IL7wt | P1AF5572-018 | 99.6 | 0.4 | 0 |

TABLE 7

Main product peak determined by non-reduced CE-SDS

| PD 1-IL7 variants | | Main peak (%) |
|---|---|---|
| PD1-IL,7-VAR4 (V15K) | P1AF5541-006 | 99 |
| PD1-IL,7-VAR18 (K81E) | P1AF5555-006 | 100 |
| PD1-IL7-VAR20 (G85K) | P1AF5557-006 | 100 |
| PD1-IL7-VAR21 (G85E) | PIAF5558-006 | 100 |
| PD1-IL7-VAR18 (K81E) VAR20 (G85K) | P1AF9696-035 | 96 |
| PD1-IL7-VAR18 (K81E) VAR21 (G85E) | P1AG0950-001 | 99 |
| PDI-IL7wt | P1AF5572-018 | 100 |

Results. The purified PD1-IL7 variants constructs were purified by ProteinA and size exclusion chromatography. The quality analysis of the purified material revealed that the monomer content was above 99% by analytical size exclusion chromatography analysis (Table6) and that the main product peak was between 96 and 100% by non-reduced capillary electrophoresis (Table 7). In conclusion, all PD1-IL7 variants could be produced in good quality.

### Example 1.6 Production an Analytics of Further PD1-IL7v Fusion Proteins (Reference Molecules)

The antibody IL7 variants fusion constructs, as in Table 8, were produced in CHO cells. The proteins were purified by ProteinA affinity chromatography and size exclusion chromatography. The end product analytics consists of monomer content determination (by analytical size exclusion chromatography) and percentage of main peak (determined by non-reduced capillary SDS electrophoresis: CE-SDS). Reference molecules 1 to 4 were produced according to the disclosure of WO 2020/127377 A1 and comprise two IL7 moieties per conjugate. For the reference molecules 5 to 8, the IL7 moities as disclosed in WO 2020/127377 A1 were put in the same format comprising one IL7 moiety per conjugate as the other variants disclosed herein.

TABLE 8

Polypeptide amino acid sequences of tested PD1-IL7 fusion proteins

| | SEQ ID NOs | IL7 variant |
|---|---|---|
| PD 1-IL7wt | 85, 86, 87 | |
| Reference Molecule 1 | 139, 140 | D74E |
| Reference Molecule 2 | 139, 141 | SS2 (C2S/C141S, C47S/C92S) |
| Reference Molecule 3 | 139, 142 | SS3 (C47S/C92S, C34S/C129S) |
| Reference Molecule 4 | 139, 143 | W142H |
| Reference Molecule 5 | 85, 86, 144 | D74E |
| Reference Molecule 6 | 85, 86, 145 | SS2 (C2S/C141S, C47S/C92S) |
| Reference Molecule 7 | 85, 86, 146 | SS3 (C47S/C92S, C34S/C129S) |
| Reference Molecule 8 | 85, 86, 147 | W 142H |

Production of IgG-like proteins in CHO cells. The antibody IL7 fusion constructs described herein were prepared either by Wuxi Biologics with expression in their proprietary CHO expression system and purification by proteinA affinity and size exclusion chromatography or were produced by Evitria using their proprietary vector system with conventional (non-PCR based) cloning techniques and using suspension-adapted CHO K1 cells (originally received from ATCC and adapted to serum-free growth in suspension culture at Evitria). For the production, Evitria used its proprietary, animal-component free and serum-free media (eviGrow and eviMake2) and its proprietary transfection reagent (eviFect). Supernatant was harvested by centrifugation and subsequent filtration (0.2 µm filter).

Purification of IgG-like proteins. Proteins were purified from filtered cell culture supernatants referring to standard protocols. In brief, Fc containing proteins were purified from cell culture supernatants by Protein A-affinity chromatography (equilibration buffer: 20 mM sodium citrate, 20 mM sodium phosphate, pH 7.5; elution buffer: 20 mM sodium citrate, pH 3.0). Elution was achieved at pH 3.0 followed by immediate pH neutralization of the sample. The protein was concentrated by centrifugation (Millipore Amicon® ULTRA-15 (Art.Nr.: UFC903096), and aggregated protein was separated from monomeric protein by size exclusion chromatography in 20 mM histidine, 140 mM sodium chloride, pH 6.0.

Analytics of IgG-like proteins. The concentrations of purified proteins were determined by measuring the absorption at 280 nm using the mass extinction coefficient calculated on the basis of the amino acid sequence according to Pace, et al., Protein Science, 1995, 4, 2411-1423. Purity and molecular weight of the proteins were analyzed by CE-SDS in the presence and absence of a reducing agent using a LabChipGXII or LabChip GX Touch (Perkin Elmer) (Perkin Elmer). Determination of the aggregate content was performed by HPLC chromatography at 25° C. using analytical size-exclusion column (TSKgel G3000 SW XL or UP-SW3000) equilibrated in running buffer (200 mM KH2PO4, 250 mM KC1 pH 6.2, 0.02% NaN3).

TABLE 9

Monomer product peak, high molecular weight (HMW) and low molecular weight (LMW) side products determined by analytical size exclusion chromatography

| PD1-IL7 variants | TAPIR ID | Monomer peak (%) | HMW peak (%) | LMW peak (%) |
|---|---|---|---|---|
| Reference molecule 1 | PIAF9655-001 | 98.8 | 1.2 | 0 |
| Reference molecule 2 | P1AF9656-001 | 98.8 | 1.2 | 0 |
| Reference molecule 3 | PIAF9657-001 | 93 | 4.1 | 2.8 |
| Reference molecule 4 | PIAF9658-001 | 99 | 1 | 0 |
| Reference molecule 5 | PIAF9647-027 | 97 | 1.9 | 1 |
| Reference molecule 6 | PIAF9648-033 | 99 | 0.9 | 0 |
| Reference molecule 7 | P1AF9649-012 | 94 | 1.9 | 4 |
| Reference molecule 8 | PIAF9650-004 | 99 | 1 | 0 |
| PDI-IL7wt | P1AF5572-018 | 99.6 | 0.4 | 0 |

TABLE 10

Main product peak determined by non-reduced CE-SDS

| PD 1-IL7 variants | | Main peak (%) |
|---|---|---|
| Reference molecule 1 | PIAF9655-001 | 94 |
| Reference molecule 2 | P1AF9656-001 | 93 |
| Reference molecule 3 | PIAF9657-001 | 94 |
| Reference molecule 4 | PIAF9658-001 | 93 |
| Reference molecule 5 | PIAF9647-027 | 99 |
| Reference molecule 6 | PIAF9648-033 | 100 |
| Reference molecule 7 | P1AF9649-012 | 68 |
| Reference molecule 8 | PIAF9650-004 | 95 |
| PDI-IL7wt | P1AF5572-018 | 100 |

### Results

The purified PD1-IL7 variants constructs were purified by ProteinA and size exclusion chromatography. The quality analysis of the purified material revealed that the monomer content was above 93% by analytical size exclusion chromatography analysis (Table 9) and that the main product peak was between 93 and 100% by non-reduced capillary electrophoresis with the exception of Reference molecule 7 that showed a pronounced shoulder in the non-reduced electropherogram, resulting in a main peak surface of only 68% (Table 10). In conclusion, all PD1-IL7 variants were produced in good quality.

### Example 1.7: FAP-IL7/IL2

Further IL7 conjugates were produced as anti-FAP (Fibroblast Activation Protein) fusions comprising IL7 variants as disclosed herein, namely FAP-IL7wt (SEQ ID NOs 148, 149 and 150), FAP-IL7-VAR3 (SEQ ID NOs 148, 149 and 151), FAP-IL7-VAR4 (SEQ ID NOs 148, 149 and 152), FAP-IL7-VAR18 (SEQ ID NOs 148, 149 and 153), FAP-IL7-VAR20 (SEQ ID NOs 148, 149 and 154), FAP-IL7-VAR21 (SEQ ID NOs 148, 149 and 155) and FAP-IL7-SS2 (SEQ ID NOs 148, 149 and 156). FAP-IL2 with the sequences SEQ ID NO 148, 149 and 157 was also produced.

### Example 1.8: Production and Analytics of Pembrolizumab-IL7 Fusion Constructs

The Pembrolizumab-IL7 fusion constructs described herein were produced in CHO cells. The proteins were purified by ProteinA affinity chromatography and size exclusion chromatography. The end product analytics consists of monomer content determination (by analytical size exclusion chromatography) and percentage of main peak (determined by non-reduced capillary SDS electrophoresis: CE-SDS). The Pembrolizumab-IL7 fusion constructs described herein were prepared by Wuxi Biologics with expression in their proprietary CHO expression system and purification by proteinA affinity and size exclusion chromatography. Proteins were purified from filtered cell culture supernatants referring to standard protocols. In brief, Fc containing proteins were purified from cell culture supernatants by using a MabSelect column (EQ/Wash1: 50 mM Tris-HCl, 150 mM NaCl, pH 7.4, Wash2: 50 mM Tris-HCl, 150 mM Neutralizer: 1 M Arg, pH 9.1). Elution was achieved at pH 3.4 followed by immediate pH neutralization of the sample. The protein was concentrated by centrifugation (Millipore Amicon® ULTRA-15 (Art.Nr.: UFC903096), and aggregated protein was separated from monomeric protein by size exclusion chromatography in 20 mM histidine, 140 mM sodium chloride, pH 6.0. The concentrations of purified proteins were determined by measuring the absorption at 280 nm using the mass extinction coefficient calculated on the basis of the amino acid sequence according to Pace, et al., Protein Science, 1995, 4, 2411-1423. Purity and molecular weight of the proteins were analyzed by CE-SDS in the presence and absence of a reducing agent using a LabChipGXII or LabChip GX Touch (Perkin Elmer) (Perkin Elmer). Deglycosylated and fully reduced mass were detected by LC-MS.

TABLE A

Purity by monomer peak SEC-HPLC and main peak non-reduced CE-SDS (%)

| Pembrolizumab-IL7 fusion construct | TAPIR ID | SEC-HPLC (%) | Caliper-SDS (%) |
|---|---|---|---|
| Pembrolizumab_huIL7 wt (SEQ ID Nos 158, 159, 160) | P1AG3437-001 | 100 | 99.4 |
| Pembrolizumab_huIL7-VAR18 (K81E) wt (SEQ ID Nos 158, 159, 161) | P1AG3438-001 | 100 | 99.4 |
| Pembrolizumab_huIL7- VAR2 1(G85E) wt (SEQ ID Nos 158, 159, 162) | P1AG3439-001 | 99.2 | 99.5 |
| Pembrolizumab_huIL7-VAR18 (K81E) VAR20(G85K) wt (SEQ ID Nos 158, 159, 163) | P1AG3440-001 | 98.1 | 99.3 |
| Pembrolizumab_huIL7-VAR18 (K81E) VAR21(G85E) wt (SEQ ID Nos 158, 159, 164) | P1AG3441-001 | 99.6 | 99.3 |

The purified Pembrolizumab-IL7 fusion constructs were purified by ProteinA and size exclusion chromatography. The quality analysis of the purified material revealed that the monomer content was above 98% by analytical size exclusion chromatography analysis, the main product peak was above 99% by non-reduced SDS capillary electrophoresis (Table A). All Pembrolizumab-IL7 fusion constructs were produced in good quality.

### Example 2

### Example 2.1. Binding Assessment of IL7 Variants to Human IL7Ra-IL2Ry-Fc Heterodimer

Surface Plasmon Resonacne (SPR) experiments were performed on a Biacore T200 with HBS-EP+ as running buffer (0.01 M HEPES pH 7.4, 0.15 M NaCl, 0.005% Surfactant P20 (BR-1006-69, GE Healthcare)). Fc specific antibodies (Roche internal) were directly immobilized by amine coupling on a CM5 chip (GE Healthcare). The PDI-IL7 constructs were captured from supernatants for 120 s at 100 nM. Human IL7Ra-IL2Rγ-Fc heterodimer were composed of SEQ ID NO: 120 and SEQ ID NO: 121. Only the extracellular domains were fused to Fc domains, produced in HEK Expi Cells and purified by two-step column chromatography including affinity purification via Protein A followed by size-exclusion chromatography. A single injection of 800 nM hu IL 7Ra-IL2Rγ-Fc heterodimer was passed over the ligand at 30 µ1/min for 240 sec to record the association phase. The dissociation phase was monitored for 360 s and triggered by switching from the sample solution to HBS-EP+. The chip surface was regenerated after every cycle using one injection of 10 mM glycine pH 2 for 60 sec. Bulk refractive index differences were corrected for by subtracting the response obtained on the reference flow cell 1. The response units after the capture step and at the end of the association phase were recorded and a ratio of binding to capture was calculated. The single binding curves were fitted in the dissociation phase to obtain a koff for ease of comparison (Biacore Evaluation software, GE Healthcare).

TABLE 11

SPR running parameters

| | |
|---|---|
| Instrumentation | Biacore T200 |
| Chip | CM5 (# 768) |
| Fc1 to 4 | Fc specific IgG (Roche internal) |
| Capture | 100 nM PD1-IL7 variants (supernatants) for 120 s, 10 µ1/min |
| Analyte | 800 nM hu IL7Ra-IL2Rγ-Fc avi biotin, single injection |
| Running buffer | HBS-EP |
| T° | 25° C. |
| Flow | 30 µ1 /min |
| Association | 240 sec |
| Dissociation | 360 sec |
| Regeneration | 10 mM glycine pH 2 for 60 sec |

The PD1-IL7 variants (PD1-IL7v) were analyzed for binding of the IL7 moiety to human IL7RαIL2Rγ-Fc. The concentration of the supernatants was determined by ProteinA binding (see Example 1.2).

### Example 2.2: Binding Assessment of IL-7 Variants (IL7v) to Human IL7Ra-IL2Rγ-Fcheterodimer

The ratio of binding to capture was calculated and the dissociation phase was fitted to a single curve to support the characterization of the off-rate. Nine variants with a ratio of binding to capture greater than 0.18 and kd greater than 9.4× 10⁻⁴ (1/s) were selected with possible reduced affinity to the IL7 receptor (see Table 12).

TABLE 12

Selected candidates with possible reduced affinity to IL7 receptor (faster dissociation)

| Sample name captured molecules | kd [1/s] | Ratio binding/capture | Reduced affinity candidate |
|---|---|---|---|
| PD1-IL7-VAR1 (E13A) | 5.55E-04 | 0.35 | |
| PD1-IL7-VAR2 (E13K) | 8.64E-04 | 0.27 | |
| PD1-IL7-VAR3 (V15A) | 1.83E-03 | 0.30 | selected |
| PD1-IL7-VAR4 (V15K) | 9.94E-03 | 0.19 | selected |
| PD1-IL7-VAR5 (V18A) | 1.13E-03 | 0.33 | selected |
| PD1-IL7-VAR6 (V18K) | 1.15E-03 | 0.30 | selected |
| PD1-IL7-VAR7 (D21A) | 6.23E-04 | 0.31 | |
| PD1-IL7-VAR8 (D21K) | 6.34E-04 | 0.29 | |
| PD1-IL7-VAR9 (Q22A) | 5.98E-04 | 0.37 | |
| PD1-IL7-VAR10 (Q22K) | 5.86E-04 | 0.33 | |
| PD1-IL7-VAR11 (D25A) | 6.87E-04 | 0.31 | |
| PD1-IL7-VAR12 (D25K) | 7.15E-04 | 0.29 | |
| PD1-IL7-VAR13 (D74A) | 6.81E-04 | 0.29 | |
| PD1-IL7-VAR14 (D74K) | 8.02E-04 | 0.25 | |
| PD1-IL7-VAR15 (L77A) | 9.45E-04 | 0.30 | selected |
| PD1-IL7-VAR16 (L77K) | 1.19E-03 | 0.27 | selected |
| PD1-IL7-VAR17 (K81A) | 6.48E-04 | 0.32 | |
| PD1-IL7-VAR18 (K81E) | 8.10E-04 | 0.28 | |
| PD1-IL7-VAR19 (E84A) | 6.11E-04 | 0.32 | |
| PD1-IL7-VAR20 (G85K) | 7.41E-03 | 0.06 | |
| PD1-IL7-VAR21 (G85E) | 4.90E-03 | 0.24 | selected |
| PD1-IL7-VAR22 (I88K) | 1.11E-03 | 0.27 | selected |
| PD1-IL7-VAR23 (Q136A) | 7.66E-04 | 0.27 | |
| PD1-IL7-VAR24 (Q136K) | 9.18E-04 | 0.27 | |
| PD1-IL7-VAR25 (K139A) | 5.06E-04 | 0.20 | |
| PD1-IL7-VAR26 (K139E) | 9.47E-04 | 0.07 | |
| PD1-IL7-VAR27 (N143K) | 1.40E-02 | 0.19 | selected |
| PD1-IL7-VAR28 (M147A) | 8.69E-04 | 0.29 | |
| PD1-IL7wt | 4.46E-04 | 0.30 | |

Based on experiments 2.1 and 2.2 nine variants of interleukin 7 with reduced affinity to the recombinant interleukin 7 receptor were selected by surface plasmon resonance (IL7-VAR3; IL7-VAR4; IL7-VAR5; IL7-VAR6; IL7-VAR15; IL7-VAR16; IL7-VAR21; IL7-VAR22; IL7-VAR27). The selection was based on the ratio of binding to capture signal and on an apparent faster dissociation of the PD1-IL7 variants from the IL7 receptor.

### Example 2.3: Binding Assessment of N-Glycosylation Knock-Out Variants of IL7 to Human IL7Rα-IL2Rγ-Fc Heterodimer

The ratio of binding to capture was calculated and the dissociation phase was fitted to a single curve to support characterization of the off-rate. All N-glycosylation knock-out variants (single and triple mutants) have an off-rate similar to wild-type IL7 (Table 13).

TABLE 13

Comparison of wild-type IL7 and N-glycosylation knock-out variants of IL7 for binding to IL7 receptor

| Sample name captured molecules | Ratio binding/capture | kd [1/s] |
|---|---|---|
| PD1-IL7-VAR29 (N-Glc KO, T72A) | 0.29 | 6.45E-04 |
| PD1-IL7-VAR30 (N-Glc KO, T93A) | 0.29 | 7.56E-04 |
| PD1-IL7-VAR31 (N-Glc KO, S118A) | 0.28 | 8.15E-04 |
| PD1-IL7-VAR32 (N-Glc KO, T72A, T93A, S118A) | 0.30 | 4.77E-04 |
| PD I-IL7wt | 0.30 | 4.46E-04 |

As illustrated in table 7 the four tested variants with one or three N-glycosylation sites removed showed similar ratios of binding to capture and dissociation constants to the wild-type IL7. The N-linked carbohydrates do not play a role in the interaction of interleukin 7 with its receptor.

### Example 2.3: Binding Assessment of Further IL7 Variants to Human IL7Rα-IL2Rγ-Fc Heterodimer

Surface Plasmon Resonance (SPR) experiments were performed on a Biacore 8 K with HBS-EP + 1 mg/ml BSA as running buffer. Anti-P329G Fc specific antibody (Roche internal) was directly immobilized by amine coupling on a C1 chip (Cytiva). The PD1-IL7 constructs were captured for 140 s at 5 nM. Duplicates of a 2-fold serial dilution series from 2.34 to 300 nM human, cynomolgus or murine IL7Ra-IL2Rg-Fc heterodimer was passed over the ligand at 30 µ1/min for 240 sec to record the association phase. The dissociation phase was monitored for 800 s and triggered by switching from the sample solution to running buffer. The chip surface was regenerated after every cycle using two injections of 10 mM glycine pH 2 for 60 sec. Bulk refractive index differences were corrected for by subtracting the response obtained on the reference flow cell (containing immobilized anti P329G Fc specific IgG only). The affinity constants were derived from the kinetic rate constants by fitting to a 1:1 Langmuir binding using the Biacore evaluation software (Cytiva).

TABLE 14

SPR running parameters

| | |
|---|---|
| Instrumentation | Biacore 8 K (Cytiva) |
| Chip | C1 (# 854) |
| Fc1 to 8 | anti-P329G Fc specific IgG (Roche internal) |
| Capture | 5 nM PD1-IL7 variants for 140 s, 10 µl/min |
| Analyte | Two-fold serial dilution from 2.34 to 300 nM of human, cynomolgus or murine IL7Ra-IL2Rg-Fc avi biotin (heterodimer of the ECD of IL7Ra and IL2Rg chains fused to an Fc) |
| Running buffer | HBS-EP (0.01 M HEPES pH 7.4, 0.15 M NaCl, 0.005% Surfactant P20 (BR-1006-69, Cytiva) + 1 mg/ml BSA |
| T° | 25° C. |
| Flow | 30 µl/min |
| Association | 240 sec |
| Dissociation | 800 sec |
| Regeneration | 10 mM glycine pH 2 for 2 × 60 sec |

The following PD1-IL7 variants were analyzed for binding to IL7Ra-IL2Rg-Fc (tables 15 and 16).

TABLE 15

Description of the samples analyzed for binding to IL7Ra-IL2Rg-Fc

| Sample name captured molecules | TAPIR ID | Conc [g/l] |
|---|---|---|
| PD1-IL7-VAR4 (V15K) | P1AF5541-006 | 2.7 |
| PD1-IL7-VAR18 (K81E) | P1AF5555-006 | 2.9 |
| PD1-IL7-VAR20 (G85K) | P1AF5557-006 | 2.6 |
| PD1-IL7-VAR21 (G85E) | P1AF5558-006 | 2.6 |
| PD1-IL7-VAR18 (K81E) VAR20 (G85K) | P1AF9696-035 | 1.8 |
| PD1-IL7-VAR18 (K81E) VAR21 (G85E) | P1AG0950-001 | 1.9 |
| PD1-ILwt | P1AF5572-018 | 4.4 |

TABLE 16

Description of IL7Ra-IL2Rg-Fc

| Sample name analytes | TAPIR ID | Conc [g/l] |
|---|---|---|
| human IL7Ra-IL2Rg-Fc biotin | P1AF4984-007 | 1.43 |
| cynomolgus IL7Ra-IL2Rg-Fc biotin | P1AF7912-003 | 0.92 |
| murine IL7Ra-IL2Rg-Fc biotin | P1AF7911-003 | 1.41 |

### Affinity Determination of IL7 Variants to Human IL7Ralpha-IL2Rgamma-Fc Heterodimer

Four IL7 single variants and two IL7 double variants were compared to IL7 wild-type for binding to human IL7 receptor (Table 17). The affinity of the IL7 variants to the IL7 receptor was determined using the recombinant heterodimer of the extracellular domains of the IL7 receptor alpha chain and the common IL2 receptor gamma chain.

TABLE 17

Binding of IL7 variants to human IL7 receptor: affinity constants determined by surface plasmon resonance at 25° C. as an average of duplicates

| | | | | |
|---|---|---|---|---|
| Sample name captured molecules | TAPIR ID | ka [1/Ms] | kd [1/s] | KD [M] |
| PD1-IL7-VAR4 (V15K) | P1AF5541-006 | 3.05E+05 | 5.35E-03 | 2.58E-08 |
| PD1-IL7-VAR18 (K81E) | P1AF5555-006 | 3.20E+05 | 7.19E-04 | 2.26E-09 |
| PD1-IL7-VAR20 (G85K) | P1AF5557-006 | 1.89E+05 | 1.43E-02 | 7.58E-08 |
| PD1-IL7-VAR21 (G85E) | P1AF5558-006 | 2.73E+05 | 3.13E-03 | 1.15E-08 |
| PD1-IL7-VAR18 (K81E) VAR20 (G85K) | P1AF9696-035 | 1.35E+05 | 1.62E-02 | 1.21E-07 |
| PD1-IL7-VAR18 (K81E) VAR21 (G85E) | P1AG0950-001 | 7.81E+04 | 2.07E-02 | 2.66E-07 |
| PD1-IL7wt | P1AF5572-018 | 4.44E+05 | 3.13E-04 | 7.68E-10 |

The mutations introduced in IL7 reduce the binding affinity to the human IL7 receptor, with the two double mutants (K81E/G85K and K81E/G85E) showing the lowest affinity.

### Affinity Determination of IL7 Variants to Cynomolgus IL7Ralpha-IL2Rgamma-Fc Heterodimer

Four IL7 single variants and two IL7 double variants were compared to IL7 wild-type for binding to cynomolgus IL7 receptor (Table 18). The affinity of the IL7 variants to the IL7 receptor was determined using the recombinant heterodimer of the extracellular domains of the IL7 receptor alpha chain and the common IL2 receptor gamma chain.

TABLE 18

Binding of IL7 variants to cynomolgus IL7 receptor: affinity constants determined by surface plasmon resonance at 25° C. as an average of duplicates

| | | | | |
|---|---|---|---|---|
| Sample name captured molecules | TAPIR ID | ka [1/Ms] | kd [1/s] | KD [M] |
| PD1-IL7-VAR4 (V15K) | P1AF5541-006 | 3.29E+05 | 1.20E-02 | 3.65E-08 |
| PD1-IL7-VAR18 (K81E) | P1AF5555-006 | 2.42E+05 | 9.51E-04 | 3.93E-09 |
| PD1-IL7-VAR20 (G85K) | P1AF5557-006 | 3.60E+05 | 5.10E-02 | 1.42E-07 |
| PD1-IL7-VAR21 (G85E) | P1AF5558-006 | 2.77E+05 | 4.31E-03 | 1.56E-08 |
| PD1-IL7-VAR18 (K81E) VAR20 (G85K) | P1AF9696-035 | 1.62E+05 | 2.51E-02 | 1.55E-07 |
| PD1-IL7-VAR18 (K81E) VAR21 (G85E) | P1AG0950-001 | 6.70E+04 | 3.50E-02 | 5.29E-07 |
| PD1-IL7wt | P1AF5572-018 | 3.45E+05 | 3.55E-04 | 1.03E-09 |

The mutations introduced in IL7 reduce the binding affinity to the cynomolgus IL7 receptor, with the G85K and the two double mutants (K81E/G85K and K81E/G85E) showing the lowest affinity.

### Affinity Determination of IL7 Variants to Murine IL7Ralpha-IL2Rgamma-Fc Heterodimer

Four IL7 single variants and two IL7 double variants were compared to IL7 wild-type for binding to murine IL7 receptor (Table 19). The affinity of the IL7 variants to the IL7 receptor was determined using the recombinant heterodimer of the extracellular domains of the IL7 receptor alpha chain and the common IL2 receptor gamma chain.

TABLE 19

Binding of IL7 variants to murine IL7 receptor: affinity constants determined by surface plasmon resonance at 25° C. as an average of duplicates

| | | | | |
|---|---|---|---|---|
| Sample name captured molecules | TAPIR ID | ka [1/Ms] | kd [1/s] | KD [M] |
| PD1-IL7-VAR4 (V15K) | P1AF5541-006 | | no binding | |
| PD1-IL7-VAR18 (K81E) | P1AF5555-006 | 3.47E+05 | 3.15E-03 | 8.88E-09 |
| PD1-IL7-VAR20 (G85K) | P1AF5557-006 | | no binding | |
| PD1-IL7-VAR21 (G85E) | P1AF5558-006 | 3.95E+05 | 1.69E-02 | 4.30E-08 |
| PD1-IL7-VAR18 (K81E) VAR20 (G85K) | P1AF9696-035 | | no binding | |
| PD1-IL7-VAR18 (K81E) VAR21 (G85E) | P1AG0950-001 | | no binding | |
| PD1-IL7wt | P1AF5572-018 | 4.26E+05 | 7.49E-04 | 1.76E-09 |

The mutations introduced in IL7 reduce strongly the binding affinity to the murine IL7 receptor and abolish binding for the variants V15K, G85K and the two double mutants (K81E/G85K and K81E/G85E).

Six variants of interleukin-7 (four single amino acid mutations and two double mutants) were compared to wild-type interleukin-7 for binding to the recombinant interleukin-7 receptor by surface plasmon resonance. The following ranking in affinity was obtained on the human IL7 receptor: IL7wt > K81E > G85E > V15K > G85K > K81E+G85K > K81E+G85E. The same ranking was observed for the binding to the cynomolgus IL7 receptor. Binding to the murine IL7 receptor also follows a similar ranking (IL7wt > K81E > G85E) except that no binding can be detected for IL7 variants carrying V15K, G85K, K81E+G85K or K81E+G85E. The six tested interleukin-7 variants have a reduced affinity to the IL7 receptor compared to IL7 wild-type and cover a range of affinities allowing to modulate the interleukin-7 response.

### Example 2.4: Affinity Determination of IL7 Variants to Human IL7Ralpha-IL2Rgamma-Fc Heterodimer

SPR experiments were performed on a Biacore 8 K with HBS-EP + 1 mg/ml BSA as running buffer. Anti-P329G Fc specific antibody (Roche internal) was directly immobilized by amine coupling on a C1 chip (Cytiva). The PD1-IL7 constructs were captured for 140 s at 5 nM. Duplicates of a 2-fold serial dilution series from 2.34 to 300 nM human IL7Ra-IL2Rg-Fc heterodimer was passed over the ligand at 30 µl/min for 240 sec to record the association phase. The dissociation phase was monitored for 800 s and triggered by switching from the sample solution to running buffer. The chip surface was regenerated after every cycle using two injections of 10 mM glycine pH 2 for 60 sec. Bulk refractive index differences were corrected for by subtracting the response obtained on the reference flow cell (containing immobilized anti P329G Fc specific IgG only). The affinity constants were derived from the kinetic rate constants by fitting to a 1:1 Langmuir binding using the Biacore evaluation software (Cytiva).

TABLE 20

SPR running parameters

| | |
|---|---|
| Instrumentation | Biacore 8 K (Cytiva) |
| Chip | C1 (# 854) |
| Fc1 to 8 | anti-P329G Fc specific IgG (Roche internal) |
| Capture | 5 nM PD1-IL7 variants for 140 s, 10 µl/min |
| Analyte | Two-fold serial dilution from 2.34 to 300 nM of human IL7Ra-IL2Rg-Fc avi biotin (heterodimer of the ECD of IL7Ra and IL2Rg chains fused to an Fc) |
| Running buffer | HBS-EP (0.01 M HEPES pH 7.4, 0.15 M NaCl, 0.005% Surfactant P20 (BR-1006-69, Cytiva) + 1 mg/ml BSA |
| T° | 25° C. |
| Flow | 30 µl/min |
| Association | 240 sec |
| Dissociation | 800 sec |
| Regeneration | 10 mM glycine pH 2 for 2 × 60 sec |

The following PD1-IL7 variants were analyzed for binding to IL7Ra-IL2Rg-Fc (table 21).

TABLE 21

Description of the samples analyzed for binding to IL7Ra-IL2Rg-Fc

| Sample name captured molecules | TAPIR ID | Conc [g/l] |
|---|---|---|
| PD1-IL7-VAR4 (V15K) | P1AF5541-006 | 2.7 |
| PD1-IL7-VAR18 (K81E) | P1AF5555-006 | 2.9 |
| PD1-IL7-VAR20 (G85K) | P1AF5557-006 | 2.6 |
| PD1-IL7-VAR21 (G85E) | P1AF5558-006 | 2.6 |
| PD1-IL7-VAR18 (K81E) VAR20 (G85K) | P1AF9696-035 | 1.8 |
| PD1-IL7-VAR18 (K81E) VAR21 (G85E) | P1AG0950-001 | 1.9 |
| Reference molecule 5 | P1AF9647-027 | 0.76 |
| Reference molecule 6 | P1AF9648-033 | 2.5 |
| Reference molecule 7 | P1AF9649-012 | 1.35 |
| Reference molecule 8 | P1AF9650-004 | 3.81 |
| PD1-IL7wt | P1AF5572-018 | 4.4 |

TABLE 22

Description of IL7Ra-IL2Rg-Fc

| Sample name analytes | TAPIR ID | Conc [g/l] |
|---|---|---|
| human IL7Ra-IL2Rg-Fc biotin | P1AF4984-007 | 1.43 |

### Affinity Determination of IL7 Variants to Human IL7Ralpha-IL2Rgamma-Fc Heterodimer

Four IL7 single variants, two IL7 double variants and IL7 wild-type were compared to four reference molecules with modified IL7 for binding to human IL7 receptor (Table 23). The affinity of the IL7 variants to the IL7 receptor was determined using the recombinant heterodimer of the extracellular domains of the IL7 receptor alpha chain and the common IL2 receptor gamma chain.

TABLE 23

Binding of IL7 variants to human IL7 receptor: affinity constants determined by surface plasmon resonance at 25° C. Average of duplicates, except for PD1-IL7-VAR20 (G85K), PD1-IL7-VAR18 (K81E) VAR21 (G85E) and reference molecule 5 with single value determination

| | | | | |
|---|---|---|---|---|
| Sample name captured molecules | TAPIR ID | ka [1/Ms] | kd [1/s] | KD [M] |
| PD1-IL7-VAR4 (V15K) | P1AF5541-006 | 2.51E+05 | 1.01E-02 | 4.02E-08 |
| PD1-IL7-VAR18 (K81E) | P1AF5555-006 | 3.92E+05 | 5.63E-04 | 1.44E-09 |
| PD1-IL7-VAR20 (G85K) | P1AF5557-006 | 1.70E+05 | 1.96E-02 | 1.16E-07 |
| PD1-IL7-VAR21 (G85E) | P1AF5558-006 | 5.04E+05 | 2.67E-03 | 5.36E-09 |
| PD1-IL7-VAR18 (K81E) VAR20 (G85K) | P1AF9696-035 | 9.64E+04 | 1.24E-02 | 1.29E-07 |
| PD1-IL7-VAR18 (K81E) VAR21 (G85E) | P1AG0950-001 | 4.33E+04 | 1.48E-02 | 3.42E-07 |
| Reference molecule 5 | P1AF9647-027 | 3.72E+05 | 2.77E-04 | 7.46E-10 |
| Reference molecule 6 | P1AF9648-033 | 1.01E+05 | 1.41E-03 | 1.41E-08 |
| Reference molecule 7 | P1AF9649-012 | | | no binding |
| Reference molecule 8 | P1AF9650-004 | 3.03E+05 | 4.41E-04 | 1.46E-09 |
| PD1-IL7wt | P1AF5572-018 | 3.89E+05 | 2.18E-04 | 5.67E-10 |

The four IL7 single variants, two IL7 double variants and the reference molecules show different level of reduction in binding affinity to the human IL7 receptor. Reference molecule 7 showed no binding. In conclusion, six variants of interleukin-7 (four single amino acid mutations and two double mutants) and wild-type interleukin-7 were compared to four reference molecules with mutations in interleukin-7 for binding to the recombinant interleukin-7 receptor by surface plasmon resonance. Following ranking in affinity was obtained on human IL7 receptor: IL7wt, Reference molecule 5 > K81E, Reference molecule 8 > G85E > Reference molecule 6 > V15K > G85K, K81E+G85K > K81E+G85E. No binding of Reference molecule 7 was observed.

### Example 2.5: Characterization of Thermal Stability of PDL1-IL7 Variants

The thermal stability of PD1-IL7 variants was measured using an Optim2 system (Avacta Group plc) as the change in scattered light intensity. In a micro cuvette array, 9 µL of the samples in 20 mM Histidine, 140 mM NaCl, pH 6.0 at a concentration of 0.75 mg/mL were heated from 25° C. to 85° C. at a rate of 0.1° Cmin. Scattered light intensity (266 nm laser) was recorded every 0.6° C. and processed with the software Optim client V2 (Avacta Group plc). The aggregation onset temperature is defined as the temperature at which the scattering intensity starts to increase. When the 3D structure of the protein unfolds this turn leads to an exposure of formerly buried tryptophanes. This results in a change of the tryptophane emission spectrum (330 nm and 350 nm). This change was monitored and analyzed by determining the Barycentric mean (Spectral Centre of Mass).

TABLE 24

Assessment of thermal stability (aggregation and melting point) for IL7-wt, non-glycosylated IL7 (Var 32) and eleven different IL7 variants

| | Tagg [°C]± 1 | Tm [°C]± 1 |
|---|---|---|
| PD1-IL7-wt | 59.5 | 59.5 |
| PD1-IL7-V15A (Var3) | 59.0 | 59.0 |
| PD1-IL7-V15K (Var4) | 59.0 | 59.5 |
| PD1-IL7-V18A (Var5) | 59.0 | 59.0 |
| PD1-IL7-V18K (Var6) | 59.0 | 59.5 |
| PD1-IL7-L77A (Var15) | 59.0 | 59.5 |
| PD1-IL7-L77A (Var16) | 59.5 | 59.5 |
| PD1-IL7-K81C (Var18) | 59.5 | 59.5 |
| PD1-IL7-G85K (Var18) | 59.5 | 59.0 |
| PD1-IL7-G85E (Var21) | 59.0 | 58.5 |
| PD1-IL7-188K (Var22) | 59.0 | 59.0 |
| PD1-IL7-N143K (Var27) | 59.5 | 59.0 |
| PD1-IL7-N-Glc KO, T72A, T93A, S118A (Var32) | 59.5 | 58.5 |

Aggregation and melting point of all variants show the same stability as compared to IL7-wt and the non-glycosylation IL-7(Var32). Therefore, the design of the IL7 is not affecting thermal stability.

### Example 3

### Example 3.1: IL-7R Signaling (STAT5-P) Upon Treatment of PD-1+ CD4 T Cells With Increasing Doses of PD1-IL7 Variants

The STAT5 phosphorylation (STAT5-P) was used here to assess the potency of different IL-7 variants based on the amount of IL-7Rα/IL-2Rγ signaling in PD-1+ CD4 T cells.

For this purpose CD4 T cells were sorted from healthy donor PBMCs with CD4 beads (130-045-101, Miltenyi) and activated for 3 days in presence of 1 µg/ml plate bound anti-CD3 (overnight pre-coated, clone OKT3, #317315, BioLegend) and 1 µg/ml of soluble anti-CD28 (clone CD28.2, #302923, BioLegend) antibodies to induce PD-1 expression. Three days later, the cells were harvested and washed several times to remove endogenous IL-2. Then, the cells were seeded into a V-bottom plate before being treated for 12 min at 37° C. with increasing concentrations of treatment antibodies (50 µl, 1:10 dilution steps with the top concentration of 66 nM). To preserve the phosphorylation state, an equal amount of Phosphoflow Fix Buffer I (100 ul, 557870, BD) was added right after 12 minutes incubation with the various constructs. The cells were then incubated for additional 30 min at 37° C. before being permeabilized overnight at -80° C. with Phosphoflow PermBuffer III (558050, BD). On the next day STAT-5 in its phosphorylated form was stained for 30 min at 4° C. by using an anti-STAT-5P antibody (47/Stat5(pY694) clone, 562076, BD).

The cells were acquired at the FACS BD-LSR Fortessa (BD Bioscience). The frequency of STAT-5P were determined with FlowJo (V10) and plotted with GraphPad Prism.

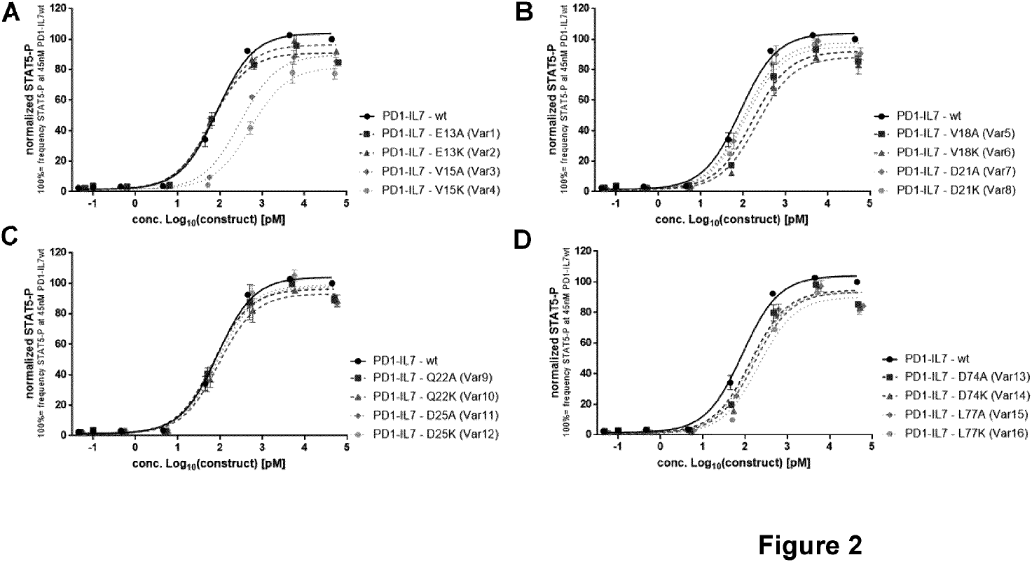

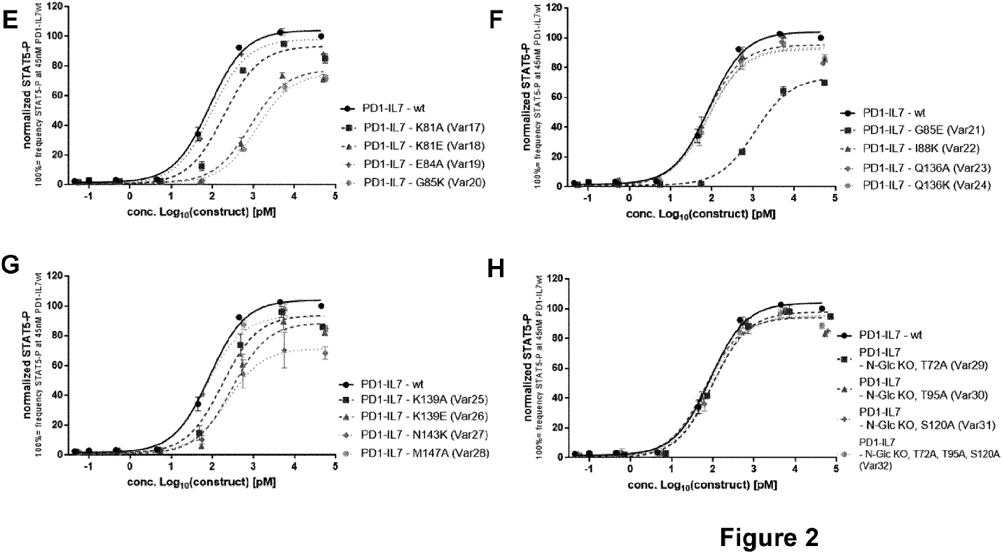

The PD1-IL7 variants carry a mutation to reduce the affinity to the IL7R. STAT5-P is depicted as normalized STAT5-P, where 100% is equal to the frequency of STAT5-P+ cells upon treatment with 66 nM of PD1-IL7 wt in FIG. 2. FIG. 2 shows that the tested PD1-IL7 variants signal in PD-1+ CD4 T cells with similar or reduced potency than PD1-IL7wt, used here as positive control.

TABLE 25

shows the EC50 and Area under the Curve (AUC) of the dose-response STAT-5 phosphorylation for the each mutant on PD-1+ CD4 T cells obtained from 2 donors

| Molecule | EC50 (mean ± SEM) | AUC (mean ± SEM) |
|---|---|---|
| PD1-IL7-VAR1 (E13A) | 66.1 ± 16.6 | 257.4 ± 0.8 |
| PD1-IL7-VAR2 (E13K) | 70.8 ± 11.9 | 265.5 ± 2.9 |
| PD1-IL7-VAR3 (V15A) | 300.3 ± 0.1 | 191.1 ± 13.4 |
| PD1-IL7-VAR4 (V15K) | 539.1 ± 65.1 | 152.4 ± 17.7 |
| PD1-IL7-VAR5 (V18A) | 171.4 ± 15.4 | 218.6 ± 23.1 |
| PD1-IL7-VAR6 (V18K) | 216.6 ± 11 | 199.2 ± 17.6 |
| PD1-IL7-VAR7 (D21A) | 107.7 ± 18.8 | 256.4 ± 21.9 |
| PD1-IL7-VAR8 (D21K) | 118.8 ± 18.7 | 238 ± 20.1 |
| PD1-IL7-VAR9 (Q22A) | 74.1 ± 16.3 | 261.5 ± 24.1 |
| PD1-IL7-VAR10 (Q22K) | 98.6 ± 24.6 | 247.4 ± 22.6 |
| PD1-IL7-VAR11 (D25A) | 97.2 ± 34.2 | 257.4 ± 19.9 |
| PD1-IL7-VAR12 (D25K) | 75.7 ± 7.3 | 274.9 ± 14 |
| PD1-IL7-VAR13 (D74A) | 136.2 ± 11.2 | 230.9 ± 8.4 |
| PD1-IL7-VAR14 (D74K) | 165.1 ± 12.2 | 223.5 ± 7.1 |
| PD1-IL7-VAR15 (L77A) | 138.9 ± 2.1 | 238.2 ± 7.7 |
| PD1-IL7-VAR16 (L77K) | 208.9 ± 5.5 | 204.2 ± 0.2 |
| PD1-IL7-VAR17 (K81A) | 192.3 ± 12.4 | 218.4 ± 0.3 |
| PD1-IL7-VAR18 (K81E) | 852.9 ± 181.1 | 133.1 ± 2.9 |
| PD1-IL7-VAR19 (E84A) | 97.5 ± 7.6 | 256.9 ± 0.5 |
| PD1-IL7-VAR20 (G85K) | 1212.5 ± 32.5 | 120.9 ± 1.3 |
| PD1-IL7-VAR21 (G85E) | 1145.5 ± 120.5 | 118.2 ± 0.4 |
| PD1-IL7-VAR22 (I88K) | 76.6 ± 19.4 | 263.5 ± 14.4 |
| PD1-IL7-VAR23 (Q136A) | 85.5 ± 20.9 | 250.7 ± 21.6 |
| PD1-IL7-VAR24 (Q136K) | 83.7 ± 3.9 | 257.3 ± 19.8 |
| PD1-IL7-VAR25 (K139A) | 181.7 ± 45.5 | 221.6 ± 22.8 |
| PD1-IL7-VAR26 (K139E) | 314.3 ± 66.1 | 189.9 ± 17.5 |
| PD1-IL7-VAR27 (N143K) | 248.7 ± 62.2 | 161.8 ± 30.7 |
| PD1-IL7-VAR28 (M147A) | 73.1 ± 2.4 | 260.6 ± 13.3 |
| PD1-IL7-VAR29 (N-Glc KO, T72A) | 98.2 ± 3.3 | 267.8 ± 15.8 |
| PD1-IL7-VAR30 (N-Glc KO, T93A) | 66.6 ± 11.4 | 265.3 ± 12.4 |
| PD1-IL7-VAR31 (N-Glc KO, S118A) | 93.2 ± 18.4 | 263 ± 10.3 |
| PD1-IL7-VAR32 (N-Glc KO, T72A, T93A, S118A) | 76.8 ± 2.4 | 251.7 ± 7.5 |
| PD1-IL7wt | 86.6 ± 13.5 | 270 ± 9.6 |

### Example 3.2: Selection of IL-7 Variants Through PD-1 Mediated Cis Delivery of IL-7R Signaling (STAT-5P) to PD-1+ CD4 T Cells

In this experiment, the STAT5 phosphorylation was used as a readout to assess whether PD1-IL7v would signal through the IL-7Rα/IL-2Rγ upon binding to PD-1 on the same CD4 T cells (cis) expressing PD-1, rather than on neighbor CD4 T cells (trans) devoided of PD-1 on their surface..

For this purpose CD4 T cells were sorted from healthy donor PBMCs with CD4 beads (130-045-101, Miltenyi) and then were divided in two groups, labelled with different membrane dyes like CFSE (5 µM, 5 min at RT; 65-0850-84, eBioscience) and Cell Trace Violet (5 µM, 5 min at RT; C34557, Thermo Scientific), before being activated for 3 days in presence of 1 µg/ml plate bound anti-CD3 (overnight pre-coated, clone OKT3, #317315, BioLegend) and 1 µg/ml of soluble anti-CD28 (clone CD28.2, #302923, BioLegend) antibodies to induce PD-1 expression. Three days later, the cells were harvested and washed several times to remove endogenous IL-2. The CFSE labelled cellswas further incubated with saturating concentration of anti-PD1 antibody (SEQ ID NOs 165, 166; 10 µg/ml) for 30 min at RT.

Following several washing steps to remove the excess unbound anti-PD-1 antibody, the anti-PD1 pre-treated (CFSE) and untreated (CTV) cells (25 µl, 6*106 cells/ml) were cocultures into a V-bottom plate before being treated for 12 min at 37° C. with 0.1 µg/ml of treatment antibodies (0.66 nM). To preserve the phosphorylation state, an equal amount of Phosphoflow Fix Buffer I (100 µl, 557870, BD) was added right after 12 minutes incubation with the various constructs. The cells were then incubated for additional 30 min at 37° C. before being permeabilized overnight at 80° C. with Phosphoflow PermBuffer III (558050, BD). On the next day STAT-5 in its phosphorylated form was stained for 30 min at 4° C. by using an anti-STAT-5P antibody (47/Stat5(pY694) clone, 562076, BD).

The cells were acquired at the FACS BD-LSR Fortessa (BD Bioscience). The frequency of STAT-5P were determined with FlowJo (V10) and plotted with GraphPad Prism.

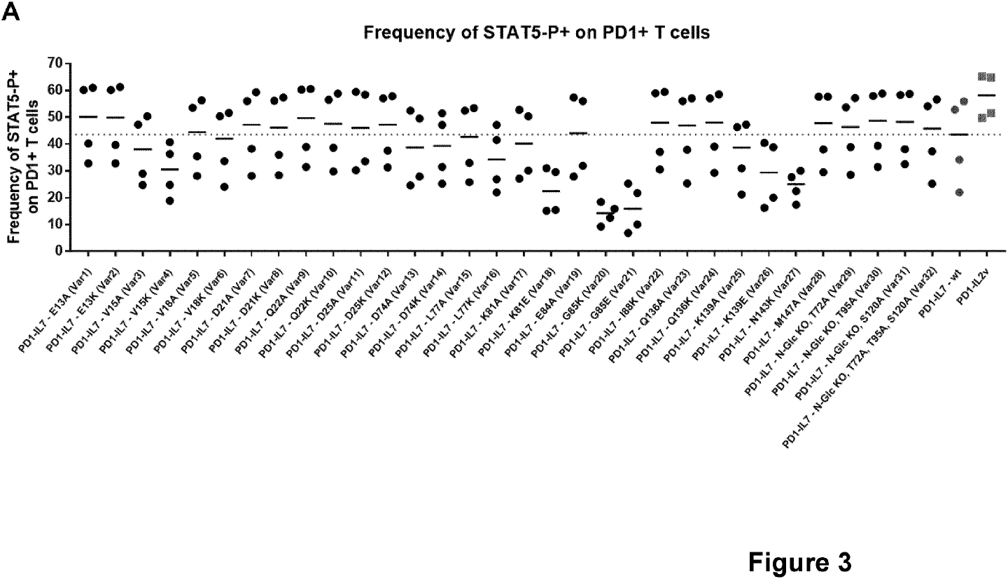

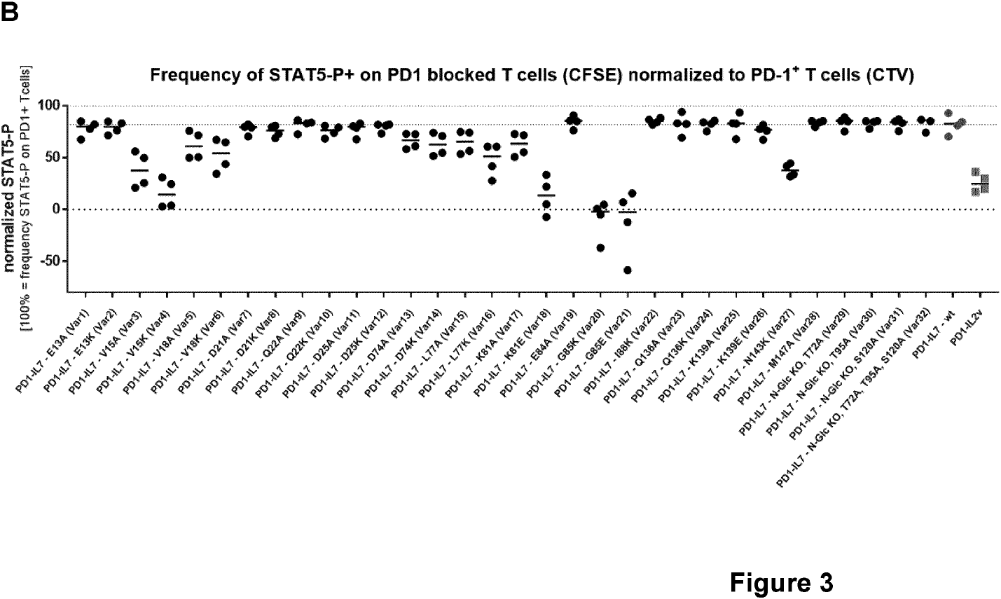

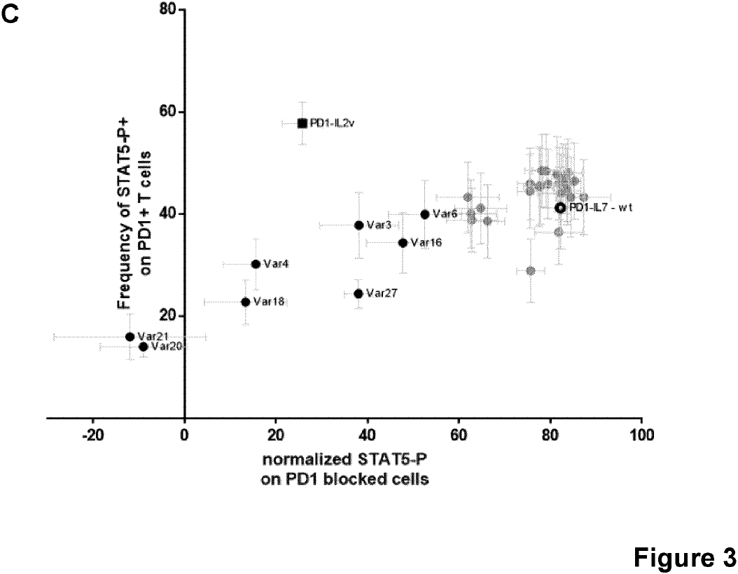

Data in FIG. 3A and FIG. 3B show the frequency of STAT-5P+ cells within the PD1+T cells, labelled with Cell Trace Violet and those labelled with CFSE pre-blocked with PD-1 antibody, upon exposure to 0.1 µg/ml of the 32 PD1-IL7 variants.

FIG. 3A indicates the potency of the IL-7R signaling as certain mutation are associated with lower activity of the IL-7 molecules. FIG. 3B represents the activity of the PD1-IL7v on T cells where PD-1 is pre-blocked and therefore what is measured is the untargeted or trans-delivery of IL7v by PD-1+ T cells in close proximity. PD1-IL7wt and PD1-IL2v (SEQ ID NOs 22, 24, 25) are used as controls where PD1-IL7wt shows 80% of activity also in PD-1 negative T cells (PD-1 pre-blocked). Conversely, PD1-IL2v is active on PD-1+ T cells and drastically loses potency on PD-1 negative T cells, indicating that the delivery of IL-2v is mainly mediated in cis by PD-1 targeting.

Therefore, a suitable PD1-IL7v molecule should also deliver IL-7v in cis to PD-1+ T cells similarly to PD1-IL2v, while retaining appreciable agonistic properties. These features are plotted in FIG. 3C in order to support the identification of those IL-7 variants with desired properties (PD1-IL7-VAR3; PD1-IL7-VAR4; PD1-IL7-VAR6; PD1-IL7-VAR16; PD1-IL7-VAR18; PD1-IL7-VAR20; PD1-IL7-VAR21; PD1-IL7-VAR27).

### Example 3.3: Selection of IL-7 Variants Through PD-1 Mediated Cis Delivery of IL-2R Signaling (STAT5-P) Upon Treatment of PD-1+ CD4 T Cells with Increasing Doses of PD1-IL7 Variants

In this experiment, the STAT5 phosphorylation was used as a readout to assess whether PD1-IL7v would signal, in a dose dependent manner, through the IL-7Rα/IL-2Rγ upon binding to PD-1 on the same CD4 T cells (cis) expressing PD-1, rather than on neighbor CD4 T cells (trans) devoided of PD-1 on their surface.

For this purpose CD4 T cells were sorted from healthy donor PBMCs with CD4 beads (130-045-101, Miltenyi) and then were divided in two groups, labelled with different membrane dyes like CFSE (5 µM, 5 min at RT; 65-0850-84, eBioscience) and Cell Trace Violet (5 µM, 5 min at RT; C34557, Thermo Scientific), before being activated for 3 days in presence of 1 µg/ml plate bound anti-CD3 (overnight pre-coated, clone OKT3, #317315, BioLegend) and 1 µg/ml of soluble anti-CD28 (clone CD28.2, #302923, BioLegend) antibodies to induce PD-1 expression. Three days later, the cells were harvested and washed several times to remove endogenous IL-2. CFSE labelled group was further incubated with saturating concentration of anti-PD1 antibody (SEQ ID NOs 165, 166; 10 µg/ml) for 30 min at RT.

Following several washing steps to remove the excess unbound anti-PD-1 antibody, the anti-PD1 pre-treated (CFSE) and untreated (CTV) cells (25 µl, 6*106 cells/ml) were cocultures into a V-bottom plate before being treated for 12 min at 37° C. with increasing concentrations of treatment antibodies (50 µl, 1:10 dilution steps with the top concentration of 66 nM). To preserve the phosphorylation state, an equal amount of Phosphoflow Fix Buffer I (100ul, 557870, BD) was added right after 12 minutes incubation with the various constructs. The cells were then incubated for additional 30 min at 37° C. before being permeabilized overnight at 80° C. with Phosphoflow PermBuffer III (558050, BD). On the next day STAT-5 in its phosphorylated form was stained for 30 min at 4° C. by using an anti-STAT-5P antibody (47/Stat5(pY694) clone, 562076, BD).

The cells were acquired at the FACS BD-LSR Fortessa (BD Bioscience). The frequency of STAT-5P were determined with FlowJo (V10) and plotted with GraphPad Prism.

TABLE 26

shows the EC50 and Area under the Curve of the dose-response STAT-5 phosphorylation for the selected mutants on PD-1+ and PD-1 pre-blocked CD4 T cells obtained from 4 donors

| | EC50 (mean ± SEM) | AUC (mean ± SEM) | EC50 PD1b1. (mean ± SEM) | AUC PD1b1. (mean ± SEM) |
|---|---|---|---|---|
| PD1-IL7-VAR3 (VISA) | 144.4 ± 12.7 | 173.3 ± 7.4 | 401.2 ± 62.4 | 141.4 ± 11.1 |
| PD1-IL7-VAR4 (V15K) | 261.8 ± 14.7 | 148.1 ± 5.1 | 1253 ± 196.5 | 105.4 ± 8.4 |
| PD1-IL7-VAR6 (V18K) | 131.9 ± 5.5 | 175.8 ± 6 | 307.1 ± 33.8 | 146.2 ± 9 |
| PD1-IL7-VAR16 (L77K) | 126.4 ± 3.7 | 174.8 ± 5.9 | 297.3 ± 33.2 | 145.2 ± 9.4 |
| PD1-IL7-VAR18 (K81E) | 375.4 ± 20.5 | 137.8 ± 4 | 1425.9 ± 239 | 100.4 ± 8.1 |
| PD1-IL7-VAR20 (G85K) | 884.3 ± 86.8 | 111.8 ± 2.5 | 7396.8 ± 1341 | 60.6 ± 6.8 |
| PD1-IL7-VAR21 (G85E) | 515.7 ± 47.9 | 128.8 ± 3.8 | 2606 ± 441 | 85.9 ± 8 |
| PD1-IL7-VAR27 (N143K) | 264.7 ± 23.9 | 124.9 ± 7 | 766.2 ± 134.1 | 94.6 ± 8.6 |
| PD1-IL7wt | 73.2 ± 4.5 | 192.4 ± 6.3 | 105 ± 13.8 | 175.6 ± 9 |

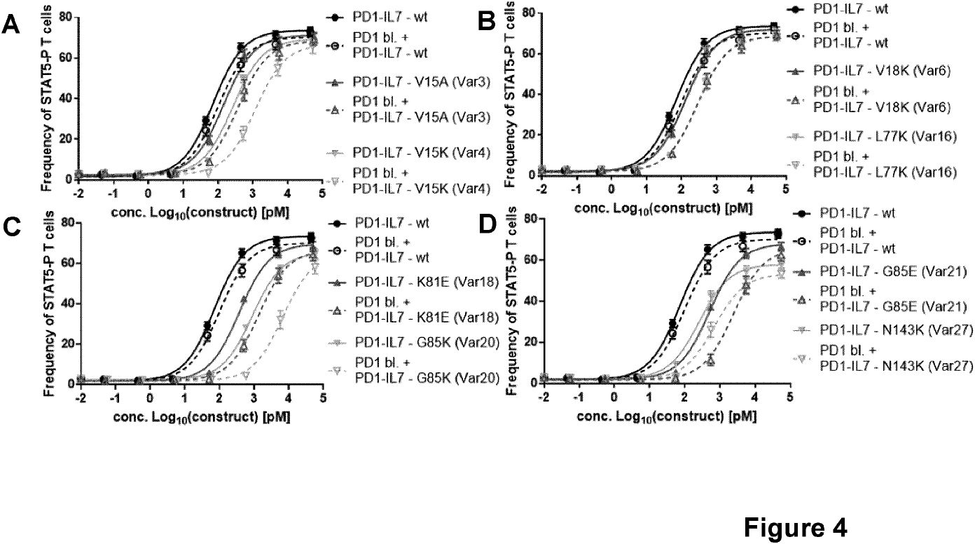

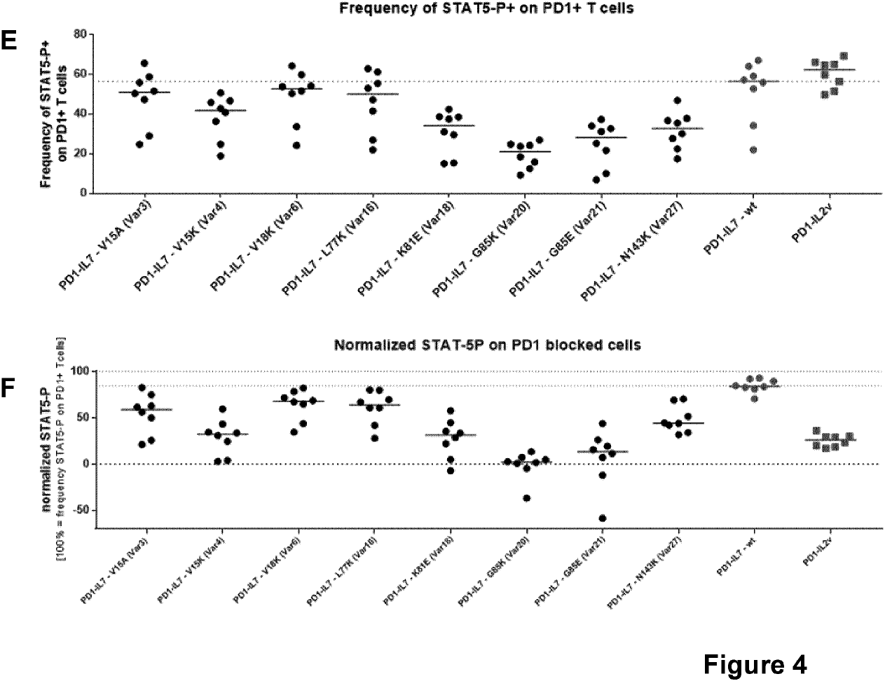

FIGS. 4A-D show the potency difference as IL-2R signaling of selected PD1-IL7 variants in PD-1+ and PD-1 pre-blocked CD4 T cells.

FIG. 4E and FIG. 4F show the frequency of STAT-5P+ cells within the PD1+T cells, labelled with Cell Trace Violet and those labelled with CFSE pre-blocked with PD-1 antibody (SEQ ID NOs 165, 166), upon exposure to 0.1 µg/ml of the 8 selected PD1-IL7 variants.

FIG. 4E indicates the potency of the IL-2R signaling as certain mutation are associated with lower activity of the IL-7 molecules. FIG. 4F represents the activity of the PD1-IL7v on T cells, where PD-1 is pre-blocked and therefore the untargeted or trans-delivery of IL7v by PD-1+ T cells in close proximity is measured. PD1-IL7wt and PD1-IL2v (SEQ ID NOs 22, 24, 25) are used as controls where PD1-IL7wt shows 80% of activity also in PD-1 negative T cells (PD-1 pre-blocked). Conversely, PD1-IL2v is active on PD-1+ T cells and drastically loses potency on PD-1 negative T cells, indicating that the delivery of IL-2v is mediated in cis by PD-1 targeting. In summary, the tested PD1-IL7 variants (PD1-IL7-VAR3; PD1-IL7-VAR4; PD1-IL7-VAR6; PD1-IL7-VAR16; PD1-IL7-VAR18; PD1-IL7-VAR20; PD1-IL7-VAR21; PD1-IL7-VAR27) show the contribution of the PD-1 in mediating/facilitating the delivery of the IL-7variants, in a dose response manner, to the IL-7Rα/IL-2Rγ on PD-1 expressing versus PD-1 devoided CD4 T cells.

### Example 3.4: IL-2R Signaling (STAT5-P) Upon Treatment of PD-1+ CD4 T Cells and PD-1 Pre-Blocked CD4 Tcells with Increasing Doses of PD1-IL7 Variants

In this experiment, the STAT5 phosphorylation was used as readout to assess the potency difference of PD1-IL7v in signalling, in a dose dependent manner, through the IL-7Rα/IL-2Rγ upon binding to PD-1 on PD-1 expressing CD4 T cells versus T cell devoided of PD-1 on their surface, where PD1-IL7v binding relies only on the binding to the IL-7Rα/IL-2Rγ.

For this purpose CD4 T cells were sorted from healthy donor PBMCs with CD4 beads (130-045-101, Miltenyi) and activated for 3 days in presence of 1 µg/ml plate bound anti-CD3 (overnight pre-coated, clone OKT3, #317315, BioLegend) and 1 µg/ml of soluble anti-CD28 (clone CD28.2, #302923, BioLegend) antibodies to induce PD-1 expression. Three days later, the cells were harvested and washed several times to remove endogenous IL-2. Then, the cells were divided in two groups, one of which was incubated with saturating concentration of anti-PD1 antibody (SEQ ID NOs 165, 166; 10 µg/ml) for 30 min at RT.

Following several washing steps to remove the excess unbound anti-PD-1 antibody, the anti-PD1 pre-treated and untreated cells (50 µl, 4*106 cells/ml) were seeded into a V-bottom plate before being treated for 12 min at 37° C. with increasing concentrations of treatment antibodies (50 µl, 1:10 dilution steps with the top concentration of 66 nM ). To preserve the phosphorylation state, an equal amount of Phosphoflow Fix Buffer I (100 µl, 557870, BD) was added right after 12 minutes incubation with the various constructs. The cells were then incubated for additional 30 min at 37° C. before being permeabilized overnight at 80° C. with Phosphoflow PermBuffer III (558050, BD). On the next day STAT-5 in its phosphorylated form was stained for 30 min at 4° C. by using an anti-STAT-5P antibody (47/Stat5(pY694) clone, 562076, BD).

The cells were acquired at the FACS BD-LSR Fortessa (BD Bioscience). The frequency of STAT-5P were determined with FlowJo (V10) and plotted with GraphPad Prism.

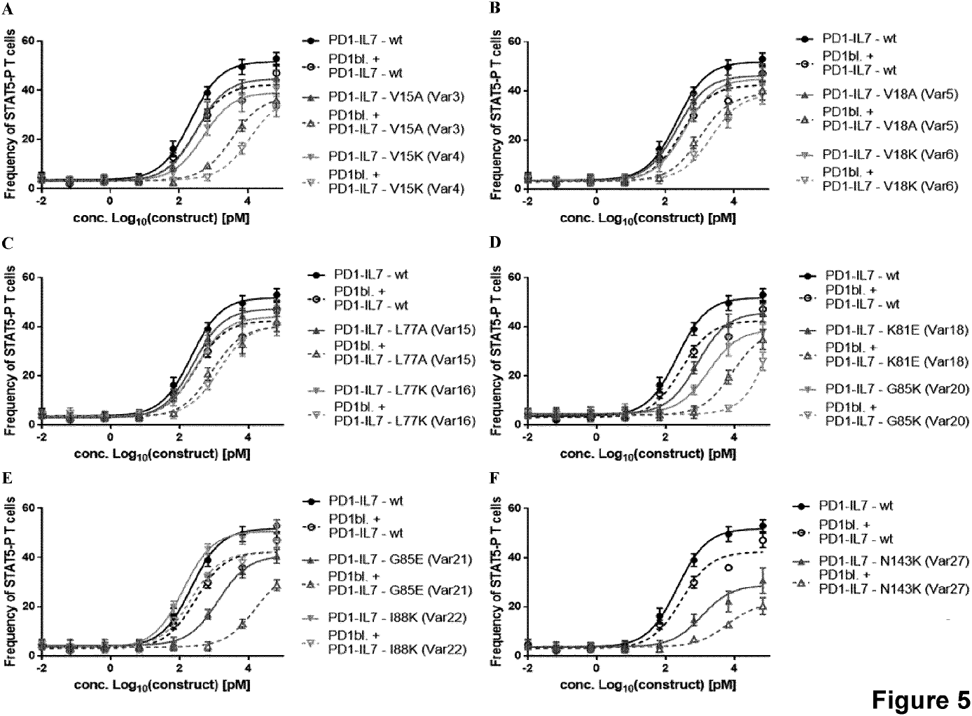

The data in the FIGS. 5A-F show the potency difference of selected PD1-IL7 variants in PD-1+ and PD-1 pre-blocked CD4 T cells. The potency measurement in the PD1+ CD4 T cells reflects the PD1-mediated delivery of IL-7 versus the PD1-independent delivery of IL-7 in PD1 pre-blocked CD4 T cells.

The STAT-5P EC50 fold increase between PD1-mediated and PD-1 independent delivery of IL-7 of each PD1-IL7v molecule was calculated by dividing the EC50 of the PD-1 pre-blocked cells by the EC50 of PD1+ T cells. This provides evidence on the strength of the PD1-dependent delivery of IL7v is for each of the IL7 mutants.

Furthermore, the EC50 fold increase between the PD1-IL7v and PD1-IL7wt was calculated by dividing the EC50 of PD1-IL7v by the EC50 of PD1-IL7wt. This indicated the loss in potency of the PD1-IL7v due to the reduced affinity to the IL-7Rα.

TABLE 27

shows the EC50, EC50 fold increase and Area under the Curve of the dose-response STAT-5 phosphorylation for the selected mutants on PD-1+ and PD-1 pre-blocked CD4 T cells obtained from 4 donors

| | EC50 (mean ± SEM) on PD1⁺ cells | EC50 (mean ± SEM) on PD1-blocked cells | fold increase in EC50 EC50 [with PD1b1] /EC50 [PD1+] | fold increase in EC50 EC50 [PD1+] /EC50 PD1-IL7wt [PD1+] | AUC (mean ± SEM) on PD1+ cells | AUC (mean ± SEM) on PD1-blocked cells |
|---|---|---|---|---|---|---|
| PD1-IL7 -V15A (Var3) | 337.8 ± 46.3 | 3559.3 ± 514.9 | 10.5 ± 0.6 | 1.7 ± 0.4 | 93.2 ± 11.1 | 46.5 ± 6.9 |
| PD1-IL7 -V15K (Var4) | 554 ± 104.6 | 14688.5 ± 4413 | 28.8 ± 8.5 | 2.5 ± 0.3 | 73.6 ± 10.4 | 30.5 ± 5.9 |
| PD1-IL7 -V18A (Var5) | 229.4 ± 33.8 | 1204.2 ± 260 | 5.5 ± 1.2 | 1.1 ± 0.2 | 102.8 ± 12.8 | 64.4 ± 10.8 |
| PD1-IL7 -V18K (Var6) | 412.7 ± 82.6 | 2940.2 ± 927.3 | 8.3 ± 3.2 | 1.9 ± 0.3 | 91.2 ± 12.3 | 53.8 ± 10.8 |
| PD1-IL7 -L77A (Var15) | 270.8 ± 52.7 | 1140.3 ± 334.2 | 4.6 ± 1.3 | 1.2 ± 0.1 | 103.9 ± 11.7 | 68 ± 11.2 |
| PD1-IL7 -L77K (Var16) | 325.9 ± 52.6 | 1617 ± 303 | 5 ± 0.7 | 1.5 ± 0.2 | 92.2 ± 13.4 | 63 ± 11.6 |
| PD1-IL7 -K81E (Var18) | 897.4 ± 191.9 | 9009 ± 1702 | 10.8 ± 2.1 | 4.3 ± 1 | 78.7 ± 9.2 | 33.8 ± 5.7 |
| PD1-IL7 -G85K (Var20) | 2158.3 ± 553 | 77889853.8 ± 53733993.1 | 25039.4 ± 15045.7 | 9.3 ± 0.7 | 56.1 ± 8.2 | 16.3 ± 4.1 |
| PD1-IL7 -G85E (Var21) | 1428.8 ± 325.2 | 18197.8 ± 6098.5 | 12 ± 1.2 | 6.4 ± 0.8 | 63 ± 6.8 | 24.8 ± 2.4 |
| PD1-IL7 - I88K(Var22) | 137 ± 25.7 | 233.5 ± 46.4 | 1.7 ± 0.2 | 0.6 ± 0.1 | 123.5 ± 12.9 | 94.6 ± 10.9 |
| PD1-IL7 -N143K (Var27) | 2210.7 ± 1373.2 | 6161 ± 2049 | 4.7 ± 2.1 | 7.8 ± 2.5 | 44.4 ± 11.4 | 23.3 ± 3.6 |
| PD1-IL7 - wt | 246.3 ± 80.6 | 318.5 ± 90.2 | 1.5 ± 0.4 | 1 ± 0 | 118 ± 11.8 | 91.8 ± 8.8 |

### Example 4

### Example 4.1: Rescue of Tconv Effector Function From Treg Suppression Upon PD1-IL7v Treatment

Here it was assessed whether PD1-IL7 mutants can reverse the Treg suppression of Tconv effector functions. Therefore, a suppressive-function assay was established, and for this purpose, Tconv and Treg were isolated and labelled as follow. CD4⁺ CD25⁺ CD127 dim Regulatory T cells (Treg) were isolated with the two-step Regulatory T cell Isolation Kit (Miltenyi, #130-094-775). In parallel the CD4⁺ CD25⁻ conventional T cells (Tconv) were isolated by collecting the negative fraction of a CD25 positive selection (Miltenyi, #130-092-983) followed by a CD4+ enrichment (Miltenyi, #130-045-101). The Tconv were labelled with CFSE (eBioscience, #65-0850-84) and the Treg were labelled with Cell Trace Violet (ThermoFisher scientific, C34557) to track the proliferation of both populations.

Tconv and Treg were then cultured together for 5 days, with or without treatment, in presence of CD4⁻ CD25⁻ PBMCs from an unrelated donor to provide an allospecific stimulation.

On day 5, the accumulation of cytokines in the Golgi complex was enhanced by applying Protein Transport Inhibitors (GolgiPlug #555029, BD and GolgiStop #554724, BD) for 5 hours prior to the FACS staining.

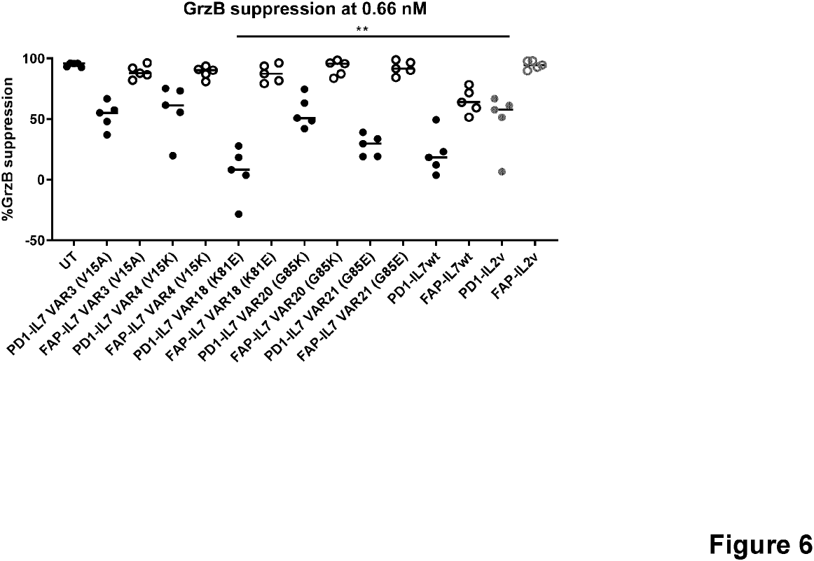

The ability of the proliferated Tconv to secrete granzyme B (GrzB; FIG. 6) in presence and absence of Treg was measured. The Treg suppression was calculated with the following formula:

$$\begin{matrix}\% c y t o k i n e s u p p r e s s i o n = \\ 100 - \left(\frac{\% c y t o k i n {e}_{\left(T c o n v + T r e g + i m m u n o c o n j u g a t e\right)}}{\% c y t o k i n {e}_{\left(T c o n v\right)}} ∗ 100\right)\end{matrix}$$

where % cytokine_{(Tconv+Treg±immunocon.jugate)} is the level of cytokine secreted by Tconv in the presence of Treg ± treatment, % cytokine_{(Tconv)} is the level of cytokine secreted by Tconv in the absence of Treg. In FIG. 6, each symbol represents a separate donor, horizontal lines indicate medians with N=5 donors, from 3 independent experiments. P was calculated using one-way ANOVA (*p<0.05, **p<0.01, ***p<0.001, ****p<0.0001).

In untreated samples, 95% of granzyme B secretion by Tconv was suppressed by Treg. Treatment with untargeted IL-2v or IL-7v did not rescue Tconv secretion of granzyme B from Treg suppression. However, untargeted IL-7wt was able to reduce Treg suppression to 63%, due to IL-7 preferential activity on Tconv given the reduced expression of IL7R by Treg, which translates in a competitive advantage for Tconv over Tregs for its consumption. Interestingly, the PD-1 mediated targeting of IL-7wt and of the mutated IL-7 resulted in a pronounced reduction of Treg suppression ranging between 61% to 8% for the mutants, depending on the single mutation, and reaching 18% for the PD1-IL7wt. PD1-IL2v was used as positive control due to its ability to overcome Treg suppression which in this case was reduced to 57%.

These results highlight the competitive advantage provided to Tconv versus Tregs by the PD-1 mediated delivery of IL-7. They also reveal those mutants with a reduced affinity for the IL-7Rα which still maintain the IL-7wt competitive advantage for Tconv versus Treg (FIG. 6).

### Example 4.2: IL-7R Signaling (STAT5-P) on Activated PD-1+ and PD-1- CD4 T Cells Upon Treatment with Increasing Doses of PD1-IL7 Mutants

In this experiment, the potency and the cis/trans-signaling of four different PD1-IL7 mutants disclosed in WO 2020/127377 A1 (Reference molecules 1 to 4) with reduced affinity for the IL-7Ra/g were measured as IL-7R signaling by treating activated PD1⁺ and PD-1⁻ (anti-PD-1 pre-treated) CD4 T cells with increasing concentration of immunoconjugates. The purpose was to determine the dependency of the PD1-IL7 mutants on the PD-1 expression of the T cells in order to deliver an IL-7R signaling.

Each of these molecules has at least one point mutation in the IL-7 with the intention to lower the affinity either to the IL7Rα or to the common gamma chain (γc).

For this purpose CD4 T cells were sorted from healthy donor PBMCs with CD4 beads (130-045-101, Miltenyi) and activated for 3 days in presence of 1 µg/ml plate bound anti-CD3 (overnight pre-coated, clone OKT3, #317315, BioLegend) and 1 µg/ml of soluble anti-CD28 (clone CD28.2, #302923, BioLegend) antibodies to induce PD-1 expression. Three days later, the cells were harvested and washed several times to remove endogenous cytokines and half of the cells were labelled with CTV (5uM, 5 min at RT; C34557, Thermo Scientific) and the other half left unlabelled.

Then, the CFSE labelled cells were incubated with a saturating concentration of a competing anti-PD-1 antibody (in-house molecule, 10 µg/ml) for 30 min at RT followed by several washing steps to remove the excess unbound anti-PD1 antibody. Thereafter the PD1 pre-blocked CFSE labelled cells (25 µl, 6*106 cells/ml) were co-cultured 1:1 with the PD1+ CTV labelled cells (25 µl, 6*106 cells/ml) in a V-bottom plate before being treated for 12 min at 37° C. with increasing concentrations of treatment antibodies (50 µl, 1:10 dilution steps). To preserve the phosphorylation state, an equal amount of Phosphoflow Fix Buffer I (100ul, 557870, BD) was added right after 12 minutes incubation with the various constructs. The cells were then incubated for additional 30 min at 37° C. before being permeabilized overnight at -80° C. with Phosphoflow PermBuffer III (558050, BD). On the next day STAT-5 in its phosphorylated form was stained for 30 min at 4° C. by using an anti-STAT-5P antibody (47/Stat5(pY694) clone, 562076, BD).

The cells were acquired at the FACS BD-LSR Fortessa (BD Bioscience). The frequency of STAT-5P was determined with FlowJo (V10) and plotted with GraphPad Prism.

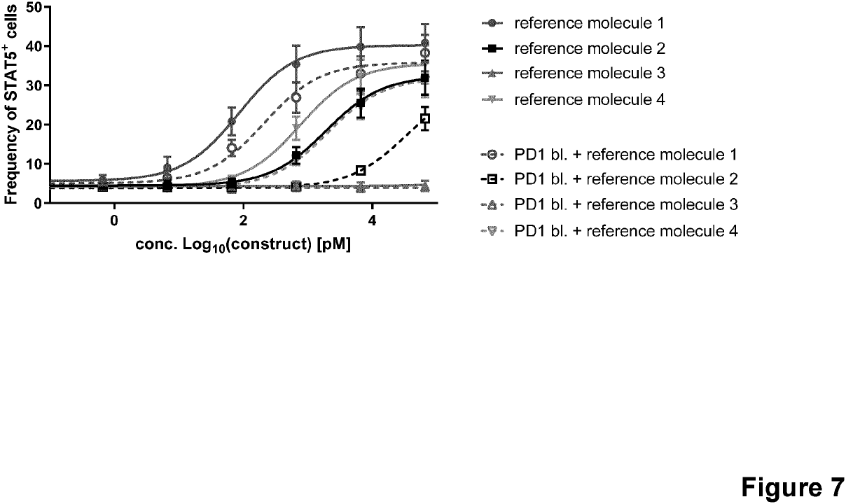

The dose-response curves on PD-1+ T cells provide information on the potency of the reference molecules 1 to 4 in signaling through the IL-7R. In addition, the dose-response curves on T cells pre-treated with a competing anti-PD-1 antibody, to prevent the PD-1 mediated delivery, show the potency of reference molecules 1 to 4 in providing IL-7R signalling independently from PD-1 expression. In this particular assay only reference molecule 2 had more than 12 fold reduced activity on T cells in absence of PD-1 binding than on PD-1+ T cells (FIG. 7, Table 28).

TABLE 28

| | EC50 (PD1+) | EC50 (PD1 blocked) | Area (PD1+) | Area (PD1 blocked) |
|---|---|---|---|---|
| Reference molecule 1 | 85.65 | 211.3 | 104.4 | 81.21 |
| Reference molecule 2 | 1920 | 33420 | 45.11 | 15.36 |
| Reference molecule 3 | nd | nd | 0.6909 | 0.7512 |
| Reference molecule 4 | 761.3 | 2019 | 60.76 | 44.14 |

### Example 4.3: IL-7R Signaling (STAT5-P) on Activated PD-1+ and PD-1- CD4 T Cells Upon Treatment with Increasing Doses of PD1-IL7 Single and Double Mutants

In the following experiment, the IL-7R signaling of 2 different PD1-IL7 mutants and 2 double mutants and Reference molecule 2 were measured by exposing activated PD1⁺ and PD-1⁻ (anti-PD-1 pre-treated) CD4 T cells to increasing concentration of immunoconjugates. Given that PD1-IL7wt acts in a PD1-independent fashion or in trans, meaning that the IL-7wt moiety can signal in PD1 negative T cells cultured in close proximity to PD1⁺ cells, this experiment would allow the selection of PD1-IL7 mutants with reduced affinity to the IL7Ra to ensure the preferential signaling in cis, meaning that the IL-7v moiety mainly binds and signals through the IL-7R on the same PD-1⁺ Tcell after PD-1 docking.

For this purpose, CD4 T cells were sorted from healthy donor PBMCs and the experiment was performed as described for Example 4.2 and FIG. 7.

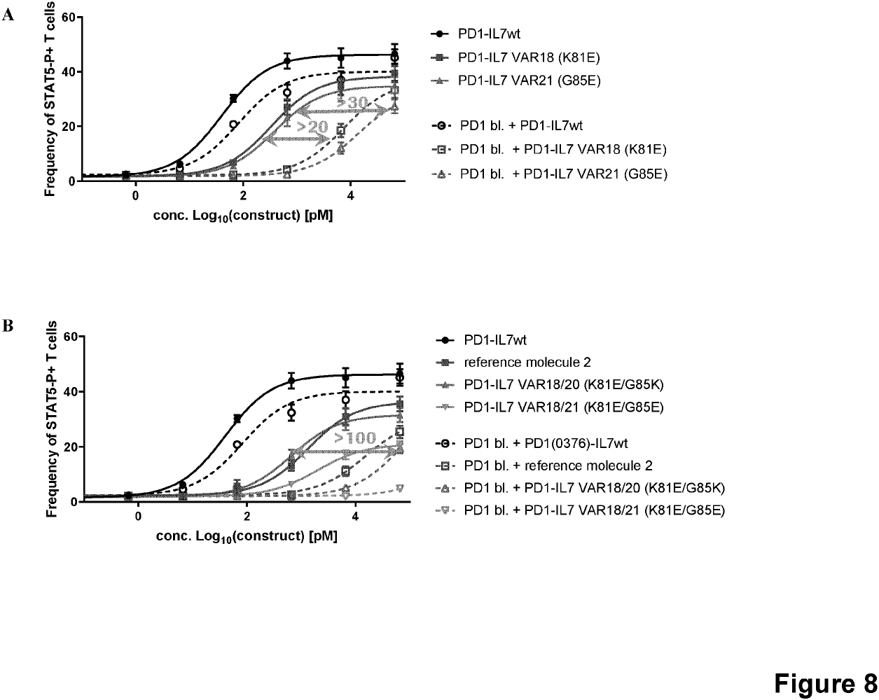

The dose-response curves on PD-1+ T cells provide information on the potency of the single and double mutants in signaling through the IL-7R. In addition, the dose-response curves on T cells pre-treated with a competing anti-PD-1 antibody, to prevent the PD-1 mediated delivery, show the potency of the single and double mutants in providing IL-7R signalling independently from the binding to PD-1. In this particular assay PD1-IL7VAR18 and PD1-IL7VAR21 had, respectively, more than 20 and 30 folds reduced potency on PD-1⁻ T cells than on PD-1+ T cells, indicative of their preferential cis-activity (FIG. 8A). Of note, the PD1-IL7VAR18/20 double mutant showed a drastic reduction in activity of roughly 100 folds on PD-1⁻ T cells when compared to PD-1⁺ T cells (FIG. 8B).

TABLE 29

| | EC50 PD1+ | EC50 PD1 blocked | EC50 [PD1blocked]/ EC50 [PD1+] | EC50 [PD1+]/ EC50[PD1-IL7wt] | AUC PD1+ | AUC PD1 blocked |
|---|---|---|---|---|---|---|
| PD1-IL7wt | 39.8 | 82.0 | 2.1 | 1.0 | 138.6 | 106.5 |
| Reference molecule 2 | 1334.0 | 15411.0 | 11.6 | 33.5 | 59.6 | 22.9 |
| PD1-IL7 VAR18/20 (K81E/G85K) | 655.6 | 71266.0 | 108.7 | 16.5 | 58.0 | 12.9 |
| PD1-IL7 VAR18/21 (K81E/G85E) | 1986.0 | ~44192470 | nd | 49.9 | 28.5 | 3.0 |
| PD1-IL7 VAR18 (K81E) | 331.4 | 7554.0 | 22.8 | 8.3 | 80.8 | 34.3 |
| PD1-IL7 VAR21 (G85E) | 382.2 | 13640.0 | 35.7 | 9.6 | 71.9 | 24.5 |

### Example 4.4: IL-7R Signaling (STAT5-P) on Activated PD-1⁺ Versus Freshly Isolated IL-7Ra⁺ CD4 T Cells Upon Treatment with Increasing Doses of PD1-IL7 Single and Double Mutants

To measure the on-target and off-target activity of the PD1-IL7 single and double mutants, the IL-7R signaling in activated PD-1⁺, representing the desired target, versus freshly isolated IL-7Ra⁺ CD4 T cells, representing the peripheral sink for an IL-7 therapy, was measured upon exposure to increasing concentration of immunoconjugates.

For this purpose, CD4 T cells were sorted from healthy donor PBMCs and activated as described for FIG. 7. Three days later, the cells were harvested and washed several times to remove endogenous cytokines and the cells were labelled with CTV (5 µM, 5 min at RT; C34557, Thermo Scientific) and co-cultured 1:1 (25 µl, 6*106 cells/ml for each population) with freshly isolated CD4 T cells from an unrelated donor in a V-bottom plate before being treated for 12 min at 37° C. with increasing concentrations of treatment antibodies (50 µl, 1:10 dilution steps). To preserve the phosphorylation state, an equal amount of Phosphoflow Fix Buffer I (100 ul, 557870, BD) was added right after 12 minutes incubation with the various constructs. The cells were then incubated for additional 30 min at 37° C. before being permeabilized overnight at -80° C. with Phosphoflow PermBuffer III (558050, BD). On the next day STAT-5 in its phosphorylated form was stained for 30 min at 4° C. by using an anti-STAT-5P antibody (47/Stat5(pY694) clone, 562076, BD).

The cells were acquired at the FACS BD-LSR Fortessa (BD Bioscience). The frequency of STAT-5P was determined with FlowJo (V10) and plotted with GraphPad Prism.

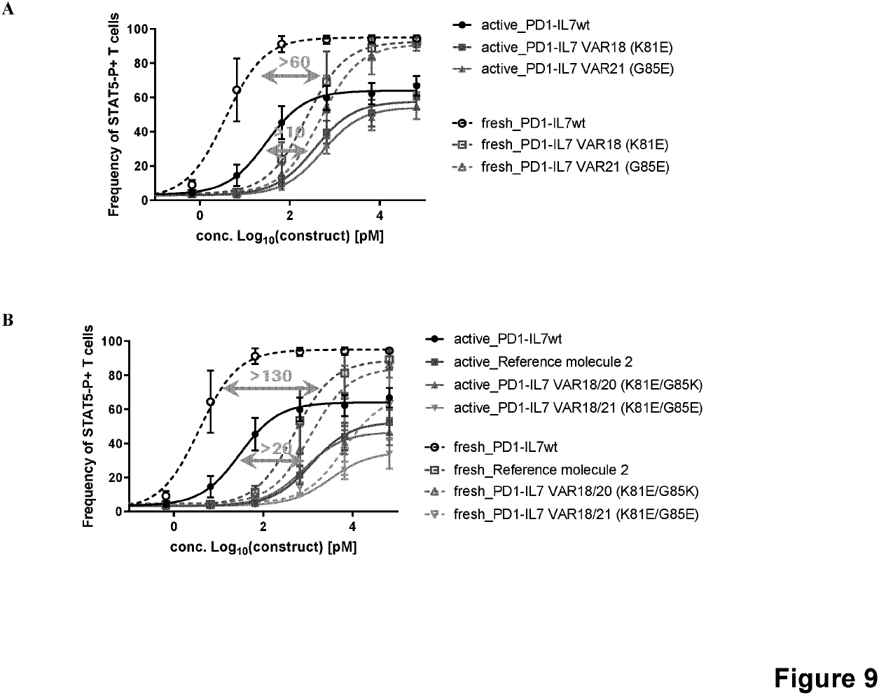

The dose-response curves on PD-1⁺ T cells provide information on the potency of the single and double mutants compared to PD1-IL7wt in signaling through the IL-7R on target T cells. Conversely, the dose-response curves on freshly isolated T cells which express high levels of IL-7Ra, show the potency of the single and double mutants compared to PD1-IL7wt in providing IL-7R signaling on off-target peripheral T cells. In this particular assay PD1-IL7VAR18 and PD1-IL7VAR21 had, respectively, more than 60 and 130 folds reduced potency on IL-7Ra⁺ T cells than PD1-IL7wt, while having only a 12 and 17 fold reduction in potency on PD-1+ T cells (FIG. 9A). Reference molecule 2 had more than 130 fold less IL-7R signaling on IL-7Ra⁺ T cells associated with a 40 fold reduction in potency on PD-1+ T cells (FIG. 9B). Of interest, the double mutants showed 300 and 1700 folds less off-target activity on IL-7Ra⁺ T cells with 27 and 106 folds , respectively, reduced activity on-target on PD1+ T cells when compared with PD1-IL7wt (FIG. 9B). This is indicative of the preferential activity of the single and, even more, of the double mutants on PD-1⁺ T cells due to the reduced affinity to the IL-7Ra and therefore reduced off-target effect.

TABLE 30

| | EC50 [active] | EC50[active] / EC50 [active PD1-IL7wt] | EC50 [fresh] | EC50[fresh]/EC50 [fresh PD1-IL7wt] | AUC [active] | AUC [fresh] |
|---|---|---|---|---|---|---|
| PD1-IL7wt | 29.99 | 1.0 | 3.705 | 1.0 | 200.2 | 376.9 |
| Reference molecule 2 | 1193 | 39.8 | 501.2 | 135.3 | 84.46 | 175.8 |
| PD1-IL7 VAR18/20 (K81E/G85K) | 821 | 27.4 | 1125 | 303.6 | 80.99 | 142.8 |
| PD1-IL7 VAR18/21 (K81E/G85E) | 3187 | 106.3 | 6377 | 1721.2 | 42.84 | 73.02 |
| PD1-IL7 VAR18 (K81E) | 360.9 | 12.0 | 233.1 | 62.9 | 119.1 | 214.2 |
| PD1-IL7 VAR21 (G85E) | 532.2 | 17.7 | 480.7 | 129.7 | 104.4 | 184.6 |

### Example 4.5: PD1-IL7 Single and Double Mutants Functional Activity on Cytotoxic Effector Functions and Proliferation of Allo-Specific PD-1⁺ CD4 T Cells

To assess the functional activity of PD1-IL7v on the effector functions of the T cells and compare it to PD1-IL7wt, CD4 T cells were co-cultured for 5 days with a B-cell lymphoblastoid tumor cell line (ARH77) to generate allo-reactive T cells. ARH77 expresses intermediate levels of PD-L1 and high levels of MHCII and induces PD-1 expression on the surface of the allo-specific CD4 T cells. This assay therefore allows for the functional assessment of the PD-1 blockade and the PD-1 mediated delivery of mutated and wt IL-7.

CD4 isolation and CTV labelling was conducted as described above. The sorted CD4⁺ T cells were co-cultured with irradiated ARH-77 (human B lymphoblast cell line) in an E:T ratio of 5:1 (100’000 T Cells: 20’000 ARH-77) in presence or absence of increasing doses of PD1-based or FAP-based IL-7 mutants and wt. The cells were co-cultured in a 96-round bottom plate for 5 days at 37° C., 5% CO₂. After 5 days the accumulation of cytokines in the Golgi complex was enhanced by applying Protein Transport Inhibitors (GolgiPlug, 555029, BD and GolgiStop, 554724, BD) for 5 hours prior to the FACS staining. The cells were first stained for CD4 and for live/dead. After fixation/ permeabilization overnight (554714, BD), the cells were stained intracellularly for Granzyme B (GrzB). The cells were acquired at the FACS BD-LSR Fortessa (BD Bioscience) and analyzed with FlowJo and GraphPad Prism. By gating on the living and proliferating CD4⁺ T cells (CTVlow), the frequency and mean fluorescence intensity of granzyme B secretion was compared between the conditions.

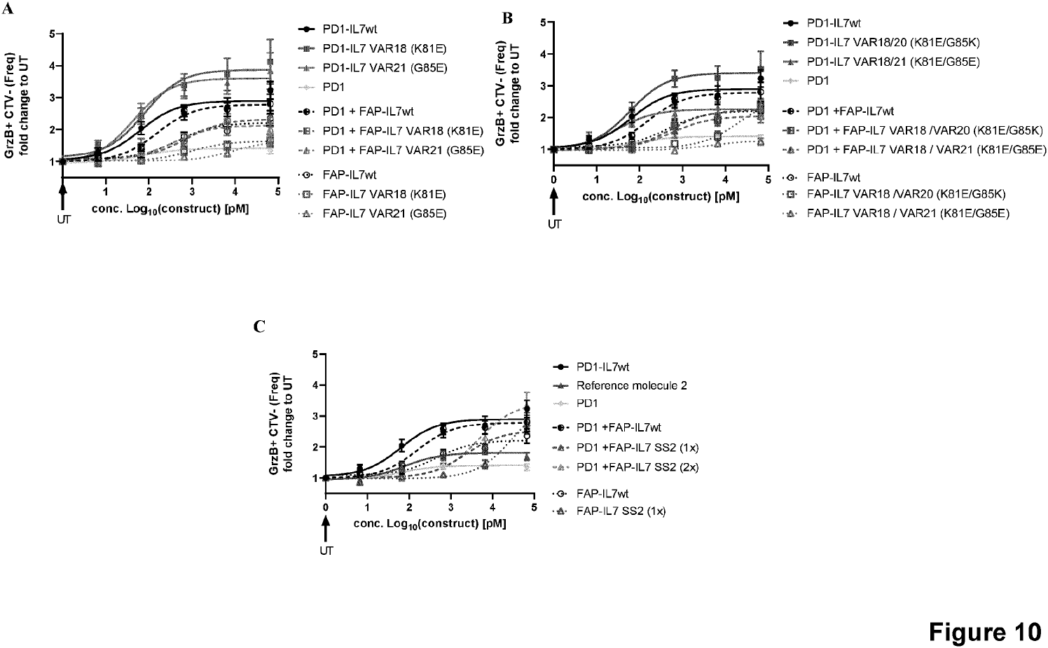

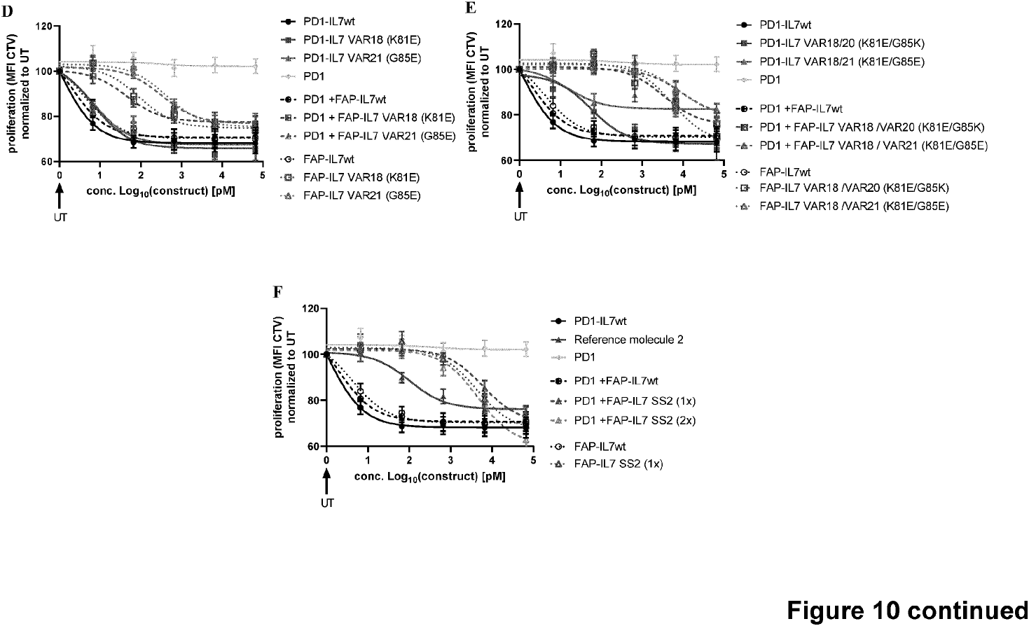

The dose-response curves indicate that the single mutants PD1-IL7VAR18, PD1-IL7VAR21 and the double mutant PD1-IL7VAR18/20 are functionally active and are even more potent than PD1-IL7wt in eliciting cytotoxic T cell effector functions while they induced comparable T cell-proliferation (FIGS. 10A-B, 10D-E). Reference molecule 2 showed lower activity on T cell effector functions (3-fold) and proliferation (2-fold) (FIGS. 10C, 10F).

Interestingly, combination of parental anti-PD1 antibody with the untargeted FAP-based IL-7 molecules did not reproduce similar results highlighting the important contribution of the PD-1 mediated targeting for an efficient delivery of the IL-7 mutants to PD-1⁺ allospecific T cells.

Tables 31 and 32 and the corresponding FIGS. 10A-F demonstrate the potency of the tested molecules and that the combination treatments do not recapitulate the effect of the fusion-proteins.

TABLE 31

Data FIGS. 10A-C

| | EC50 | Total Area | | EC50 | Total Area | | EC50 | Total Area |
|---|---|---|---|---|---|---|---|---|
| PD1-IL7wt | 63 | 5.6 | PD1 + FAP-IL7wt | 155 | 4.7 | FAP-IL7wt | 458 | 2.8 |
| PD1-IL7 VAR18/20 (K81E/G85K) | 60 | 7.2 | PD1 + FAP-IL7 VAR18 /VAR20 (K81E/G85K) | 516 | 2.5 | FAP-IL7 VAR18 /VAR20 (K81E/G85K) | 20299 | 1.3 |
| PD1-IL7VAR18/21 (K81E/G85E) | 31 | 4.2 | PD1 + FAP-IL7 VAR18 / VAR21 (K81E/G85E) | 579 | 2.2 | FAP-IL7 VAR18 / VAR21 (K81E/G85E) | 4446 | 0.3 |
| PD1-IL7 VAR18 (K81E) | 78 | 8.6 | PD1 + FAP-IL7 VAR18 (K81E) | 726 | 3.0 | FAP-IL7 VAR18 (K81E) | 959 | 1.2 |
| PD1-IL7 VAR21 (G85E) | 44 | 8.1 | PD1 + FAP-IL7 VAR21 (G85E) | 374 | 2.8 | FAP-IL7 VAR21 (G85E) | 9333 | 0.8 |
| Reference molecule 2 | 77 | 2.3 | PD1 + FAP-IL7 SS2 (1x) | 2249 | 2.4 | FAP-IL7 SS2 (1x) | 29811 | 1.6 |
| PD1 | 65 | 1.2 | PD1 + FAPIL7 SS2 (2x) | 4457 | 3.2 | | | |

TABLE 32

Data FIGS. 10D-F

| | EC50 | Total Area | | EC50 | Total Area | | EC50 | Total Area |
|---|---|---|---|---|---|---|---|---|
| PD1-IL7wt | 1 | 131 | PD1 + FAP-IL7wt | 2 | 118 | FAP-IL7wt | 4 | 115 |
| PD1-IL7 VAR18/20 (K81E/G85K) | 57 | 104 | PD1 + FAP-IL7 VAR18 /VAR20 (K81E/G85K) | 3375 | 35 | FAP-IL7 VAR18 /VAR20 (K81E/G85K) | 5443 | 39 |
| PD1-IL7VAR18/21 (K81E/G85E) | 15 | 59 | PD1 + FAP-IL7 VAR18 / VAR21 (K81E/G85E) | 9489 | 19 | FAP-IL7 VAR18 /VAR21 (K81E/G85E) | 7501 | 23 |
| PD1-IL7 VAR18 (K81E) | 7 | 129 | PD1 + FAP-IL7 VAR18 (K81E) | 45 | 69 | FAP-IL7 VAR18 (K81E) | 91 | 69 |
| PD1-IL7 VAR21 (G85E) | 7 | 121 | PD1 + FAP-IL7 VAR21 (G85E) | 323 | 51 | FAP-IL7 VAR21 (G85E) | 422 | 54 |
| Reference molecule 2 | 101 | 65 | PD1 + FAP-IL7 SS2 (1x) | 5940 | 34 | FAP-IL7 SS2 (1x) | 4224 | 43 |
| PD1 | 357 | 18 | PD1 + FAPIL7 SS2 (2x) | 4555 | 50 | | | |

### Example 4.6: Targeting of Stem-Like T Cells, Tregs and Naïve T Cells by PD1-Based Versus Untargeted IL-7 Mutants and Wt

As proof of concept experiment to assess whether the mutations introduced in the IL-7, to reduce its affinity for the IL-7Ra, improve the PD-1 mediated delivery and therefore the targeting of PD-1⁺ T cells, the PD1-IL7 mutants and PD1-IL7wt were tested in a binding assay on healthy donor PBMCs, containing abundant off-target T cells like naive and Tregs, and a small target population naturally expressing PD-1 .

Healthy donor PBMCs were incubated for 30 minutes at 37° C. with either PD1-IL7VAR18, PD1-IL7VAR21, PD1-IL7wt or FAP-IL7VAR18, FAP-IL7VAR21, FAP-IL7wt. After removal of the unbound constructs, the PBMCs were stained with a directly labelled anti-PGLALA antibody able to specifically detect the mutated Fc-portion of the immuno-conjugates. The cells were further stained for CD4 and CD8, before fixation, and permeabilized and stained with FOXP-3, PD-1 and TCF-1.

Based on the surface and intracellular markers the T cells were divided in the following subpopulations: Tregs (CD4⁺FOXP3⁺), CD8 naive (CD8⁺PD-1⁻TCF-1⁺) and CD8 stem-like T cells (CD8⁺PD-1⁺TCF-1⁺). The frequency of PGLALA+ cells were then measured and calculated for each T cell subsets across the treatment conditions.

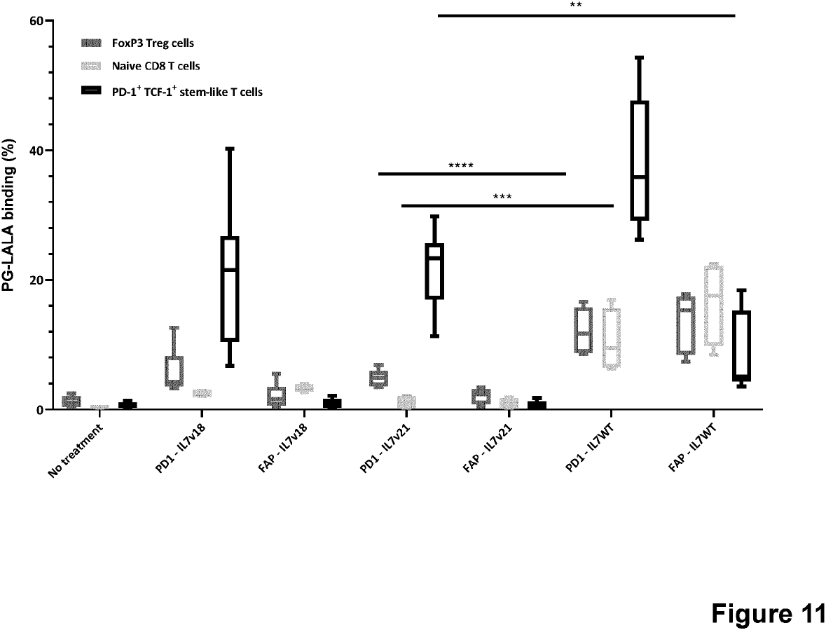

FIG. 11 demonstrates that FAP-IL7wt bind to naive T cells, followed by Tregs and as last to stem-like T cells, in agreement with the decreasing expression levels of IL-7Ra on the three subsets. Conversely, the PD-1 mediated targeting of PD1-IL7wt, while leaving unchanged the binding to naive and Tregs, drastically increased the targeting towards stem-like T cells (FIG. 11). Notably, both PD1-IL7VAR18 and VAR21 maintained the targeting towards the stem-like T cells, however showed a drastic reduction in off-target binding to both Tregs and naive T cells (FIG. 11).

### Example 4.7: Cross-Reactivity of PD1-IL7 Single, Double Mutants and Wt to Mouse IL-7Ra and IL-2Rg of Human PD-1 Transgenic Mice

In order to perform in-vivo efficacy studies in mice, the IL7 single, double mutants and wild type fused to the blocking anti-PD1 antibody were tested for cross-reactivity to the murine IL-7Ra and IL-2Rg of activated splenocytes from human PD-1 transgenic mice.

For this purpose CD4 T cells were isolated from the single cell suspension of the spleens of two human PD-1 transgenic mice by using CD4 beads (130-104-454, Miltenyi) and activated for 3 days in presence of 5 µg/ml plate bound anti-CD3 (overnight pre-coated, clone 145-2C11, BioLegend) and 5 µg/ml of plate bound anti-CD28 (overnight pre-coated, clone 37.51, BioLegend) antibodies to induce PD-1 expression. Three days later, the cells were harvested and washed several times to remove endogenous cytokines. The PD1⁺ CD4 T cells (50 µl, 4*106 cells/ml) were seeded in a V-bottom plate before being treated for 30 minutes at 37° C. with increasing concentrations of treatment antibodies (50 µl, 1:10 dilution steps). To preserve the phosphorylation state, an equal amount of Phosphoflow Fix Buffer I (100 ul, 557870, BD) was added right after 30 minutes incubation with the various constructs. The cells were then incubated for additional 30 min at 37° C. before being permeabilized overnight at -80° C. with Phosphoflow PermBuffer III (558050, BD). On the next day STAT-5 in its phosphorylated form was stained for 30 min at 4° C. by using an anti-STAT-5P antibody (47/Stat5(pY694) clone, 562076, BD).

The cells were acquired at the FACS BD-LSR Fortessa (BD Bioscience). The frequency of STAT-5P was determined with FlowJo (V10) and plotted with GraphPad Prism.

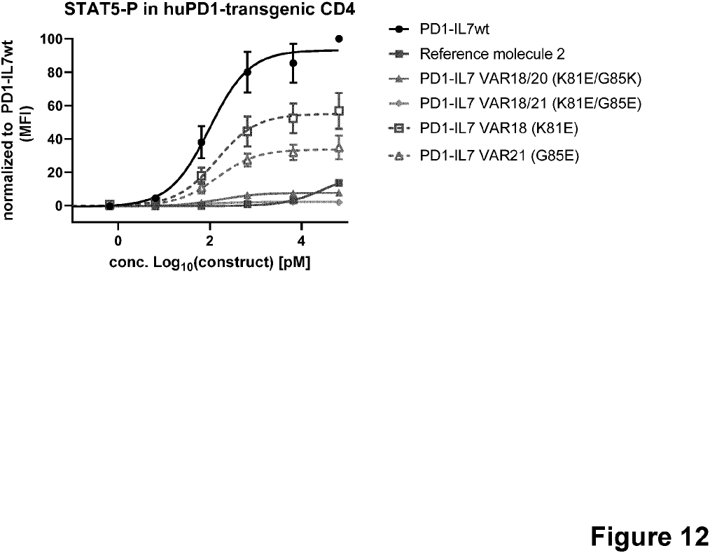

PD1-IL7wt showed to be mouse cross reactive and to signal in a dose dependent way through the IL-7R of activated CD4 T cells achieving plateau at a 10 folds lower concentration than on human CD4 T cells. Also the single mutants PD1-IL7VAR18 and PD1-IL7VAR21 induced a dose response signaling of the IL-7R with a comparable potency to the PD1-IL7wt but with a reduced Cmax. Conversely, both double mutants as well as Reference molecule 2 did not elicit any signaling in the activated PD-1⁺ CD4 T cells (FIG. 12).

### Example 4.8: IL-7R Signaling (STAT5-P) on Activated PD-1⁺ and PD-1- CD4 T Cells Upon Treatment with Increasing Doses of IL-7 VAR18 (K81E), VAR21 and Wild Type Fused to C-and N-Terminus of the PD-1 Blocking Antibody

In the following experiment PD1-IL7 Var18, Var21 and wild type, fused on the C-terminus versus the N-terminus of the PD-1 blocking antibody, were assessed to investigate the impact of C- and N-terminus on the activity of the immunoconjugates. These molecules were then tested in a dose dependent manner on activated PD-1⁺ and PD-1⁻ (pre-treated with a competing anti-PD-1 antibody) CD4 T cells. For this purpose the same experiment as described in Example 4.2 and FIG. 7 was performed.

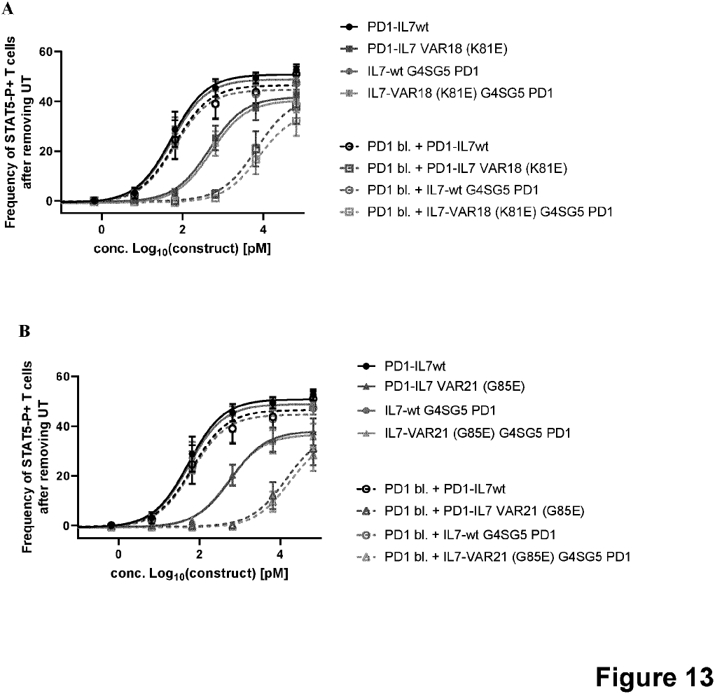

The dose-response curves on PD-1⁺ T cells provide information on the potency of the different PD1-IL7 Var18, Var21 and wt molecules in the C- and N-terminus format which appears to be similar (FIGS. 13A+B).

### Example 4.9: IL-7R Signaling (STAT5-P) on Activated PD-1⁺ and PD-1- CD4 T Cells Upon Treatment with Increasing Doses of PD1-IL7 Constituted by IL7 Mutants (Reference Molecules 5-8) Fused to PD1 Binder

In this experiment, the potency and the cis/trans-signaling of four different PD1-IL7 mutants, which were generated by fusing one mutated IL7v to PD1 binder (Reference molcules 5 to 8 as described above), were measured as IL-7R signaling by treating activated PD1⁺ and PD-1⁻ (anti-PD-1 pre-treated) CD4 T cells with increasing concentration of immunoconjugates. The purpose was to determine the dependency of the PD1-IL7 mutants on the PD-1 expression of the T cells in order to deliver an IL-7R signaling. For this purpose, CD4 T cells were sorted from healthy donor PBMCs and the experiment was performed as described above.

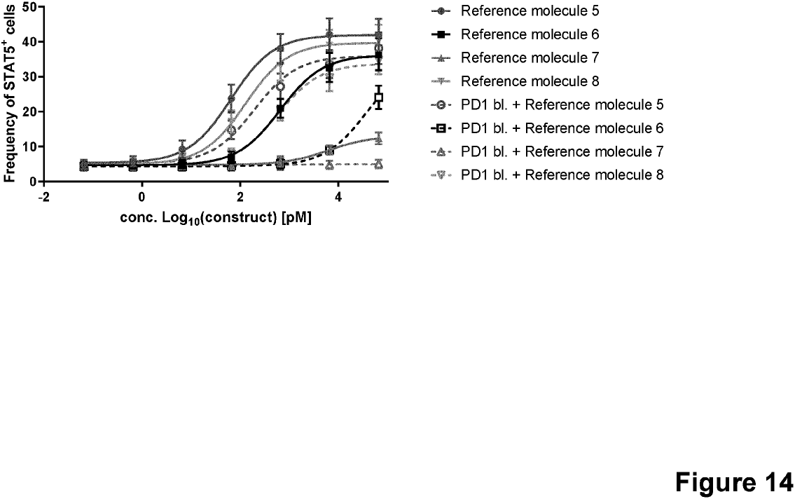

The dose-response curves on PD-1⁺ T cells provide information on the potency of the reference molecules in signaling through the IL-7R. In addition, the dose-response curves on T cells pre-treated with a competing anti-PD-1 antibody, to prevent the PD-1 mediated delivery, show the potency of the reference molecules in providing IL-7R signaling independently from PD-1 expression. In this particular assay, only Reference molecule 6 had more than 60 fold reduced activity on T cells in absence of PD-1 binding than on PD-1⁺ T cells (Table 33, FIG. 14).

Notably, reference molecule 6 has a stronger potency difference on PD1⁺ and PD1 pre-blocked cells in comparison to reference molecule 2 (FIG. 2), suggesting that either the avidity of the used PD-1 binder is higher and/or having one IL-7v molecule fused to an anti-PD-1 antibody allows PD-1 mediated targeting.

TABLE 33

| | EC50 (PD1+) | EC50 (PD1 blocked) | Area (PD 1+) | Area (PD1 blocked) |
|---|---|---|---|---|
| Reference molecule 5 | 65.2 | 194.4 | 113.4 | 81.6 |
| Reference molecule 6 | 649.1 | 38244 | 62.5 | 15.3 |
| Reference molecule 7 | 6199 | nd | 9.0 | 1.5 |
| Reference molecule 8 | 128.8 | 569.7 | 96.1 | 62.1 |

### Example 5

### In Vivo Efficacy of PD1-IL7v Variant 18 and 21 Immuno-Conjugates, in a Syngeneic Model of Mouse Tumor Cell Line, in Comparison to PD1-IL7wt Mab

PD1-IL7v variant 18 and 21 immune-conjugates were tested as single agents in comparison to PD1-IL7wt antibody for its anti-tumoral efficacy in one syngeneic model. The murine surrogate PD1-IL2v immune-conjugate was tested in the mouse colorectal MC38 cell line subcutaneously injected into Black 6 mice.

Panc02-H7 cells (mouse pancreatic carcinoma) were originally obtained from the MD Anderson cancer center (Texas, USA) and after expansion deposited in the Roche-Glycart internal cell bank. Panc02-H7-Fluc cell line was produced in house by calcium transfection and sub-cloning techniques. Panc02-H7-Fluc was cultured in RPMI medium containing 10% FCS (Sigma), 500 µg/ml hygromicin and 1% of Glutamax. The cells were cultured at 37° C. in a water-saturated atmosphere at 5 % CO². Passage 18 was used for transplantation. Cell viability was 92.6 %. 2×10⁵ cells per animal were injected subcutaneously in 100 µl of RPMI cell culture medium (Gibco) into the flank of mice using a 1 ml tuberculin syringe (BD Biosciences).

Female Black 6-huPD1 transgenics mice, aged 8-10 weeks at the start of the experiment (Charles Rivers, Lyon, France) were maintained under specific-pathogen-free condition with daily cycles of 12 h light / 12 h darkness according to committed guidelines (GV-Solas; Felasa; TierschG). After arrival, animals were maintained for one week to get accustomed to the new environment and for observation. Continuous health monitoring was carried out on a regular basis.

Mice were injected subcutaneously on study day 0 with 2×10⁵ of Panc02-Fluc cells, randomized and weighed. Twelve days after the tumor cell injection (tumor volume > 150 mm³), mice were injected i.v. with PD1-IL7v variant 18, variant 21, PD-IL7wt or vehicle once a week for two weeks. All mice were injected i.v. with 200 µl of the appropriate solution. The mice in the Vehicle group were injected with Histidine Buffer and the treatment groups with the PD1-IL7v variant 21 construct with 1 iv qw or the PD1-IL7v variant 18 with 1 mg/kg iv qw or the PD1-IL7wt with 1 mg/kg iv qw for 2 weeks. To obtain the appropriate amount of immunoconjugates per 200 µl, the stock solutions were diluted with Histidine Buffer when necessary.

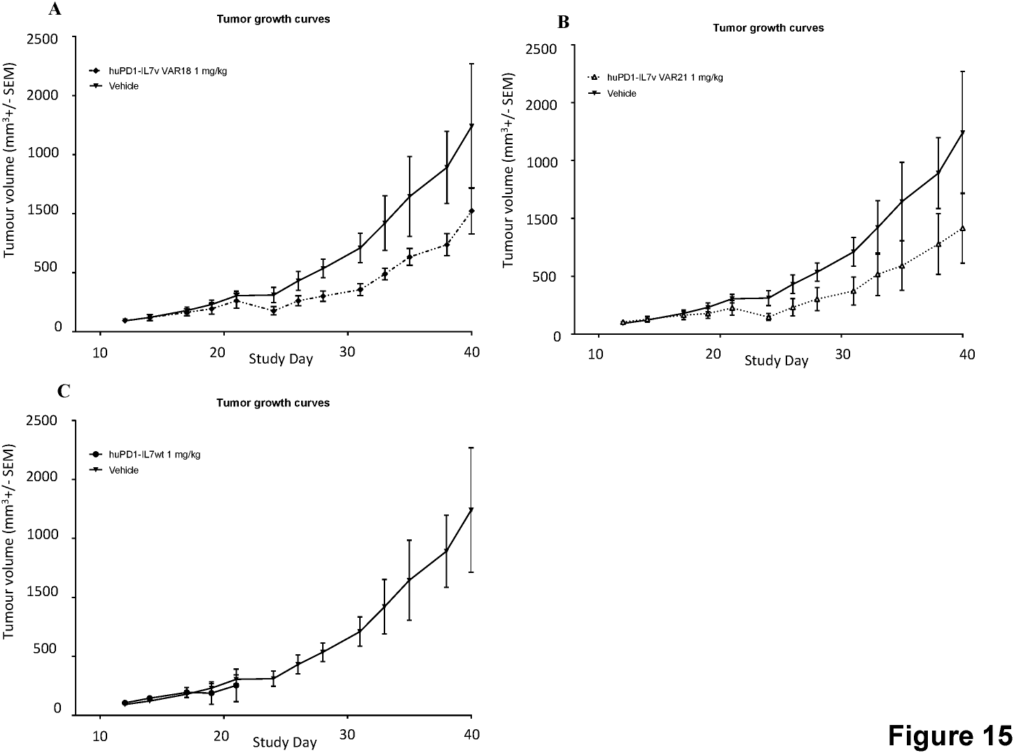

FIGS. 15A-C shows that the PD1-IL7v variants 18 and 21 mediated superior efficacy in terms of tumor growth inhibition compared to the vehicle group. The PD1-IL7v variants injected mice tolerated well the treatment. The PD1-IL7wt molecule was not well tolerated and the mice need to be sacrificed after the second administration, thus TGI could not be calculated.

TABLE 34

| | | | |
|---|---|---|---|
| Compound | Batch ID | Dose/mouse | Concentration (mg/mL) |
| PD1-IL7v var18 | P1AF5555_006 | 20 µg | 2.9 (= stock solution) |
| PD1-IL7v var 21 | P1AF5558_006 | 20 µg | 2.6 (= stock solution) |
| PD1-IL7wt | P1AF5572_014 | 20 µg | 2 (= stock solution) |

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A mutant interleukin-7 (IL-7) polypeptide, comprising at least one amino acid substitution in a position selected from the group of E13, V15, V18, D21, Q22, D25, T72, L77, K81, E84, G85, I88, , Q136, K139, N143 and M147 of human IL-7 according to SEQ ID NO: 52.

2. The mutant interleukin-7 polypeptide of claim 1 wherein said amino acid substitution is selected from the group of E13A, E13K, V15A, V15K, V18A, V18K, D21A, D21K, Q22A, Q22K, D25A, D25K, T72A, L77A, L77K, K81A, K81E, E84A, G85K, G85E, I88K, Q136A, Q136K, K139A, K139E, N143K and M147A.

3. The mutant interleukin-7 polypetide of claim 1 or 2, wherein said amino acid substitution is selected from the group of V15A, V15K, V18A, V18K, L77A, L77K, K81E, G85K, G85E, I88K and N143K.

4. The mutant interleukin-7 polypetide of any of claims 1 to 3, comprising at least the amino acid substitutions K81E and G85K or K81E and G85E.

5. An immunoconjugate comprising (i) a mutant IL-7 polypeptide of any one of claims 1 to 4 and (ii) an antibody that binds to PD-1.

6. An immunoconjugate according to claim 5, wherein the antibody comprises (a) a heavy chain variable region (VH) comprising a HVR-H1 comprising the amino acid sequence of SEQ ID NO:1, a HVR-H2 comprising the amino acid sequence of SEQ ID NO:2, a HVR-H3 comprising the amino acid sequence of SEQ ID NO:3, and a FR-H3 comprising the amino acid sequence of SEQ ID NO:7 at positions 71-73 according to Kabat numbering, and (b) a light chain variable region (VL) comprising a HVR-L1 comprising the amino acid sequence of SEQ ID NO:4, a HVR-L2 comprising the amino acid sequence of SEQ ID NO:5, and a HVR-L3 comprising the amino acid sequence of SEQ ID NO:6.

7. An immunoconjugate according to claim 5, wherein the antibody comprises (a) a heavy chain variable region (VH) comprising a HVR-H1 comprising the amino acid sequence of SEQ ID NO:8, a HVR-H2 comprising the amino acid sequence of SEQ ID NO:9, and a HVR-H3 comprising the amino acid sequence of SEQ ID NO:10, and (b) a light chain variable region (VL) comprising a HVR-L1 comprising the amino acid sequence of SEQ ID NO:11, a HVR-L2 comprising the amino acid sequence of SEQ ID NO:12, and a HVR-L3 comprising the amino acid sequence of SEQ ID NO:13.

8. An immunoconjugate according to claim 5, wherein the antibody comprises (a) a heavy chain variable region (VH) comprising an amino acid sequence that is at least about 95%, 96%, 97%, 98%, 99% or 100% identical to the amino acid sequence of SEQ ID NO:14, and (b) a light chain variable region (VL) comprising an amino acid sequence that is at least about 95%, 96%, 97%, 98%, 99% or 100% identical to an amino acid sequence selected from the group consisting of SEQ ID NO:15, SEQ ID NO:16, SEQ ID NO: 17, and SEQ ID NO:18.

9. The immunoconjugate of any one of claims 5 to 8, wherein the immunoconjugate comprises not more than one mutant IL-7 polypeptide.

10. The immunoconjugate of any one of claims 5 to 9, wherein the antibody comprises an Fc domain composed of a first and a second subunit.

11. The immunoconjugate of claim 10, wherein the Fc domain is an IgG class, particularly an IgGi subclass, Fc domain.

12. The immunoconjugate of claim 10 or 11, wherein the Fc domain is a human Fc domain.

13. The immunoconjugate of any one of claims 5 to 12, wherein the antibody is an IgG class, particularly an IgGi subclass immunoglobulin.

14. The immunoconjugate of any one of claims 10 to 13, wherein the Fc domain comprises a modification promoting the association of the first and the second subunit of the Fc domain.

15. The immunoconjugate of any one of claims 10 to 14, wherein in the CH3 domain of the first subunit of the Fc domain an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

16. The immunoconjugate of any one of claims 10 to 15, wherein in the first subunit of the Fc domain the threonine residue at position 366 is replaced with a tryptophan residue (T366W), and in the second subunit of the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numberings according to Kabat EU index).

17. The immunoconjugate of claim 16, wherein in the first subunit of the Fc domain additionally the serine residue at position 354 is replaced with a cysteine residue (S354C) or the glutamic acid residue at position 356 is replaced with a cysteine residue (E356C), and in the second subunit of the Fc domain additionally the tyrosine residue at position 349 is replaced by a cysteine residue (Y349C) (numberings according to Kabat EU index).

18. The immunoconjugate of any one of claims 10 to 17, wherein the mutant IL-7 polypeptide is fused at its amino-terminal amino acid to the carboxy-terminal amino acid of one of the subunits of the Fc domain, particularly the first subunit of the Fc domain, optionally through a linker peptide.

19. The immunoconjugate of claim 18, wherein the linker peptide has the amino acid sequence of SEQ ID NO:21.

20. The immunoconjugate of any one of claims 10 to 18, wherein the Fc domain comprises one or more amino acid substitution that reduces binding to an Fc receptor, particularly an Fcγ receptor, and/or effector function, particularly antibody-dependent cell-mediated cytotoxicity (ADCC).

21. The immunoconjugate of claim 20, wherein said one or more amino acid substitution is at one or more position selected from the group of L234, L235, and P329 (Kabat EU index numbering).

22. The immunoconjugate of any one of claims 10 to 21, wherein each subunit of the Fc domain comprises the amino acid substitutions L234A, L235A and P329G (Kabat EU index numbering).

23. The immunoconjugate of any one of claims 5 to 22, comprising a polypeptide comprising an amino acid sequence that is at least about 80%, 85%, 90%, 95%, 96%, 97%, 98%, 99% or 100% identical to the sequence of SEQ ID NO: 85, a polypeptide comprising an amino acid sequence that is at least about 80%, 85%, 90%, 95%, 96%, 97%, 98%, 99% or 100% identical to the sequence of SEQ ID NO: 86, and a polypeptide comprising an amino acid sequence that is at least about 80%, 85%, 90%, 95%, 96%, 97%, 98%, 99% or 100% identical to a sequence selected from the group of SEQ ID NO: 90, SEQ ID NO: 91, SEQ ID NO: 92, SEQ ID NO: 93, SEQ ID NO: 102, SEQ ID NO: 103, SEQ ID NO: 105, SEQ ID NO: 107, SEQ ID NO: 108, SEQ ID NO: 109, SEQ ID NO: 114, SEQ ID NO: 137 and SEQ ID NO:138.

24. The immunoconjugate of any one of claims 5 to 23, essentially consisting of a mutant IL-7 polypeptide and an IgGi immunoglobulin molecule, joined by a linker sequence.

25. One or more isolated polynucleotide encoding the mutant IL-7 polpypeptyde according to any one of claims 1 to 4 or the immunoconjugate of any one of claims 5 to 24.

26. One or more vector, particularly expression vector, comprising the polynucleotide(s) of claim 23.

27. A host cell comprising the polynucleotide(s) of claim 23 or the vector(s) of claim 24.

28. A method of producing a mutant IL-7 polypeptide or an immunoconjugate comprising a mutant IL-7 polypeptide and an antibody that binds to PD-1, comprising (a) culturing the host cell of claim 26 under conditions suitable for the expression of the mutant IL-7 polypetide or the immunoconjugate, and optionally (b) recovering the mutant IL-7 polypetide or the immunoconjugate.

29. A mutant IL-7 polypetide or an immunoconjugate comprising a mutant IL-7 polypeptide and an antibody that binds to PD-1, produced by the method of claim 28.

30. A pharmaceutical composition comprising the mutant IL-7 polypetide of any one of claims 1 to 4 or 29 or the immunoconjugate of any one of claims 5 to 24 or 29 and a pharmaceutically acceptable carrier.

31. The mutant IL-7 polypetide of any one of claims 1 to 4 or 29 or the immunoconjugate of any one of claims 5 to 24 or 29 for use as a medicament.

32. The mutant IL-7 polypetide of any one of claims 1 to 4 or 29 or the immunoconjugate of any one of claims 5 to 24 or 29 for use in the treatment of a disease.

33. The mutant IL-7 polypeptide or the immunoconjugate for use in the treatment of a disease of claim 32, wherein said disease is cancer.

34. Use of the mutant IL-7 polypeptide of any one of claims 1 to 4 or 29 or the immunoconjugate of any one of claims 5 to 24 or 29 in the manufacture of a medicament for the treatment of a disease.

35. The use of claim 34, wherein said disease is cancer.

36. A method of treating a disease in an individual, comprising administering to said individual a therapeutically effective amount of a composition comprising the mutant IL-7 polypeptide of any one of claims 1 to 5 or 29 or the immunoconjugate of any one of claims 4 to 24 or 29 in a pharmaceutically acceptable form.

37. The method of claim 36, wherein said disease is cancer.

38. A method of stimulating the immune system of an individual, comprising administering to said individual an effective amount of a composition comprising the mutant IL-7 polypetide of any of claims 1 to 4 and 29 or the immunoconjugate of any one of claims 5 to 24 or 29 in a pharmaceutically acceptable form.

39. The invention as described hereinbefore.


SEQUENCE LISTING (166 sequences)

SEQ ID NO: 1 - protein, 4 aa, Artificial Sequence
  Synthetic Construct
SSYT

SEQ ID NO: 2 - protein, 8 aa, Artificial Sequence
  Synthetic Construct
SGGGRDIY

SEQ ID NO: 3 - protein, 5 aa, Artificial Sequence
  Synthetic Construct
GRVYF

SEQ ID NO: 4 - protein, 6 aa, Artificial Sequence
  Synthetic Construct
TSDNSF

SEQ ID NO: 5 - protein, 7 aa, Artificial Sequence
  Synthetic Construct
RSSTLES

SEQ ID NO: 6 - protein, 6 aa, Artificial Sequence
  Synthetic Construct
NYDVPW

SEQ ID NO: 7 - protein, 3 aa, Artificial Sequence
  Synthetic Construct
RDN

SEQ ID NO: 8 - protein, 7 aa, Artificial Sequence
  Synthetic Construct
GFSFSSY

SEQ ID NO: 9 - protein, 3 aa, Artificial Sequence
  Synthetic Construct
GGR

SEQ ID NO: 10 - protein, 9 aa, Artificial Sequence
  Synthetic Construct
TGRVYFALD

SEQ ID NO: 11 - protein, 11 aa, Artificial Sequence
  Synthetic Construct
SESVDTSDNSF

SEQ ID NO: 12 - protein, 3 aa, Artificial Sequence
  Synthetic Construct
RSS

SEQ ID NO: 13 - protein, 6 aa, Artificial Sequence
  Synthetic Construct
NYDVPW

SEQ ID NO: 14 - protein, 120 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSS

SEQ ID NO: 15 - protein, 111 aa, Artificial Sequence
  Synthetic Construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIK

SEQ ID NO: 16 - protein, 111 aa, Artificial Sequence
  Synthetic Construct
DVVMTQSPLSLPVTLGQPASISCRASESVDTSDNSFIHWYQQRPGQSPRLLIYRSSTLESGVPDRFSGSGSGTDFTLKISRVEAEDVGVYYCQQNYDVPWTFGQGTKVEIK

SEQ ID NO: 17 - protein, 111 aa, Artificial Sequence
  Synthetic Construct
EIVLTQSPATLSLSPGERATLSCRASESVDTSDNSFIHWYQQKPGQSPRLLIYRSSTLESGIPARFSGSGSGTDFTLTISSLEPEDFAVYYCQQNYDVPWTFGQGTKVEIK

SEQ ID NO: 18 - protein, 111 aa, Artificial Sequence; identical to SEQ ID NO: 17
  Synthetic Construct

SEQ ID NO: 19 - protein, 133 aa, Homo sapiens [sequence omitted]

SEQ ID NO: 20 - protein, 133 aa, Artificial Sequence
  Synthetic Construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 21 - protein, 15 aa, Artificial Sequence
  Synthetic Construct
GGGGSGGGGSGGGGS

SEQ ID NO: 22 - protein, 597 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 23 - protein, 448 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 24 - protein, 448 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSP

SEQ ID NO: 25 - protein, 218 aa, Artificial Sequence
  Synthetic Construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 20 aa, Homo sapiens
MYRMQLLSCIALSLALVTNS

SEQ ID NO: 27 - protein, 268 aa, Homo sapiens
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 28 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 29 - protein, 133 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 30 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 31 - protein, 107 aa, Homo sapiens [sequence omitted]

SEQ ID NO: 32 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 33 - protein, 328 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 34 - protein, 607 aa, Artificial Sequence
  Synthetic Construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGGGSGGGGSGGGGSAPASSSTSSSTAEAQQQQQQQQQQQQHLEQLLMDLQELLSRMENYRNLKLPRMLTAKFALPKQATELKDLQCLEDELGPLRHVLDGTQSKSFQLEDAENFISNIRVTVVKLKGSDNTFECQFDDESATVVDFLRRWIAFAQSIISTSPQ

SEQ ID NO: 35 - protein, 442 aa, Artificial Sequence
  Synthetic Construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSP

SEQ ID NO: 36 - protein, 218 aa, Artificial Sequence
  Synthetic Construct
DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 37 - protein, 595 aa, Artificial Sequence
  Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 38 - protein, 446 aa, Artificial Sequence
  Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 39 - protein, 214 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 605 aa, Artificial Sequence
  Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFGAPIEKTISKTKGRPKAPQVYTIPPPKEQMAKDKVSLTCMITDFFPEDITVEWQWNGQPAENYDNTQPIMDTDGSYFVYSDLNVQKSNWEAGNTFTCSVLHEGLHNHHTEKSLSHSPGGGGGSGGGGSGGGGSAPASSSTSSSTAEAQQQQQQQQQQQQHLEQLLMDLQELLSRMENYRNLKLPRMLTAKFALPKQATELKDLQCLEDELGPLRHVLDGTQSKSFQLEDAENFISNIRVTVVKLKGSDNTFECQFDDESATVVDFLRRWIAFAQSIISTSPQ

SEQ ID NO: 41 - protein, 440 aa, Artificial Sequence
  Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFGAPIEKTISKTKGRPKAPQVYTIPPPKKQMAKDKVSLTCMITDFFPEDITVEWQWNGQPAENYKNTQPIMKTDGSYFVYSKLNVQKSNWEAGNTFTCSVLHEGLHNHHTEKSLSHSP

SEQ ID NO: 42 - protein, 214 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 43 - protein, 150 aa, Homo sapiens [sequence omitted]

SEQ ID NO: 44 - protein, 608 aa, Artificial Sequence
  Synthetic Construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGGGSGGGGSGGGGSAPASSSTSSSTAEAQQQQQQQQQQQQHLEQLLMDLQELLSRMENYRNLKLPRMLTAKFALPKQATELKDLQCLEDELGPLRHVLDGTQSKSFQLEDAENFISNIRVTVVKLKGSDNTFECQFDDESATVVDFLRRWIAFAQSIISTSPQ

SEQ ID NO: 45 - protein, 445 aa, Artificial Sequence
  Synthetic Construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGK

SEQ ID NO: 46 - protein, 215 aa, Artificial Sequence
  Synthetic Construct
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 47 - protein, 604 aa, Artificial Sequence
  Synthetic Construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGGGSGGGGSGGGGSAPASSSTSSSTAEAQQQQQQQQQQQQHLEQLLMDLQELLSRMENYRNLKLPRMLTAKFALPKQATELKDLQCLEDELGPLRHVLDGTQSKSFQLEDAENFISNIRVTVVKLKGSDNTFECQFDDESATVVDFLRRWIAFAQSIISTSPQ

SEQ ID NO: 48 - protein, 441 aa, Artificial Sequence
  Synthetic Construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGK

SEQ ID NO: 49 - protein, 215 aa, Artificial Sequence
  Synthetic Construct
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 50 - protein, 450 aa, Artificial Sequence
  Synthetic Construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSASTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 51 - protein, 218 aa, Artificial Sequence; identical to SEQ ID NO: 36
  Synthetic Construct

SEQ ID NO: 52 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 53 - protein, 152 aa, Artificial Sequence
  Synthetic Construct
DCDIEGKDGKQYASVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 54 - protein, 152 aa, Artificial Sequence
  Synthetic Construct
DCDIEGKDGKQYKSVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 55 - protein, 152 aa, Artificial Sequence
  Synthetic Construct
DCDIEGKDGKQYESALMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 56 - protein, 152 aa, Artificial Sequence
  Synthetic Construct
DCDIEGKDGKQYESKLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 57 - protein, 152 aa, Artificial Sequence
  Synthetic Construct
DCDIEGKDGKQYESVLMASIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 58 - protein, 152 aa, Artificial Sequence
  Synthetic Construct
DCDIEGKDGKQYESVLMKSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 59 - protein, 152 aa, Artificial Sequence
  Synthetic Construct
DCDIEGKDGKQYESVLMVSIAQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 60 - protein, 152 aa, Artificial Sequence
  Synthetic Construct
DCDIEGKDGKQYESVLMVSIKQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 61 - protein, 152 aa, Artificial Sequence
  Synthetic Construct
DCDIEGKDGKQYESVLMVSIDALLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 62 - protein, 152 aa, Artificial Sequence
  Synthetic Construct
DCDIEGKDGKQYESVLMVSIDKLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 63 - protein, 152 aa, Artificial Sequence
  Synthetic Construct
DCDIEGKDGKQYESVLMVSIDQLLASMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 64 - protein, 152 aa, Artificial Sequence
  Synthetic Construct
DCDIEGKDGKQYESVLMVSIDQLLKSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 65 - protein, 152 aa, Artificial Sequence
  Synthetic Construct
DCDIEGKDGKQYESVLMVSIDQLLDSMKEIGSNCLNNEFNFFKRHICDANKEGMFLFRAARKLRQFLKMNSTGAFDLHLLKVSEGTTILLNCTGQVKGRKPAALGEAQPTKSLEENKSLKEQKKLNDLCFLKRLLQEIKTCWNKILMGTKEH

SEQ ID NO: 66 - protein, 152 aa, Artificial Sequence
  Synthetic Construct
DCDIEGKDGKQYESVLMVSIDQLLDSMKEIGSNCLNNEFNFFKRHICDANKEGMFLFRAARKLRQFLKMNSTGKFDLHLLKVSEGTTILLNCTGQVKGRKPAALGEAQPTKSLEENKSLKEQKKLNDLCFLKRLLQEIKTCWNKILMGTKEH

SEQ ID NO: 67 - protein, 152 aa, Artificial Sequence
  Synthetic Construct
DCDIEGKDGKQYESVLMVSIDQLLDSMKEIGSNCLNNEFNFFKRHICDANKEGMFLFRAARKLRQFLKMNSTGDFDAHLLKVSEGTTILLNCTGQVKGRKPAALGEAQPTKSLEENKSLKEQKKLNDLCFLKRLLQEIKTCWNKILMGTKEH

SEQ ID NO: 68 - protein, 152 aa, Artificial Sequence
  Synthetic Construct
DCDIEGKDGKQYESVLMVSIDQLLDSMKEIGSNCLNNEFNFFKRHICDANKEGMFLFRAARKLRQFLKMNSTGDFDKHLLKVSEGTTILLNCTGQVKGRKPAALGEAQPTKSLEENKSLKEQKKLNDLCFLKRLLQEIKTCWNKILMGTKEH

SEQ ID NO: 69 - protein, 152 aa, Artificial Sequence
  Synthetic Construct
DCDIEGKDGKQYESVLMVSIDQLLDSMKEIGSNCLNNEFNFFKRHICDANKEGMFLFRAARKLRQFLKMNSTGDFDLHLLAVSEGTTILLNCTGQVKGRKPAALGEAQPTKSLEENKSLKEQKKLNDLCFLKRLLQEIKTCWNKILMGTKEH

SEQ ID NO: 70 - protein, 152 aa, Artificial Sequence
  Synthetic Construct
DCDIEGKDGKQYESVLMVSIDQLLDSMKEIGSNCLNNEFNFFKRHICDANKEGMFLFRAARKLRQFLKMNSTGDFDLHLLEVSEGTTILLNCTGQVKGRKPAALGEAQPTKSLEENKSLKEQKKLNDLCFLKRLLQEIKTCWNKILMGTKEH

SEQ ID NO: 71 - protein, 152 aa, Artificial Sequence
  Synthetic Construct
DCDIEGKDGKQYESVLMVSIDQLLDSMKEIGSNCLNNEFNFFKRHICDANKEGMFLFRAARKLRQFLKMNSTGDFDLHLLKVSAGTTILLNCTGQVKGRKPAALGEAQPTKSLEENKSLKEQKKLNDLCFLKRLLQEIKTCWNKILMGTKEH

SEQ ID NO: 72 - protein, 152 aa, Artificial Sequence
  Synthetic Construct
DCDIEGKDGKQYESVLMVSIDQLLDSMKEIGSNCLNNEFNFFKRHICDANKEGMFLFRAARKLRQFLKMNSTGDFDLHLLKVSEKTTILLNCTGQVKGRKPAALGEAQPTKSLEENKSLKEQKKLNDLCFLKRLLQEIKTCWNKILMGTKEH

SEQ ID NO: 73 - protein, 152 aa, Artificial Sequence
  Synthetic Construct
DCDIEGKDGKQYESVLMVSIDQLLDSMKEIGSNCLNNEFNFFKRHICDANKEGMFLFRAARKLRQFLKMNSTGDFDLHLLKVSEETTILLNCTGQVKGRKPAALGEAQPTKSLEENKSLKEQKKLNDLCFLKRLLQEIKTCWNKILMGTKEH

SEQ ID NO: 74 - protein, 152 aa, Artificial Sequence
  Synthetic Construct
DCDIEGKDGKQYESVLMVSIDQLLDSMKEIGSNCLNNEFNFFKRHICDANKEGMFLFRAARKLRQFLKMNSTGDFDLHLLKVSEGTTKLLNCTGQVKGRKPAALGEAQPTKSLEENKSLKEQKKLNDLCFLKRLLQEIKTCWNKILMGTKEH

SEQ ID NO: 75 - protein, 152 aa, Artificial Sequence
  Synthetic Construct
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLAEIKTCWNKILMGTKEH

SEQ ID NO: 76 - protein, 152 aa, Artificial Sequence
  Synthetic Construct
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLKEIKTCWNKILMGTKEH

SEQ ID NO: 77 - protein, 152 aa, Artificial Sequence
  Synthetic Construct
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIATCWNKILMGTKEH

SEQ ID NO: 78 - protein, 152 aa, Artificial Sequence
  Synthetic Construct
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIETCWNKILMGTKEH

SEQ ID NO: 79 - protein, 152 aa, Artificial Sequence
  Synthetic Construct
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWKKILMGTKEH

SEQ ID NO: 80 - protein, 152 aa, Artificial Sequence
  Synthetic Construct
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILAGTKEH

SEQ ID NO: 81 - protein, 152 aa, Artificial Sequence
  Synthetic Construct
DCDIEGKDGKQYESVLMVSIDQLLDSMKEIGSNCLNNEFNFFKRHICDANKEGMFLFRAARKLRQFLKMNSAGDFDLHLLKVSEGTTILLNCTGQVKGRKPAALGEAQPTKSLEENKSLKEQKKLNDLCFLKRLLQEIKTCWNKILMGTKEH

SEQ ID NO: 82 - protein, 152 aa, Artificial Sequence
  Synthetic Construct
DCDIEGKDGKQYESVLMVSIDQLLDSMKEIGSNCLNNEFNFFKRHICDANKEGMFLFRAARKLRQFLKMNSTGDFDLHLLKVSEGTTILLNCAGQVKGRKPAALGEAQPTKSLEENKSLKEQKKLNDLCFLKRLLQEIKTCWNKILMGTKEH

SEQ ID NO: 83 - protein, 152 aa, Artificial Sequence
  Synthetic Construct
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKALKEQKKLNDLCFLKRLLQEIKTCWNKILMGTKEH

SEQ ID NO: 84 - protein, 152 aa, Artificial Sequence
  Synthetic Construct
DCDIEGKDGKQYESVLMVSIDQLLDSMKEIGSNCLNNEFNFFKRHICDANKEGMFLFRAARKLRQFLKMNSAGDFDLHLLKVSEGTTILLNCAGQVKGRKPAALGEAQPTKSLEENKALKEQKKLNDLCFLKRLLQEIKTCWNKILMGTKEH

SEQ ID NO: 85 - protein, 218 aa, Artificial Sequence; identical to SEQ ID NO: 25
  Synthetic Construct

SEQ ID NO: 86 - protein, 450 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 87 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 88 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYASVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 89 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYKSVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 90 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESALMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 91 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESKLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 92 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMASIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 93 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMKSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 94 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIAQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 95 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIKQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 96 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDALLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 97 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDKLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 98 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLASMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 99 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLKSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 100 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGAFDLHLLKVSEGTTILLNCTGQVKGRKPAALGEAQPTKSLEENKSLKEQKKLNDLCFLKRLLQEIKTCWNKILMGTKEH

SEQ ID NO: 101 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGKFDLHLLKVSEGTTILLNCTGQVKGRKPAALGEAQPTKSLEENKSLKEQKKLNDLCFLKRLLQEIKTCWNKILMGTKEH

SEQ ID NO: 102 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDAHLLKVSEGTTILLNCTGQVKGRKPAALGEAQPTKSLEENKSLKEQKKLNDLCFLKRLLQEIKTCWNKILMGTKEH

SEQ ID NO: 103 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDKHLLKVSEGTTILLNCTGQVKGRKPAALGEAQPTKSLEENKSLKEQKKLNDLCFLKRLLQEIKTCWNKILMGTKEH

SEQ ID NO: 104 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLAVSEGTTILLNCTGQVKGRKPAALGEAQPTKSLEENKSLKEQKKLNDLCFLKRLLQEIKTCWNKILMGTKEH

SEQ ID NO: 105 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLEVSEGTTILLNCTGQVKGRKPAALGEAQPTKSLEENKSLKEQKKLNDLCFLKRLLQEIKTCWNKILMGTKEH

SEQ ID NO: 106 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSAGTTILLNCTGQVKGRKPAALGEAQPTKSLEENKSLKEQKKLNDLCFLKRLLQEIKTCWNKILMGTKEH

SEQ ID NO: 107 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEKTTILLNCTGQVKGRKPAALGEAQPTKSLEENKSLKEQKKLNDLCFLKRLLQEIKTCWNKILMGTKEH

SEQ ID NO: 108 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEETTILLNCTGQVKGRKPAALGEAQPTKSLEENKSLKEQKKLNDLCFLKRLLQEIKTCWNKILMGTKEH

SEQ ID NO: 109 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTKLLNCTGQVKGRKPAALGEAQPTKSLEENKSLKEQKKLNDLCFLKRLLQEIKTCWNKILMGTKEH

SEQ ID NO: 110 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLAEIKTCWNKILMGTKEH

SEQ ID NO: 111 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLKEIKTCWNKILMGTKEH

SEQ ID NO: 112 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIATCWNKILMGTKEH

SEQ ID NO: 113 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIETCWNKILMGTKEH

SEQ ID NO: 114 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWKKILMGTKEH

SEQ ID NO: 115 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILAGTKEH

SEQ ID NO: 116 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSAGDFDLHLLKVSEGTTILLNCTGQVKGRKPAALGEAQPTKSLEENKSLKEQKKLNDLCFLKRLLQEIKTCWNKILMGTKEH

SEQ ID NO: 117 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCAGQVKGRKPAALGEAQPTKSLEENKSLKEQKKLNDLCFLKRLLQEIKTCWNKILMGTKEH

SEQ ID NO: 118 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKALKEQKKLNDLCFLKRLLQEIKTCWNKILMGTKEH

SEQ ID NO: 119 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSAGDFDLHLLKVSEGTTILLNCAGQVKGRKPAALGEAQPTKSLEENKALKEQKKLNDLCFLKRLLQEIKTCWNKILMGTKEH

SEQ ID NO: 120 - protein, 467 aa, Artificial Sequence
  Synthetic Construct
ESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDASGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHE

SEQ ID NO: 121 - protein, 471 aa, Artificial Sequence
  Synthetic Construct
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAASGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 122 - protein, 218 aa, Artificial Sequence; identical to SEQ ID NO: 25
  Synthetic Construct

SEQ ID NO: 123 - protein, 449 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 124 - protein, 616 aa, Artificial Sequence; identical to SEQ ID NO: 87
  Synthetic Construct

SEQ ID NO: 125 - protein, 218 aa, Artificial Sequence; identical to SEQ ID NO: 25
  Synthetic Construct

SEQ ID NO: 126 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 127 - protein, 218 aa, Artificial Sequence; identical to SEQ ID NO: 25
  Synthetic Construct

SEQ ID NO: 128 - protein, 449 aa, Artificial Sequence; identical to SEQ ID NO: 123
  Synthetic Construct

SEQ ID NO: 129 - protein, 384 aa, Artificial Sequence
  Synthetic Construct
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 130 - protein, 218 aa, Artificial Sequence; identical to SEQ ID NO: 25
  Synthetic Construct

SEQ ID NO: 131 - protein, 449 aa, Artificial Sequence; identical to SEQ ID NO: 123
  Synthetic Construct

SEQ ID NO: 132 - protein, 617 aa, Artificial Sequence
  Synthetic Construct
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSEVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 133 - protein, 218 aa, Artificial Sequence; identical to SEQ ID NO: 25
  Synthetic Construct

SEQ ID NO: 134 - protein, 617 aa, Artificial Sequence
  Synthetic Construct
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSEVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 135 - protein, 152 aa, Artificial Sequence
  Synthetic Construct
DCDIEGKDGKQYESVLMVSIDQLLDSMKEIGSNCLNNEFNFFKRHICDANKEGMFLFRAARKLRQFLKMNSTGDFDLHLLEVSEKTTILLNCTGQVKGRKPAALGEAQPTKSLEENKSLKEQKKLNDLCFLKRLLQEIKTCWNKILMGTKEH

SEQ ID NO: 136 - protein, 152 aa, Artificial Sequence
  Synthetic Construct
DCDIEGKDGKQYESVLMVSIDQLLDSMKEIGSNCLNNEFNFFKRHICDANKEGMFLFRAARKLRQFLKMNSTGDFDLHLLEVSEETTILLNCTGQVKGRKPAALGEAQPTKSLEENKSLKEQKKLNDLCFLKRLLQEIKTCWNKILMGTKEH

SEQ ID NO: 137 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLEVSEKTTILLNCTGQVKGRKPAALGEAQPTKSLEENKSLKEQKKLNDLCFLKRLLQEIKTCWNKILMGTKEH

SEQ ID NO: 138 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLEVSEETTILLNCTGQVKGRKPAALGEAQPTKSLEENKSLKEQKKLNDLCFLKRLLQEIKTCWNKILMGTKEH

SEQ ID NO: 139 - protein, 219 aa, Artificial Sequence
  Synthetic Construct
DVVMTQSPLSLPVTLGQPASISCRSSQSLVHANTNTYLEWYQQRPGQSPRLLIYKVSNRFSGVPDRFSGSGSGTDFTLKISRVEAEDVGVYYCFQGTHVPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - protein, 614 aa, Artificial Sequence
  Synthetic Construct
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDCDIEGKDGKQYESVLMVSIDQLLDSMKEIGSNCLNNEFNFFKRHICDANKEGMFLFRAARKLRQFLKMNSTGEFDLHLLKVSEGTTILLNCTGQVKGRKPAALGEAQPTKSLEENKSLKEQKKLNDLCFLKRLLQEIKTCWNKILMGTKEH

SEQ ID NO: 141 - protein, 614 aa, Artificial Sequence
  Synthetic Sequence
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDSDIEGKDGKQYESVLMVSIDQLLDSMKEIGSNCLNNEFNFFKRHISDANKEGMFLFRAARKLRQFLKMNSTGDFDLHLLKVSEGTTILLNSTGQVKGRKPAALGEAQPTKSLEENKSLKEQKKLNDLCFLKRLLQEIKTSWNKILMGTKEH

SEQ ID NO: 142 - protein, 614 aa, Artificial Sequence
  Synthetic Construct
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDCDIEGKDGKQYESVLMVSIDQLLDSMKEIGSNSLNNEFNFFKRHISDANKEGMFLFRAARKLRQFLKMNSTGDFDLHLLKVSEGTTILLNSTGQVKGRKPAALGEAQPTKSLEENKSLKEQKKLNDLSFLKRLLQEIKTCWNKILMGTKEH

SEQ ID NO: 143 - protein, 614 aa, Artificial Sequence
  Synthetic Construct
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCHNKILMGTKEH

SEQ ID NO: 144 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGEFDLHLLKVSEGTTILLNCTGQVKGRKPAALGEAQPTKSLEENKSLKEQKKLNDLCFLKRLLQEIKTCWNKILMGTKEH

SEQ ID NO: 145 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDSDIEGKDGKQYESVLMVSIDQLLDSMKEIGSNCLNNEFNFFKRHISDANKEGMFLFRAARKLRQFLKMNSTGDFDLHLLKVSEGTTILLNSTGQVKGRKPAALGEAQPTKSLEENKSLKEQKKLNDLCFLKRLLQEIKTSWNKILMGTKEH

SEQ ID NO: 146 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNSLNNEFNFFKRHISDANKEGMFLFRAARKLRQFLKMNSTGDFDLHLLKVSEGTTILLNSTGQVKGRKPAALGEAQPTKSLEENKSLKEQKKLNDLSFLKRLLQEIKTCWNKILMGTKEH

SEQ ID NO: 147 - protein, 616 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCHNKILMGTKEH

SEQ ID NO: 148 - protein, 215 aa, Artificial Sequence
  Synthetic Construct
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 149 - protein, 447 aa, Artificial Sequence
  Synthetic Construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 150 - protein, 613 aa, Artificial Sequence
  Synthetic Construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 151 - protein, 613 aa, Artificial Sequence
  Synthetic Construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESALMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 152 - protein, 613 aa, Artificial Sequence
  Synthetic Construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESKLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 153 - protein, 613 aa, Artificial Sequence
  Synthetic Construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLEVSEGTTILLNCTGQVKGRKPAALGEAQPTKSLEENKSLKEQKKLNDLCFLKRLLQEIKTCWNKILMGTKEH

SEQ ID NO: 154 - protein, 613 aa, Artificial Sequence
  Synthetic Construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEKTTILLNCTGQVKGRKPAALGEAQPTKSLEENKSLKEQKKLNDLCFLKRLLQEIKTCWNKILMGTKEH

SEQ ID NO: 155 - protein, 613 aa, Artificial Sequence
  Synthetic Construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEETTILLNCTGQVKGRKPAALGEAQPTKSLEENKSLKEQKKLNDLCFLKRLLQEIKTCWNKILMGTKEH

SEQ ID NO: 156 - protein, 613 aa, Artificial Sequence
  Synthetic Construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDSDIEGKDGKQYESVLMVSIDQLLDSMKEIGSNCLNNEFNFFKRHISDANKEGMFLFRAARKLRQFLKMNSTGDFDLHLLKVSEGTTILLNSTGQVKGRKPAALGEAQPTKSLEENKSLKEQKKLNDLCFLKRLLQEIKTSWNKILMGTKEH

SEQ ID NO: 157 - protein, 594 aa, Artificial Sequence
  Synthetic Construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 158 - protein, 218 aa, Artificial Sequence
  Synthetic Construct
DIVLTQSPASLAVSLGQRAAISCRASKGVSTSGYSYLHWYQQKPGQSPKLLIYLASYLESGVPARFSGSGSGTDFTLNIHPVEEEDAATYYCQHSRDLPLTFGTGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 159 - protein, 449 aa, Artificial Sequence
  Synthetic Construct
QVQLQQPGAELVKPGTSVKLSCKASGYTFTNYYMYWVKQRPGQGLEWIGGINPSNGGTNFNEKFKNKATLTVDSSSSTTYMQLSSLTSEDSAVYYCTRRDYRFDMGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 160 - protein, 615 aa, Artificial Sequence
  Synthetic Construct
QVQLQQPGAELVKPGTSVKLSCKASGYTFTNYYMYWVKQRPGQGLEWIGGINPSNGGTNFNEKFKNKATLTVDSSSSTTYMQLSSLTSEDSAVYYCTRRDYRFDMGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 161 - protein, 615 aa, Artificial Sequence
  Synthetic Construct
QVQLQQPGAELVKPGTSVKLSCKASGYTFTNYYMYWVKQRPGQGLEWIGGINPSNGGTNFNEKFKNKATLTVDSSSSTTYMQLSSLTSEDSAVYYCTRRDYRFDMGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGDCDIEGKDGKQYESVLMVSIDQLLDSMKEIGSNCLNNEFNFFKRHICDANKEGMFLFRAARKLRQFLKMNSTGDFDLHLLEVSEGTTILLNCTGQVKGRKPAALGEAQPTKSLEENKSLKEQKKLNDLCFLKRLLQEIKTCWNKILMGTKEH

SEQ ID NO: 162 - protein, 615 aa, Artificial Sequence
  Synthetic Construct
QVQLQQPGAELVKPGTSVKLSCKASGYTFTNYYMYWVKQRPGQGLEWIGGINPSNGGTNFNEKFKNKATLTVDSSSSTTYMQLSSLTSEDSAVYYCTRRDYRFDMGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGDCDIEGKDGKQYESVLMVSIDQLLDSMKEIGSNCLNNEFNFFKRHICDANKEGMFLFRAARKLRQFLKMNSTGDFDLHLLKVSEETTILLNCTGQVKGRKPAALGEAQPTKSLEENKSLKEQKKLNDLCFLKRLLQEIKTCWNKILMGTKEH

SEQ ID NO: 163 - protein, 615 aa, Artificial Sequence
  Synthetic Construct
QVQLQQPGAELVKPGTSVKLSCKASGYTFTNYYMYWVKQRPGQGLEWIGGINPSNGGTNFNEKFKNKATLTVDSSSSTTYMQLSSLTSEDSAVYYCTRRDYRFDMGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGDCDIEGKDGKQYESVLMVSIDQLLDSMKEIGSNCLNNEFNFFKRHICDANKEGMFLFRAARKLRQFLKMNSTGDFDLHLLEVSEKTTILLNCTGQVKGRKPAALGEAQPTKSLEENKSLKEQKKLNDLCFLKRLLQEIKTCWNKILMGTKEH

SEQ ID NO: 164 - protein, 615 aa, Artificial Sequence
  Synthetic Construct
QVQLQQPGAELVKPGTSVKLSCKASGYTFTNYYMYWVKQRPGQGLEWIGGINPSNGGTNFNEKFKNKATLTVDSSSSTTYMQLSSLTSEDSAVYYCTRRDYRFDMGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGDCDIEGKDGKQYESVLMVSIDQLLDSMKEIGSNCLNNEFNFFKRHICDANKEGMFLFRAARKLRQFLKMNSTGDFDLHLLEVSEETTILLNCTGQVKGRKPAALGEAQPTKSLEENKSLKEQKKLNDLCFLKRLLQEIKTCWNKILMGTKEH

SEQ ID NO: 165 - protein, 218 aa, Artificial Sequence; identical to SEQ ID NO: 25
  Synthetic Construct

SEQ ID NO: 166 - protein, 450 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK